(19) United States
(12) Patent Application Publication
(10) Pub. No.: US 2023/0159992 A1
(43) Pub. Date: May 25, 2023

(54) HIGH THROUGHPUT SINGLE-CHAMBER PROGRAMMABLE NUCLEASE ASSAY

(71) Applicant: Mammoth Biosciences, Inc., Brisbane, CA (US)

(72) Inventors: Timothy James PATNO, Barrington, IL (US); Jesus CHING, Saratoga, CA (US); James Paul BROUGHTON, South San Francisco, CA (US); Xin MIAO, Mountain View, CA (US); Janice Sha CHEN, San Francisco, CA (US); Clare Louise FASCHING, Redwood City, CA (US); Alexander HIRSCHI, Redwood City, CA (US); Raymond Weibang MEI, Castro Valley, CA (US); Wesley Wendell ADDISON, II, Oakland, CA (US); Nazmiye Emel ALPAY, Mountain View, CA (US); Farzaneh TONDNEVIS, San Jose, CA (US); Sara ANSALONI, Mountain View, CA (US); Deepika VERMA, Belmont, CA (US)

(21) Appl. No.: 18/155,675

(22) Filed: Jan. 17, 2023

Related U.S. Application Data
(63) Continuation of application No. PCT/US2021/042437, filed on Jul. 20, 2021.
(60) Provisional application No. 63/213,126, filed on Jun. 21, 2021.
(60) Provisional application No. 63/173,282, filed on Apr. 9, 2021.
(60) Provisional application No. 63/146,505, filed on Feb. 5, 2021.
(60) Provisional application No. 63/138,304, filed on Jan. 15, 2021.
(60) Provisional application No. 63/136,018, filed on Jan. 11, 2021.
(60) Provisional application No. 63/106,841, filed on Oct. 28, 2020.
(60) Provisional application No. 63/106,307, filed on Oct. 27, 2020.
(60) Provisional application No. 63/054,202, filed on Jul. 20, 2020.

(51) Int. Cl. C12Q 1/6823; C12Q 1/6806; C12Q 1/6883; C12Q 1/70

(52) U.S. Cl. CPC C12Q 1/6823; C12Q 1/6806; C12Q 1/6883; C12Q 1/701; C12Q 2600/156

(57) ABSTRACT

Disclosed herein are systems and methods for providing a high-throughput DETECTR assay in a single chamber. The single chamber may be one well of a microplate, and multiple assays may be conducted in a staggered fashion in separate chambers. The methods described herein implement a process including lysing a sample, isolating nucleic acid molecules, eluting the nucleic acid molecules, amplifying the nucleic acid molecules, and detecting a presence of a target nucleic acid.

### CROSS-REFERENCE

This application claims the benefit of U.S. Provisional Patent Application No. 63/054,202, filed on Jul. 20, 2020; U.S. Provisional Patent Application No. 63/106,307, filed on Oct. 27, 2020; U.S. Provisional Patent Application No. 63/106,841, filed on Oct. 28, 2020; U.S. Provisional Patent Application No. 63/136,018, filed on Jan. 11, 2021; U.S. Provisional Patent Application No. 63/138,304, filed on Jan. 15, 2021; U.S. Provisional Patent Application No. 63/146,505, filed on Feb. 5, 2021;U.S. Provisional Patent Application No. 63/173,282, filed on Apr. 9, 2021; and U.S. Provisional Patent Application No. 63/213,126, filed on Jun. 21, 2021, all of which are incorporated herein by reference in their entirety.

### STATEMENT REGARDING FEDERALLY SPONSORED RESEARCH OR DEVELOPMENT

This invention was made with government support under Grant No. 75N92020C00011 awarded by the National Institute of Biomedical Imaging and Bioengineering, National Institutes of Health, and Department of Health and Human Services. The government of the United States of America has certain rights in the invention.

### BACKGROUND

Various communicable diseases can easily spread from an individual or environment to an individual. These diseases may be caused by viruses that include but are not limited to respiratory viruses such as SARS-CoV-2, influenza, and the like. Other diseases can be caused by sexually transmitted viruses such as herpes simplex virus and the like. The detection of the ailments, especially at the early stages of infection, may provide guidance on treatment or intervention to reduce the progression or transmission of the ailment. During large-scale virus outbreaks, large-scale testing for viruses may need to be conducted rapidly.

### SUMMARY

The device disclosed herein addresses a need to conduct over one million tests per day. Proposed herein is a solution for semi-automated, high throughput testing using a combination of CRISPR reagents, robotics, and trained users. The solution described executes a CRISPR detection enzymatic assay all in a single chamber. The system disclosed herein implements the steps of providing a nucleic acid, lysis, elution, amplification, and detection in a single vessel.

In an aspect, a high-throughput single-chamber process for detecting a presence of a target nucleic acid is disclosed. The high-throughput single-chamber process comprises (a) providing a single chamber. The process also comprises (b) binding a plurality of nucleic acids with a microparticle within the single chamber to form a microparticle complex. The process also comprises (c) isolating the microparticle complex within the single chamber. The process also comprises (d) amplifying the plurality of nucleic acids within the single chamber to form an amplified product. The process also comprises (e) contacting the amplified product with a guide nucleic acid complexed to a programmable nuclease within the single chamber such that, when the amplified product comprises a target nucleic acid, the guide nucleic acid contacts the target nucleic acid to form an activated programmable nuclease, thereby cleaving a reporter molecule by the activated programmable nuclease to produce a cleaved reporter molecule. The process also comprises (f) assaying for a detectable signal emitted within the single chamber by the cleaved reporter molecule, thereby detecting a presence or absence of the target nucleic acid.

In some embodiments, the process further comprises lysing a sample to release the plurality of nucleic acids within the single-chamber, thereby enabling the plurality of nucleic acids to bind with the microparticle.

In some embodiments, the pathogenic viruses comprise SARS-CoV-2 variants.

In some embodiments, the variants are B.1.1.7, B.1.351, B.1.617,.2, B.1.427, B.1.429, P.1., or SARS-CoV-2 wild-type.

In some embodiments, the single chamber is a well of a microplate or a tube.

In some embodiments, the target nucleic acid comprises a gene.

In some embodiments, the gene is a SARS-CoV-2 N-gene.

In some embodiments, the plurality of nucleic acids is collected from nasopharyngeal swabs or from nasal, mid-turbinate, or oropharyngeal sources.

In some embodiments, the pathogenic viruses comprise SARS-CoV-2 mutations.

In some embodiments, the mutations are L452R, E484K, or N501Y.

In some embodiments, (c) comprises adding a wash solution to the single chamber.

In some embodiments, (d) further comprises adding mineral oil to prevent evaporation.

In some embodiments, (a) is performed in a laboratory, hospital, physician office, clinic, a remote site, or in a home.

In some embodiments, the process further comprises eluting the plurality of nucleic acids from the microparticle complex.

In some embodiments, the eluting is performed using an elution buffer.

In some embodiments, the process further comprises removing waste liquid from the single chamber prior to eluting the nucleic acid molecules from the microparticle.

In some embodiments, eluting the nucleic acid molecules is performed using pipette mixing or using a plate mixer.

In some embodiments, the guide nucleic acid binds with a segment of the target nucleic acid.

In some embodiments, the microparticle remains in the single chamber during steps (d)-(f).

In some embodiments, (a) is performed at 37+/−2° C., (d) is performed at 57+/−2° C. or 62+/−2° C., and (e) is performed at 37+/−2° C.

In some embodiments, the microparticles comprise silica-coated magnetic beads, carbohydrate copolymers, hydroxy functionalized copolymers, carboxylic acid functionalized copolymers, or a combination thereof.

In some embodiments, the target nucleic acid is an antigen or fragment thereof.

In some embodiments, the antigen is a viral antigen, a bacterial antigen, or a cancer antigen.

In some embodiments, the process is performed in the single chamber as it is transported to between one and six stations.

In some embodiments, (a)-(b) are performed at a first station, (c) is performed at a second station, eluting the plurality of nucleic acids from the microparticle complex is performed at a third station, (d) is performed at a fourth station, and (e)-(f) is performed at a fifth station.

In some embodiments, a robot moves the single chamber between stations.

In some embodiments, (a)-(f) are performed at one station.

In some embodiments, (a) is performed between an ambient temperature and 95+2/−5° C., (d) is performed at a temperature of 57+/−2° C. or 62+/−2° C., and (e)-(f) is performed at a temperature from 37+/−2° C.

In some embodiments, isolating the microparticle complex comprises capturing the microparticle with a magnet.

In some embodiments, capturing comprises bringing the magnet in magnetic contact with the chamber and changing a temperature of the chamber to about 57° C. or about 62° C. prior to eluting the nucleic acid molecules from the microparticle.

In some embodiments, capturing comprises bringing the chamber in magnetic contact with the magnet and changing the temperature to an ambient temperature.

In some embodiments, the reporter molecule comprises a detection moiety for generating the signal.

In some embodiments, the detection moiety comprises a fluorophore.

In some embodiments, the reporter molecule comprises a protein for generating the signal.

In some embodiments, amplifying the nucleic acid molecules comprises performing RT-LAMP.

In some embodiments, the signal comprises a fluorescence signal.

In some embodiments, assaying for the detectable signal comprises obtaining a fluorescence value periodically via a detector.

In some embodiments, obtaining the fluorescence value periodically comprises obtaining a fluorescence value every 20 seconds to produce a plurality of obtained fluorescence values.

In some embodiments, detecting the presence of the target nucleic acid comprises plotting slope values from the plurality of obtained fluorescence values.

In some embodiments, the process further comprises comparing the slope values to slope values of a positive control and to slope values of a negative control.

In some embodiments, assaying for the detectable signal comprises detecting the fluorescence signal and obtaining a fluorescence value after a predetermined period of time via a detector.

In some embodiments, (a)-(f) are completed in under about 40 minutes.

In some embodiments, (a) is completed in under about one minute, wherein (b) is completed between about four and about ten minutes, wherein (c) is completed in under about one minute, wherein eluting the plurality of nucleic acids from the microparticle complex is completed in between about four and about ten minutes, wherein (d) is completed in about 20-30 minutes, and wherein (e)-(f) is completed in about 5-10 minutes.

In some embodiments, the cleaved reporter molecule is RNA or DNA.

In some embodiments, the single chamber is a first well in a microplate.

In some embodiments, the process further comprises, in a second well of the microplate, performing steps (a)-(f) on an additional sample.

In some embodiments, performing steps (a)-(f) on the additional sample in the second well occurs after a period of time from initiating (a) in the first well.

In some embodiments, the period is less than or equal to half of a length of time for completion of steps (a)-(f) in the first well.

In some embodiments, the period is about ten minutes.

In some embodiments, the programmable nuclease comprises a CRISPR/Cas enzyme.

In some embodiments, the guide nucleic acid is supplied as a complex with the programmable nuclease.

In some embodiments, the complex of the guide nucleic acid and the programmable nuclease is a ribonucleoprotein complex.

In some embodiments, the guide nucleic acid is supplied in situ with the programmable nuclease.

In some embodiments, the guide nucleic acid comprises a guide RNA.

In some embodiments, the signal is associated with a physical, chemical, electrochemical change or reaction, or combinations thereof.

In some embodiments, the signal comprises an optical signal.

In some embodiments, the signal comprises a potentiometric or amperometric signal.

In some embodiments, the signal comprises a piezoelectric signal.

In some embodiments, the signal is associated with a change in an index of refraction of a solid or gel volume in which the programmable nuclease probe is disposed.

In some embodiments, the process further comprises providing the programmable nuclease, the reporter molecule, the guide nucleic acid, or a combination thereof, through a detection reagent.

In some embodiments, the process further comprises using the signal to detect pathogenic viruses, pathogenic bacteria, pathogenic worms, pathogenic fungi, or cancer biomarkers.

In some embodiments, the pathogenic viruses are respiratory viruses, adenoviruses, parainfluenza viruses, severe acute respiratory syndrome (SARS), coronavirus, SARS-CoV, SARS-CoV-2, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, papillomavirus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, or a combination thereof.

In some embodiments, the pathogenic bacteria are selected from the group consisting of Mycobacterium tuberculosis, Klebsiella pneumoniae, Acinetobacter baumannii, Burkholderia cepacia, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Bordetella parapertussis, Bordetella pertussis, Chlamydia pneumoniae, Mycoplasma pneumoniae, Mycobacterium leprae, and Brucella abortus.

In some embodiments, the pathogenic worms are selected from the group consisting of roundworms, heartworms, phytophagous nematodes, flukes, Acanthocephala, and tapeworms.

In some embodiments, the pathogenic fungi are selected from the group consisting of Cryptococcus neoformans, Histoplasma capsulatum, Coccidioides immitis, Blastomyces dermatitidis, Chlamydia trachomatis, and Candida albicans.

In some embodiments, the cancer biomarkers are selected from the group consisting of lung cancer biomarkers and prostate cancer biomarkers.

In some embodiments, the programmable nuclease is a programmable Cas12 nuclease, a programmable Cas13 nuclease, a programmable Cas14 nuclease, a programmable thermostable Cas nuclease, or a CasΦ nuclease.

In some embodiments, the target nucleic acid is DNA.

In some embodiments, the target nucleic acid is RNA.

In some embodiments, steps (a)-(g) are performed in a high-throughput manner.

In some embodiments, the high-throughput manner comprises detecting about 400 target nucleic acids in 1.75 hours or detecting about 192 target nucleic acids in 110 minutes.

In an aspect, a high-throughput single-chamber system for detecting a presence of a target nucleic acid is disclosed. The system comprises (a) a lysis agent for lysing a sample, thereby releasing nucleic acid molecules. The system also comprises (b) one or more microparticles for binding with the nucleic acid molecules to form one or more microparticle complexes therewith. The system also comprises (c) an isolator to isolate the one or more microparticle complexes in the single chamber. The system also comprises (d) an elutor to elute the nucleic acid molecules from the one or more microparticle complexes. The system also comprises (e) an amplification agent for amplifying the nucleic acid molecules via contact thereto, resulting in amplified nucleic acid molecules. The system also comprises (f) a programmable nuclease. The system also comprises (g) a reporter molecule, and a guide nucleic acid that is capable of binding at least a segment of a target nucleic acid when present in the amplified nucleic acid molecules. The guide nucleic acid is coupled to the programmable nuclease. Binding of the guide nucleic acid to the target nucleic acid activates the programmable nuclease, thereby cleaving the reporter molecule via the programmable nuclease to produce a cleaved reporter molecule. A signal is configured to be emitted using the cleaved reporter molecule, wherein the signal corresponds to a presence of the target nucleic acid. The system also comprises (h) a single chamber configured to i) lyse the sample via the lysis agent, ii) form the one or more microparticle complexes, iii) isolate the one or more microparticle complexes, iv) elute the nucleic acid molecules from the one or more microparticle complexes, v) amplify the nucleic acid molecules while the one or microparticles remain in the single chamber, and vi) detect the signal while the one or more microparticles remain in the single chamber.

In some embodiments, the single chamber is a well of a microplate.

In some embodiments, the microplate has at least 384 wells.

In some embodiments, the microplate has at least 96 wells.

In some embodiments, the single chamber has from about a 250 to about a 300 μL fill volume.

In some embodiments, the method further comprises a multi-tip pipette head that delivers the elutor or the amplification agent to the single chamber.

In some embodiments, the system further comprises a heating element.

In some embodiments, the heating element is capable of shifting between a first temperature and a second temperature in under two minutes.

In some embodiments, the reporter molecule comprises a detection moiety configured to generate the signal.

In some embodiments, the detection moiety comprises a fluorophore.

In some embodiments, the system further comprises a tube for holding a positive control and a tube for holding a negative control.

In some embodiments, the system further comprises a detector for detecting the emitted signal.

In some embodiments, the detector comprises a fluorimeter.

In some embodiments, the system further comprises a computing device to identify the presence or an absence of the target nucleic acid via the signal.

In some embodiments, the computing device identifies a presence or absence of the target nucleic acid by comparing a signal slope against a signal slope from a positive control and a signal slope from a negative control.

In some embodiments, the computing device is in operative communication with a detector for detecting the emitted signal.

In some embodiments, the lysis agent comprises a physical, mechanical, thermal, enzymatic agent, or a combination thereof.

In some embodiments, the lysis agent comprises a lysis buffer solution.

In some embodiments, the lysis buffer solution comprises a chaotropic agent, detergent, salt, or a combination thereof.

In some embodiments, the lysis buffer solution comprises 4 M guanidinium isothiocyanate, 25 mM sodium citrate.2H20, 0.5% (w/v) sodium lauryl sarcosinate, and 0.1 M β-mercaptoethanol.

In some embodiments, the microparticles comprise silica-coated beads or magnetized beads.

In some embodiments, the elutor comprises a buffer solution.

In some embodiments, the elutor comprises a chaotropic salt or a detergent.

In some embodiments, the elutor comprises a detergent, wherein the detergent comprises Tween 20, Triton X-100, Deoxycholate, Sodium laurel sulfate, 3-[(3-cholamidopropyl)dimethylammonio]-1-propanesulfonate (CHAPS), or combinations thereof.

In some embodiments, the amplification agent comprises a DNA sequence, dNTPs, a forward primer, a reverse primer, a polymerase, or combinations thereof.

In some embodiments, the amplification agent comprises a reagent for RT-LAMP amplification.

In some embodiments, the amplification agent includes an RNA, a plurality of primers (e.g., four, five, or six primers), a primer having a T7 promoter, dNTPs, NTPs, a polymerase enzyme, a reverse transcriptase enzyme, a RNA polymerase, or combinations thereof.

In some embodiments, the RNA polymerase is T7 RNA polymerase.

In some embodiments, the programmable nuclease comprises a CRISPR/Cas enzyme.

In some embodiments, the CRISPR/Cas enzyme is a Cas12, a Cas13, a Cas14, a programmable thermostable Cas nuclease, or a CasΦ effector protein.

In some embodiments, the guide nucleic acid is sgRNA.

In some embodiments, the reporter molecule is ssDNA-FQ reporter and the detection moiety is a fluorophore or a quencher.

In some embodiments, the signal comprises a calorimetric, potentiometric, amperometric, fluorescent, or colorimetric signal.

In some embodiments, the signal comprises a fluorometric signal generated using a fluorophore.

In some embodiments, the signal is generated using a nucleic acid conjugated to an affinity molecule and the affinity molecule conjugated to a fluorophore.

In some embodiments, the system comprises a concentration of 100 nM CasΦ polypeptide or variant thereof, 125 nM sgRNA, and 50 nM ssDNA-FQ reporter in a total reaction volume of 20 μL.

In some embodiments, the reporter molecule comprises a protein configured to generate the signal.

In some embodiments, the signal enables the detection of pathogenic viruses, pathogenic bacteria, pathogenic worms, pathogenic fungi, or cancer biomarkers.

In some embodiments, the pathogenic viruses are respiratory viruses, adenoviruses, parainfluenza viruses, severe acute respiratory syndrome (SARS), coronavirus, SARS-CoV, SARS-CoV-2, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, papillomavirus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, or a combination thereof.

In some embodiments, the pathogenic bacteria are selected from the group consisting of Mycobacterium tuberculosis, Klebsiella pneumoniae, Acinetobacter baumannii, Burkholderia cepacia, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Bordetella parapertussis, Bordetella pertussis, Chlamydia pneumoniae, Mycoplasma pneumoniae, Mycobacterium leprae, and Brucella abortus.

In some embodiments, the pathogenic worms are selected from the group consisting of roundworms, heartworms, phytophagous nematodes, flukes, Acanthocephala, and tapeworms.

In some embodiments, the pathogenic fungi are selected from the group consisting of Cryptococcus neoformans, Histoplasma capsulatum, Coccidioides immitis, Blastomyces dermatitidis, Chlamydia trachomatis, and Candida albicans.

In some embodiments, the cancer biomarkers are selected from the group consisting of lung cancer biomarkers and prostate cancer biomarkers.

In some embodiments, the pathogenic viruses comprise SARS-CoV-2 variants.

In some embodiments, the variants are B.1.1.7, B.1.351, B.1.617,.2, B.1.427, B.1.429, P.1., or SARS-CoV-2 wild-type.

In some embodiments, the pathogenic viruses comprise SARS-CoV-2 mutations.

In some embodiments, the mutations are L452R, E484K, or N501Y.

In an aspect, a high-throughput single-chamber process for detecting a presence of a target nucleic acid is disclosed. The high-throughput single-chamber process comprises (a) providing a single chamber. The process also comprises (b) binding the plurality of nucleic acids with a microparticle within the single chamber to form a microparticle complex. The process also comprises (c) isolating the microparticle complex within the single chamber. The process also comprises (d) amplifying the plurality of nucleic acids within the single chamber to form an amplified product while the microparticle remains within the single chamber. The process also comprises (e) assaying the amplified product for a detectable signal emitted within the single chamber, thereby detecting a presence or absence of the target nucleic acid, while the microparticle remains within the single chamber.

In some embodiments, the process further comprises, prior to (b), lysing a sample to release the plurality of nucleic acids within the single chamber, thereby enabling the plurality of nucleic acids to bind with the microparticle.

In some embodiments, the process further comprises, prior to (d), eluting the plurality of nucleic acids from the microparticle complex.

In some embodiments, the process further comprises, prior to (e), contacting the amplified product with a guide nucleic acid complexed to a programmable nuclease within the single chamber such that, when the amplified product comprises a target nucleic acid, the guide nucleic acid contacts the target nucleic acid to form an activated programmable nuclease, thereby cleaving a reporter molecule by the activated programmable nuclease to produce a cleaved reporter molecule.

In some embodiments, the reporter molecule comprises a detection moiety for generating the signal.

In some embodiments, the detection moiety comprises a fluorophore.

In some embodiments, (b) is performed at 37+/−2° C., (d) is performed at 57+/−2° C., and (e) is performed at 37+/−2° C.

In some embodiments, (b) is performed at 95+/−2° C., (d) is performed at 62+/−2° C., and (e) is performed at 37+/−2° C.

In some embodiments, (b) is performed at between 20° C. and 95° C., (d) is performed at between 52° C. and 67° C., and (e) is performed at 37+/−2° C.

In some embodiments, the programmable nuclease is a programmable Cas12 nuclease, a programmable Cas13 nuclease, a programmable Cas14 nuclease, a programmable thermostable Cas nuclease, or a CasΦ nuclease.

In some embodiments, isolating the microparticle complex comprises capturing the microparticle with a magnet.

In some embodiments, amplifying the nucleic acid molecules comprises performing RT-LAMP.

In some embodiments, the signal comprises a fluorescence signal.

In some embodiments, the single chamber is a first well in a microplate.

In some embodiments, the high-throughput manner comprises detecting about 400 target nucleic acids in 1.75 hrs or detecting about 192 target nucleic acids in 110 minutes.

In some embodiments, the process further comprises lysing a sample to release the plurality of nucleic acids within the single-chamber, thereby enabling the plurality of nucleic acids to bind with the microparticle.

In some embodiments, the process further comprises eluting the plurality of nucleic acids from the microparticle complex.

In some embodiments, the eluting is performed using an elution buffer.

In some embodiments, the process further comprises removing waste liquid from the single chamber prior to eluting the nucleic acid molecules from the microparticle.

In some embodiments, eluting the nucleic acid molecules is performed using pipette mixing or using a plate mixer.

In some embodiments, the guide nucleic acid binds with a segment of the target nucleic acid.

In some embodiments, the microparticle remains in the single chamber during steps (d)-(f).

In some embodiments, (a) is performed at 37+/−2° C., (d) is performed at 57+/−2° C. or 62+/−2° C., and (e) is performed at 37+/−2° C.

In some embodiments, the microparticles comprise silica-coated magnetic beads, carbohydrate copolymers, hydroxy functionalized copolymers, carboxylic acid functionalized copolymers, or a combination thereof.

In some embodiments, the target nucleic acid is an antigen or fragment thereof.

In some embodiments, the antigen is a viral antigen, a bacterial antigen, or a cancer antigen.

In some embodiments, the process is performed in the single chamber as it is transported to between one and six stations.

In some embodiments, a robot moves the single chamber between stations.

In some embodiments, (a) is performed between an ambient temperature and 95+2/−5° C., (d) is performed at a temperature of 57+/−2° C. or 62+/−2° C., and (e)-(f) is performed at a temperature from 37+/−2° C.

In some embodiments, isolating the microparticle complex comprises capturing the microparticle with a magnet.

In some embodiments, capturing comprises bringing the magnet in magnetic contact with the chamber and changing a temperature of the chamber to about 57° C. or about 62° C. prior to eluting the nucleic acid molecules from the microparticle.

In some embodiments, capturing comprises bringing the chamber in magnetic contact with the magnet and changing the temperature to an ambient temperature.

In some embodiments, the reporter molecule comprises a detection moiety for generating the signal.

In some embodiments, the detection moiety comprises a fluorophore.

In some embodiments, the reporter molecule comprises a protein for generating the signal.

In some embodiments, amplifying the nucleic acid molecules comprises performing RT-LAMP.

In some embodiments, the signal comprises a fluorescence signal.

In some embodiments, assaying for the detectable signal comprises obtaining a fluorescence value periodically via a detector.

In some embodiments, obtaining the fluorescence value periodically comprises obtaining a fluorescence value every 20 seconds to produce a plurality of obtained fluorescence values.

In some embodiments, detecting the presence of the target nucleic acid comprises plotting slope values from the plurality of obtained fluorescence values.

In some embodiments, the process further comprises comparing the slope values to slope values of a positive control and to slope values of a negative control.

In some embodiments, assaying for the detectable signal comprises detecting the fluorescence signal and obtaining a fluorescence value after a predetermined period of time via a detector.

In some embodiments, the cleaved reporter molecule is RNA or DNA.

In some embodiments, the single chamber is a first well in a microplate.

In some embodiments, the period is about ten minutes.

In some embodiments, the programmable nuclease comprises a CRISPR/Cas enzyme.

In some embodiments, the guide nucleic acid is supplied as a complex with the programmable nuclease.

In some embodiments, the complex of the guide nucleic acid and the programmable nuclease is a ribonucleoprotein complex.

In some embodiments, the guide nucleic acid is supplied in situ with the programmable nuclease.

In some embodiments, the guide nucleic acid comprises a guide RNA.

In some embodiments, the signal is associated with a physical, chemical, electrochemical change or reaction, or combinations thereof.

In some embodiments, the signal comprises an optical signal.

In some embodiments, the signal comprises a potentiometric or amperometric signal.

In some embodiments, the signal comprises a piezoelectric signal.

In some embodiments, the signal is associated with a change in an index of refraction of a solid or gel volume in which the programmable nuclease probe is disposed.

In some embodiments, the process further comprises providing the programmable nuclease, the reporter molecule, the guide nucleic acid, or a combination thereof, through a detection reagent.

In some embodiments, the process further comprises using the signal to detect pathogenic viruses, pathogenic bacteria, pathogenic worms, pathogenic fungi, or cancer biomarkers.

In some embodiments, the pathogenic viruses are respiratory viruses, adenoviruses, parainfluenza viruses, severe acute respiratory syndrome (SARS), coronavirus, SARS-CoV, SARS-CoV-2, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, papillomavirus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, or a combination thereof.

In some embodiments, the pathogenic bacteria are selected from the group consisting of Mycobacterium tuberculosis, Klebsiella pneumoniae, Acinetobacter baumannii, Burkholderia cepacia, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Bordetella parapertussis, Bordetella pertussis, Chlamydia pneumoniae, Mycoplasma pneumoniae, Mycobacterium leprae, and Brucella abortus.

In some embodiments, the pathogenic worms are selected from the group consisting of roundworms, heartworms, phytophagous nematodes, flukes, Acanthocephala, and tapeworms.

In some embodiments, the pathogenic fungi are selected from the group consisting of Cryptococcus neoformans, Histoplasma capsulatum, Coccidioides immitis, Blastomyces dermatitidis, Chlamydia trachomatis, and Candida albicans.

In some embodiments, the cancer biomarkers are selected from the group consisting of lung cancer biomarkers and prostate cancer biomarkers.

In some embodiments, the programmable nuclease is a programmable Cas12 nuclease, a programmable Cas13 nuclease, a programmable Cas14 nuclease, a programmable thermostable Cas nuclease, or a CasΦ nuclease.

In some embodiments, the target nucleic acid is DNA.

In some embodiments, the target nucleic acid is RNA.

In some embodiments, the high-throughput manner comprises detecting about 400 target nucleic acids in 1.75 hours or detecting about 192 target nucleic acids in 110 minutes.

In an aspect, a high-throughput single-chamber process for detecting a presence of a target nucleic acid is disclosed. The high-throughput single-chamber process comprises (a) providing a lysis agent and microparticles in a single chamber. The process also comprises (b) providing a sample in the single chamber and lysing the sample by contacting the lysis agent with the sample, thereby releasing nucleic acid molecules. The process also comprises (c) allowing the nucleic acid molecules to bind to the microparticles to produce complexes comprising the nucleic acid molecules and the microparticles. The process also comprises (d) isolating the complexes comprising the nucleic acid molecules and the microparticles in the single chamber. The process also comprises (e) eluting the nucleic acid molecules from the complexes. The process also comprises (f) amplifying the nucleic acid molecules to form an amplified product, wherein the amplifying is by contacting the nucleic acid molecules with an amplification agent. The process also comprises (g) contacting, in the single chamber, the amplified product with: a programmable nuclease, a reporter molecule, and a guide nucleic acid that is capable of binding with a target nucleic acid. In the presence of the target nucleic acid in the amplified product, the guide nucleic acid binds with the target nucleic acid, such that the programmable nuclease cleaves the reporter molecule to produce a cleaved reporter molecule, and a detectable signal is emitted by the cleaved reporter molecule, wherein the detectable signal is indicative of the presence or absence of the target nucleic acid.

In some embodiments, the process further comprises lysing a sample to release the plurality of nucleic acids within the single-chamber, thereby enabling the plurality of nucleic acids to bind with the microparticle.

In some embodiments, the process further comprises eluting the plurality of nucleic acids from the microparticle complex.

In some embodiments, the eluting is performed using an elution buffer.

In some embodiments, the process further comprises removing waste liquid from the single chamber prior to eluting the nucleic acid molecules from the microparticle.

In some embodiments, eluting the nucleic acid molecules is performed using pipette mixing or using a plate mixer.

In some embodiments, the guide nucleic acid binds with a segment of the target nucleic acid.

In some embodiments, the microparticle remains in the single chamber during steps (d)-(f).

In some embodiments, the microparticles comprise silica-coated magnetic beads, carbohydrate copolymers, hydroxy functionalized copolymers, carboxylic acid functionalized copolymers, or a combination thereof.

In some embodiments, the target nucleic acid is an antigen or fragment thereof.

In some embodiments, the antigen is a viral antigen, a bacterial antigen, or a cancer antigen.

In some embodiments, the process is performed in the single chamber as it is transported to between one and six stations.

In some embodiments, isolating the microparticle complex comprises capturing the microparticle with a magnet.

In some embodiments, capturing comprises bringing the magnet in magnetic contact with the chamber and changing a temperature of the chamber to about 57° C. or about 62° C. prior to eluting the nucleic acid molecules from the microparticle.

In some embodiments, capturing comprises bringing the chamber in magnetic contact with the magnet and changing the temperature to an ambient temperature.

In some embodiments, the reporter molecule comprises a detection moiety for generating the signal.

In some embodiments, the detection moiety comprises a fluorophore.

In some embodiments, the reporter molecule comprises a protein for generating the signal.

In some embodiments, amplifying the nucleic acid molecules comprises performing RT-LAMP.

In some embodiments, the signal comprises a fluorescence signal.

In some embodiments, assaying for the detectable signal comprises obtaining a fluorescence value periodically via a detector.

In some embodiments, obtaining the fluorescence value periodically comprises obtaining a fluorescence value every 20 seconds to produce a plurality of obtained fluorescence values.

In some embodiments, detecting the presence of the target nucleic acid comprises plotting slope values from the plurality of obtained fluorescence values.

In some embodiments, the process further comprises comparing the slope values to slope values of a positive control and to slope values of a negative control.

In some embodiments, assaying for the detectable signal comprises detecting the fluorescence signal and obtaining a fluorescence value after a predetermined period of time via a detector.

In some embodiments, the cleaved reporter molecule is RNA or DNA.

In some embodiments, the single chamber is a first well in a microplate.

In some embodiments, the process further comprises, in a second well of the microplate, performing steps (a)-(f) on an additional sample.

In some embodiments, the period is about ten minutes.

In some embodiments, the programmable nuclease comprises a CRISPR/Cas enzyme.

In some embodiments, the guide nucleic acid is supplied as a complex with the programmable nuclease.

In some embodiments, the complex of the guide nucleic acid and the programmable nuclease is a ribonucleoprotein complex.

In some embodiments, the guide nucleic acid is supplied in situ with the programmable nuclease.

In some embodiments, the guide nucleic acid comprises a guide RNA.

In some embodiments, the signal is associated with a physical, chemical, electrochemical change or reaction, or combinations thereof.

In some embodiments, the signal comprises an optical signal.

In some embodiments, the signal comprises a potentiometric or amperometric signal.

In some embodiments, the signal comprises a piezoelectric signal.

In some embodiments, the signal is associated with a change in an index of refraction of a solid or gel volume in which the programmable nuclease probe is disposed.

In some embodiments, the process further comprises providing the programmable nuclease, the reporter molecule, the guide nucleic acid, or a combination thereof, through a detection reagent.

In some embodiments, the process further comprises using the signal to detect pathogenic viruses, pathogenic bacteria, pathogenic worms, pathogenic fungi, or cancer biomarkers.

In some embodiments, the pathogenic viruses are respiratory viruses, adenoviruses, parainfluenza viruses, severe acute respiratory syndrome (SARS), coronavirus, SARS-CoV, SARS-CoV-2, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, papillomavirus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, or a combination thereof.

In some embodiments, the pathogenic bacteria are selected from the group consisting of Mycobacterium tuberculosis, Klebsiella pneumoniae, Acinetobacter baumannii, Burkholderia cepacia, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Bordetella parapertussis, Bordetella pertussis, Chlamydia pneumoniae, Mycoplasma pneumoniae, Mycobacterium leprae, and Brucella abortus.

In some embodiments, the pathogenic worms are selected from the group consisting of roundworms, heartworms, phytophagous nematodes, flukes, Acanthocephala, and tapeworms.

In some embodiments, the pathogenic fungi are selected from the group consisting of Cryptococcus neoformans, Histoplasma capsulatum, Coccidioides immitis, Blastomyces dermatitidis, Chlamydia trachomatis, and Candida albicans.

In some embodiments, the cancer biomarkers are selected from the group consisting of lung cancer biomarkers and prostate cancer biomarkers.

In some embodiments, the programmable nuclease is a programmable Cas12 nuclease, a programmable Cas13 nuclease, a programmable Cas14 nuclease, a programmable thermostable Cas nuclease, or a CasΦ nuclease.

In some embodiments, the target nucleic acid is DNA.

In some embodiments, the target nucleic acid is RNA.

In some embodiments, steps (a)-(g) are performed in a high-throughput manner.

In some embodiments, the high-throughput manner comprises detecting about 400 target nucleic acids in 1.75 hrs or detecting about 192 target nucleic acids in 110 minutes.

In some embodiments, the microparticle remains in the single chamber during steps (f)-(g).

In some embodiments, the programmable nuclease is a programmable Cas12 nuclease, a programmable Cas13 nuclease, a programmable Cas14 nuclease, a programmable thermostable Cas nuclease, or a CasΦ nuclease.

In some embodiments, isolating the microparticle complex comprises capturing the microparticle with a magnet.

In some embodiments, amplifying the nucleic acid molecules comprises performing RT-LAMP.

In some embodiments, the signal comprises a fluorescence signal.

In some embodiments, (a)-(g) are completed in under about 40 minutes.

In some embodiments, the single chamber is a first well in a microplate.

In some embodiments, the process further comprises, in a second well of the microplate, performing steps (a)-(f) on an additional sample.

In some embodiments, (f) and (g) occur simultaneously.

In some embodiments, steps (a)-(g) are performed in a high-throughput manner.

In some embodiments, the high-throughput manner comprises detecting about 400 target nucleic acids in 1.75 hours or detecting about 192 target nucleic acids in 110 minutes.

In an aspect, a high-throughput single-chamber process for detecting the presence of a first target nucleic acid and a second target nucleic acid in a sample is disclosed. The process comprises (a) providing a single chamber. The process also comprises (b) binding a plurality of nucleic acids with a microparticle within the single chamber to form a microparticle complex. The process also comprises (c) isolating the microparticle complex within the single chamber. The process also comprises (d) contacting, in the single chamber, the plurality of nucleic acid molecules with a first probe, wherein the first probe is configured for binding with the first target nucleic acid. The process also comprises (e) amplifying the plurality of nucleic acids within the single chamber to form an amplified product. A first detectable signal is emitted i) prior to amplifying the plurality of nucleic acids, ii) while amplifying the plurality of nucleic acids, iii) after forming the amplified product, or iv) a combination thereof, thereby detecting the presence of the first target nucleic acid. The process also comprises (e) contacting the amplified product with a second probe complexed to a programmable nuclease within the single chamber such that, when the amplified product comprises the second target nucleic acid, the second probe contacts the target nucleic acid to form an activated programmable nuclease, thereby cleaving a reporter molecule by the activated programmable nuclease to produce a cleaved reporter molecule. The process also comprises (f) assaying for a second detectable signal emitted within the single chamber by the cleaved reporter molecule, thereby detecting the presence of the second target nucleic acid. In some embodiments, i) the first target nucleic acid comprises RNAse P, ii) the second target nucleic acid comprises SARS-CoV-2 N gene, or iii) a combination thereof.

In some embodiments, the first probe comprises a dye configured to produce a colorimetric signal when the pH changes during amplification of the plurality of nucleic acids.

In some embodiments, the first probe comprises a label configured to produce a fluorescent signal at a first wavelength.

In some embodiments, the second probe comprises a guide nucleic acid.

In some embodiments, the one or both of the first signal and the second signal comprises a fluorescent signal.

In some embodiments, when both the first signal and the second signal comprises a fluorescent signal, the second signal comprises a wavelength different from the first signal.

In some embodiments, the microparticle remains in the single chamber during steps (d)-(g).

In some embodiments, the process further comprises, prior to (b), lysing a sample to release the plurality of nucleic acids within the single chamber, thereby enabling the plurality of nucleic acids to bind with the microparticle.

In some embodiments, the process further comprises, prior to (d), eluting the plurality of nucleic acids from the microparticle complex.

In some embodiments, the programmable nuclease is a programmable Cas12 nuclease, a programmable Cas13 nuclease, a programmable Cas14 nuclease, a programmable thermostable Cas nuclease, or a CasΦ nuclease.

In some embodiments, isolating the microparticle complex comprises capturing the microparticle with a magnet.

In some embodiments, amplifying the nucleic acid molecules comprises performing RT-LAMP.

In some embodiments, the signal comprises a fluorescence signal.

In some embodiments, (a)-(g) are completed in under about 40 minutes.

In some embodiments, the single chamber is a first well in a microplate.

In some embodiments, the process further comprises in a second well of the microplate, performing steps (a)-(f) on an additional sample.

In some embodiments, (f) and (g) occur simultaneously.

In some embodiments, steps (a)-(g) are performed in a high-throughput manner.

In some embodiments, the high-throughput manner comprises detecting about 400 target nucleic acids in 1.75 hours or detecting about 192 target nucleic acids in 110 minutes.

In some embodiments, the process further comprises lysing a sample to release the plurality of nucleic acids within the single-chamber, thereby enabling the plurality of nucleic acids to bind with the microparticle.

In some embodiments, the process further comprises eluting the plurality of nucleic acids from the microparticle complex.

In some embodiments, the eluting is performed using an elution buffer.

In some embodiments, the process further comprises removing waste liquid from the single chamber prior to eluting the nucleic acid molecules from the microparticle.

In some embodiments, eluting the nucleic acid molecules is performed using pipette mixing or using a plate mixer.

In some embodiments, the guide nucleic acid binds with a segment of the target nucleic acid.

In some embodiments, the microparticle remains in the single chamber during steps (d)-(f).

In some embodiments, the microparticles comprise silica-coated magnetic beads, carbohydrate copolymers, hydroxy functionalized copolymers, carboxylic acid functionalized copolymers, or a combination thereof.

In some embodiments, the target nucleic acid is an antigen or fragment thereof.

In some embodiments, the antigen is a viral antigen, a bacterial antigen, or a cancer antigen.

In some embodiments, the process is performed in the single chamber as it is transported to between one and six stations.

In some embodiments, isolating the microparticle complex comprises capturing the microparticle with a magnet.

In some embodiments, capturing comprises bringing the magnet in magnetic contact with the chamber and changing a temperature of the chamber to about 57° C. or about 62° C. prior to eluting the nucleic acid molecules from the microparticle.

In some embodiments, capturing comprises bringing the chamber in magnetic contact with the magnet and changing the temperature to an ambient temperature.

In some embodiments, the reporter molecule comprises a detection moiety for generating the signal.

In some embodiments, the detection moiety comprises a fluorophore.

In some embodiments, the reporter molecule comprises a protein for generating the signal.

In some embodiments, amplifying the nucleic acid molecules comprises performing RT-LAMP.

In some embodiments, the signal comprises a fluorescence signal.

In some embodiments, assaying for the detectable signal comprises obtaining a fluorescence value periodically via a detector.

In some embodiments, obtaining the fluorescence value periodically comprises obtaining a fluorescence value every 20 seconds to produce a plurality of obtained fluorescence values.

In some embodiments, detecting the presence of the target nucleic acid comprises plotting slope values from the plurality of obtained fluorescence values.

In some embodiments, the process further comprises comparing the slope values to slope values of a positive control and to slope values of a negative control.

In some embodiments, assaying for the detectable signal comprises detecting the fluorescence signal and obtaining a fluorescence value after a predetermined period of time via a detector.

In some embodiments, the cleaved reporter molecule is RNA or DNA.

In some embodiments, the single chamber is a first well in a microplate.

In some embodiments, the process further comprises, in a second well of the microplate, performing steps (a)-(f) on an additional sample.

In some embodiments, performing steps (a)-(f) on the additional sample in the second well occurs after a period of time from initiating (a) in the first well.

In some embodiments, the period is less than or equal to half of a length of time for completion of steps (a)-(f) in the first well.

In some embodiments, the period is about ten minutes.

In some embodiments, the programmable nuclease comprises a CRISPR/Cas enzyme.

In some embodiments, the guide nucleic acid is supplied as a complex with the programmable nuclease.

In some embodiments, the complex of the guide nucleic acid and the programmable nuclease is a ribonucleoprotein complex.

In some embodiments, the guide nucleic acid is supplied in situ with the programmable nuclease.

In some embodiments, the guide nucleic acid comprises a guide RNA.

In some embodiments, the signal is associated with a physical, chemical, electrochemical change or reaction, or combinations thereof.

In some embodiments, the signal comprises an optical signal.

In some embodiments, the signal comprises a potentiometric or amperometric signal.

In some embodiments, the signal comprises a piezoelectric signal.

In some embodiments, the signal is associated with a change in an index of refraction of a solid or gel volume in which the programmable nuclease probe is disposed.

In some embodiments, the process further comprises providing the programmable nuclease, the reporter molecule, the guide nucleic acid, or a combination thereof, through a detection reagent.

In some embodiments, the process further comprises using the signal to detect pathogenic viruses, pathogenic bacteria, pathogenic worms, pathogenic fungi, or cancer biomarkers.

In some embodiments, the pathogenic viruses are respiratory viruses, adenoviruses, parainfluenza viruses, severe acute respiratory syndrome (SARS), coronavirus, SARS-CoV, SARS-CoV-2, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, papillomavirus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, or a combination thereof.

In some embodiments, the pathogenic bacteria are selected from the group consisting of Mycobacterium tuberculosis, Klebsiella pneumoniae, Acinetobacter baumannii, Burkholderia cepacia, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Bordetella parapertussis, Bordetella pertussis, Chlamydia pneumoniae, Mycoplasma pneumoniae, Mycobacterium leprae, and Brucella abortus.

In some embodiments, the pathogenic worms are selected from the group consisting of roundworms, heartworms, phytophagous nematodes, flukes, Acanthocephala, and tapeworms.

In some embodiments, the pathogenic fungi are selected from the group consisting of Cryptococcus neoformans, Histoplasma capsulatum, Coccidioides immitis, Blastomyces dermatitidis, Chlamydia trachomatis, and Candida albicans.

In some embodiments, the cancer biomarkers are selected from the group consisting of lung cancer biomarkers and prostate cancer biomarkers.

In some embodiments, the programmable nuclease is a programmable Cas12 nuclease, a programmable Cas13 nuclease, a programmable Cas14 nuclease, a programmable thermostable Cas nuclease, or a CasΦ nuclease.

In some embodiments, the target nucleic acid is DNA.

In some embodiments, the target nucleic acid is RNA.

In some embodiments, steps (a)-(g) are performed in a high-throughput manner.

In some embodiments, the high-throughput manner comprises detecting about 400 target nucleic acids in 1.75 hours or detecting about 192 target nucleic acids in 110 minutes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIGS. 1A, 1B, 1C, 1D, and 1E each show a process flow chart for a high-throughput programmable nuclease-based assay.

FIG. 2 shows the process of FIGS. 1A, 1B, 1C, 1D, and 1E implemented on two chambers in a staggered fashion.

FIG. 3 illustrates a system for implementing a high-throughput programmable nuclease-based assay.

FIGS. 4A and 4B illustrate a programmable nuclease probe comprising a programmable nuclease and a guide nucleic acid complexed with the programmable nuclease before and after a complementary binding event, as described herein.

FIGS. 5A and 5B show a programmable nuclease probe before and after a complementary binding event and the generation of a signal indicating a presence of a target sequence or target nucleic acid, as described herein.

FIG. 6 illustrates an additional embodiment of a high-throughput single-chamber detection assay.

FIG. 7 illustrates a comparison of four different viral lysis buffer solutions.

FIG. 8 illustrates fluorescence responses to temperature for two different viral lysis buffer solutions VLB 3 and VLB 4.

FIG. 9 shows the efficacies of different lysis buffer solutions at promoting enhanced detection of RNase P and N virus titers.

FIG. 10 illustrates fluorescence signals detected from using varying amounts of DETECTR solution on 20 μL of samples that have been amplified using RT-LAMP, enabling testers to infer preferred ratios of RT-LAMP:DETECTR.

FIG. 11 additionally illustrate detection results from using varying amounts of DETECTR solution on 20 μL of RT-LAMP amplified samples at different ratios of RT-LAMP to DETECTR, minoring the results from FIG. 10.

FIG. 12A and 12B illustrate enhancements to efficacy of the DETECTR solution with integrated buffer systems.

FIG. 13 illustrates fluorescence signal generation for varying concentrations of virus in DETECTR reactions.

FIGS. 14A and 14B illustrate fluorescence signals of different concentrations of N-gene at small volumes (25 μL) and large volumes (50 μL) of RT-LAMP and corresponding 100 μL and 200 μL of DETECTR reactions for 8 replicates each.

FIGS. 15A and 15B illustrates aggregate data corresponding to FIGS. 14A and 14B.

FIGS. 16A and 16B illustrate fluorescence results from using RT-LAMP solutions (top) and DETECTR solutions (bottom) comprising 25 μL of virus sample and 25 μL of a master mix at varying sample concentrations.

FIGS. 16C and 16D illustrate fluorescence results from using a master mix comprising two sub-master mixes, with the top plot showing results for RT-LAMP and the bottom showing DETECTR results for varying sample concentrations.

FIGS. 17A and 17B illustrate fluorescence results from using a master mix separating salts and enzymes at 2× or 5× concentration for RT-LAMP and DETECTR reactions.

FIG. 18 illustrates results for determining a minimal wash condition for the RT-LAMP reaction. As shown from the plots, the minimal wash condition for 2000 copies was 1×W1+1×W2 under the conditions tested.

FIG. 19 illustrates sample preparation results for an RT-LAMP reaction using MagMAX magnetic beads for purification. The plots show that the MagMAX beads were able to prepare samples with at least 2000 copies for downstream amplification using both Twist and SeraCare AccuPlex SARS-CoV-2 positive controls.

FIG. 20A illustrates a set of plots showing samples treated with ChargeSwitch RNA purification kit were able to be purified.

FIG. 20B illustrates sample preparation using a method of nucleic acid purification. Detection using the purification method was effective using samples containing universal transport media (UTM) in the lysis buffer.

FIG. 21 illustrates results from different sequences of sample preparation. When the samples contained UTM, fluorescence was maximized by adding a binding buffer to the sample before adding beads.

FIG. 22 illustrates RNA capture with ChargeSwitch magnetic beads using saliva and nasal matrices using the nucleic acid purification method. RNA was captured from both the nasal matrix and the saliva matrix.

FIG. 23 illustrates effectiveness of using DETECTR in a 384-deep well plate format. For reactions in randomly-spaced wells, the test effectively detected strong fluorescence signals. For reactions in adjacent wells, minimal bleed-through fluorescence in the adjacent well was detected.

FIG. 24 illustrates DETECTR results from amplified samples prepared from SeraCare encapsulated nucleic acid molecules at different concentration of target. At concentrations of 100 copies or above, the test produced significant levels of raw fluorescence.

FIG. 25 illustrates results from the assay workflow starting with SeraCare encapsulated target nucleic acid molecules and using UTM+nasal and UTM+saliva matrices during sample preparation. The test produced significant fluorescence readings for both RT-LAMP and DETECTR reactions for the replicates tested.

FIG. 26 illustrates results from the assay workflow starting with SeraCare encapsulated target nucleic acid molecules and using VTM+nasal and VTM+saliva matrices during sample preparation. The test produced significant fluorescence readings for both RT-LAMP and DETECTR reactions for the replicates tested.

FIG. 27A illustrates RT-LAMP performance for reactions with high numbers of copies of N-gene. As is shown, a high number of copies of N-gene does not produce a significant effect on fluorescence.

FIG. 27B illustrates plots showing the effects on detection signals when large copy numbers of N-gene are used in DETECTR reactions.

FIG. 28 illustrates results of an RT-LAMP reaction using a KOAc+Tris buffer solution. The test produced strong fluorescence results at 200 copies per reaction for all conditions tested.

FIG. 29 illustrates results of a DETECTR reaction using a buffer solution including Tris pH8 following the reaction of FIG. 28. The test produced strong fluorescence results at 200 copies per reaction for all conditions tested.

FIG. 30 illustrates effects of freeze-thaw cycles on reagent stability in an RT-LAMP reaction. After six freeze-thaw cycles, testing obtained strong fluorescence results, indicating reagent stability.

FIG. 31 illustrates results when DETECTR master mix was added following the freeze-thaw cycles of FIG. 30. The plots show that all replicates were captured.

FIG. 32 illustrates that RT-LAMP amplification results when washing was reduced compared to the standard MagMAX protocol. All wash conditions were effective in producing strong results. The test obtained stronger results using 2× W1 than 1× W1+1× W2 under the conditions tested.

FIG. 33 illustrates that detection with DETECTR after the RT-LAMP reaction shown in FIG. 32 was effective when washing is reduced. All wash conditions were effective in producing strong results. The test obtains the strongest results using 1× W1+1× W2.

FIG. 34 illustrates stability testing results from using a magnetic bead kit in an RT-LAMP reaction. In a concentration of 200 copies, RT-LAMP detected 6/6 positives, indicating good results for both freshly prepared beads and older beads. The reaction remained stable even after the beads were stored at room temperature for up to six days.

FIG. 35 illustrates stability testing results from using the amplified sample of FIG. 34 in a DETECTR reaction. In a concentration of 200 copies, RT-LAMP detected 6/6 positives, indicating good results for both freshly prepared beads and older beads. The reaction remained stable even after the beads were stored at room temperature for up to six days.

FIG. 36 illustrates results showing successful use of a 5× acetate lysis binding buffer for ChargeSwitch sample preparation from nasal and saliva matrix samples prior to both RT-LAMP and DETECTR reactions.

FIG. 37 illustrates RT-LAMP reaction results of reduced washing during sample prep with the MagMAX magnetic bead kit and UTM samples with a nasal matrix.

FIG. 38 illustrates DETECTR reaction results of of FIG. 37 with reduced washing during sample prep with the MagMAX magnetic bead kit and UTM samples with a nasal matrix.

FIG. 39 illustrates RT-LAMP reaction results of a 2×W1+2×W2 wash during sample prep with the MagMAX magnetic bead kit and VTM samples with a nasal matrix.

FIG. 40 illustrates DETECTR reactions results of FIG. 39 with a 2×W1+2×W2 wash during sample prep with the MagMAX magnetic bead kit and VTM samples and a nasal matrix.

FIG. 41 illustrates RT-LAMP reaction results of different wash conditions during sample prep with the magnetic bead kit and no matrix. 2× W1 was the best wash condition with VTM.

FIG. 42 illustrates DETECTR reaction results of FIG. 41 with different wash conditions during sample with the magnetic bead kit and no matrix. 2× W1 was the best wash condition with VTM.

FIG. 43 illustrates results from testing performed with the MagMAX magnetic bead kit in UTM with no matrix used. The best results for both RT-LAMP and DETECTR were obtained using 2×W1+2×W2 and 2×W1. 62 C lysis reduced extraction efficiency under the conditions tested.

FIG. 44 illustrates results from testing performed with the magnetic bead kit in UTM with a nasal matrix used. The best results for both RT-LAMP and DETECTR were obtained using 2×W1+2×W2 and 2×W1. 62 C lysis reduced extraction efficiency under the conditions tested.

FIG. 45 illustrates results from testing performed with the magnetic bead kit in UTM with a saliva matrix used. The best results for both RT-LAMP and DETECTR were obtained using 2×W1+2×W2, as well as with 2×W1. 2 C lysis reduced extraction efficiency under the conditions tested.

FIG. 46 illustrates results from testing performed with the magnetic bead kit in VTM with a nasal matrix used. The best results for both RT-LAMP and DETECTR were obtained using 2×W1+2×W2 and 2×W1.

FIG. 47 illustrates results from testing performed with the magnetic bead kit in VTM with a nasal matrix used. The best results for both RT-LAMP and DETECTR were obtained using 2×W1+2×W2 and 2×W1.

FIG. 48 illustrates results from a reduced volume workflow for the magnetic bead kit showing strong RT-LAMP and DETECTR signals at 75 copies of N-gene target/reaction.

FIG. 49 shows RT-LAMP and DETECTR test results after reducing lysis buffer volume to sample volume and/or the amount of IPA in the lysis buffer. The best results were obtained at 50% IPA and 25% IPA.

FIG. 50 illustrates shipping stability for N-gene reagents. After incubation with dry ice overnight, the reagents still yielded strong fluorescence results.

FIG. 51 illustrates that reagents incubated on ice and at room temperature were stable and produced strong fluorescence values from testing.

FIG. 52 illustrates a results from a reduced volume magnetic bead kit workflow for RT-LAMP and DETECTR reactions.

FIG. 53 illustrates optimal wash steps for sample preparation prior to RT-LAMP and DETECTR reactions with the MagMAX magnetic bead kit. Positive samples were successfully captured with 1× W1 at 75 copies per reaction.

FIG. 54 illustrates DETECTR reaction results for samples prepared under different % IPA titrations. Titrations yielding the strongest results were 110:100 sample:lysis volumes at 50% IPA and 60% IPA.

FIG. 55 illustrates that reducing bead concentration in DETECTR reactions may improve signal to noise and produce better fluorescence results when beads are retained in the chamber during the RT-LAMP and DETECTR reactions.

FIG. 56 illustrates RT-LAMP and DETECTR detection results after reducing bead concentration during sample preparation. In a nasal matrix, 5/6 replicates were detected, while in a saliva matrix, 6/6 replicates were detected with the beads retained in the chamber during the RT-LAMP and DETECTR reactions.

FIG. 57 illustrates RT-LAMP and DETECTR results from RT-LAMP temperature guardbanding. Replicates were amplified for 200 copies of twist at temperatures of 55° C.-59° C.

FIG. 58 illustrates RT-LAMP and DETECTR results from DETECTR temperature guardbanding. Replicates were detected for 200 copies of twist at temperatures of 25° C.-41° C.

FIG. 59 illustrates the effect of evaporation on RT-LAMP and DETECTR reactions. The plots illustrate that strong fluorescence results were found, indicating little or no evaporation-related issues occurred.

FIG. 60 illustrates a reproducibility study for an automated high-throughput assay using the workflow of FIG. 1D.

FIGS. 61 and 62 illustrate performance of an automated high-throughput assay using the workflow of FIG. 1D using different sample media and different target concentrations.

FIG. 63 illustrates fluorescence DETECTR data from a automated high-throughput assay using the workflow of FIG. 1D using nasal and saliva samples and different target concentrations.

FIG. 64 shows an exemplary microplate configuration for multiplexed target detection.

FIG. 65 shows an exemplary workflow including an RNase P internal control for RT-LAMP and experimental results showing detection of RNase P and N gene in a single well.

### DETAILED DESCRIPTION

The capability to quickly and accurately detect the presence of a target nucleic acid can provide valuable information associated with the presence of the target nucleic acid. For example, the capability to quickly and accurately detect the presence or absence of a nucleic acid in a sample can provide valuable information and leads to actions to reduce the progression or transmission of the disease or ailment. The 2020 COVID-19 pandemic is an example of how large-scale (e.g., population-wide), rapid detection of a nucleic acid (e.g., a SARS-CoV-2) can be essential for screening and testing to prevent disease spread. Detection of a target nucleic acid molecule encoding a specific sequence using a programmable nuclease provides a method for efficiently and accurately detecting the presence of the nucleic acid molecule of interest. There exists a need for highly efficient, rapid, and accurate reactions for detecting whether a target nucleic acid is present in a sample. The reaction can be sometimes referred to as a DETECTR reaction or a programmable nuclease-based test wherein detectable signal arising from cleavage of a reporter (also referred to herein as a detector nucleic acid) by the programmable nuclease is detected.

The present disclosure provides a method for a single-chamber, rapid, high-throughput programmable nuclease-based test which may quickly assess whether a target nucleic acid is present in a sample by using a programmable nuclease that can detect the presence of a nucleic acid of interest (e.g., a deoxyribonucleic acid or a deoxyribonucleic acid amplicon of the nucleic acid of interest, which can be the target deoxyribonucleic acid) and generating a detectable signal indicating the presence of said nucleic acid of interest. The methods or reagents may be used as a point of care (POC) diagnostic or as a lab test for detection of a target nucleic acid and, thereby, detection of a condition in a subject from which the sample was taken. The methods or reagents may be used in various sites or locations, such as in laboratories, in hospitals, in physician offices/laboratories (POLs or POCs), in clinics (e.g., POC), at remote sites (e.g., Point of Need (PON)), or over the counter to be used at home or other location (e.g., PON). In some embodiments, the methods and/or reagents disclosed herein are designed to be done manually, such as by a laboratory technician. In some embodiments, the methods and/or reagents disclosed herein are designed to be used with a liquid handling machine, such as an automated or semi-automated liquid handling machine.

The present disclosure provides a method for manual or automated sample preparation within a single chamber. Sample preparation (including lysis, inactivation, isolation, and optional elution) may be performed in the sample chamber as downstream reactions including amplification and/or DETECTR reactions. Single-chamber detection from sample preparation to detection may be non-trivial, as many of the reagents for sample preparation may be incompatible with amplification and/or detection reactions. While removing eluted sample nucleic acids from the chamber into a fresh chamber may reduce the likelihood of carryover of inhibitors, contaminating elements, and/or other complications (such as quenching from microparticles used for nucleic isolation), overcoming these obstacles to provide a single chamber solution may have significant benefits in terms of liquid handling, sample-to-readout timing, cross-contamination reduction, and/or high-throughput workflow functionality.

The high-throughput programmable nuclease DETECTR test method may be implemented within multiple wells on a microplate, with a single well within the plate serving as a single chamber for one or more reactions to occur to detect the presence or absence of a nucleic acid or a plurality of nucleic acids. In an embodiment, a lysis agent and a set of magnetic microparticles are dispensed into the chamber. Then, a sample containing nucleic acid molecules is placed in the chamber. The chamber can be heated, e.g., to approximately 95° C., to promote inactivation, lysis, and binding of the magnetic microparticles to the nucleic acid molecules in the sample. Then, the bound nucleic acid molecules are isolated by contacting the chamber with a magnet while simultaneously aspirating waste liquid from the top of the chamber as the nucleic acid molecules are pulled toward the magnet. Following the isolation and waste removal steps, the chamber is lowered in temperature, e.g., to 57-62° C., and the sample is eluted to separate the microparticles from the nucleic acid molecules. Although the microparticles are separated from the nucleic acid molecules, they may remain in the single chamber during amplification and detection of the nucleic acid molecules. The nucleic acid molecules are then amplified at the same temperature, using RT-LAMP. During the detection stage, the temperature is lowered and a DETECTR mix is added to the sample. The DETECTR mix includes a guide nucleic acid and a programmable nuclease either together as a complex or in situ. If the target nucleic acid exists in the sample, the guide nucleic acid binds to it and cleaves a nucleic acid (which may be a reporter molecule). This produces a signal that is read by a fluorescent plate reader. The fluorescent plate reader takes readings periodically, e.g., every 20 seconds, and a computing device calculates the slope of these readings and compares it to a control.

The disclosed method may be performed in multiple chambers in parallel in a staggered fashion. For example, a first assay may be performed at a first time, and a second assay may be performed at a second time that is 10 minutes from the start time of the first assay, or after the first assay has been halfway completed. The disclosed method can have a limit of detection of less than 500 copies/mL. The disclosed method can provide a test with a sensitivity of above 95% and a specificity of 100%. Implementing the test on a workstation can provide a testing capacity of greater than 1500 tests per 8 hour period or greater than 4500 tests per 24 hour period. The high-throughput testing system can provide about 400 results every 1.75 hours. Users may receive first results from 192 samples in under 110 minutes. The disclosed method may be performed in labs with one hour full-time equivalent (FTE) time in an 8-hour period.

The teachings herein are for a high-throughput system but may be used for other assay systems and devices. For example, the methods disclosed herein may be used with a variety of tube or well-based assays. The methods disclosed may be used with pneumatic valve devices, sliding valve devices, rotating valve devices, or other devices comprising one or more discrete volumes which may serve as reaction chambers. In addition to high-throughput systems, the systems and methods disclosed herein may be used in endpoint assays or kinetic assays.

Within this disclosure, the terms “nucleic acid” and “nucleic acid molecule” may be used interchangeably. The terms “nucleic acids” and “nucleic acid molecules” may be used interchangeably.

### Detection Chamber

A number of support mediums are consistent with the devices, systems, fluidic devices, kits, and methods disclosed herein. These support mediums are, for example, consistent with fluidic devices disclosed herein for detection of a target nucleic acid within the sample, wherein the fluidic device may comprise multiple reservoirs and/or chambers for sample preparation, amplification of a target nucleic acid within the sample, mixing with a programmable nuclease, and detection of a detectable signal arising from cleavage of reporters by the programmable nuclease within the fluidic system itself. A support medium may comprise one or more vessels for holding samples. The vessels may be sealable with lids. The vessels may be chambers such as wells, tubes, or containers. These support mediums are compatible with the samples, reagents, and fluidic devices described herein for detection of one or more viruses, an ailment, such as a disease, cancer, or genetic disorder, or genetic information, such as for phenotyping, genotyping, or determining ancestry. A support medium described herein can provide a way to present the results from the activity between the reagents and the sample. The support medium provides a medium to present the detectable signal in a detectable format. The support mediums can present the results of the assay and indicate the presence or absence of the disease of interest targeted by the target nucleic acid. The result on the support medium can be read by eye or using a machine. The support medium helps to stabilize the detectable signal generated by the cleaved detector molecule on the surface of the support medium. In some instances, the support medium is a plate (e.g., sometimes referred to as a PCR plate). The plate can have 96 wells or 384 wells. The plate can have a subset number of wells of a 96 well plate or a 384 well plate. A subset number of wells of a 96 well plate is, for example, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, or 95 wells. For example, a subset plate can have 4 wells wherein a well is the size of a well from a 96 well plate (e.g., a 4 well subset plate wherein the wells are the size of a well from a 96 well plate). A subset number of wells of a 384 well plate is, for example, 1, 2, 3, 4, 5, 6, 7, 8, 9, 10, 11, 12, 13, 14, 15, 16, 17, 18, 19, 20, 21, 22, 23, 24, 25, 30, 32, 35, 40, 45, 50, 55, 60, 64, 65, 70, 75, 80, 85, 90, 95, 100, 110, 120, 128, 130, 140, 150, 160, 170, 180, 190, 200, 220, 240, 256, 260, 280, 300, 320, 340, 360, or 380 wells. For example, a subset plate can have 20 wells wherein a well is the size of a well from a 384 well plate (e.g., a 20 well subset plate wherein the wells are the size of a well from a 384 well plate). The plate or subset plate can be paired with a fluorescent light reader, a visible light reader, or other imaging device. Often, the imaging device is a digital camera, such a digital camera on a mobile device. The mobile device may have a software program or a mobile application that can capture an image of the plate or subset plate, identify the assay being performed, detect the individual wells and the sample therein, provide image properties of the individuals wells comprising the assayed sample, analyze the image properties of the contents of the individual wells, and provide a result. In some embodiments, non-imaging detection methods are used, including electrical or electrochemical monitoring. For example, the systems and methods disclosed herein may use an ion-sensitive field-effect transistor (ISFET) to measure pH changes. Additionally, the systems and methods disclosed herein may measure electrochemical reactions using a potentiostat or biosensor.

The systems disclosed may include one or more droppers or pipettes. The dropper or the pipette may dispense a predetermined volume. In some cases, the predetermined volume may range from about 1 μl to about 1000 μl, about 1 μl to about 500 μl, about 1 μl to about 100 μl, or about 1 μl to about 50 μl. In some cases, the predetermined volume may be at least 1 μl, 2 μl, 3 μl, 4 μl, 5 μl, 6 μl, 7 μl, 8 μl, 9 μl, 10 μl, 25 μl, 50 μl, 75 μl, 100 μl, 250 μl, 500 μl, 750 μl, or 1000 μl. The predetermined volume may be no more than 5 μl, 10 μl, 25 μl, 50 μl, 75 μl, 100 μl, 250 μl, 500 μl, 750 μl, or 1000 μl. The predetermined volume may be from 1 μL to 1000 μL, from 5 μL to 1000 μL, from 10 μL to 1000 μL, from 20 μL to 1000 μL, from 50 μL to 1000 μL, from 100 μL to 1000 μL, from 200 μL to 1000 μL, from 500 μL to 1000 μL, from 750 μL to 1000 μL, from 1 μL to 750 μL, from 5 μL to 750 μL, from 10 μL to 750 μL, from 20 μL to 750 μL, from 50 μL to 750 μL, from 100 μL to 750 μL, from 200 μL to 750 μL, from 500 μL to 750 μL, from 1 μL to 500 μL, from 5 μL to 500 μL, from 10 μL to 500 μL, from 20 μL to 500 μL, from 50 μL to 500 μL, from 100 μL to 500 μL, from 200 μL to 500 μL, from 1 μL to 200 μL, from 5 μL to 200 μL, from 10 μL to 200 μL, from 20 μL to 200 μL, from 50 μL to 200 μL, from 100 μL to 200 μL, from 1 μL to 100 μL, from 5 μL to 100 μL, from 10 μL to 100 μL, from 20 μL to 100 μL, from 50 μL to 100 μL, from 1 μL to 50 μL, from 5 μL to 50 μL, from 10 μL to 50 μL, from 20 μL to 50 μL, from 1 μL to 20 μL, from 5 μL to 20 μL, from 10 μL to 20 μL, from 1 μL to 10 μL, from 5 μL to 10 μL, or from 1 μL to 5 μL. The dropper or the pipette may be disposable or be single-use. In some embodiments, the methods disclosed herein, e.g., single reaction well, can reduce the number of dropper or pipette tips required for running a reaction. In one non-limiting example, there can be a 3× reduction in number of disposable tips used from a RT-PCR based molecular diagnostic assay. In another non-limiting example, there can be a 2×, 3×, 4×, 5× or more reduction in number of disposable tips used from a RT-PCR based molecular diagnostic assay.

The systems described herein may also comprise a positive control sample to determine the activity of at least one of programmable nuclease, a guide nucleic acid, or a reporter. Often, the positive control sample comprises a target nucleic acid that binds to the guide nucleic acid. The positive control sample is contacted with the reagents in the same manner as the test sample and visualized using the support medium. The visualization of the positive control sample provides a validation of the reagents and the assay.

### Sample

A number of samples are consistent with the devices, systems, fluidic devices, kits, and methods disclosed herein. These samples are, for example, consistent with fluidic devices disclosed herein for detection of a target nucleic acid within the sample, wherein the fluidic device may comprise multiple pumps, valves, reservoirs, and chambers for sample preparation, amplification of a target nucleic acid within the sample, mixing with a programmable nuclease, and detection of a detectable signal arising from cleavage of reporters by the programmable nuclease within the fluidic system itself. These samples can comprise a target nucleic acid for detection of a virus, an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample can be obtained to test for presence of a viral infection, disease, cancer, genetic disorder, or any mutation of interest. A biological sample from the individual may be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample may be dissociated or liquified prior to application to detection system of the present disclosure. A sample from an environment may be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system or be applied neat to the detection system. Sometimes, the sample is contained in no more 20 μl. The sample, in some cases, is contained in no more than 1, 5, 10, 15, 20, 25, 30, 35 40, 45, 50, 55, 60, 65, 70, 75, 80, 90, 100, 200, 300, 400, 500 μl, or any of value from 1 μl to 500 μl. In some cases, the sample is contained in from 1 μL to 500 μL, from 10 μL to 500 μL, from 50 μL to 500 μL, from 100 μL to 500 μL, from 200 μL to 500 μL, from 300 μL to 500 μL, from 400 μL to 500 μL, from 1 μL to 200 μL, from 10 μL to 200 μL, from 50 μL, to 200 μL, from 100 μL to 200 μL, from 1 μL to 100 μL, from 10 μL to 100 μL, from 50 μL to 100 μL, from 1 μL to 50 μL, from 10 μL to 50 μL, from 1 μL to 20 μL, from 10 μL to 20 μL, or from 1 μL to 10 μL. Sometimes, the sample is contained in more than 500 μl.

In some instances, a device described herein can detect less than 10 copies of virus per sample, less than 20 copies/sample, less than 30 copies/sample, less than 40 copies/sample, less than 50 copies/sample, less than 60 copies/sample, less than 70 copies/sample, less than 80 copies/sample, less than 90 copies/sample, less than 100 copies/sample, less than 200 copies/sample, less than 300 copies/sample, less than 400 copies/sample, less than 500 copies/sample, less than 1000 copies/sample, or less than 2000 copies/sample. The virus may be an RNA virus, such as Dengue virus, Ebolavirus, a hantavirus, a Hepatitis virus, an influenza virus, West Nile virus. The virus may be a coronavirus, such as MERS-CoV, SARS-CoV, or SARS CoV-2. In some instances, the limit of detection (LoD) of the test (e.g., for SARS-CoV-2) may be concentrations from under 200 copies/mL, under 300 copies/mL, under 400 copies/mL, under 500 copies/mL, under 600 copies/mL, under 700 copies/mL, under 800 copies/mL, under 900 copies/mL, under 1000 copies/mL, under 2000 copies/mL, under 3000 copies/mL, under 4000 copies/mL, under 5000 copies/mL, to under 6000 copies/mL.

In some instances, the sample is taken from single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell. The sample may be a lower nasal swab sample or a saliva sample.

In some embodiments, a sample is taken from one or more sources (e.g., humans). In some embodiments a plurality of human samples are pooled. In some embodiments pooling of human samples can provide for a more efficient method for a population-wide screening. In the example of detection of a nucleic acid, (e.g., a virus such as SARS-CoV-2), 2, 3, 4, 5, 6, 7, 8, 9, 10 human samples can be pooled together in a single well. Detection of the presence or absence of a nucleic acid in a pooled sample can provide for efficient methods of screening a large population. In the case of a virus such as SARS-CoV-2, pooling samples can provide for cost-effective and accurate methods of screening populations such as students, workers, patients, etc. In some embodiments, 4, 5, 6, 7, or 8 samples can be pooled. In some embodiments, samples can be pooled in a manner that a one positive sample in the pool is detectable. In some embodiments, one positive sample may have 75 copies or more of nucleic acid to be detected.

The sample used for disease testing may comprise at least one target sequence that can bind to a guide nucleic acid of the reagents described herein. In some cases, the target sequence is a portion of a target nucleic acid. A target nucleic acid can be from a genomic locus, a transcribed mRNA, or a reverse transcribed cDNA. A target nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A target nucleic acid can be from 10 to 90, from 20 to 80, from 30 to 70, or from 40 to 60 nucleotides in length. A nucleic acid sequence can be from 10 to 95, from 20 to 95, from 30 to 95, from 40 to 95, from 50 to 95, from 60 to 95, from 10 to 75, from 20 to 75, from 30 to 75, from 40 to 75, from 50 to 75, from 5 to 50, from 15 to 50, from 25 to 50, from 35 to 50, or from 45 to 50 nucleotides in length. A target nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target nucleic acid can be reverse complementary to a guide nucleic acid. In some cases, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides of a guide nucleic acid can be reverse complementary to a target nucleic acid.

A target nucleic may be an antigen or a fragment thereof. In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is a portion of a nucleic acid from sepsis, in the sample. These diseases may include but are not limited to SARS-CoV-2 (including variants B.1.1.7, B.1.351, P.1, B.1.617.2, B. 1429, B.1.427, CAL.20C, P.2, B.1.525, P.3, B.1.526, B.1.617.1, C.37, B.1.1.,207, B.1.620), human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus, human coronavirus, adenovirus (e.g., C1 Ad. 71), human coronavirus, human Metapneumovirus (hMPV), Human coronavirus HKU1, Parainfluenza virus 1-4, Human coronavirus NL63, Influenza A & B, SARS-coronavirus, Enterovirus (e.g., EV68), MERS-coronavirus, Respiratory syncytial virus, Rhinovirus and the like. Pathogens include, e.g., HIV virus, Mycobacterium tuberculosis, Klebsiella pneumoniae, Acinetobacter baumannii, Burkholderia cepacia, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Bordetella parapertussis, Haemophilus influenzae type b, Haemophilus parainfluenzae Z492 Bordetella pertussis, Chlamydia pneumoniae, Mycoplasma pneumoniae, Mycobacterium leprae, Mycoplasma pneumoniae, Pneumocystis jirovecii, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Mycoplasma pneumoniae, Pneumocystis jirovecii (PJP), Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Klebsiella aerogenes, Proteus vulgaris, Serratia marcescens, Enterococcus faecalis, Enterococcus faecium, Streptococcus intermedius, Streptococcus pneumoniae, Staphylococcus epidermis, Pseudomonas aeruginosa, Candida albicans, and Streptococcus pyogenes. Often the target nucleic acid comprises a sequence from a virus or a bacterium or other agents responsible for a disease that can be found in the sample. In some cases, the target nucleic acid is a portion of a nucleic acid from a genomic locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, SARS, MERS, influenza and the like, adenovirus, coronavirus HKU1, coronavirus NL63, coronavirus 229E, coronavirus OC43, human metapneumovirus, human rhinovirus, human enterovirus, influenza A, influenza A/H1, influenza A/H3, influenza A/H1-2009, influenza B, parainfluenza virus 1, parainfluenza virus 2, parainfluenza virus 3, parainfluenza virus 4, respiratory syncytial virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the Coronavirus HKU1 sequence is a target of an assay. In some embodiments, the Coronavirus NL63 sequence is a target of an assay. In some embodiments, the Coronavirus 229E sequence is a target of an assay. In some embodiments, the Coronavirus OC43 sequence is a target of an assay. In some embodiments, the SARS-CoV-1 sequence is a target of an assay. In some embodiments, the MERS sequence is a target of an assay. In some embodiments, the SARS-CoV-2 sequence is a target of an assay. In some embodiments, the Respiratory Syncytial Virus A sequence is a target of an assay. In some embodiments, the Respiratory Syncytial Virus B sequence is a target of an assay. In some embodiments, the Influenza A sequence is a target of an assay. In some embodiments, the Influenza B sequence is a target of an assay. In some embodiments, the Human Metapneumovirus sequence is a target of an assay. In some embodiments, the Human Rhinovirus sequence is a target of an assay. In some embodiments, the Human Enterovirus sequence is a target of an assay. In some embodiments, the Parainfluenza Virus 1 sequence is a target of an assay. In some embodiments, the Parainfluenza Virus 2 sequence is a target of an assay. In some embodiments, the Parainfluenza Virus 3 sequence is a target of an assay. In some embodiments, the Parainfluenza Virus 4 sequence is a target of an assay. In some embodiments, the Alphacoronavirus genus sequence is a target of an assay. In some embodiments, the Betacoronavirus genus sequence is a target of an assay. In some embodiments, the Sarbecovirus subgenus sequence is a target of an assay. In some embodiments, the SARS-related virus species sequence is a target of an assay. In some embodiments, the Gammacoronavirus Genus sequence is a target of an assay. In some embodiments, the Deltacoronavirus Genus sequence is a target of an assay. In some embodiments, the Influenza B-Victoria V1 sequence is a target of an assay. In some embodiments, the Influenza B-Yamagata Y1 sequence is a target of an assay. In some embodiments, the Influenza A H1 sequence is a target of an assay. In some embodiments, the Influenza A H2 sequence is a target of an assay. In some embodiments, the Influenza A H3 sequence is a target of an assay. In some embodiments, the Influenza A H4 sequence is a target of an assay. In some embodiments, the Influenza A H5 sequence is a target of an assay. In some embodiments, the Influenza A H6 sequence is a target of an assay. In some embodiments, the Influenza A H7 sequence is a target of an assay. In some embodiments, the Influenza A H8 sequence is a target of an assay. In some embodiments, the Influenza A H9 sequence is a target of an assay. In some embodiments, the Influenza A H10 sequence is a target of an assay. In some embodiments, the Influenza A H11 sequence is a target of an assay. In some embodiments, the Influenza A H12 sequence is a target of an assay.

In some embodiments, the Influenza A H13 sequence is a target of an assay. In some embodiments, the Influenza A H14 sequence is a target of an assay. In some embodiments, the Influenza A H15 sequence is a target of an assay. In some embodiments, the Influenza A H16 sequence is a target of an assay. In some embodiments, the Influenza A N1 sequence is a target of an assay. In some embodiments, the Influenza A N2 sequence is a target of an assay. In some embodiments, the Influenza A N3 sequence is a target of an assay. In some embodiments, the Influenza A N4 sequence is a target of an assay. In some embodiments, the Influenza A N5 sequence is a target of an assay. In some embodiments, the Influenza A N6 sequence is a target of an assay. In some embodiments, the Influenza A N7 sequence is a target of an assay. In some embodiments, the Influenza A N8 sequence is a target of an assay. In some embodiments, the Influenza A N9 sequence is a target of an assay. In some embodiments, the Influenza A N10 sequence is a target of an assay. In some embodiments, the Influenza A N11 sequence is a target of an assay. In some embodiments, the Influenza A/H1-2009 sequence is a target of an assay. In some embodiments, the Human endogenous control 18S rRNA sequence is a target of an assay. In some embodiments, the Human endogenous control GAPDH sequence is a target of an assay. In some embodiments, the Human endogenous control HPRT1 sequence is a target of an assay. In some embodiments, the Human endogenous control GUSB sequence is a target of an assay. In some embodiments, the Human endogenous control RNase P sequence is a target of an assay. In some embodiments, the Influenza A oseltamivir resistance sequence is a target of an assay. In some embodiments, the Human Bocavirus sequence is a target of an assay. In some embodiments, the SARS-CoV-2 85Δ sequence is a target of an assay. In some embodiments, the SARS-CoV-2 T1001I sequence is a target of an assay. In some embodiments, the SARS-CoV-2 3675-3677Δ sequence is a target of an assay. In some embodiments, the SARS-CoV-2 P4715L sequence is a target of an assay. In some embodiments, the SARS-CoV-2 S5360L sequence is a target of an assay. In some embodiments, the SARS-CoV-2 69-70Δ sequence is a target of an assay. In some embodiments, the SARS-CoV-2 Tyr144fs sequence is a target of an assay. In some embodiments, the SARS-CoV-2 242-244Δ sequence is a target of an assay. In some embodiments, the SARS-CoV-2 Y453F sequence is a target of an assay. In some embodiments, the SARS-CoV-2 S477N sequence is a target of an assay. In some embodiments, the SARS-CoV-2 E848K sequence is a target of an assay. In some embodiments, the SARS-CoV-2 N501Y sequence is a target of an assay. In some embodiments, the SARS-CoV-2 D614G sequence is a target of an assay. In some embodiments, the SARS-CoV-2 P681R sequence is a target of an assay. In some embodiments, the SARS-CoV-2 P681H sequence is a target of an assay. In some embodiments, the SARS-CoV-2 L21F sequence is a target of an assay. In some embodiments, the SARS-CoV-2 Q27Stop sequence is a target of an assay. In some embodiments, the SARS-CoV-2 M1fs sequence is a target of an assay. In some embodiments, the SARS-CoV-2 R203fs sequence is a target of an assay. In some embodiments, the Human adenovirus-pan assay sequence is a target of an assay. In some embodiments, the Bordetella parapertussis sequence is a target of an assay. In some embodiments, the Bordetella pertussis sequence is a target of an assay. In some embodiments, the Chlamydophila pneumoniae sequence is a target of an assay. In some embodiments, the Mycoplasma pneumoniae sequence is a target of an assay. In some embodiments, the Legionella pneumophila sequence is a target of an assay. In some embodiments, the Bordetella bronchiseptica sequence is a target of an assay. In some embodiments, the Bordetella holmesii sequence is a target of an assay. In some embodiments, the Human adenovirus Type A sequence is a target of an assay. In some embodiments, the Human adenovirus Type B sequence is a target of an assay. In some embodiments, the Human adenovirus Type C sequence is a target of an assay. In some embodiments, the Human adenovirus Type D sequence is a target of an assay. In some embodiments, the Human adenovirus Type E sequence is a target of an assay. In some embodiments, the Human adenovirus Type F sequence is a target of an assay. In some embodiments, the Human adenovirus Type G sequence is a target of an assay. In some embodiments, the MERS-CoV sequence is a target of an assay. In some embodiments, the human metapneumovirus sequence is a target of an assay. In some embodiments, the human parainfluenza 1 sequence is a target of an assay. In some embodiments, the human parainfluenza 2 sequence is a target of an assay. In some embodiments, the human parainfluenza 4 sequence is a target of an assay. In some embodiments, the hCoV-OC43 sequence is a target of an assay. In some embodiments, the human parainfluenza 3 sequence is a target of an assay. In some embodiments, the RSV-A sequence is a target of an assay. In some embodiments, the RSV-B sequence is a target of an assay. In some embodiments, the hCoV-229E sequence is a target of an assay. In some embodiments, the hCoV-HKU1 sequence is a target of an assay. In some embodiments, the hCoV-NL63 sequence is a target of an assay. In some embodiments, the Gammacoronavirus sequence is a target of an assay. In some embodiments, the Deltacoronavirus sequence is a target of an assay. In some embodiments, the Alphacoronavirus sequence is a target of an assay. In some embodiments, the Rhinovirus C sequence is a target of an assay. In some embodiments, the Betacoronavirus sequence is a target of an assay. In some embodiments, the Influenza A sequence is a target of an assay. In some embodiments, the Influenza B sequence is a target of an assay. In some embodiments, the SARS-CoV-2 sequence is a target of an assay. In some embodiments, the SARS-CoV-1 sequence is a target of an assay. In some embodiments, the Sarbecovirus subgenus sequence is a target of an assay. In some embodiments, the SARS-related viruses sequence is a target of an assay. In some embodiments, the MS2 sequence is a target of an assay.

In some embodiments, the one or more targets may be at a concentration of 1 copy/reaction, at least about 2 copies/reaction, at least about 3 copies/reaction, at least about 4 copies/reaction, at least about 5 copies/reaction, at least about 6 copies/reaction, at least about 7 copies/reaction, at least about 8 copies/reaction, at least about 9 copies/reaction, at least about 10 copies/reaction, at least about 20 copies/reaction, at least about 30 copies/reaction, at least about 40 copies/reaction, at least about 50 copies/reaction, at least about 60 copies/reaction, at least about 70 copies/reaction, at least about 80 copies/reaction, at least about 90 copies/reaction, at least about 100 copies/reaction, at least about 200 copies/reaction, at least about 300 copies/reaction, at least about 400 copies/reaction, at least about 500 copies/reaction, at least about 600 copies/reaction, at least about 700 copies/reaction, at least about 800 copies/reaction, at least about 900 copies/reaction, at least about 1000 copies/reaction, at least about 2000 copies/reaction, at least about 3000 copies/reaction, at least about 4000 copies/reaction, at least about 5000 copies/reaction, at least about 6000 copies/reaction, at least about 7000 copies/reaction, at least about 8000 copies/reaction, at least about 9000 copies/reaction, at least about 10000 copies/reaction, at least about 20000 copies/reaction, at least about 30000 copies/reaction, at least about 40000 copies/reaction, at least about 50000 copies/reaction, at least about 60000 copies/reaction, at least about 70000 copies/reaction, at least about 80000 copies/reaction, at least about 90000 copies/reaction, or at least about 100000 copies/reaction.

The sample used may comprise at least one target nucleic acid segment that can bind to a guide nucleic acid of the reagents described herein. The target nucleic acid segment, in some cases, is a portion of a nucleic acid from a gene with a mutation associated with disease or disorder (e.g., cancer), from a gene whose overexpression is associated with a disease or disorder (e.g., cancer, a tumor suppressor gene, an oncogene, a checkpoint inhibitor gene, a gene associated with cellular growth, a gene associated with cellular metabolism, or a gene associated with cell cycle). Sometimes, the target nucleic acid encodes for a disease indicator, such as a cancer biomarker, such as a prostate cancer biomarker or non-small cell lung cancer. In some cases, the assay can be used to detect “hotspots” in target nucleic acids that can be predictive of lung cancer. In some cases, the target nucleic acid is a portion of a nucleic acid that is associated with a blood fever. In some cases, the target nucleic acid segment is a portion of a nucleic acid from a genomic locus, a transcribed mRNA, or a reverse transcribed cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1.

The sample used for a disorder (e.g., a genetic disorder) testing may comprise at least one target nucleic acid segment that can bind to a guide nucleic acid of the reagents described herein. In some embodiments, the genetic disorder is hemophilia, sickle cell anemia, β-thalassemia, Duchenne muscular dystrophy, severe combined immunodeficiency, or cystic fibrosis. The target nucleic acid segment, in some cases, is a portion of a nucleic acid from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid segment is a portion of a nucleic acid from a genomic locus, a transcribed mRNA, or a reverse transcribed cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GSA, GBE1, GCDH, GFM1, GJB 1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing may comprise at least one target nucleic acid segment that can bind to a guide nucleic acid of the reagents described herein. The target nucleic acid segment, in some cases, is a portion of a nucleic acid from a gene associated with a phenotypic trait. The sample used for genotyping testing may comprise at least one target nucleic acid segment that can bind to a guide nucleic acid of the reagents described herein. The target nucleic acid segment, in some cases, is a portion of a nucleic acid from a gene associated with a genotype. The sample used for ancestral testing may comprise at least one target nucleic acid segment that can bind to a guide nucleic acid of the reagents described herein. The target nucleic acid segment, in some cases, is a portion of a nucleic acid from a gene associated with a geographic region of origin or ethnic group. The sample can be used for identifying a disease status. For example, a sample is any sample described herein, and is obtained from a subject for use in identifying a disease status of a subject. The disease can be a cancer or genetic disorder. Sometimes, a method comprises obtaining a serum sample from a subject; and identifying a disease status of the subject. Often, the disease status is prostate disease status.

In some instances, the target nucleic acid is a single stranded nucleic acid. Alternatively or in combination, the target nucleic acid is a double stranded nucleic acid and is prepared into single stranded nucleic acids before or upon contacting the reagents. The target nucleic acid may be a RNA, DNA, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the methods and compositions disclosed herein. Some methods described herein can detect a target nucleic acid present in the sample in various concentrations or amounts as a target nucleic acid population. In some cases, the sample has at least 2 copies of a target nucleic acid. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 copies of the target nucleic acid. In some cases, the sample has from 1 to 10,000, from 100 to 8000, from 400 to 6000, from 500 to 5000, from 1000 to 4000, or from 2000 to 3000 target nucleic acid copies. In some cases, the sample has from 100 to 9500, from 100 to 9000, from 100 to 8500, from 100 to 8000, from 100 to 7500, from 100 to 7000, from 100 to 6500, from 100 to 6000, from 100 to 5500, from 100 to 5000, from 250 to 9500, from 250 to 9000, from 250 to 8500, from 250 to 8000, from 250 to 7500, from 250 to 7000, from 250 to 6500, from 250 to 6000, from 250 to 5500, from 250 to 5000, from 2500 to 9500, from 2500 to 9000, from 2500 to 8500, from 2500 to 8000, from 2500 to 7500, from 2500 to 7000, from 2500 to 6500, from 2500 to 6000, from 2500 to 5500, or from 2500 to 5000 target nucleic acid copies. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acid populations are consistent with the methods and compositions disclosed herein. Some methods described herein can detect two or more different target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has from 3 to 50, from 5 to 40, or from 10 to 25 target nucleic acid populations. In some cases, the sample has from 2 to 50, from 5 to 50, from 10 to 50, from 2 to 25, from 3 to 25, from 4 to 25, from 5 to 25, from 10 to 25, from 2 to 20, from 3 to 20, from 4 to 20, from 5 to 20, from 10 to 20, from 2 to 10, from 3 to 10, from 4 to 10, from 5 to 10, from 6 to 10, from 7 to 10, from 8 to 10, or from 9 to 10 target nucleic acid populations. In some cases, the method detects target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

Additionally, a target nucleic acid can be amplified before binding to a guide nucleic acid, for example a crRNA of a CRISPR enzyme. This amplification can be, e.g., PCR amplification, isothermal amplification such as RT-LAMP, or the like. This nucleic acid amplification of the sample can improve at least one of sensitivity, specificity, or accuracy of the detection the target RNA. The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification is performed for from 1 to 60, from 5 to 55, from 10 to 50, from 15 to 45, from 20 to 40, or from 25 to 35 minutes. Sometimes, the nucleic acid amplification is performed for from 5 to 60, from 10 to 60, from 15 to 60, from 30 to 60, from 45 to 60, from 1 to 45, from 5 to 45, from 10 to 45, from 30 to 45, from 1 to 30, from 5 to 30, from 10 to 30, from 15 to 30, from 1 to 15, from 5 to 15, or from 10 to 15 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C. The nucleic acid amplification reaction can be performed at a temperature of at least 20° C., 25° C., 30° C., 35° C., 37° C., 40° C., or 45° C. In some cases, the nucleic acid amplification reaction is performed at a temperature of from 20° C. to 45° C., from 25° C. to 40° C., from 30° C. to 40° C., or from 35° C. to 40° C. In some cases, the nucleic acid amplification reaction is performed at a temperature of from 20° C. to 45° C., from 25° C. to 45° C., from 30° C. to 45° C., from 35° C. to 45° C., from 40° C. to 45° C., from 20° C. to 37° C., from 25° C. to 37° C., from 30° C. to 37° C., from 35° C. to 37° C., from 20° C. to 30° C., from 25° C. to 30° C., from 20° C. to 25° C., or from 22° C. to 25° C. The nucleic acid amplification reaction can be performed at a temperature no greater than 20° C., 25° C., 30° C., 35° C., 37° C., 40° C., 45° C., 50° C., 55° C., 60° C., 65° C., or 70° C. The nucleic acid amplification reaction can be performed at a temperature of at least 20° C., 25° C., 30° C., 35° C., 37° C., 40° C., 45° C., 50° C., 55° C., 60° C., or 65° C. In some cases, the nucleic acid amplification reaction is performed at a temperature of from 20° C. to 45° C., from 25° C. to 40° C., from 30° C. to 40° C., or from 35° C. to 40° C. In some cases, the nucleic acid amplification reaction is performed at a temperature of from 45° C. to 65° C., from 50° C. to 65° C., from 55° C. to 65° C., or from 60° C. to 65° C. In some cases, the nucleic acid amplification reaction can be performed at a temperature that ranges from about 20° C. to 45° C., from 25° C. to 45° C., from 30° C. to 45° C., from 35° C. to 45° C., from 40° C. to 45° C., from 20° C. to 37° C., from 25° C. to 37° C., from 30° C. to 37° C., from 35° C. to 37° C., from 20° C. to 30° C., from 25° C. to 30° C., from 20° C. to 25° C., or from about 22° C. to 25° C. In some cases, the nucleic acid amplification reaction can be performed at a temperature that ranges from about 40° C. to 65° C., from 45° C. to 65° C., from 50° C. to 65° C., from 55° C. to 65° C., from 60° C. to 65° C., from 65° C. to 70° C., from 40° C. to 60° C., from 45° C. to 60° C., from 50° C. to 60° C., from 55° C. to 60° C., from 40° C. to 55° C., from 45° C. to 55° C., from 50° C. to 55° C., from 40° C. to 50° C., or from about 45° C. to 50° C.

Any of the above disclosed samples are consistent with the systems, assays, and programmable nucleases disclosed herein and can be used as a diagnostic with any of the diseases disclosed herein (e.g., SARS-CoV-2, RSV, sepsis, flu), or can be used in reagent kits, point-of-care diagnostics, or over-the-counter diagnostics.

### Reporters

Reporters, which can be referred to interchangeably as reporter molecules or detector nucleic acids, described herein are compatible for use in the devices described herein and may be used in conjunction with compositions disclosed herein (e.g., programmable nucleases, guide nucleic acids, reagents for in vitro transcription, reagents for amplification, reagents for reverse transcription, reporters, or any combination thereof) to carry out highly efficient, rapid, and accurate reactions for detecting whether a target nucleic acid is present in a sample (e.g., DETECTR reactions). The reporter can be suspended in solution or immobilized on a surface. For example, the reporter can be immobilized on the surface of a chamber in a device as disclosed herein. In some cases, the reporter can be immobilized on beads, such as magnetic beads, in a chamber of a device as disclosed herein where they are held in position by a magnet placed below the chamber. The reporter can be capable of being cleaved by the activated programmable nuclease, thereby generating a detectable signal.

Described herein is a reporter comprising a single stranded reporter comprising a detection moiety, wherein the reporter is capable of being cleaved by an activated programmable nuclease, thereby generating a first detectable signal. As used herein, a reporter is used interchangeably with reporter or reporter molecule. In some cases, the reporter is a single-stranded nucleic acid comprising deoxyribonucleotides. In other cases, the reporter is a single-stranded nucleic acid comprising ribonucleotides. The reporter can be a single-stranded nucleic acid comprising at least one deoxyribonucleotide and at least one ribonucleotide. In some cases, the reporter is a single-stranded nucleic acid comprising at least one ribonucleotide residue at an internal position that functions as a cleavage site. In some cases, the reporter comprises at least 2, 3, 4, 5, 6, 7, 8, 9, or 10 ribonucleotide residues at an internal position. In some cases, the reporter comprises from 2 to 10, from 3 to 9, from 4 to 8, or from 5 to 7 ribonucleotide residues at an internal position. In some cases, the reporter comprises from 3 to 10, from 4 to 10, from 5 to 10, from 6 to 10, from 7 to 10, from 8 to 10, from 9 to 10, from 2 to 8, from 3 to 8, from 5 to 8, from 6 to 8, from 7 to 8, from 2 to 5, from 3 to 5, or from 4 to 5 ribonucleotide residues at an internal position. Sometimes the ribonucleotide residues are continuous. Alternatively, the ribonucleotide residues are interspersed in between non-ribonucleotide residues. In some cases, the reporter has only ribonucleotide residues. In some cases, the reporter has only deoxyribonucleotide residues. In some cases, the reporter comprises nucleotides resistant to cleavage by the programmable nuclease described herein. In some cases, the reporter comprises synthetic nucleotides. In some cases, the reporter comprises at least one ribonucleotide residue and at least one non-ribonucleotide residue. In some cases, the reporter is 5-20, 5-15, 5-10, 7-20, 7-15, or 7-10 nucleotides in length. In some cases, the reporter is from 3 to 20, from 4 to 20, from 5 to 20, from 6 to 20, from 7 to 20, from 8 to 20, from 9 to 20, from 10 to 20, from 15 to 20, from 3 to 15, from 4 to 15, from 5 to 15, from 6 to 15, from 7 to 15, from 8 to 15, from 9 to 15, from 10 to 15, from 3 to 10, from 4 to 10, from 5 to 10, from 6 to 10, from 7 to 10, from 8 to 10, from 9 to 10, from 3 to 8, from 4 to 8, from 5 to 8, from 6 to 8, or from 7 to 8 nucleotides in length. In some cases, the reporter comprises at least one uracil ribonucleotide. In some cases, the reporter comprises at least two uracil ribonucleotides. Sometimes the reporter has only uracil ribonucleotides. In some cases, the reporter comprises at least one adenine ribonucleotide. In some cases, the reporter comprises at least two adenine ribonucleotides. In some cases, the reporter has only adenine ribonucleotides. In some cases, the reporter comprises at least one cytosine ribonucleotide. In some cases, the reporter comprises at least two cytosine ribonucleotide. In some cases, the reporter comprises at least one guanine ribonucleotide. In some cases, the reporter comprises at least two guanine ribonucleotide. A reporter can comprise only unmodified ribonucleotides, only unmodified deoxyribonucleotides, or a combination thereof. In some cases, the reporter is from 5 to 12 nucleotides in length. In some cases, the reporter is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the reporter is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For example, for cleavage by a programmable nuclease comprising Cas13, a reporter can be 5, 8, or 10 nucleotides in length. For example, for cleavage by a programmable nuclease comprising Cas12, a reporter can be 10 nucleotides in length.

The single stranded reporter can comprise a detection moiety capable of generating a first detectable signal. Sometimes the reporter comprises a protein capable of generating a signal. A signal can be a calorimetric, potentiometric, amperometric, optical (e.g., fluorescent, colorimetric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the reporter. Sometimes the detection moiety is at the 3′ terminus of the reporter. In some cases, the detection moiety is at the 5′ terminus of the reporter. In some cases, the quenching moiety is at the 3′ terminus of the reporter. In some cases, the single-stranded reporter is at least one population of the single-stranded nucleic acid capable of generating a first detectable signal. In some cases, the single-stranded reporter is a population of the single stranded nucleic acid capable of generating a first detectable signal. Optionally, there are more than one population of single-stranded reporter. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded reporters capable of generating a detectable signal. In some cases there are from 2 to 50, from 3 to 40, from 4 to 30, from 5 to 20, or from 6 to 10 different populations of single-stranded reporters capable of generating a detectable signal. In some cases there are from 2 to 50, from 5 to 50, from 10 to 50, from 15 to 50, from 20 to 50, from 25 to 50, from 30 to 50, from 35 to 50, from 40 to 50, from 2 to 40, from 5 to 40, from 10 to 40, from 15 to 40, from 20 to 40, from 25 to 40, from 30 to 40, from 35 to 40, from 2 to 30, from 5 to 30, from 10 to 30, from 15 to 30, from 20 to 30, from 25 to 30, from 2 to 20, from 5 to 20, from 10 to 20, from 15 to 20, from 2 to 10, or from 5 to 10 different populations of single-stranded reporters capable of generating a detectable signal.

A detection moiety can be an infrared fluorophore. A detection moiety can be a fluorophore that emits fluorescence in the range of from 500 nm and 720 nm. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 680 to 690, 690 to 700, 700 to 710, 710 to 720, or 720 to 730 nm. In some cases, the detection moiety emits fluorescence in the range from 450 nm to 750 nm, from 500 nm to 750 nm, from 550 nm to 750 nm, from 600 nm to 750 nm, from 650 nm to 750 nm, from 700 nm to 750 nm, from 450 nm to 700 nm, from 500 nm to 700 nm, from 550 nm to 700 nm, from 600 nm to 700 nm, from 650 nm to 700 nm, from 450 nm to 650 nm, from 500 nm to 650 nm, from 550 nm to 650 nm, from 600 nm to 650 nm, from 450 nm to 600 nm, from 500 nm to 600 nm, from 550 nm to 600 nm, from 450 nm to 550 nm, from 500 nm to 550 nm, or from 450 nm to 500 nm. A detection moiety can be a fluorophore that emits a fluorescence in the same range as 6-Fluorescein, IRDye 700, TYE 665, Alex Fluor, or ATTO TM 633 (NHS Ester). A detection moiety can be fluorescein amidite, 6-Fluorescein, IRDye 700, TYE 665, Alex Fluor 594, or ATTO TM 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A detection moiety can be fluorescein amidite, 6-Fluorescein (Integrated DNA Technologies), a digoxigenin, IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Other fluorophores consistent with the present disclosure include Alexa Fluor 405, Alexa 488, Cy2, Cy3, Cy3.5, Cy5, Cy5.5, Cy7, Alexa Fluor 647, or other suitable fluorophores. Optimum excitation and emission wavelengths for Cy5 may be 643 nm and 672 nm, respectively. Any of the detection moieties described herein can be from any commercially available source, can be an alternative with a similar function, a generic, or a non-tradename of the detection moieties listed.

The generation of the detectable signal from the release of the detection moiety indicates that cleavage by the programmable nuclease has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively or in combination, the detection moiety comprises a polypeptide. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

In some embodiments, the reporter comprises a nucleic acid conjugated to an affinity molecule and the affinity molecule conjugated to the fluorophore (e.g., nucleic acid-affinity molecule-fluorophore) or the nucleic acid conjugated to the fluorophore and the fluorophore conjugated to the affinity molecule (e.g., nucleic acid-fluorophore-affinity molecule). In some embodiments, a linker conjugates the nucleic acid to the affinity molecule. In some embodiments, a linker conjugates the affinity molecule to the fluorophore. In some embodiments, a linker conjugates the nucleic acid to the fluorophore. A linker can be any suitable linker known in the art. In some embodiments, the nucleic acid of the reporter can be directly conjugated to the affinity molecule and the affinity molecule can be directly conjugated to the fluorophore or the nucleic acid can be directly conjugated to the fluorophore and the fluorophore can be directly conjugated to the affinity molecule. In this context, “directly conjugated” indicated that no intervening molecules, polypeptides, proteins, or other moieties are present between the two moieties directly conjugated to each other. For example, if a reporter comprises a nucleic acid directly conjugated to an affinity molecule and an affinity molecule directly conjugated to a fluorophore—no intervening moiety is present between the nucleic acid and the affinity molecule and no intervening moiety is present between the affinity molecule and the fluorophore. The affinity molecule can be biotin, avidin, streptavidin, or any similar molecule. Additional examples of affinity molecules are biotin, glutathione, maltose, or chitin.

A detection moiety can be any moiety capable of generating a calorimetric, potentiometric, amperometric, optical (e.g., fluorescent, colorimetric, etc.), or piezo-electric signal. A reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the reporters. Sometimes, a calorimetric signal is heat absorbed after cleavage of the reporters. A potentiometric signal, for example, is electrical potential produced after cleavage of the reporters. An amperometric signal can be movement of electrons produced after the cleavage of reporter. Often, the signal is an optical signal, such as a colorimetric signal or a fluorescence signal. An optical signal is, for example, a light output produced after the cleavage of the reporters. Sometimes, an optical signal is a change in light absorbance between before and after the cleavage of reporters. Often, a piezo-electric signal is a change in mass between before and after the cleavage of the reporter.

In some embodiments, the reporter may comprise a quenching moiety. In some embodiments, a quenching moiety is any entity that decreases the fluorescence intensity of a given substance. Exemplary embodiments of reporters, labels, quenchers, chemical functionalities, detection moieties, dendrimers, quenching moieties and other reporter elements are described in: PCT/US21/33271 (755.601); PCT/US21/35031 (754.601), and U.S. Provisional Patent Application No. 63/187,298 (780.101), all of which are herein incorporated by reference in their entirety.

### Reagents

A number of reagents are consistent with the devices, systems, fluidic devices, kits, and methods disclosed herein. These reagents are, for example, consistent for use within various fluidic devices disclosed herein for detection of a target nucleic acid within the sample, wherein the fluidic device may comprise multiple pumps, valves, reservoirs, and chambers for sample preparation, amplification of a target nucleic acid within the sample, mixing with a programmable nuclease, and detection of a detectable signal arising from cleavage of reporters by the programmable nuclease within the fluidic system itself. These reagents are compatible with the samples, fluidic devices, methods of detection, and support mediums as described herein for detection of an ailment, such as a disease, cancer, or genetic disorder, or genetic information, such as for phenotyping, genotyping, or determining ancestry. The reagents described herein for detecting a disease, cancer, or genetic disorder comprise a guide nucleic acid targeting the target nucleic acid segment indicative of a disease, cancer, or genetic disorder. Reagents of this disclosure can include guide nucleic acids, substrate nucleic acids, detection reagents, signal reagents, buffers, and/or programmable nucleases.

### Lysis Agent

Lysis may be implemented using a lysis buffer solution. Active ingredients of the solution can be chaotropic agents, detergents, salts, and can be of high osmolality, ionic strength and pH. Chaotropic agents or chaotropes are substances that disrupt the three-dimensional structure in macromolecules such as proteins, DNA, or RNA. One example protocol comprises a 4 M guanidinium isothiocyanate, 25 mM sodium citrate dihydrate, 0.5% (w/v) sodium lauryl sarcosinate, and 0.1 M β-mercaptoethanol, but numerous commercial buffers for different cellular targets can also be used. Alkaline buffers can also be used for cells with hard shells, particularly for environmental samples. Detergents such as sodium dodecyl sulphate (SDS) and cetyl trimethylammonium bromide (CTAB) can also be implemented to chemical lysis buffers. Lysis buffers may comprise HEPES, MES, TCEP, EGTA, Tween 20, KC1, MgCl², glycerol, or any combination thereof. In some instances, a lysis buffer may comprise Tris-HCl pH 8.8, VLB, EGTA, CH3COOH, TCEP, IsoAmp, (NH4)2SO4, KCl, MgSO4, Tween20, KOAc, MgOAc, BSA, or any combination thereof. A lysis buffer may be an NP-40 lysis buffer, a RadioImmunoPrecipitation Assay (RIPA) lysis buffer, a sodium dodecyl sulfate (SDS) lysis buffer, or an ammonium-chloride potassium (ACK) lysis buffer.

Cell lysis can also be performed by physical, mechanical, thermal or enzymatic means, in addition to or alternatively to chemically-induced cell lysis mentioned previously. In some cases, depending on the type of sample, nanoscale barbs, nanowires, acoustic generators, integrated lasers, integrated heaters, and/or microcapillary probes can be used to perform lysis.

### Elution Agent

A sample preparation protocol includes elution of a sample into a buffer that will induce dissociation of the sample into its macromolecule components releasing the genomic nucleic acids. Buffer conditions used to induce the dissociation include any or all of the following: pH change, chaotropic salts and a detergent (Tween 20, Triton X-100, Deoxycholate, Sodium laurel sulfate or 3-[(3-cholamidopropyl)dimethylammonio]-1-propanesulfonate (CHAPS)). An example elution solution is 10 mM Tris HCl with pH 8.0. Elution buffers may comprise HEPES, MES, TCEP, EGTA, Tween 20, KC1, MgCl², glycerol, or any combination thereof. In some instances, an elution buffer may comprise Tris-HCl pH 8.8, VLB, EGTA, CH3COOH, TCEP, IsoAmp, (NH4)2SO4, KCl, MgSO4, Tween20, KOAc, MgOAc, BSA, or any combination thereof. Example elution buffers may be 100 mM glycine, pH 2.5, 1M triethanolamine, 4M MgCl2, 1M NaCl/PBS.

### Amplification Agent

In some embodiments, reagents for amplification can comprise a DNA sequence, dNTPs, a forward primer, a reverse primer, and a polymerase. In some embodiments, reagents for RT-RPA amplification may comprise a target DNA or RNA, RPA primers, deoxynucleotide triphosphates (dNTPs), a polymerase, and a reverse transcriptase enzyme. In some embodiments, reagents for an in vitro transcription (IVT) reaction may comprise a target DNA, NTPs, and an RNA polymerase enzyme (e.g, T7 RNA polymerase). In some embodiments, reagents for an RT-RPA-IVT combined amplification and transcription reaction may comprise a target DNA or RNA sequence, RPA primers, an RPA primer having a T7 promoter, a reverse transcriptase enzyme, dNTPs, NTPs, a recombinase, an RNA polymerase enzyme (e.g, T7 RNA polymerase), or any combination thereof. In some embodiments, reagents for LAMP amplification may comprise a target DNA, a plurality of primers (e.g., four, five, or six primers), dNTPs, and a polymerase. In some embodiments, reagents for RT-LAMP amplification may comprise a target RNA, a plurality of primers (e.g., four, five, or six primers), dNTPs, a polymerase, and a reverse transcriptase enzyme. In some embodiments, reagents for RT-LAMP-IVT may comprise a target RNA, a plurality of primers (e.g., four, five, or six primers), a primer having a T7 promoter, dNTPs, NTPs, a polymerase enzyme, a reverse transcriptase enzyme, and an RNA polymerase (e.g., T7 RNA polymerase). For example, an RT-LAMP Master Mix may include Bst 2.0 DNA polymerase, RNase inhibitor, Murine, an elution buffer, 100 mM dATP, 100 mM dCTP, 100 mM dGTP, and 100 mM dTTP. In some embodiments, reagents for SIBA amplification may comprise a target DNA having a protospacer adjacent motif (PAM), dNTPs, and a polymerase enzyme. In some embodiments, reagents for RT-SIBA amplification may comprise a target RNA having a protospacer adjacent motif (PAM), primers, dNTPs, a polymerase enzyme, and a reverse transcriptase enzyme. In some embodiments, the present disclosure provides devices and methods that allow for rapid reverse transcription, amplification, and/or in vitro transcription of target nucleic acids of interest, in one step. Thus, the general reagents for reverse transcription, amplification, and/or in vitro transcription can be combined regardless of the specific method of amplification used.

The amplification agents disclosed herein may be compatible with buffers. Compatible buffers and buffer components may include HEPES, KCl, MgCl2, glycerol, Igepal Ca-630, BSA, and imidazole. Amplification buffers may comprise HEPES, MES, TCEP, EGTA, Tween 20, KC1, MgCl², glycerol, or any combination thereof. In some instances, an amplification buffer may comprise Tris-HCl pH 8.8, VLB, EGTA, CH3COOH, TCEP, IsoAmp, (NH4)2SO4, KCl, MgSO4, Tween20, KOAc, MgOAc, BSA, or any combination thereof. Amplification buffers may additionally, 2-(N-morpholino), 3-(N-morpholino)propanesulfonic acid (MOPS), citrate buffers, and phosphate buffers.

The methods disclosed may use RT-LAMP activator solutions. An example activator solution includes KOAc, MgOAc, NH₄OAc, Tris HCl, pH 9.0, Tween 20, Primer F3, Primer B3, Primer FIP, Primer BIP, Primer LF, and Primer LB. Activator buffers may comprise HEPES, MES, TCEP, EGTA, Tween 20, KC1, MgCl², glycerol, or any combination thereof. In some instances, an activator buffer may comprise Tris-HCl pH 8.8, VLB, EGTA, CH3COOH, TCEP, IsoAmp, (NH4)2SO4, KCl, MgSO4, Tween20, KOAc, MgOAc, BSA, or any combination thereof. An activation buffer may be an EDAC solution or an S-NHS solution.

### Detection

The devices, systems, fluidic devices, kits, and methods described herein may comprise a generation of a signal in response to the presence or absence of a target nucleic acid in a sample which may be detected using detection methods described herein. The present disclosure provides methods of assaying for a target nucleic acid as described herein wherein a signal is detected. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal or a presence of the signal near background indicates an absence of the target nucleic acid in the sample.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomolar (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 aM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, from 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases the threshold of detection is in a range of from 2 aM to 100 pM, from 20 aM to 50 pM, from 50 aM to 20 pM, from 200 aM to 5 pM, or from 500 aM to 2 pM. In some cases the threshold of detection is in a range of from 1 aM to 2 nM, from 10 aM to 2 nM, from 100 aM to 2 nM, from 1 fM to 2 nM, from 10 fM to 2 nM, from 100 fM to 2 nM, from 1 pM to 2 nM, from 10 pM to 2 nM, from 100 pM to 2 nM, from 1 aM to 200 pM, from 10 aM to 200 pM, from 100 aM to 200 pM, from 1 fM to 200 pM, from 10 fM to 200 pM, from 100 fM to 200 pM, from 1 pM to 200 pM, from 10 pM to 200 pM, from 1 aM to 20 pM, from 10 aM to 20 pM, from 100 aM to 20 pM, from 1 fM to 20 pM, from 10 fM to 20 pM, from 100 fM to 20 pM, from 1 pM to 20 pM, from 1 aM to 2 pM, from 10 aM to 2 pM, from 100 aM to 2 pM, from 1 fM to 2 pM, from 10 fM to 2 pM, from 100 fM to 2 pM, from 1 aM to 200 fM, from 10 aM to 200 fM, from 100 aM to 200 fM, from 1 fM to 200 fM, from 10 fM to 200 fM, from 1 aM to 20 fM, from 10 aM to 20 fM, from 100 aM to 20 fM, from 1 fM to 20 fM, from 1 aM to 2 fM, from 10 aM to 2 fM, from 100 aM to 2 fM, from 1 aM to 200 aM, from 10 aM to 200 aM, or from 1 aM to 20 aM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single stranded target nucleic acid is detected in a sample is in a range of from 2 aM to 100 pM, from 20 aM to 50 pM, from 50 aM to 20 pM, from 200 aM to 5 pM, or from 500 aM to 2 pM. In some cases, the minimum concentration at which a single stranded target nucleic acid is detected in a sample is in a range of from 1 aM to 2 nM, from 10 aM to 2 nM, from 100 aM to 2 nM, from 1 fM to 2 nM, from 10 fM to 2 nM, from 100 fM to 2 nM, from 1 pM to 2 nM, from 10 pM to 2 nM, from 100 pM to 2 nM, from 1 aM to 200 pM, from 10 aM to 200 pM, from 100 aM to 200 pM, from 1 fM to 200 pM, from 10 fM to 200 pM, from 100 fM to 200 pM, from 1 pM to 200 pM, from 10 pM to 200 pM, from 1 aM to 20 pM, from 10 aM to 20 pM, from 100 aM to 20 pM, from 1 fM to 20 pM, from 10 fM to 20 pM, from 100 fM to 20 pM, from 1 pM to 20 pM, from 1 aM to 2 pM, from 10 aM to 2 pM, from 100 aM to 2 pM, from 1 fM to 2 pM, from 10 fM to 2 pM, from 100 fM to 2 pM, from 1 aM to 200 fM, from 10 aM to 200 fM, from 100 aM to 200 fM, from 1 fM to 200 fM, from 10 fM to 200 fM, from 1 aM to 20 fM, from 10 aM to 20 fM, from 100 aM to 20 fM, from 1 fM to 20 fM, from 1 aM to 2 fM, from 10 aM to 2 fM, from 100 aM to 2 fM, from 1 aM to 200 aM, from 10 aM to 200 aM, or from 1 aM to 20 aM. In some cases, the minimum concentration at which a single stranded target nucleic acid can be detected in a sample is in a range of from 1 aM to 100 pM. In some cases, the minimum concentration at which a single stranded target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

When a guide nucleic acid binds to a target nucleic acid or an amplicon thereof, the programmable nuclease's trans cleavage activity can be initiated, and reporters can be cleaved, resulting in the detection of fluorescence. Some methods as described herein can a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal or a presence of the signal near background indicates an absence of the target nucleic acid in the sample. The cleaving of the reporter using the programmable nuclease may cleave with an efficiency of 50% as measured by a change in a signal that is calorimetric, potentiometric, amperometric, optical (e.g., fluorescent, colorimetric, etc.), or piezo-electric, as non-limiting examples. Some methods as described herein can be a method of detecting a target nucleic acid in a sample comprising contacting the sample comprising the target nucleic acid with a guide nucleic acid targeting a target nucleic acid segment, a programmable nuclease capable of being activated when complexed with the guide nucleic acid and the target nucleic acid segment, a single stranded reporter comprising a detection moiety, wherein the reporter is capable of being cleaved by the activated programmable nuclease, thereby generating a first detectable signal, cleaving the single stranded reporter using the programmable nuclease that cleaves as measured by a change in color, and measuring the first detectable signal on the support medium. The cleaving of the single stranded reporter using the programmable nuclease may cleave with an efficiency of 50% as measured by a change in color. In some cases, the cleavage efficiency is at least 40%, 50%, 60%, 70%, 80%, 90%, or 95% as measured by a change in color. In some embodiments, the cleavage efficiency is from 40% to 95%, from 50% to 95%, from 60% to 95%, from 65% to 95%, from 75% to 95%, from 80% to 95%, from 90% to 95%, from 40% to 90%, from 50% to 90%, from 60% to 90%, from 65% to 90%, from 75% to 90%, from 80% to 90%, from 40% to 80%, from 50% to 80%, from 60% to 80%, from 65% to 80%, from 75% to 80%, from 40% to 75%, from 50% to 75%, from 60% to 75%, from 65% to 75%, from 40% to 60%, from 50% to 60%, or from 40% to 50% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target nucleic acid segment, a programmable nuclease capable of being activated when complexed with the guide nucleic acid and the target nucleic acid segment, and a single stranded reporter comprising a detection moiety, wherein the reporter is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample. In some embodiments, the first detectable signal can be detectable within from 1 to 120, from 5 to 100, from 10 to 90, from 15 to 80, from 20 to 60, or from 30 to 45 minutes of contacting the sample. In some embodiments, the first detectable signal can be detectable within from 15 minutes to 120 minutes, from 30 minutes to 120 minutes, from 45 minutes to 120 minutes, from 60 minutes to 120 minutes, from 75 minutes to 120 minutes, from 90 minutes to 120 minutes, from 105 minutes to 120 minutes, from 5 minutes to 90 minutes, from 15 minutes to 90 minutes, from 30 minutes to 90 minutes, from 45 minutes to 90 minutes, from 60 minutes to 90 minutes, from 75 minutes to 90 minutes, from 5 minutes to 75 minutes, from 15 minutes to 75 minutes, from 30 minutes to 75 minutes, from 45 minutes to 75 minutes, from 60 minutes to 75 minutes, from 5 minutes to 60 minutes, from 15 minutes to 60 minutes, from 30 minutes to 60 minutes, from 45 minutes to 60 minutes, from 5 minutes to 45 minutes, from 15 minutes to 45 minutes, from 30 minutes to 45 minutes, from 5 minutes to 30 minutes, from 15 minutes to 30 minutes, or from 5 minutes to 15 minutes.

In some cases, the devices, systems, fluidic devices, kits, and methods described herein detect a target single-stranded nucleic acid in a sample where the sample is contacted with the reagents for a predetermined length of time sufficient for the trans cleavage to occur or cleavage reaction to reach completion. In some cases, the devices, systems, fluidic devices, kits, and methods described herein detect a target single-stranded nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes. In some cases, the sample is contacted with the reagents for from 5 minutes to 120 minutes, from 5 minutes to 100 minutes, from 10 minutes to 90 minutes, from 15 minutes to 45 minutes, or from 20 minutes to 35 minutes. In some cases, the sample is contacted with the reagents for from 15 minutes to 120 minutes, from 30 minutes to 120 minutes, from 45 minutes to 120 minutes, from 60 minutes to 120 minutes, from 75 minutes to 120 minutes, from 90 minutes to 120 minutes, from 105 minutes to 120 minutes, from 5 minutes to 90 minutes, from 15 minutes to 90 minutes, from 30 minutes to 90 minutes, from 45 minutes to 90 minutes, from 60 minutes to 90 minutes, from 75 minutes to 90 minutes, from 5 minutes to 75 minutes, from 15 minutes to 75 minutes, from 30 minutes to 75 minutes, from 45 minutes to 75 minutes, from 60 minutes to 75 minutes, from 5 minutes to 60 minutes, from 15 minutes to 60 minutes, from 30 minutes to 60 minutes, from 45 minutes to 60 minutes, from 5 minutes to 45 minutes, from 15 minutes to 45 minutes, from 30 minutes to 45 minutes, from 5 minutes to 30 minutes, from 15 minutes to 30 minutes, or from 5 minutes to 15 minutes. In some cases, the devices, systems, fluidic devices, kits, and methods described herein can detect a target nucleic acid in a sample in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some cases, the devices, systems, fluidic devices, kits, and methods described herein can detect a target nucleic acid in a sample in from 5 minutes to 10 hours, from 10 minutes to 8 hours, from 15 minutes to 6 hours, from 20 minutes to 5 hours, from 30 minutes to 10 hours, from 1 hour to 10 hours, from 2 hours to 10 hours, from 4 hours to 10 hours, from 5 hours to 10 hours, from 6 hours to 10 hours, from 8 hours to 10 hours, from 30 minutes to 8 hours, from 1 hour to 8 hours, from 2 hours to 8 hours, from 4 hours to 8 hours, from 5 hours to 8 hours, from 6 hours to 8 hours, from 30 minutes to 6 hours, from 1 hour to 6 hours, from 2 hours to 6 hours, from 4 hours to 6 hours, from 5 hours to 6 hours, from 30 minutes to 5 hours, from 1 hours to 5 hours, from 2 hours to 5 hours, from 4 hours to 5 hours, from 30 minutes to 4 hours, from 1 hour to 4 hours, from 2 hours to 4 hours, from 30 minutes to 2 hours, from 1 hour to 2 hours, from 5 minutes to 1 hour, from 15 minutes to 1 hour, from 30 minutes to 1 hour, or from 45 minutes to 1 hour. In some embodiments, the devices, systems, fluidic devices, kits, and methods described herein can perform up to 100, 200, 300, 400, 500, 600, 700, 800, 900, 1000, 1100, 1200, 1300, 1400, or 1500 tests in 8 hours.

A number of detection devices and methods are consistent with methods disclosed herein. For example, any device that can measure or detect a calorimetric, potentiometric, amperometric, optical (e.g., fluorescent, colorimetric, etc.), or piezo-electric signal. Often a calorimetric signal is heat produced after cleavage of the reporters. Sometimes, a calorimetric signal is heat absorbed after cleavage of the reporters. A potentiometric signal, for example, is electrical potential produced after cleavage of the reporters. An amperometric signal can be movement of electrons produced after the cleavage of reporter. Often, the signal is an optical signal, such as a colorimetric signal or a fluorescence signal. An optical signal is, for example, a light output produced after the cleavage of the reporters. Sometimes, an optical signal is a change in light absorbance between before and after the cleavage of reporters. Often, a piezo-electric signal is a change in mass between before and after the cleavage of the reporter. Sometimes, the reporter is protein-nucleic acid. Often, the protein-nucleic acid is an enzyme-nucleic acid.

The results from the detection region from a completed assay can be detected and analyzed in various ways. In some cases a detection signal is visible to the human eye and can be read by the user. For example, the results from the detection region from a completed assay can be detected and analyzed by a glucometer. In some cases, a detection signal is visualized by an imaging device or other device depending on the type of signal. Often, the imaging device is a digital camera, such a digital camera on a mobile device. The mobile device may have a software program or a mobile application that can capture an image of the sample vessel, identify the assay being performed, detect the detection signal, provide image properties of the detection signal, analyze the image properties of the detection spot, and provide a result. Alternatively or in combination, the imaging device can capture fluorescence, ultraviolet (UV), infrared (IR), or visible wavelength signals. The imaging device may have an excitation source to provide the excitation energy and captures the emitted signals. In some cases, the excitation source can be a camera flash and optionally a filter. In some cases, the imaging device is used together with an imaging box that is placed over the support medium to create a dark room to improve imaging. The imaging box can be a cardboard box that the imaging device can fit into before imaging. In some instances, the imaging box has optical lenses, mirrors, filters, or other optical elements to aid in generating a more focused excitation signal or to capture a more focused emission signal. Often, the imaging box and the imaging device are small, handheld, and portable to facilitate the transport and use of the assay in remote or low resource settings.

The assay described herein can be visualized and analyzed by a mobile application (app) or a software program. Using the graphic user interface (GUI) of the app or program, an individual can take an image of the support medium, including the detection region, barcode, reference color scale, and fiduciary markers on the housing, using a camera on a mobile device. A microplate may include one or more barcodes or QR codes disposed on a side opposite to the one in which the samples are placed. A mobile device may be able to scan these barcodes or QR codes from underneath the device. A tube-based array may include one or more barcodes on a side of a tube, or on an apparatus holding the one or more tubes. The program or app reads the barcode or identifiable label for the test type, locate the fiduciary marker to orient the sample, and read the detectable signals, compare against the reference color grid, and determine the presence or absence of the target nucleic acid, which indicates the presence of the gene, virus, or the agent responsible for the disease, cancer, or genetic disorder. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, and/or clean-up of an environment.

An example detection solution is a DETECTR solution. An example DETECTR solution may include a Guide RNA, a Cas12 protein, a reporter, a Tris HCl solution, a KOAc buffer solution, 1M MgOAc, glycerol, and tween. Detection buffers may comprise HEPES, MES, TCEP, EGTA, Tween 20, KCl, MgCl², glycerol, or any combination thereof. In some instances, a detection buffer may comprise Tris-HCl pH 8.8, VLB, EGTA, CH3COOH, TCEP, IsoAmp, (NH4)2SO4, KCl, MgSO4, Tween20, KOAc, MgOAc, BSA, or any combination thereof.

### Programmable Nuclease

Disclosed herein are programmable nucleases and uses thereof, e.g., detection and editing of target nucleic acids. In some instances, programmable nucleases comprise a Type V CRISPR/Cas protein. In some instances, Type V CRISPR/Cas proteins comprise nucleic acid cleavage activity. In some instances, Type V CRISPR/Cas proteins cleave or nick single-stranded nucleic acids, double, stranded nucleic acids, or a combination thereof. In some cases, Type V CRISPR/Cas proteins cleave single-stranded nucleic acids. In some cases, Type V CRISPR/Cas proteins cleave double-stranded nucleic acids. In some cases, Type V CRISPR/Cas proteins nick double-stranded nucleic acids. Typically, guide nucleic acids of Type V CRISPR/Cas proteins hybridize to ssDNA or dsDNA. However, the trans cleavage activity of Type V CRISPR/Cas protein is typically directed towards ssDNA. In some cases, the Type V CRISPR/Cas protein comprises a catalytically inactive nuclease domain. A catalytically inactive domain of a Type V CRISPR/Cas protein may comprise at least 1, at least 2, at least 3, at least 4, or at least 5 mutations relative to a wild type nuclease domain of the Type V CRISPR/Cas protein. Said mutations may be present within a cleaving or active site of the nuclease. The Type V CRISPR/Cas protein may be a Cas14 protein. The Cas 14 protein may be a Cas14a.1 protein. The Cas14a.1 protein may be represented by SEQ ID NO: 1, presented in Table 1. The Cas14 protein may comprise an amino acid sequence that is at least 70%, at least 75%, at least 80%, at least 85%, at least 90%, at least 95%, at least 98%, at least 99%, or 100% identical to SEQ ID NO: 1. The Cas14 protein may consist of an amino acid sequence that is at least 70%, at least 75%, at least 80%, at least 85%, at least 90%, at least 95%, at least 98%, at least 99%, or 100% identical to SEQ ID NO: 1. The Cas14 protein may comprise at least about 50, at least about 100, at least about 150, at least about 200, at least about 250, at least about 300, at least about 350, at least about 400, at least about 450, at least about 500 consecutive amino acids of SEQ ID NO: 1.

TABLE 1
Exemplary Protein Sequences

| SEQ ID | SEQUENCE |
|---|---|
| SEQ ID: 1 | MADLSQFTHKYQVPKTLRFELIPQGKTLENLSAYGMVADDKQRSENYKKLK |
| | PVIDRIYKYFIEESLKNTNLDWNPLYEAIREYRKEKTTATITNLKEQQDIC |
| | RRAIASRFEGKVPDKGDKSVKDFNKKQSKLFKELFGKELFTDSVLEQLPGV |
| | SLSDEDKALLKSFDKFTTYFVGFYDNRKNVFSSDDISTGIPHRLVQENFPK |
| | FIDNCDDYKRLVLVAPELKEKLEKAAEATKIFEDVSLDEIFSIKFYNRLLQ |
| | QNQIDQFNQLLGGIAGAPGTPKIQGLNETLNLSMQQDKTLEQKLKSVPHRF |
| | SPLYKQILSDRSSLSFIPESFSCDAEVLLAVQEYLDNLKTEHVIEDLKEVF |
| | NRLTTLDLKHIYVNSTKVTAFSQALFGDWNLCREQLRVYKMSNGNEKITKK |
| | ALGELESWLKNSDIAFTELQEALADEALPAKVNLKVQEAISGLNEQMAKSL |
| | PKELKIPEEKEELKALLDAIQEVYHTLEWFIVSDDVETDTDFYVPLKETLQ |
| | IIQPIIPLYNKVRNFATQKPYSVEKFKLNFANPTLADGWDENKEQQNCAVL |
| | FQKGNNYYLGILNPKNKPDFDNVDTEKQGNCYQKMVYKQFPDFSKMMPKCT |
| | TQLKEVKQHFEGKDSDYILNNKNFIKPLTITREVYDLNNVLYDGKKKFQTD |
| | YLRKTKDEDGYYTALHTWTDFAKKFVASYKSTSTYDTSTILPPEKYEKLNE |
| | FYGALDNLFYQIKFENIPEEIIDTYVEDGKLFLFQIYNKDFAAGATGAPNL |
| | HTIYWKAVFDPENVKDVVVKLNGQAELFYRPKSNMDVIRHKVGEKLVNRTL |
| | KDGSILTDELHKELYLYANGSLKKGLSEDAKIILDKNLAVIYDVHHEIVKD |
| | RRFTTDKFFFHVPLTLNYKCDKNPVKFNAEVQEYLKENPDTYVTGTDRGER |
| | NLTYAVVIDPKGRTVEQKSFNVTNGFDYHGKLDQREKERVKARQAWTAVGK |
| | IKELKQGYLSLVVHEISKMMVRYQAVVVLENLNVGFKRVRSGIAEKAVYQQ |
| | FEKMLINKLNYLMFKDAGGTEPGSVLNAYQLTDRFESFAKMGLQTGFLFYI |
| | PAAFTSKIDPATGFVDPFRWGAIKTLADKREFLSGFESLKFDSTTGNFILH |
| | FDVSKNKNFQKKLEGFVPDWDIIIEANKMKTGKGATYIAGKRIEFVRDNNS |
| | QGHYEDYLPCNALAETLRQCDIPYEEGKDILPLILEKNDSKLLHSVFKVVR |
| | LTLQMRNSNAETGEDYISSPVEDVSGSCFDSRMENEKLPKDADANGAYHIA |
| | LKGMLALERLRKDEKMAISNNDWLNYIQEKRA* |
| SEQ ID: 2 | MAGKKKDKDVINKTLSVRIIRPRYSDDIEKEISDEKAKRKQDGKTGELDRA |
| | FFSELKSRNPDIITNDELFPLFTEIQKNLTEIYNKSISLLYMKLIVEEEGG |
| | STASALSAGPYKECKARFNSYISLGLRQKIQSNFRRKELKGFQVSLPTAKS |
| | DRFPIPFCHQVENGKGGFKVYETGDDFIFEVPLTKYTATNKKSTSGKNYTK |
| | VQLNNPPVPMNVPLLLSTMRRRQTKKGMQWNKDEGTNAELRRVMSGEYKVS |
| | YAEIIRRTRFGKHDDWFVNFSIKFKNKTDELNQNVRGGIDIGVSNPLVCAV |
| | TNGLDRYIVANNDIMAFNERAMARRRTLLRKNRFKRSGHGAKNKLEPITVL |
| | TEKNERFRKSILQRWAREVAEFFKRTSASVVNMEDLSGITEREDFFSTKLR |
| | TTWNYRLMQTTIENKLKEYGIAVNYISPKYTSQTCHSCGKRNDYFTFSYRS |
| | ENNYPPFECKECNKVKCNADFNAAKNIALKVVL |
| SEQ ID: 3 | MAKNTITKTLKLRIVRPYNSAEVEKIVADEKNNREKIALEKNKDKVKEACS |
| | KHLKVAAYCTTQVERNACLFCKARKLDDKFYQKLRGQFPDAVFWQEISEIF |
| | RQLQKQAAEIYNQSLIELYYEIFIKGKGIANASSVEHYLSDVCYTRAAELF |
| | KNAAIASGLRSKIKSNFRLKELKNMKSGLPTTKSDNFPIPLVKQKGGQYTG |
| | FEISNHNSDFIIKIPFGRWQVKKEIDKYRPWEKFDFEQVQKSPKPISLLLS |
| | TQRRKRNKGWSKDEGTEAEIKKVMNGDYQTSYIEVKRGSKIGEKSAWMLNL |
| | SIDVPKIDKGVDPSIIGGIDVGVKSPLVCAINNAFSRYSISDNDLFHFNKK |
| | MFARRRILLKKNRHKRAGHGAKNKLKPITILTEKSERFRKKLIERWACEIA |
| | DFFIKNKVGTVQMENLESMKRKEDSYFNIRLRGFWPYAEMQNKIEFKLKQY |
| | GIEIRKVAPNNTSKTCSKCGHLNNYFNFEYRKKNICFPHFKCEKCNFKENA |
| | DYNAALNISNPKLKSTKEEP |
| SEQ ID: 4 | MATLVSFTKQYQVQKTLRFELIPQGKTQANIDAKGFINDDLKRDENYMKVK |
| | GVIDELHKNFIEQTLVNVDYDWRSLATAIKNYRKDRSDTNKKNLEKTQEAA |
| | RKEIIAWFEGKRGNSAFKNNQKSFYGKLFKKELFSEILRSDDLEYDEETQD |
| | AIACFDKFTTYFVGFHENRKNMYSTEAKSTSVAYRVVNENFSKFLSNCEAF |
| | SVLEAVCPNVLVEAEQELHLHKAFSDLKLSDVFKVEAYNKYLSQTGIDYYN |
| | QIIGGISSAEGVRKIRGVNEVVNNAIQQNDELKVALRNKQFTMVQLFKQIL |
| | SDRSTLSFVSEQFTSDQEVITVVKQFNDDIVNNKVLAVVKTLFENFNSYDL |
| | EKIYINSKELASVSNALLKDWSKIRNAVLENKIIELGANPPKTKISAVEKE |
| | VKNKDFSIAELASYNDKYLDKEGNDKEICSIANVVLEAVGALEIMLAESLP |
| | ADLKTLENKNKVKGILDAYENLLHLLNYFKVSAVNDVDLAFYGAFEKVYVD |
| | ISGVMPLYNKVRNYATKKPYSVEKFKLNFAMPTLADGWDKNKERDNGSIIL |
| | LKDGQYYLGVMNPQNKPVIDNAVCNDAKGYQKMVYKMFPEISKMVTKCSTQ |
| | LNAVKAHFEDNTNDFVLDDTDKFISDLTITKEIYDLNNVLYDGKKKFQIDY |
| | LRNTGDFAGYHKALETWIDFVKEFLSKYRSTATYDLTTLLPTNYYEKLDVF |
| | YSDVNNLCYKIDYENTSVEQVNEWVEEGNLYLFKIYNKDFATGSTGKPNLH |
| | TMYWNAVFAEENLHDVVVKLNGGAELFYRPKSNMPKVEHRVGEKLVNRKNV |
| | NGEPIADSVHKEIYAYANGKISKSELSENAQEELPLAIIKDVKHNITKDKR |
| | YLSDKYFFHVPITLNYKANGNPSAFNTKVQAFLKNNPDVNIIGIDRGERNL |
| | LYVVVIDQQGNIIDKKQVSYNKVNGYDYYEKLNQREKERTEARQSWGAVGK |
| | TKELKEGYLSLVVREIADMMVKYNAIVVMENLNAGFKRVRGGIAEKAVYQK |
| | FEKMLIDKLNYLVFKDVEAKEAGGVLNAYQLTDKFDSFEKMGNQSGFLFYV |
| | PAAYTSKIDPVTGFANVFSTKHITNTEAKKEFICSFNSLRYDEAKDKFVLE |
| | CDLNKFKIVANSHIKNWKFIIGGKRIVYNSKNKTYMEKYPCEDLKATLNAS |
| | GIDFSSSEIINLLKNVPANREYGKLFDETYWAIMNTLQMRNSNALTGEDYI |
| | ISAVADDNEKVFDSRTCGAELPKDADANGAYHIALKGLYLLQRIDISEEGE |
| | KVDLSIKNEEWFKFVQQKEYAR* |
| SEQ ID: 5 | MCMKITKIDGISHKKYKEKGKLIKNNDTAKDIIEERFNDIEKKTKELFQKT |
| | LDFYVKNYEKCKEQNKERREKAKNYFSKVKILVDNKKITTCNENTEKMEIE |
| | DFNEYDVRSGKYFNVLNKILNGENYTEEDLEVFENDLQKRTGRIKSIKNSL |
| | EENKAHFKKESINNNIIYDRVKGNNKKSLFYEYYRISSKHQEYVNNIFEAF |
| | DKLYSNSHEAMNNLFSEITKDSKDRNIRKIREAYHEILNKNKTEFGEELYK |
| | KIQDNRNNFDKLLEIEPEIKELTKSQIFYKYYIDKVNLDETSIKHCFCHLV |
| | EIEVNQLLKNYVYSKRNTNKEKLENTFEYCKLKNLIKNKLVNKLNNYIRNC |
| | GKYNAYISNNDVVVNSEKISEIRTKEAFLRSIIGVSSSAYFSLRNILNTDN |
| | TQDITNKVDKEVDKLYQENKKIELEERLKLFFGNYFDINNQQEIKVFLMNI |
| | DKIISSIRHEIIHFKMETNAQNIFDFNNVNLGNTAKNIFSNEINEEKIKFK |
| | IFKQLNSANVFDYLSNKDITEYMDKVVFSFTNRNVSFVPSFTKIYNRVQDL |
| | ANSLEIKKWKIPDKSEGKDAQIYLLKNIYYGKFLDEFLNEENGIFISIKDK |
| | IIELNRNQNKRTGFYKLEKFEKIEETNPKKYLEIIQSLYMINIEEIDSEGK |
| | NFFLDFIQKIFLKGFFEFIKNNYNYLLELKKIQDKKNIFDSEMSEYIAGEK |
| | TLEDIGEINEIIQDIKITEIDKILNQTDKINCFYLLLKLLNYKEITELKGN |
| | LEKYQILSKTNVYEKELMLLNIVNLDNNKVKIENFKILAEEIGKFIEKINI |
| | EEINKNKKIKTFEELRNFEKGENTGEYYNIYSDDKNIKNIRNLYNIKKYGM |
| | LDLLEKISEKTNYCIKKKDLEEYSELRKQLEDEKTNFYKIQEYLHSKYQQK |
| | PKKILLKNNKNDYEKYKKSIENIEKYVHLKNKIEFNELNLLQSLLLKILHR |
| | LVGFTSIWERDLRFRLIGEFPDELDVEDIFDHRKRYKGTGKGICKKYDRFI |
| | NTHTEYKNNNKMENVKFADNNPVRNYIAHFNYLPNPKYSILKMMEKLRKLL |
| | DYDRKLKNAVMKSIKDILEEYGFKAEFIINSDKEIILNLVKSVEIIHLGKE |
| | DLKSRRNSEDLCKLVKAMLEYSK* |
| SEQ ID: 6 | MEDKQFLERYKEFIGLNSLSKTLRNSLIPVGSTLKHIQEYGILEEDSLRAQ |
| | KREELKGIMDDYYRNYIEMHLRDVHDIDWNELFEALTEVKKNQTDDAKKRL |
| | EKIQEKKRKEIYQYLSDDAVFSEMFKEKMISGILPDFIRCNEGYSEEEKEE |
| | KLKTVALFHRFTSSFNDFFLNRKNVFTKEAIVTAIGYRVVHENAEIFLENM |
| | VAFQNIQKSAESQISIIERKNEHYFMEWKLSHIFTADYYMMLMTQKAIEHY |
| | NEMCGVVNQQMREYCQKEKKNWNLYRMKRLHKQILSNASTSFKIPEKYEND |
| | AEVYESVNSFLQNVMEKTVMERIAVLKNSTDNFDLSKIYITAPYYEKISNY |
| | LCGSWNTITDCLTHYYEQQIAGKGARKDQKVKAAVKADKWKSLSEIEQLLK |
| | EYARAEEVKRKPEEYIAEIENIVSLKEAHLLEYHPEVNLIENEKYATEIKD |
| | VLDNYMELFHWMKWFYIEEAVEKEVNFYGELDDLYEEIKDIVPLYNKVRNY |
| | VTQKPYSDTKIKLNFGTPTLANGWSKSKEYDYNAILLQKDGKYYMGIFNPI |
| | QKPEKEIIEGHSQPLEGNEYKKMVYYYLPSANKMLPKVLLSKKGMEIYQPS |
| | EYIINGYKERRHIKSEEKFDLQFCHDLIDYFKSGIERNSDWKVFGFDFSDT |
| | DTYQDISGFYREVEDQGYKIDWTYIKEADIDRLNEEGKLYLFQIYNKDFSE |
| | KSTGRENLHTMYLKNLFSEENVREQVLKLNGEAEIFFRKSSVKKPIIHKKG |
| | TMLVNRTYMEEVNGNSVRRNIPEKEYQEIYNYKNHRLKGELSTEAKKYLEK |
| | AVCHETKKDIVKDYRYSVDKFFIHLPITINYRASGKETLNSVAQRYIAHQN |
| | DMHVIGIDRGERNLIYVSVINMQGEIKEQKSFNIINEFNYKEKLKEREQSR |
| | GAARRNWKEIGQIKDLKEGYLSGVIHEIAKMMIKYHAIIAMEDLNYGFKRG |
| | RFKVERQVYQKFENMLIQKLNYLVFKDRPADEDGGVLRGYQLAYIPDSVKK |
| | MGRQCGMIFYVPAAFTSKIDPTTGFVDIFKHKVYTTEQAKREFILSFDEIC |
| | YDVERQLFRFTFDYANFVTQNVTLARNNWTIYTNGTRAQKEFGNGRMRDKE |
| | DYNPKDKMVELLESEGIEFKSGKNLLPALKKVSNAKVFEELQKIVRFTVQL |
| | RNSKSEENDVDYDHVISPVLNEEGNFFDSSKYKNKEEKKESLLPVDADANG |
| | AYCIALKGLYIMQAIQKNWSEEKALSPDVLRLNNNDWFDYIQNKRYR* |
| SEQ ID: 7 | MEEKKMSKIEKFIGKYKISKTLRFRAVPVGKTQDNIEKKGILEKDKKRSED |
| | YEKVKAYLDSLHRDFIENTLKKVKLNELNEYACLFFSGTKDDGDKKKMEKL |
| | EEKMRKTISNEFCNDEMYKKIFSEKILSENNEEDVSDIVSSYKGFFTSLNG |
| | YVNNRKNLYVSDAKPTSIAYRCINENLPKFLRNVECYKKVVQVIPKEQTEY |
| | MSNNLNLSPYRTEDCFNTDFFEFCLSQGGTDLYNTFTGGYSKKDGTKVQGI |
| | NEIVNLYNQKNKKDKEKYKLPQFTPLFKQILSDRDTKSFSIEKLENIYEVV |
| | ELVKKSYSDEMFDDIETVFSNLNYYDASGIYVKNGPAITHISMNLTKDWAT |
| | IRNNWNYEYDEKHSTKKNKNIEKYEDTRNTMYKKIDSFTLEYISRLVGKDI |
| | DELVKYFENEVANFVMDIKKTYSKLTPLFDRCQKENFDISEDEVNDIKGYL |
| | DNVKLLESFMKSFTINGKENNIDYVFYGKFTDDYDKLHEFDHIYNKVRNYI |
| | TTSRKPYKLDKYKLYFDNPQLLGGWDINKEKDYRTVMLTKDGKYYFAIIDK |
| | GEHPFDNIPKDYFDNNGYYKKIIYRQIPNAAKYLSSKQIVPQNPPEEVKRI |
| | LDKKKADSKSLTEEEKNIFIDYIKSDFLKNYKLLFDKNNNPYFNFAFRESS |
| | TYESLNEFFEDVERQAYSVRYENLPADYIDNLVNEGKIYLFEIYSKDFSEY |
| | SKGTNNLHTMYFKALFDNDNLKNTVFKLSGNAELFIRPASIKKDELVIHPK |
| | NQLLQNKNPLNPKKQSIFDYDLVKDKRFFENQYMLHISIEINKNERDAKKI |
| | KNINEMVRKELKDSDDNYIIGIDRGERNLLYVCVINSAGKIVEQMSLNEII |
| | NEYNGIKHTVDYQGLLDKCEKERNAQRQSWKSIENIKELKDGYISQVVHKL |
| | CQLVEKYDAIIAMENLNGGFKRGRTKFEKQVYQKFENKLINKMEYMADKKR |
| | KTTENGGILRGYQLTNGCINNSYQNGFIFYVPAWLTSKIDPTTGFVDLLKP |
| | KYTNVEEAHLWINKFNSITYDKKLDMFAFNINYSQFPRADIDYRKTWTFYT |
| | NGYRIETFRNSEKNNEFDWKEVHLTSVIKKLLEEYQINYISGKNIIDDLIQ |
| | IKDKPFWNSFIKYIRLTLQMRNSITGRTDVDYIISPVINNEGTFYDSRKDL |
| | DEITLPQDADANGAYNIARKALWIIEKLKESPDEELNKVKLAITQREWLEY |
| | AQINI* |
| SEQ ID: 8 | MEKIKKPSNRNSIPSIIISDYDANKIKEIKVKYLKLARLDKITIQDMEIVD |
| | NIVEFKKILLNGVEHTIIDNQKIEFDNYEITGCIKPSNKRRDGRISQAKYV |
| | VTITDKYLRENEKEKRFKSTERELPNNTLLSRYKQISGFDTLTSKDIYKIK |
| | RYIDFKNEMLFYFQFIEEFFNPLLPKGKNFYDLNIEQNKDKVAKFIVYRLN |
| | DDFKNKSLNSYITDTCMIINDFKKIQKILSDFRHALAHFDFDFIQKFFDDQ |
| | LDKNKFDINTISLIETLLDQKEEKNYQEKNNYIDDNDILTIFDEKGSKFSK |
| | LHNFYTKISQKKPAFNKLINSFLSQDGVPNEEFKSYLVTKKLDFFEDIHSN |
| | KEYKKIYIQHKNLVIKKQKEESQEKPDGQKLKNYNDELQKLKDEMNTITKQ |
| | NSLNRLEVKLRLAFGFIANEYNYNFKNFNDEFTNDVKNEQKIKAFKNSSNE |
| | KLKEYFESTFIEKRFFHFSVNFFNKKTKKEETKQKNIFNSIENETLEELVK |
| | ESPLLQIITLLYLFIPRELQGEFVGFILKIYHHTKNITSDTKEDEISIEDA |
| | QNSFSLKFKILAKNLRGLQLFHYSLSHNTLYNNKQCFFYEKGNRWQSVYKS |
| | FQTSHNQDEFDTHLVTPVIKYYINLNKLMGDFETYALLKYADKNSTTVKLS |
| | DITSRDDLKYNGHYNFATLLFKTFGIDTNYKQNKVSIQNIKKTRNNLAHQN |
| | IENMLKAFENSEIFAQREEIVNYLQTEHRMQEVLHYNPINDFTMKTVQYLK |
| | SLSVHSQKEGKIADIHKKESLVPNDYYLIYKLKAIELLKQKVIEVIGESED |
| | EKKIKNAIAKEEQIKKGNN |
| | MEKSLNDFIGLYSVSKTLRFELKPVSETLENIKKFHFLEEDKKKANDYKDV |
| | KKIIDNYHKYFIDDVLKNASFNWKKLEEAIREYNKNKSDDSALVAEQKKLG |
| | DAILKLFTSDKRYKALTAATPKELFESILPDWFGEQCNQDLNKAALKTFQK |
| | FTSYFTGFQENRKNVYSAEAIPTAVPYRIVNDNFPKFLQNVLIFKTIQEKC |
| | PQIIDEVEKELSSYLGKEKLAGIFTLESFNKYLGQGGKENQRGIDFYNQII |
| | GGVVEKEGGINLRGVNQFLNLYWQQHPDFTKEDRRIKMVPLYKQILSDRSS |
| | LSFKIESIENDEELKNALLECADKLELKNDEKKSIFEEVCDLFSSVKNLDL |
| | SGIYINRKDINSVSRILTGDWSWLQSRMNVYAEEKFTTKAEKARWQKSLDD |
| | EGENKSKGFYSLTDLNEVLEYSSENVAETDIRITDYFEHRCRYYVDKETEM |
| | FVQGSELVALSLQEMCDDILKKRKAMNTVLENLSSENKLREKTDDVAVIKE |
| | YLDAVQELLHRIKPLKVNGVGDSTFYSVYDSIYSALSEVISVYNKTRNYIT |
| | KKAASPEKYKLNFDNPTLADGWDLNKEQANTSVILRKDGMFYLGIMNPKNK |
| | PKFAEKYDCGNESCYEKMIYKQFDATKQIPKCSTQKKEVQKYFLSGATEPY |
| | ILNDKKSFKSELIITKDIWFMNNHVWDGEKFVPKRDNETRPKKFQIGYFKQ |
| | TGDFDGYKNALSNWISFCKNFLQSYLSATVYDYNFKNSEEYEGLDEFYNYL |
| | NATCYKLNFINIPETEINKMVSEGKLYLFQIYNKDFASGSTGMPNMHTLYW |
| | KNLFSDENLKNVCLKLNGEAELFYRPAGIKEPVIHKEGSYLVNRTTEDGES |
| | IPEKIYFEIYKNANGKLEKLSDEAQNYISNHEVVIKKAGHEIIKDRHYTEP |
| | KFLFHVPLTINFKASGNSYSINENVRKFLKNNPDVNIIGLDRGERHLIYLS |
| | LINQKGEIIKQFTFNEVERNKNGRTIKVNYHEKLDQREKERDAARKSWQAI |
| | GKIAELKEGYLSAVIHQLTKLMVEYNAVVVMEDLNFGFKRGRFHVEKQVYQ |
| | KFEHILIDKSNYLVFKDRGLNEPGGVLNGYQIAGQFESFQKLGKQSGMLFY |
| | VPAGYTSKIDPKTGFVSMMNFKDLTNVHKKRDFFSKFDNIHYDEANGSFVF |
| | TFDYKKFDGKAKEEMKLTKWSVYSRDKRIVYFAKTKSYEDVLPTEKLQKIF |
| | ESNGIDYKSGNNIQDSVMAIGADLKEGAKPSKEISDFWDGLLSNFKLILQM |
| | RNSNARTGEDYIISPVMADDGTFFDSREEFKKGEDAKLPLDADANGAYHIA |
| | LKGLSLINKINLSKDEELKKFDMKISNADWFKFAQEKNYAK* |
| SEQ ID: 9 | MENYGGFTGLYPLQKTLKFELRPQGRTMEHLVSSNFFEEDRDRAEKYKIVK |
| | KVIDNYHKDFINECLSKRSFDWTPLMKTSEKYYASKEKNGKKKQDLDQKII |
| | PTIENLSEKDRKELELEQKRMRKEIVSVFKEDKRFKYLFSEKLFSILLKDE |
| | DYSKEKLTEKEILALKSFNKFSGYFIGLHKNRANFYSEGDESTAIAYRIVN |
| | ENFPKFLSNLKKYREVCEKYPEIIQDAEQSLAGLNIKMDDIFPMENFNKVM |
| | TQDGIDLYNLAIGGKAQALGEKQKGLNEFLNEVNQSYKKGNDRIRMTPLFK |
| | QILSERTSYSYILDAFDDNSQLITSINGFFTEVEKDKEGNTFDRAVGLIAS |
| | YMKYDLSRVYIRKADLNKVSMEIFGSWERLGGLLRIFKSELYGDVNAEKTS |
| | KKVDKWLNSGEFSLSDVINAIAGSKSAETFDEYILKMRVARGEIDNALEKI |
| | KCINGNFSEDENSKMIIKAILDSVQRLFHLFSSFQVRADFSQDGDFYAEYN |
| | EIYEKLFAIVPLYNRVRNYLTKNNLSMKKIKLNFKNPALANGWDLNKEYDN |
| | TAVIFLREGKYYLGIMNPSKKKNIKFEEGSGTGPFYKKMAYKLLPDPNKML |
| | PKVFFAKKNINYYNPSDEIVKGYKAGKYKKGENFDIDFCHKLIDFFKESIQ |
| | KNEDWRAFNYLFSATESYKDISDFYSEVEDQGYRMYFLNVPVANIDEYVEK |
| | GDLFLFQIYNKDFASGAKGNKDMHTIYWNAAFSDENLRNVVVKLNGEAELF |
| | YRDKSIIEPICHKKGEMLVNRTCFDKTPVPDKIHKELFDYHNGRAKTLSIE |
| | AKGYLDRVGVFQASYEIIKDRRYSENKMYFHVPLKLNFKADGKKNLNKMVI |
| | EKFLSDKDVHIIGIDRGERNLLYYSVIDRRGNIIDQDSLNIIDGFDYQKKL |
| | GQREIERREARQSWNSIGKIKDLKEGYLSKAVHKVSKMVLEYNAIVVLEDL |
| | NFGFKRGRFKVEKQVYQKFEKMLIDKLNYLVFKEVLDSRDAGGVLNAYQLT |
| | TQLESFNKLGKQSGILFYVPAAYTSKIDPTTGFVSLFNTSRIESDSEKKDF |
| | LSGFDSIVYSAKDGGIFAFKFDYRNRNFQREKTDHKNIWTVYTNGDRIKYK |
| | GRMKGYEITSPTKRIKDVLSSSGIRYDDGQELRDSIIQSGNKVLINEVYNS |
| | FIDTLQMRNSDGEQDYIISPVKNRNGEFFRTDPDRRELPVDADANGAYHIA |
| | LRGELLMQKIAEDFDPKSDKFTMPKMEHKDWFEFMQTRGD* |
| SEQ ID: 10 | MEVQKTVMKTLSLRILRPLYSQEIEKEIKEEKERRKQAGGTGELDGGFYKK |
| | LEKKHSEMFSFDRLNLLLNQLQREIAKVYNHAISELYIATIAQGNKSNKHY |
| | ISSIVYNRAYGYFYNAYIALGICSKVEANFRSNELLTQQSALPTAKSDNFP |
| | IVLHKQKGAEGEDGGFRISTEGSDLIFEIPIPFYEYNGENRKEPYKWVKKG |
| | GQKPVLKLILSTFRRQRNKGWAKDEGTDAEIRKVTEGKYQVSQIEINRGKK |
| | LGEHQKWFANFSIEQPIYERKPNRSIVGGLDVGIRSPLVCAINNSFSRYSV |
| | DSNDVFKFSKQVFAFRRRLLSKNSLKRKGHGAAHKLEPITEMTEKNDKFRK |
| | KIIERWAKEVTNFFVKNQVGIVQIEDLSTMKDREDHFFNQYLRGFWPYYQM |
| | QTLIENKLKEYGIEVKRVQAKYTSQLCSNPNCRYWNNYFNFEYRKVNKFPK |
| | FKCEKCNLEISADYNAARNLSTPDIEKFVAKATKGINLPEK* |
| SEQ ID: 11 | MIIHNCYTGGSFMKKTDSFTNCYSLSKTLRFKLTPTGATQSNFDLNKMLDE |
| | DKKRAENYSKAKSIIDKYHRFFIDKVLSSVTENKAFDSFLEDVRAYAELYY |
| | RSNKDDSDKASMKTLESKMRKFIALALQSDEGFKDLFGQNLIKKTLPEFLE |
| | SDTDKEIIAEFDGFSTYFTGFFNNRKNMYSADDQPTAISYRCINDNLPKFL |
| | DNVRTFKNSDVASILNDNLKILNEDFDGIYGTSAEDVFNVDYFPFVLSQKG |
| | IEAYNSILGGYTNSDGSKIKGLNEYINLYNQKNENIEIRIPKMKQLFKQIL |
| | SERESVSFIPEKFDSDDDVLSSnYDYYLERDGGKVLSIEKTVEKIEKLFSA |
| | VTDYSTDGIFVKNAAELTAVCSGAFGYWGTVQNAWNNEYDALNGYKETEKY |
| | IDKRKKAYKSIESFSLADIQKYADVSESSETNAEVTEWLRNEIKEKCNLAV |
| | QGYESSKDLISKPYTESKKLFNNDNAVELIKNALDSVKELENVLRLLLGTG |
| | KEESKDENFYGEFLPCYERICEVDSLYDKVRNYMTQKLYKTDKIKLNFQNP |
| | QFLGGWDRNKEADYSAVLLRRNSLYYIAIMPSGYKRVFEKIPAPKADETVY |
| | EKVIYKLLPGPNKMLPKVFFSKKGIETFNPPKEILEKYELGTHKTGDGFNL |
| | DDCHALIDYFKSALDVHSDWSNFGFRFSDTSTYKNIADFYNEVKNQGYKIT |
| | FCDVPQSYINELVDEGKLYLFQLYNKDFSEHSKGTPNLHTLYFKMLFDERN |
| | LENVVFKLNGEAEMFYREASISKDDMIVHPKNQPIKNKNEQNSRKQSTFEY |
| | DIVKDRRYTVDQFMLHIPITLNFTANGGTNINNEVRKALKDCDKNYVTGID |
| | RGERNLLYTCVVDSEGRTTEQYSLNETTNEYNGNTYSTDYHALLDKKEKER |
| | LESRKAWKTVENIKELKEGYISQVVHKICELVEKYDAVIVMEDLNLGFKQG |
| | RSGKFEKSVYQKFEKMLIDKLNYFADKKKSPEEIGSVLNAYQLTNAFESFE |
| | KMGKQNGFIFYVPAYLTSKIDPTTGFADLLHPSSKQSKESMRDFVGRFDSI |
| | TFNKTENYFEFELDYNKFPRCNTDYRKKWTVCTYGSRIKTFRNPEKNSEWD |
| | NKTVELTPAFMALFEKYSTDVNGDTKAQTMSVDKKDFFVELIGLLRLTLQM |
| | RNSETGKVDRDYLISPVKNSEGVFYNSDDYKGIENASLPKDADANGAYNIA |
| | RKGLWIIEQIKACENDAELNKIRLAISNAEWLEYAQKK* |
| SEQ ID: 12 | MKDYIRKTLSLRILRPYYGEEIEKEIAAAKKKSQAEGGDGALDNKFWDRLK |
| | AEHPEIISSREFYDLLDAIQRETTLYYNRAISKLYHSLIVEREQVSTAKAL |
| | SAGPYHEFREKFNAYISLGLREKIQSNFRRKELARYQVALPTAKSDTFPIP |
| | IYKGFDKNGKGGFKVREIENGDFVIDLPLMAYHRVGGKAGREYIELDRPPA |
| | VLNVPVILSTSRRRANKTWFRDEGTDAEIRRVMAGEYKVSWVEILQRKRFG |
| | KPYGGWYVNFTIKYQPRDYGLDPKVKGGIDIGLSSPLVCAVTNSLARLTIR |
| | DNDLVAFNRKAMARRRTLLRQNRYKRSGHGSANKLKPIEALTEKNELYRKA |
| | IMRRWAREAADFFRQHRAATVNMEDLTGnCDREDYFSQMLRCYWNYSQLQT |
| | MLENKLKEYGIAVKYIEPKDTSKTCHSCGHVNEYFDFNYRSAHKFPMFKCE |
| | KCGVECGADYNAARNIAQA |
| SEQ ID: 13 | MKEQFINRYPLSKTLRFSLIPVGETENNFNKNLLLKKDKQRAENYEKVKCY |
| | IDRFHKEYIESVLSKARTEKVNEYANLYWKSNKDDSDTKANTESLENDMRK |
| | QISKQLTSTEIYKKRLFGKELICEDLPSFLTDKDERETVECFRSFTTYFKG |
| | FNTNRENMYSSDGKSTAIAYRCINDNLPRFLDNVKSFQKVFDNLSDETITK |
| | LNTDLYNIFGRNIEDIFSVDYFEFVLTQSGIEIYNSMIGGYTCSDKTKIQG |
| | LNECINLYNQQVAKNEKSKKLPLMKPLYKQILSEKDSVSFIPEKFNSDNEV |
| | LHAIDDYYTGHIGDFDLLTELLQSLNTYNANGIFVKSGVAITDISNGAFNS |
| | WNVLRSAWNEKYEALHPVTSKTKIDKYIEKQDKIYKAIKSFSLFELQSLGN |
| | ENGNEITDWYISSINESNSKIKEAYLQAQKLLNSDYEKSYNKRLYKNEKAT |
| | ELVKNLLDAIKEFQKLIKPLNGTGKEENKDELFYGKFTSYYDSIADIDRLY |
| | DKVRNYITQKPYSKDKIKLNFDNPQLLGGWDKNKESDYRTVLLHKDGLYYL |
| | AVMDKSHSKAFVDAPEITSDDKDYYEKMEYKLLPGPNKMLPKVFFASKNID |
| | TFQPSDRILDIRKRESFKKGATFNKAECHEFIDYFKDSIKKHDDWSQFGFK |
| | FSPTESYNDISEFYREISDQGYSVRFNKISKNYIDGLVNNGYIYLFQIYNK |
| | DFSKYSKGTPNLHTLYFKMLFDERNLSNVVYKLNGEAEMFYREASIGDKEK |
| | ITHYANQPIKNKNPDNEKKESVFEYDIVKDKRFTKRQFSLHLPITINFKAH |
| | GQEFLNYDVRKAVKYKDDNYVIGIDRGERNLIYISVINSNGEIVEQMSLNE |
| | IISDNGHKVDYQKLLDTKEKERDKARKNWTSVENIKELKEGYISQVVHKIC |
| | ELVIKYDAVIAMEDLNFGFKRGRFPVEKQVYQKFENMLISKLNLLIDKKAE |
| | PTEDGGLLRAYQLTNKFDGVNKAKQNGIIFYVPAWDTSKIDPATGFVNLLK |
| | PKCNTSVPEAKKLFETIDDIKYNANTDMFEFYIDYSKFPRCNSDFKKSWTV |
| | CTNSSRILTFRNKEKNNKWDNKQIVLTDEFKSLFNEFGIDYKGNLKDSILS |
| | ISNADFYRRLIKLLSLTLQMRNSITGSTLPEDDYLISPVANKSGEFYDSRN |
| | YKGTNAALPCDADANGAYNIARKALWAINVLKDTPDDMLNKAKLSITNAEW |
| | LEYTQK* |
| SEQ ID: 14 | MKEQFVNQYPISKTLRFSLIPIGKTEENFNKNLLLKEDEKKAEEYQKVKGY |
| | IDRYHKFFIETALCNINFEGFEEYSLLYYKCSKDDNDLKTMEDIEIKLRKQ |
| | ISKTMTSHKLYKDLFGENMIKTILPNFLDSDEEKNSLEMFRGFYTYFSGFN |
| | TNRKNMYTEEAKSTSIAYRCINDNLPKFLDNSKSFEKIKCALNKEELKAKN |
| | EEFYEIFQIYATDIFNIDFFNFVLTQPGIDKYNGIIGGYTCSDGTKVQGLN |
| | EIINLYNQQIAKDDKSKRLPLLKMLYKQILSDRETVSFIPEKFSSDNEVLE |
| | SINNYFSKNVSNAIKSLKELFQGFEAYNMNGIFISSGVAITDLSNAVFGDW |
| | NAISTAWEKAYFETNPPKKNKSQEKYEEELKANYKKIKSFSLDEIQRLGSI |
| | AKSPDSIGSVAEYYKITVTEKIDNITELYDGSKELLNCNYSESYDKKLIKN |
| | DTVIEKVKTLLDAVKSLEKLIKPLVGTGKEDKDELFYGTFLPLYTSLSAVD |
| | RLYDKVRNYATQKPYSKDKIKLNFNCSSFLSGWATDYSSNGGLIFEKDGLY |
| | YLGIVNKKFTTEEIDYLQQNADENPAQRIVYDFQKPDNKNTPRLFIRSKGT |
| | NYSPSVKEYNLPVEEIVELYDKRYFTTEYRNKNPELYKASLVKLIDYFKLG |
| | FTRHESYRHYDFKWKKSEEYNDISEFYKDVEISCYSLKQEKINYNTLLNFV |
| | AENRIYLFQIYNKDFSKYSKGTPNLHTRYFKALFDENNLSDVVFKLNGGSE |
| | MFFRKASIKDNEKVVHPANQPIDNKNPDNSKKQSTFDYELIKDKRFTKHQF |
| | SIHIPITMNFKARGRDFINNDIRKAIKSEYKPYVIGIDRGERNLIYISVIN |
| | NNGEIVEQMSLNDIISDNGYKVDYQRLLDRKEKERDNARKSWGTIENIKEL |
| | KEGYISQVIHKICELVIKYDAVIAMEDLNFGFKRGRFNVEKQVYQKFENML |
| | ISKLNYLCDKKSEANSEGGLLKAYQLTNKFDGVNKGKQNGIIFYVPAWLTS |
| | KIDPVTGFVDLLHPKYISVEETHSLFEKLDDIRYNFEKDMFEFDIDYSKLP |
| | KCNADFKQKWTVCTNADRIMTFRNSEKNNEWDNKRILLSDEFKRLFEEFGI |
| | DYCHNLKNKILSISNKDFCYRFIKLFALTMQMRNSITGSTNPEDDYLISPV |
| | RDENGVFYDSRNFIGSKAGLPIDADANGAYNIARKGLWAINAIKSTADDML |
| | DKVDLSISNAKWLEYVQK* |
| SEQ ID: 15 | MKITKIDGILHKKYIKEGKLVKSTSEENKTDERLSELLTIRLDTYIKNPDN |
| | ASEEENRIRRETLKEFFSNKVLYLKDSILYLKDRREKNQLQNKNYSEEDIS |
| | EYDLKNKNSFLVLKKILLNEDINSEELEIFRNDFEKKLDKINSLKYSLEEN |
| | KANYQKINENNIKKVEGKSKRNIFYNYYKDSAKRNDYINNIQEAFDKLYKK |
| | EDIENLFFLIENSKKHEKYKIRECYHKIIGRKNDKENFATIIYEEIQNVNN |
| | MKELIEKVPNVSELKKSQVFYKYYLNKEKLNDENIKYVFCHFVEIEMSKLL |
| | KNYVYKKPSNISNDKVKRIFEYQSLKKLIENKLLNKLDTYVRNCGKYSFYL |
| | QDGEIATSDFIVGNRQNEAFLRNIIGVSSTAYFSLRNILETENENDITGRI |
| | KGKTVKNKKGEEKYISGEIDKLYDNNKQNEVKKNLKMFYSYDFNMNRKKEI |
| | EDFFSNIDEAISSIRHGIVHFNLELEGKDIFTFKNIVPSQISKKMFQNEIN |
| | EKKLKLKIFRQLNSANVFRYLEKYKILNYLNRTRFEFVNKNIPFVPSFTKL |
| | YSRIDDLKNSLCIYWKIPKANDNNKTKEITDAQIYLLKNIYYGEFLNYFMS |
| | NNGNFFEIIKEIIELNKNDKRNLKTGFYKLQKFENLQEKTPKEYLANIQSF |
| | YMIDAGNKDEEEKDAYIDFIQKIFLKGFMTYLANNGRLSLMYIGNDEQINT |
| | SLAGKKQEFDKFLKKYEQNNNIEIPHEINEFVREIKLGKILKYTESLNMFY |
| | LILKLLNHKELTNLKGSLEKYQSANKEEAFSDQLELINLLNLDNNRVTEDF |
| | ELEADEIGKFLDFNGNKVKDNKELKKFDTNKIYFDGENIIKHRAFYNIKKY |
| | GILNLLEKISDEAKYKISIEELKNYSNKKIEIEKNHTTQENLHRKYARPRK |
| | DEKFNDEDYKKYEKTIRNIQQYTHLKNKVEFNELNLLQSLLLRILHRLVGY |
| | TSIWERDLRFRLKGEFPENQYIEEIFNFDNSKNVKYKNGQIVEKYISFYKE |
| | LYKDDMEKISIYSDKKVKELKKEKKDLYIRNYIAHFNYIPNAEVSLLEVLE |
| | NLRKLLSYDRKLKNAIMKSIVDILKEYGFVVTFKIEKDKKIRIESLKSEEV |
| | VHLKKLKLKDNDKKKEPIKTYRNSKELCKLVKVMFEYKMKEKKSEN* |
| SEQ ID: 16 | MKITKIDGISHKKYIKEGKLVKSTSEENKTDERLSELLTIRLDTYIKNPDN |
| | ASEEENRIRRENLKEFFSNKVLYLKDGILYLKDRREKNQLQNKNYSEEDIS |
| | EYDLKNKNSFLVLKKILLNEDINSEELEIFRKDVEAKLNKINSLKYSFEEN |
| | KANYQKINENNVEKVGGKSKRNIIYDYYRESAKRNDYINNVQEAFDKLYKK |
| | EDIEKLFFL1ENSKKHEKYK1RECYHKIIGRKNDKENFAKIIYEEIQNVNN |
| | IKELIEKVPDMSELKKSQVFYKYYLDKEELNDKNIKYAFCHFVEIEMSQLL |
| | KNYVYKRLSNISNDKIKRIFEYQNLKKLIENKLLNKLDTYVRNCGKYNYYL |
| | QDGEIATSDFIAGNRQNEAFLRNIIGVSSVAYFSLRNILETENKDDITGKM |
| | RGKTRIDSKTGEEKYIPGEVDQIYYENKQNEVKNKLKMFYGYDFDMDNKKE |
| | IEDFFANIDEAISSIRHGIVHFNLDLDGKDIFAFKNIVPSEISKKMFQNEI |
| | NEKKLKLKIFRQLNSANVFRYLEKYKILNYLKRTRFEFVNKNIPFVPSFTK |
| | LYSRIDDLKNSLGIYWKTPKTNDDNKTKEIIDAQIYLLKNIYYGEFLNYFM |
| | SNNGNFFEISREIIELNKNDKRNLKTGFYKLQKFEDIQEKTPKKYLANIQS |
| | LYMINAGNQDEEEKDTYIDFIQKIFLKGFMTYLANNGRLSLMYIGNDEQIN |
| | TSLAGKKQEFDKFLKKYEQNNNIEIPHEINEFLREIKLGKILKYTESLNMF |
| | YLILKLLNHKELTNLKGSLEKYQSANKEETFSDELELINLLNLDNNRVTED |
| | FELEANEIGKFLDFNGNKIKDRKELKKFDTKKIYFDGENIIKHRAFYNIKK |
| | YGMLNLLEKIADKAKYKISLKELKEYSNKKNEIEKNYTMQQNLHRKYARPK |
| | KDEKFNDEDYKEYEKAIGNIQKYTHLKNKVEFNELNLLQGLLLKILHRLVG |
| | YTSIWERDLRFRLKGEFPENQYIEEIFNFDNSKNVKYKSGQIVEKYINFYK |
| | ELYKDNVEKRSIYSDKKVKKLKQEKKDLYIRNYIAHFNY1PHAEISLLEVL |
| | ENLRKLLSYDRKLKNA1MKSVVDILKEYGFVATFKIGADKKIGIQTLESEK |
| | IVHLKNLKKKKLMTDRNSEELCKLVKVMFEYKMEEKNLKTKKCKVI* |
| SEQ ID: 17 | MKKIDNFVGCYPVSKTLRFKAIPIGKTQENIEKKRLVEEDEVRAKDYKAVK |
| | KLIDRYHREFIEGVLDNVKLDGLEEYYMLFNKSDREESDNKKIEIMEERFR |
| | RVISKSFKNNEEYKKIFSKKIIEEILPNYIKDEEEKELVKGFKGFYTAFVG |
| | YAQNRENMYSDEKKSTAISYRIVNENMPRFITNIKVFEKAKSILDVDKINE |
| | INEYILNNDYYVDDFFNIDFFNYVLNQKGIDIYNAIIGGIVTGDGRKIQGL |
| | NECINLYNQENKKIRLPQFKPLYKQILSESESMSFYIDEIESDDMLIDMLK |
| | ESLQIDSTINNAIDDLKVLFNNIFDYDLSGIFINNGLPITTISNDVYGQWS |
| | TISDGWNERYDVLSNAKDKESEKYFEKRRKEYKKVKSFSISDLQELGGKDL |
| | SICKKINEIISEMIDDYKSKIEEIQYLFDIKELEKPLVTDLNKIELIKNSL |
| | DGLKRIERYVIPFLGTGKEQNRDEVFYGYFIKCIDAIKEIDGVYNKTRNYL |
| | TKKPYSKDKFKLYFENPQLMGGWDRNKESDYRSTLLRKNGKYYVAIIDKSS |
| | SNCMMNIEEDENDNYEKINYKLLPGPNKMLPKVFFSKKNREYFAPSKEIER |
| | IYSTGTFKKDTNFVKKDCENLITFYKDSLDRHEDWSKSFDFSFKESSAYRD |
| | ISEFYRDVEKQGYRVSFDLLSSNAVNTLVEEGKLYLFQLYNKDFSEKSHGI |
| | PNLHTMYFRSLFDDNNKGNIRLNGGAEMFMRRASLNKQDVTVHKANQPIKN |
| | KNLLNPKKTTTLPYDVYKDKRFTEDQYEVHIPITMNKVPNNPYKFNHMVRE |
| | QLVKDDNPYVIGIDRGERNLIYVVVVDGQGHIVEQLSLNEIINENNGISIR |
| | TDYHTLLDAKERERDESRKQWKQIENIKELKEGYISQVVHKICELVEKYDA |
| | VIALEDLNSGFKNSRVKVEKQVYQKFEKMLITKLNYMVDKKKDYNKPGGVL |
| | NGYQLTTQFESFSKMGTQNGIMFYIPAWLTSKMDPTTGFVDLLKPKYKNKA |
| | DAQKFFSQFDSIRYDNQEDAFVFKVNYTKFPRTDADYNKEWEIYTNGERIR |
| | VFRNPKKNNEYDYETVNVSERMKELFDSYDLLYDKGELKETICEMEESKFF |
| | EELIKLFRLTLQMRNSISGRTDVDYLISPVKNSNGYFYNSNDYKKEGAKYP |
| | KDADANGAYNIARKVLWAIEQFKMADEDKLDKTKISIKNQEWLEYAQTHCE |
| SEQ ID: 18 | MKKIDSFVNYYPLSKTLRFSLIPVGKTEDNFNAKLLLEEDEKRAIEYEKVK |
| | RYIDRYHKHFIETVLANFHLDDVNEYAELYYKAGKDDKDLKYMEKLEGKMR |
| | KSISAAFTKDKKYKEIFGQEIIKNILPEFLENEDEKESVKMFQGFFTYFTG |
| | FNDNRKNMYTHEAQTTAISYRCINENLPKFLDNVQSFAKIKESISSDIMNK |
| | LDEVCMDLYGVYAQDMFCTDYFSFVLSQSGIDRYNNIIGGYVDDKGVKIQG |
| | INEYINLYNQQVDEKNKRLPLMKKLYKQILIEKESISFIPEKFESDNIVIN |
| | AISDYYHNNVENLFDDFNKLFNEFSEYDDNGIFVTSGLAVTDISNAVFGSW |
| | NIISDSWNEEYKDSHPMKKTTNAEKYYEDMKKEYKKNLSFTIAELQRLGEA |
| | GCNDECKGDIKEYYKTTVAEKIENIKNAYEISKDLLASDYEKSNDKKLCKN |
| | DSAISLLKNLLDSIKDLEKTIKPLLGTGKEENKDDVFYGKFTNLYEMISEI |
| | DRLYDKVRNYVTQKPYSKDKIKLNFENPQHLGGWDKNKERDYRSVLLKKED |
| | KYYLATMDKSNNKAFIDFPDDGECYEKIEYKLLPGPNKMLPKVFFASSNIE |
| | YFAPSKKILEIRSRESFKKGDMFNLKDCHEFIDFFKESIKKHEDWSQFGFE |
| | FSPTEKYNDISEFYNEVKIQGYSLKYKNVSKKYIDELIECGQLYLFQIYNK |
| | DFSVYAKGNPNLHTMYFKMLFDERNLANVVYQLNGGAEMFYRKASIKDSEK |
| | IVHHANQPIKNKNADNVKKESVFEYDIIKDKRFTKRQFSIHIPTTLNFKAK |
| | GQNFINNDVRMALKKADENYVIGIDRGERNLLYICVINSKGEIVEQKSLNE |
| | IIGDNGYRVDYHKLLDKKEAERDEARKSWGTIENIKELKEGYLSQIVHEIS |
| | KLVIKYDAVIAIEDLNSGFKKGRFKVEKQVYQKFENMLCTKLNYLVDKNAD |
| | ANECGGLLKAYQLTNKEDGANRGRQNGIIFSVPAWLTSKIDPVTGFADLLR |
| | PKYKSVSESVEFISKIDNIRYNSKEDYFEFDIDYSKFPNSTASYKKKWTVC |
| | TYGERIINVRNKEKNNMWDNKTIVLTDEFKKLFADFGVDVSKNIKESVLAI |
| | DSKDFYYRFINLLANTLQLRNSEVGNVDVDYLISPVKGVDGSFYDSRLVKE |
| | KTLPENADANGAYNIARKALWAIDVLKQTKDEELKNANLSIKNAEWLEYVQ |
| | K* |
| SEQ ID: 19 | MKNQNTLPSNPTDILKDKPFWAAFFNLARHNVYLTVNHINKLLDLEKLYNK |
| | DKHKEIFEHEDIFNISDDVMNDVNSNGKKRKLDIKKIWANLDTDLTRKYQL |
| | RELILKHFPFIQPAIIGAQTKERTTIDKDKRSTSTSNDSLKPTGEGDINDP |
| | LSLSNVKSIFFRLLQMLEQLRNYYSHVKHSKSATMPNFDEGLLKSMYNIFI |
| | DSVNKVKEDYSSNSVIDPNTSFSHLISKDEQGEIKPCRYSFTSKDGSINAS |
| | GLLFFVSLFLEKQDSIWMQKKIPGFKKTSENYMKMTNEVFCRNHILLPKMR |
| | LETVYDKDWMLLDMLNEVVRCPLSLYKRLAPADQNKFKVPEKSSDNANRQE |
| | DDNPFSRILVRHQNRFPYFALRFFDLNEVFTTLRFQINLGCYHFAICKKQI |
| | GDKKEVHHLTRTLYGFSRLQNFTQNTRPEEWNTLVKTTEPSSGNDGKTVQG |
| | VPLPYISYTIPHYQIENEKIGIKIFDGDTAVDTDIWPSVSTEKQLNKPDKY |
| | TLTPGFKADVFLSVHELLPMMFYYQLLLCEGMLKTDAGNAVEKVLIDTRNA |
| | IFNLYDAFVQEKINTITDLENYLQDKPILIGHLPKQMIDLLKGHQRDMLKA |
| | VEQKKAMLIKDTERRLERLNKQPEQKPNVAAKNTGTLLRNGQIADWLVKDM |
| | MRFQPVKRDKEGNPINCSKANSTEYQMLQRAFAFYTTDSYRLPRYFEQLHL |
| | INCDNSHLFLSRFEYDKQPNLIAFYAAYLEAKLEFLNELQPQNWASDNYFL |
| | LLRAPKNDRQKLAEGWKNGFNLPRGLFTEKIKTWFNEHKTIVDISDCDIFK |
| | NRVGQVARLIPVFFDKKFKDHSQPFYTYNFNVGNVSKITEANYLSKEKREN |
| | LFKSYQNKFKNNIPAEKTKEYREYKNFSSWKKFERELRLIKNQDILTWLMC |
| | KNLFDEKIKPKKDILEPRIAVSYIKLDSLQTNTSTAGSLNALAKVVPMTLA |
| | IHIDSPKPKGKAGNNEKENKEFTVYIKEEGTKLLKWGNFKTLLADRRIKGL |
| | FSYIEHDDINLEKYPLTKYQVDSELDLYQKYRIDIFKQTLDLEAQLLDKYS |
| | DLNTDNFNQMLSGWSEKEGIPRNIKQDVAFLIGVRNGFSHNQYPDSKRIAF |
| | SRIKKFNPKTSSLQESEGLNIAKQMYEEAQQVVNKIKNIESFD* |
| SEQ ID: 20 | MKVTKIDGISHKKFEDEGKLVKFTGHFNIKNEMKERLEKLKELKLSNYIKN |
| | PENVKNKDKNKEKETKSRRENLKKYFSEIILRKKEEKYLLKKTRKFKNITE |
| | EINYDDIKKRENQQKIFDVLKELLEQRINENDKEEILNFDSVKLKEAFEED |
| | FIKKELKIKAIEESLEKNRADYRKDYVELENEKYEDVKGQNKRSLVFEYYK |
| | NPENREKFKENIKYAFENLYTEENIKNLYSEIKEIFEKVHLKSKVRYFYQN |
| | EIIGESEFSEKDEEGISILYKQIINSVEKKEKFIEFLQKVKIKDLTRSQIF |
| | YKYFLENEELNDENIKYVFSYFVEIEVNKLLKENVYKTKKFNEGNKYRVKN |
| | IFNYDKLKNLVVYKLENKLNNYVRNCGKYNYHMENGDIATSDINMKNRQTE |
| | AFLRSILGVSSFGYFSLRNILGVNDDDFYKIEKDERKNENFILKKAKEDFT |
| | SKNIFEKVVDKSFEKKGIYQIKENLKMFYGNSFDKVDKDELKKFFVNMLEA |
| | ITSVRHRIVHYNINTNSENIFDFSNIEVSKLLKNIFEKEIDTRELKLKIFR |
| | QLNSAGVFDYWESWVIKKYLENVKFEFVNKNVPFVPSFKKLYDRIDNLKGW |
| | NALKLGNNINIPKRKEAKDSQIYLLKNIYYGEFVEKFVNDNKNFEKTVKET |
| | TEINRGAGTNKKTGFYKLEKFETLKANTPTKYLEKLQSLHKTSYDKEKIEE |
| | DKDVYVDFVQKIFLKGFVNYLKKLDSLKSLNLLNLRKDETITDKKSVHDEK |
| | LKLWENSGSNLSKMPEEIYEYVKKIKISNINYNDRMSIFYLLLKLIDYREL |
| | TNLRGNLEKYESMNKNKIYSEELTIINLVNLDNNKVRTNFSLEAEDIGKFL |
| | KSSITIKNIAQLNNFSKIFADGENVIKHRSFYNIKKYGILDLLEKTVAKAD |
| | LKTTKEETKKYENLQNELKRNDFYKTQEQIHRNYNQKPFSIKKIENKKDFE |
| | KYKKVIEKIQDYTQLKNKIEFNDLNLLQSLIFRILHRLAGYTSLWERDLQF |
| | KLKGEFPEDKYIDEIFNSDGNNNQKYKHGGIADKYANFLIEKKEEKSGEIL |
| | NKKQRKKKIKEDLEIRNYIAHFNYLPNAEKSILEILEELRELLKHDRKLKN |
| | AVMKSIKDIFREYGFIVEFTISHTKNGKKIKVCSVKSEKIKHLKNNELITT |
| | RNSEDLCELVKIMLEHKELQK* |
| SEQ ID: 21 | MKVTKVGGISHKKYTSEGRLVKSESEENRTDERLSALLNMRLDMYIKNPSS |
| | TETKENQKRIGKLKKFFSNKMVYLKDNTLSLKNGKKENIDREYSETDILES |
| | DVRDKKNFAVLKKIYLNENVNSEELEVFRNDIKKKLNKINSLKYSFEKNKA |
| | NYQKINENNIEKVEGKSKRNIIYDYYRESAKRDAYVSNVKEAFDKLYKEED |
| | IAKLVLEIENLTKLEKYKIREFYHEIIGRKNDKENFAKIIYEEIQNVNNMK |
| | ELIEKVPDMSELKKSQVFYKYYLDKEELNDKNIKYAFCHFVEIEMSQLLKN |
| | YVYKRLSNISNDKIKRIFEYQNLKKLIENKLLNKLDTYVRNCGKYNYYLQD |
| | GEIATSDFIARNRQNEAFLRNIIGVSSVAYFSLRNILETENENDITGRMRG |
| | KTVKNNKGEEKYVSGEVDKIYNENKKNEVKENLKMFYSYDFNMDNKNEIED |
| | FFANIDEAISSIRHGIVHFNLELEGKDIFAFKNIAPSEISKKMFQNEINEK |
| | KLKLKIFRQLNSANVFRYLEKYKILNYLKRTRFEFVNKNIPFVPSFTKLYS |
| | RIDDLKNSLGIYWKTPKTNDDNKTKEIIDAQIYLLKNIYYGEFLNYFMSNN |
| | GNFFEISKEIIELNKNDKRNLKTGFYKLQKFEDIQEKIPKEYLANIQSLYM |
| | INAGNQDEEEKDTYIDFIQKIFLKGFMTYLANNGRLSLIYIGSDEETNTSL |
| | AEKKQEFDKFLKKYEQNNNIKIPYEINEFLREIKLGNILKYTERLNMFYLI |
| | LKLLNHKELTNLKGSLEKYQSANKEEAFSDQLELINLLNLDNNRVTEDFEL |
| | EADEIGKFLDFNGNKVKDNKELKKFDTNKIYFDGENIIKHRAFYNIKKYGM |
| | LNLLEKIADKAGYKISIEELKKYSNKKNEIEKNHKMQENLHRKYARPRKDE |
| | KFTDEDYESYKQAIENIEEYTHLKNKVEFNELNLLQGLLLRILHRLVGYTS |
| | IWERDLRFRLKGEFPENQYIEEIFNFENKKNVKYKGGQIVEKYIKFYKELH |
| | QNDEVKINKYSSANIKVLKQEKKDLYIRNYIAHFNYIPHAEISLLEVLENL |
| | RKLLSYDRKLKNAVMKSVVDILKEYGFVATFKIGADKKIGIQTLESEKIVH |
| | LKNLKKKKLMTDRNSEELCKLVKIMFEYKMEEKKSEN |
| SEQ ID: 22 | MNELVKNRCKQTKTICQKLIPIGKTRETIEKYNLMEIDRKIAANKELMNKL |
| | FSLIAGKHINDTLSKCTDLDFEPLLTSLSSLNNAKENDRDNLREYYDSVFE |
| | EKKTLAEEISSRLTAVKFAGKDFFTKNIPDFLETYEGDDKNEMSELVSLVI |
| | ENTVTAGYVKKLEKIDRSMEYRLVSGTVVKRVLTDNADIYEKNIEKAKDFD |
| | YGVLNIDEASQFTTLVAKDYANYLTADGIAIYNVGIGKINLALNEYCQKNK |
| | EYSYNKLALLPLQKMLYGEKLSLFEKLEDFTSDEELINSYNKFAKTVNESG |
| | LAEIIKKAVPSYDEIVIKPNKISNYSNSITGHWSLVNRIMKDYLENNGIKN |
| | ADKYMEKGLTLSEIGDALENKNIKHSDFISNLINDLGHTYTEIKENKESLK |
| | KDESVNALIIKKELDMLLSILQNLKVFDIDNEMFDTGFGIEVSKAIEILGY |
| | GVPLYNKIRNYITKKPDPKKKFMTKFGSATIGTGITTSVEGSKKATFLKDG |
| | DAVFLLLYNTAGCKANNVSVSNLADLINSSLEIENSGKCYQKMIYQTPGDI |
| | KKQIPRVFVYKSEDDDLIKDFKAGLHKTDLSFLNGRLIPYLKEAFATHETY |
| | KNYTFSYRNSYESYDEFCEHMSEQAYILEWKWIDKKLIDDLVEDGSLLMFR |
| | VWNRFMKKKEGKISKHAKIVNELFSDENASNAAIKLLSVFDIFYRDKQIDN |
| | PIVHKAGTTLYNKRTKDGEVIVDYTTMVKNKEKRPNVYTTTKKYDIIKDRR |
| | YTEEQFEIHLHVNIGKEENKEKLETSKVINEKKNTLVVTRSNEHLLYVVIF |
| | DENDNILLKKSLNTVKGMNFKSKLEVVEIQKKENMQSWKTVGSNQALMEGY |
| | LSFAIKEIADLVKEYDAILVLEQNSVGKNILNERVYTRFKEMLITNLSLDV |
| | DYENKDFYSYTELGGKVASWRDCVTNGICIQVPSAYKYKDPTTSFSTISMY |
| | AKTTAEKSKKLKQIKSFKYNRERGLFELVIAKGVGLENNIVCDSFGSRSII |
| | ENDISKEVSCTLKIEKYLIDAGIEYNDEKEVLKDLDTAAKTDAVHKAVTLL |
| | LKCFNESPDGRYYISPCGEHFTLCDAPEVLSAINYYIRSRYIREQIVEGVK |
| | KMEYKKTILLAK* |
| SEQ ID: 23 | MNGNRIIVYREFVGVTPVAKTLRNELRPIGHTQEHIIHNGLIQEDELRQEK |
| | STELKNIMDDYYREYIDKSLSGVTDLDFTLLFELMNLVQSSPSKDNKKALE |
| | KEQSKMREQICTHMQSDSNYKNIFNAKFLKEILPDFIKNYNQYDAKDKAGK |
| | LETLALFNGFSTYFTDFFEKRKNVFTKEAVSTSIAYRIVHENSLTFLANMT |
| | SYKKISEKALDEIEVIEKNNQDKMGDWELNQIFNPDFYNMVLIQSGIDFYN |
| | EICGVVNAHMNLYCQQTKNNYNLFKMRKLHKQILAYTSTSFEVPKMFEDDM |
| | SVYNAVNAFIDETEKGNIIGKLKDIVNKYDELDEKRIYISKDFYETLSCFM |
| | SGNWNLITGCVENFYDENIHAKGKSKEEKVKKAVKEDKYKSINDVNDLVEK |
| | YIDEKERNEFKNSNAKQYIREISNIITDTETAHLEYDEHISLIESEEKADE |
| | MKKRLDMYMNMYHWAKAFIVDEVLDRDEMFYSDIDDIYNILENIVPLYNRV |
| | RNYVTQKPYNSKKIKLNFQSPTLANGWSQSKEFDNNAIILIRDNKYYLAIF |
| | NAKNKPDKKIIQGNSDKKNDNDYKKMVYNLLPGANKMLPKVFLSKKGIETF |
| | KPSDYIISGYNAHKHIKTSENFDISFCRDLIDYFKNSIEKHAEWRKYEFKF |
| | SATDSYNDISEFYREVEMQGYRIDWTYISEADINKLDEEGKIYLFQIYNKD |
| | FAENSTGKENLHTMYFKNIFSEENLKDIIIKLNGQAELFYRRASVKNPVKH |
| | KKDSVLVNKTYKNQLDNGDVVRIPIPDDIYNEIYKMYNGYIKENDLSEAAK |
| | EYLDKVEVRTAQKDIVKDYRYTVDKYFIHTPITINYKVTARNNVNDMAVKY |
| | IAQNDDIHVIGIDRGERNLIYISVIDSHGNIVKQKSYNILNNYDYKKKLVE |
| | KEKTREYARKNWKSIGNIKELKEGYISGVVHEIAMLMVEYNAIIAMEDLNY |
| | GFKRGRFKVERQVYQKFESMLINKLNYFASKGKSVDEPGGLLKGYQLTYVP |
| | DNIKNLGKQCGVIFYVPAAFTSKIDPSTGFISAFNFKSISTNASRKQFFMQ |
| | FDEIRYCAEKDMFSFGFDYNNFDTYNITMSKTQWTVYTNGERLQSEFNNAR |
| | RTGKTKSINLTETIKLLLEDNEINYADGHDVRIDMEKMDEDKNSEFFAQLL |
| | SLYKLTVQMRNSYTEAEEQEKGISYDKIISPVINDEGEFFDSDNYKESDDK |
| | ECKMPKDADANGAYCIALKGLYEVLKIKSEWTEDGFDRNCLKLPHAEWLDF |
| | IQNKRYE* |
| SEQ ID: 24 | MNKDIRKNFTDFVGISEIQKTLRFILIPIGKTAQNIDKYNMFEDDEIRHEY |
| | YPTLKEACDDFYRNHTDQQFENLELDWSKLDEALASEDRDLTNETRATYRQ |
| | VLFNRLKNSVDIKGDSKKNKTLSLESSDKNLGKKKTKNTFQYNFNDLFKAK |
| | LIKAILPLYIEYIYEGEKLENAKKALKMYNRFTSRLSNFWQARANIFTDDE |
| | ISTGSPYRLVNDNFTIFRINNSIYTKNKPFIEEDILEFEKKLKSKKIIKDF |
| | ESVDDYFTVNAFNKLCTQNGIDKYNSILGGFTTKEREKVKGLNELFNLAQQ |
| | SINKGKKGEYRKNIRLGKLTKLKKQILAISDSTSFLIEQIEDDQDLYNKIK |
| | DFFELLLKEEIENENIFTQYANLQKLIEQADLSKIYINAKHLNKISHQVTG |
| | KWDSLNKGIALLLENININEESKEKSEVISNGQTKDISSEAYKRYLQIQSE |
| | EKDIERLRTQIYFSLEDLEKALDLVLIDENMDRSDKSILSYVQSPDLNVNF |
| | ERDLTDLYSRIMKLEENNEKLLANHSAIDLIKEFLDLIMLRYSRWQILFCD |
| | SNYELDQTFYPIYDAVMEILSNIIRLYNLARNYLSRKPDRMKKKKINFNNP |
| | TLADGWSESKIPDNSSMLFIKDGMYYLGIIKNRAAYSELLEAESLQSSEKK |
| | KSENSSYERMNYHFLPDAFRSIPKSSIAMKAVKEHFEINQKTADLLLDTDK |
| | FSKPLRITKEIFDMQYVDLHKNKKKYQVDYLRDTGDKKGYRKALNTWLNFC |
| | KDFISKYKGRNLFDYSKIKDADHYETVNEFYNDVDKYSYHIFFTSVAETTV |
| | EKFISEGKLYLFQLYNKDFSPHSTGKPNLHTIYWRALFSEENLTSKNIKLN |
| | GQAEIFFRPKQIETPFTHKKGSILVNRFDVNGNPIPINVYQEIKGFKNNVI |
| | KWDDLNKTTQEGLENDQYLYFESEFEIIKDRRYTEDQLFFHVPISFNWDIG |
| | SNPKINDLATQYIVNSNDIHIIGIDRGENHLIYYSVIDLQGAIVEQGSLNT |
| | ITEYTENKFLNNKTNNLRKIPYKDILQQREDERADARIKWHAIDKIKDLKD |
| | GYLGQIVHFLAKLIIKYNAIVILEDLNYGFKRGRFKVERQVYQKFEMALMK |
| | KLNVLVFKDYDIDEIGGPLKPWQLTRPIDSYERMGRQNGILFYVPAAYTSA |
| | VDPVTGFANLFYLNNVKNSEKFHFFSKFESIKYHSDQDMFSFAFDYNNFGT |
| | TTRINDLSKSKWQVFTNHERSVWNNKEKNYVTQNLTDLIKKLLRTYNIEFK |
| | NNQNVLDSILKIENNTDKENFARELFRLFRLTIQLRNTTVNENNTEITENE |
| | LDYIISPVKDKNGNFFDSRDELKNLPDNGDANGAYNIARKGLLYIEQLQES |
| | IKTGKLPTLSISTLDWFNYIMK* |
| SEQ ID: 25 | MNKGGYVIMEKMTEKNRWENQFRITKTIKEEIIPTGYTKVNLQRVNMLKRE |
| | MERNEDLKKMKEICDEYYRNMIDVSLRLEQVRTLGWESLIHKYRMLNKDEK |
| | EIKALEKEQEDLRKKISKGFGEKKAWTGEQFIKKILPQYLMDHYTGEELEE |
| | KLRIVKKFKGCTMFLSTFFKNRENIFTDKPIHTAVGHRITSENAMLFAANI |
| | NTYEKMESNVTLEIERLQREFWRRGINISEIFTDAYYVNVLTQKQIEAYNK |
| | ICGDINQHMNEYCQKQKLKFSEFRMRELKKQILAVVGEHFEIPEKIESTKE |
| | VYRELNEYYESLKELHGQFEELKSVQLKYSQIYVQKKGYDRISRYIGGQWD |
| | LIQECMKKDCASGMKGTKKNHDAKIEEEVAKVKYQSIEHIQKLVCTYEEDR |
| | GHKVTDYVDEFIVSVCDLLGADHIITRDGERIELPLQYEPGTDLLKNDTIN |
| | QRRLSDIKTILDWHMDMLEWLKTFLVNDLVIKDEEFYMAIEELNERMQCVI |
| | SVYNRIRNYVTQKGYEPEKIRICFDKGTILTGWTTGDNYQYSGFLLMRNDK |
| | YYLGIINTNEKSVRKILDGNEECKDENDYIRVGYHLINDASKQLPRIFVMP |
| | KAGKKSEILMKDEQCDYIWDGYCHNKHNESKEFMRELIDYYKRSIMNYDKW |
| | EGYCFKFSSTESYDNMQDFYKEVREQSYNISFSYINENVLEQLDKDGKIYL |
| | FQVYNKDFAAGSTGTPNLHTMYLQNLFSSQNLELKRLRLGGNAELFYRPGT |
| | EKDVTHRKGSILVDRTYVREEKDGIEVRDTVPEKEYLEIYRYLNGKQKGDL |
| | SESAKQWLDKVHYREAPCDIIKDKRYAQEKYFLHFSVEINPNAEGQTALND |
| | NVRRWLSEEEDIHVIGIDRGERNLIYVSLMDGKGRIKDQKSYNIVNSGNKE |
| | PVDYLAKLKVREKERDEARRNWKAIGKIKDIKTGYLSYVVHEIVEMAVREK |
| | AIIVMEDLNYGFKRGRFKVERQVYQKFEEMLINKLNYVVDKQLSVDEPGGL |
| | LRGYQLAFIPKDKKSSMRQNGIVFYVPAGYTSKIDPTTGFVNIFKFPQFGK |
| | GDDDGNGKDYDKIRAFFGKFDEIRYECDEKVTADNTREVKERYRFDFDYSK |
| | FETHLVHMKKTKWTVYAEGERIKRKKVGNYWTSEVISDIALRMSNTLNIAG |
| | IEYKDGHNLVNEICALRGKQAGIILNELLEIVRLTVQLRNSTTEGDVDERD |
| | EIISPVLNEKYGCFYHSTEYKQQNGDVLPKDADANGAYCIGLKGIYEIRQI |
| | KNKWKEDMTKGEGKALNEGMRISHDQWFEFIQNMNKGE* |
| SEQ ID: 26 | MNNPRGAFGGFTNLYSLSKTLRFELKPYLEIPEGEKGKLFGDDKEYYKNCK |
| | TYTEYYLKKANKEYYDNEKVKNTDLQLVNFLHDERIEDAYQVLKPVFDTLH |
| | EEFITDSLESAEAKKIDFGNYYGLYEKQKSEQNKDEKKKIDKPLETERGKL |
| | RKAFTPIYEAEGKNLKNKAGKEKKDKDILKESGFKVLIEAGILKYIKNNID |
| | EFADKKLKNNEGKEITKKDIETALGAENIEGIFDGFFTYFSGFNQNRENYY |
| | STEEKATAVASRIVDENLSKFCDNILLYRKNENDYLKIFNFLKNKGKDLKL |
| | KNSKFGKENEPEFIPAYDMKNDEKSFSVADFVNCLSQGEIEKYNAKIANAN |
| | YLINLYNQNKDGNSSKLSMFKILYKQIGCGEKKDFIKTIKDNAELKQILEK |
| | ACEAGKKYFIRGKSEDGGVSNIFDFTDYIQSHENYKGVYWSDKAINTISGK |
| | YFANWDTLKNKLGDAKVFNKNTGEDKADVKYKVPQAVMLSELFAVLDDNAG |
| | EDWREKGIFFKASLFEGDQNKSEIIKNANRPSQALLKMICDDMESLAKNFI |
| | DSGDKILKISDRDYQKDENKQKIKNWLDNALWINQILKYFKVKANKIKGDS |
| | IDARIDSGLDMLVFSSDNPAEDYDMIRNYLTQKPQDEINKLKLNFENSSLA |
| | GGWDENKEKDNSCIILKDEQDKQYLAVMKYENTKVFEQKNSQLYIADNAAW |
| | KKMIYKLVPGASKTLPKVFFSKKWTANRPTPSDIVEIYQKGSFKKENVDFN |
| | DKKEKDESRKEKNREKIIAELQKTCWMDIRYNIDGKIESAKYVNKEKLAKL |
| | IDFYKENLKKYPSEEESWDRLFAFGFSDTKSYKSIDQFYIEVDKQGYKLEF |
| | VTINKARLDEYVRDGKIYLFEIRSRDNNLVNGEEKTSAKNLQTIYWNAAFG |
| | GDDNKPKLNGEAEIFYRPAIAENKLNKKKDKNGKEIIDGYRFSKEKFIFHC |
| | PITLNFCLKETKINDKLNAALAKPENGQGVYFLGIDRGEKHLAYYSLVNQK |
| | GEILEQGTLNLPFLDKNGKSRSIKVEKKSFEKDSNGGIIKDKDGNDKIKIE |
| | FVECWNYNDLLDARAGDRDYARKNWTTIGTIKELKDGYISQVVRKIVDLSI |
| | YKNTETKEFREMPAFIVLEDLNIGFKRGRQKIEKQVYQKLELALAKKLNFL |
| | VDKKADIGEIGSVTKAIQLTPPVNNFGDMENRKQFGNMLYIRADYTSQTDP |
| | ATGWRKSIYLKSGSESNVKEQIEKSFFDIRYESGDYCFEYRDRHGKMWQLY |
| | SSKNGVSLDRFHGERNNSKNVWESEKQPLNEMLDILFDEKRFDKSKSLYEQ |
| | MFKGGVALTRLPKEINKKDKPAWESLRFVIILIQQIRNTGKNGDDRNGDFI |
| | QSPVRDEKTGEHFDSRIYLDKEQKGEKADLPTSGDANGAYNIARKGIVVAE |
| | HIKRGFDKLYISDEEWDTWLAGDEIWDKWLKENRESLTKTRK* |
| SEQ ID: 27 | MNYKTGLEDFIGKESLSKTLRNALIPTESTKIHMEEMGVIRDDELRAEKQQ |
| | ELKEIMDDYYRTFIEEKLGQIQGIQWNSLFQKMEETMEDISVRKDLDKIQN |
| | EKRKEICCYFTSDKRFKDLFNAKLITDILPNFIKDNKEYTEEEKAEKEQTR |
| | VLFQRFATAFTNYFNQRRNNFSEDNISTAISFRIVNENSEIHLQNMRAFQR |
| | IEQQYPEEVCGMEEEYKDMLQEWQMKHIYSVDFYDRELTQPGIEYYNGICG |
| | KINEHMNQFCQKNRINKNDFRMKKLHKQILCKKSSYYEIPFRFESDQEVYD |
| | ALNEFIKTMKKKEIIRRCVHLGQECDDYDLGKIYISSNKYEQISNALYGSW |
| | DTIRKCIKEEYMDALPGKGEKKEEKAEAAAKKEEYRSIADIDKIISLYGSE |
| | MDRTISAKKCITEICDMAGQISIDPLVCNSDIKLLQNKEKTTEIKTILDSF |
| | LHVYQWGQTFIVSDIIEKDSYFYSELEDVLEDFEGITTLYNHVRSYVTQKP |
| | YSTVKFKLHFGSPTLANGWSQSKEYDNNAILLMRDQKFYLGIFNVRNKPDK |
| | QIIKGHEKEEKGDYKKMIYNLLPGPSKMLPKVFITSRSGQETYKPSKHILD |
| | GYNEKRHIKSSPKFDLGYCWDLIDYYKECIHKHPDWKNYDFHFSDTKDYED |
| | ISGFYREVEMQGYQIKWTYISADEIQKLDEKGQIFLFQIYNKDFSVHSTGK |
| | DNLHTMYLKNLFSEENLKDIVLKLNGEAELFFRKASIKTPIVHKKGSVLVN |
| | RSYTQTVGNKEIRVSIPEEYYTEIYNYLNHIGKGKLSSEAQRYLDEGKIKS |
| | FTATKDIVKNYRYCCDHYFLHLPITINFKAKSDVAVNERTLAYIAKKEDIH |
| | IIGIDRGERNLLYISVVDVHGNIREQRSFNIVNGYDYQQKLKDREKSRDAA |
| | RKNWEEIEKIKELKEGYLSMVIHYIAQLVVKYNAVVAMEDLNYGFKTGRFK |
| | VERQVYQKFETMLIEKLHYLVFKDREVCEEGGVLRGYQLTYIPESLKKVGK |
| | QCGFIFYVPAGYTSKIDPTTGFVNLFSFKNLTNRESRQDFVGKFDEIRYDR |
| | DKKMFEFSFDYNNYIKKGTILASTKWKVYTNGTRLKKIVVNGKYTSQSMEV |
| | ELTDAMEKMLQRAGIEYHDGKDLKGQIVEKGIEAEIIDIFRLTVQMRNSRS |
| | ESEDREYDRLISPVLNDKGEFFDTATADKTLPQDADANGAYCIALKGLYEV |
| | KQIKENWKENEQFPRNKLVQDNKTWFDFMQKKRYL* |
| SEQ ID: 28 | MRISKTLSLRIVRPFYTPEVEAGIKAEKDKREAQGQTRSLDAKFFNELKKK |
| | HSEIILSSEFYSLLSEVQRQLTSIYNHAMSNLYHKIIVEGEKTSTSKALSN |
| | IGYDECKAIFPSYMALGLRQKIQSNFRRRDLKNFRMAVPTAKSDKFPIPIY |
| | RQVDGSKGGFKISENDGKDFIVELPLVDYVAEEVKTAKGRFTKINISKPPK |
| | IKNIPVILSTLRRRQSGQWFSDDGTNAEIRRVISGEYKVSWIEIVRRTRFG |
| | KHDDWFVNMVIKYDKPEEGLDSKWGGIDVGVSSPLVCALNNSLDRYFVKSS |
| | DIIAFNKRAMARRRTLLRQNKYKRSGHGSKNKLEPITVLTEKNERFKKSIM |
| | QRWAKEVAEFFRGKGASVVRMEELSGLKEKDNFFSSYLRMYWNYGQLQQII |
| | ENKLKEYGIKVNYVSPKDTSKKCHSCTHINEFFTFEYRQKNNFPLFKCEKC |
| | GVECSADYNAAKNMATA |
| SEQ ID: 29 | MRTMVTFEDFTKQYQVSKTLRFELIPQGKTLENMKRDGIISVDRQRNEDYQ |
| | KAKGILDKLYKYILDFTMETVVIDWEALATATEEFRKSKDKKTYEKVQSKI |
| | RTALLEHVKKQKVGTEDLFKGMFSSKIITGEVLAAFPEIRLSDEENLILEK |
| | FKDFTTYFTGFFENRKNVFTDEALSTSFTYRLVNDNFIKFFDNCIVFKNVV |
| | NISPHMAKSLETCASDLGIFPGVSLEEVFSISFYNRLLTQTGIDQFNQLLG |
| | GISGKEGEHKQQGLNEIINLAMQQNLEVKEVLKNKAHRFTPLFKQILSDRS |
| | TMSFIPDAFADDDEVLSAVDAYRKYLSEKNIGDRAFQLISDMEAYSPELMR |
| | IGGKYVSVLSQLLFYSWSEIRDGVKAYKESLITGKKTKKELENIDKEIKYG |
| | VTLQEIKEALPKKDIYEEVKKYAMSVVKDYHAGLAEPLPEKIETDDERASI |
| | KHIMDSMLGLYRFLEYFSHDSIEDTDPVFGECLDTILDDMNETVPLYNKVR |
| | NFSTRKVYSTEKFKLNFNNSSLANGWDKNKEQANGAILLRKEGEYFLGIFN |
| | SKNKPKLVSDGGAGIGYEKMIYKQFPDFKKMLPKCTISLKDTKAHFQKSDE |
| | DFTLQTDKFEKSIVITKQIYDLGTQTVNGKKKFQVDYPRLTGDMEGYRAAL |
| | KEWIDFGKEFIQAYTSTAIYDTSLFRDSSDYPDLPSFYKDVDNICYKLTFE |
| | WIPDAVIDDCIDDGSLYLFKLHNKDFSSGSIGKPNLHTLYWKALFEEENLS |
| | DVVVKLNGQAELFYRPKSLTRPVVHEEGEVIINKTTSTGLPVPDDVYVELS |
| | KFVRNGKKGNLTDKAKNWLDKVTVRKMPHAITKDRRFTVDKFFFHVPITLN |
| | YKADSSPYRFNDFVRQYIKDCSDVKIIGIDRGERNLIYAVVIDGKGNIIEQ |
| | RSFNTVGTYNYQEKLEQKEKERQTARQDWATVTKIKDLKKGYLSAVVHELS |
| | KMIVKYKAIVALENLNVGFKRMRGGIAERSVYQQFEKALIDKLNYLVFKDE |
| | EQSGYGGVLNAYQLTDKFESFSKMGQQTGFLFYVPAAYTSKIDPLTGFINP |
| | FSWKHVKNREDRRNFLNLFSKLYYDVNTHDFVLAYHHSNKDSKYTIKGNWE |
| | IADWDILIQENKEVFGKTGTPYCVGKRIVYMDDSTTGHNRMCAYYPHTELK |
| | KLLSEYGIEYTSGQDLLKIIQEFDDDKLVKGLFYIIKAALQMRNSNSETGE |
| | DYISSPIEGRPGICFDSRAEADTLPYDADANGAFHIAMKGLLLTERIRNDD |
| | KLAISNEEWLNYIQEMRG* |
| SEQ ID: 30 | MSKLEKFTNCYSLSKTLRFKAIPVGKTQENIDNKRLLVEDEKRAEDYKGVK |
| | KLLDRYYLSFINDVLHSIKLKNLNNYISLFRKKTRTEKENKELENLEINLR |
| | KETAKAFKGNEGYKSLFKKDTIETILPEFLDDKDETALVNSFNGFTTAFTG |
| | FFDNRENMFSEEAKSTSIAFRCINENLTRYISNMDIFEKVDAIFDKHEVQE |
| | IKEKILNSDYDVEDFFEGEFFNFVLTQEGIDVYNAIIGGFVTESGEKIKGL |
| | NEYINLYNQKTKQKLPKFKPLYKQVLSDRESLSFYGEGYTSDEEVLEVFRN |
| | TLNKNSEIFSSIKKLEKLFKNFDEYSSAGIFVKNGPAISTISKDIFGEWNV |
| | IRDKWNAEYDDTHLKKKAVVTEKYEDDRRKSFKKTGSFSLEQLQEYADADL |
| | SVVEKLKEIIIQKVDEIYKVYGSSEKLFDADFVLEKSLKKNDAVVAIMKDL |
| | LDSVKSFENYIKAFFGEGKETNRDESFYGDFVLAYDILLKVDHIYDAIRNY |
| | VTQKPYSKDKFKLYFQNPQFMGGWDKDKETDYRATILRYGSKYYLAIMDKK |
| | YAKCLQKIDKDDVNGNYEKINYKLLPGPNKMLPKVFFSKKWMAYYNPSEDI |
| | QKIYKNGTFKKGDMFNLNDCHKLIDFFKDSISRYPKWSNAYDFNFSETEKY |
| | KDIAGFYREVEEQGYKVSFESASKKEVDKLVEEGKLYMFQIYNKDFSDKSH |
| | GTPNLHTMYFKLLFDENNHGQIRLSGGAELFMRRASLKKEELVVHPANSPI |
| | ANKNPDNPKKTTTLSYDVYKDKRFSEDQYELHIPIAINKCPKNIFKINTEV |
| | RVLLKHDDNPYVIGIDRGERNLLYIVVVDGKGNIVEQYSLNEIINNFNGIR |
| | IKTDYHSLLDKKEKERFEARQNWTSIENIKELKAGYISQVVHKICELVEKY |
| | DAVIALEDLNSGFKNSRVKVEKQVYQKFEKMLIDKLNYMVDKKSNPCATGG |
| | ALKGYQITNKFESFKSMSTQNGFIFYIPAWLTSKIDPSTGFVNLLKTKYTS |
| | IADSKKFISSFDRIMYVPEEDLFEFALDYKNFSRTDADYIKKWKLYSYGNR |
| | IRIFRNPKKNNVFDWEEVCLTSAYKELFNKYGINYQQGDIRALLCEQSDKA |
| | FYSSFMALMSLMLQMRNSITGRTDVDFLISPVKNSDGIFYDSRNYEAQENA |
| | ILPKNADANGAYNIARKVLWAIGQFKKAEDEKLDKVKTATSNKEWLEYAQT |
| | SVKH |
| SEQ ID: 31 | MTNFDNFTKKYVNSKTIRLEAIPVGKTLKNIEKMGFIAADRQRDEDYQKAK |
| | SVIDHIYKAFMDDCLKDLFLDWDPLYEAVVACWRERSPEGRQALQIMQADY |
| | RKKIADRFRNHELYGSLFTKKIFDGSVAQRLPDLEQSAEEKSLLSNFNKFT |
| | SYFRDFFDKRKRLFSDDEKHSAIAYRLINENFLKFVANCEAFRRMTERVPE |
| | LREKLQNTGSLQVYNGLALDEVFSADFYNQLIVQKQIDLYNQLIGGIAGEP |
| | GTPNIQGLNATINLALQGDSSLHEKLAGIPHRFNPLYKQILSDVSTLSFVP |
| | SAFQSDGEMLAAVRGFKVQLESGRVLQNVRRLFNGLETEADLSRVYVNNSK |
| | LAAFSSMFFGRWNLCSDALFAWKKGKQKKITNKKLTEIKKWLKNSDIAIAE |
| | IQEAFGEDFPRGKINEKIQAQADALHSQLALPIPENLKALCAKDGLKSMLD |
| | TVLGLYRMLQWFIVGDDNEKDSDFYFGLGKILGSLDPVLVLYNRVRNYITK |
| | KPYSLTKFRLNFDNSQLLNGWDENNLDTNCASIFIKDGKYYLGISNKNNRP |
| | QFDTVATSGKSGYQRMVYKQFANWGRDLPHSTTQMKKVKKHFSASDADYVL |
| | DGDKFIRPLIITKEIFDLNNVKFNGKKKLQVDYLRNTGDREGYTHALHTWI |
| | NFAKDFCACYKSTSIYDISCLRPTDQYDNLMDFYADLGNLSHRIVWQTIPE |
| | EAIDNYVEQGQLFLFQLYNKDFAPGADGKPNLHTLYWKAVFNPENLEDVVV |
| | KLNGKAELFYRPRSNMDVVRHKVGEKLVNRKLKNGLTLPSRLHEEIYRYVN |
| | GTLNKDLSADARSVLPLAVVRDVQHEIIKDRRFTADKFFFHASLTFNFKSS |
| | DKPVGFNEDVREYLREHPDTYVVGVDRGERNLIYIVVIDPQGNIVEQRSFN |
| | MINGIDYWSLLDQKEKERVEAKQAWETVGKIKDLKCGYLSFLIHEITKIII |
| | KYHAVVILENLSLGFKRVRTGIAEKAVYQQFERMLVTKLGYVVFKDRAGKA |
| | PGGVLNAYQLTDNTRTAENTGIQNGFLFYVPAAFTSRVDPATGFFDFYDWG |
| | KIKTATDKKNFIAGFNSVRYERSTGDFIVHVGAKNLAVRRVAEDVRTEWDI |
| | VIEANVRKMGIDGNSYISGKRIRYRSGEQGHGQYENHLPCQELIRALQQYG |
| | IQYETGKDILPAILQQDDAKLTDTVFDVFRLALQMRNTSAETGEDYFNSVV |
| | RDRSGRCFDTRRAEAAMPKEADANDAYHIALKGLFVLEKLRKGESIGIKNT |
| | EWLRYVQQRHS* |
| SEQ ID: 32 | MTPIFCNFVVYQIMLFNNNININVKTMNKKHLSDFTNLFPVSKTLRFRLEP |
| | QGKTMENTVKAQTIETDEERSHDYEKTKEYTDDYHRQFTDDTLDKFAFKVE |
| | STGNNDSLQDYLDAYLSANDNRTKQTEEIQTNLRKAIVSAFKMQPQFNLLF |
| | KKEMVKHLLPQFVDTDDKKRIVAKFNDFTTYFTGFFTNRENMYSDEAKSTS |
| | IAYRIVNQNLIKFVENMLTFKSHILPILPQEQLATLYDDFKEYLNVASIAE |
| | MFELDHFSIVLTQRQIEVYNSVIGGRKDENNKQIKPGLNQYINQHNQAVKD |
| | KSARLPLLKPLFNQILSEKAGVSFLPKQFKSASEVVKSLNEAYAELSPVLA |
| | AIQDVVTNITDYDCNGIFIKNDLGLTDIAQRFYGNYDAVKRGLRNQYELET |
| | PMHNGQKAEKYEEQVAKHLKSIESVSLAQINQVVTDGGDICDYFKAFGATD |
| | DGDIQRENLLASINNAHTAISPVLNKENANDNELRKNTMLIKDLLDAIKRL |
| | QWFAKPLLGAGDETNKDQVFYGKFEPLYNQLDETISPLYDKVRSYLTKKPY |
| | SLDKFKINFEKSNLLGGWDPGADRKYQYNAVILRKDNDFYLGIMRDEATSK |
| | RKCIQVLDCNDEGLDENFEKVEYKQIKPSQNMPRCAFAKKECEENADIMEL |
| | KRKKNAKSYNTNKDDKNALIRHYQRYLDRTYPEFGFVYKDADEYDTVKAFT |
| | DSMDSQDYKLSFLQVSETGLNKLVDEGDLYLFKITNKDFSSYAKGRPNLHT |
| | IYWRMLFDPKNLANVVYKLEGKAEVFFRRKSLASTTTHKAKQAIKNKSRYN |
| | EAVKPQSTFDYDIIKDRRFTADKFEFHVPIKMNFKAAGWNSTRLTNEVREF |
| | IKSQGVRHIIGIDRGERHLLYLTMIDMDGNIVKQCSLNAPAQDNARASEVD |
| | YHQLLDSKEADRLAARRNWGTIENIKELKQGYLSQVVHLLATMMVDNDAIL |
| | VLENLNAGFMRGRQKVEKSVYQKFEKMLIDKLNYIVDKGQSPDKPTGALHA |
| | VQLTGLYSDFNKSNMKRANVRQCGFVFYIPAWNTSKIDPVTGFVNLFDTHL |
| | SSMGEIKAFFSKFDSIRYNQDKGWFEFKFDYSRFTTRAEGCRTQWTVCTYG |
| | ERIWTHRSKNQNNQFVNDTVNVTQQMLQLLQDCGTDPNGNLKEATANTDSK |
| | KSLETLLHLFKLTVQMRNSVTGSEVDYMISPVADERGHFFDSRESDEHLPA |
| | NADANGAFNIARKGLMVVRQIMATDDVSKIKFAVSNKDWLRFAQHIDD* |
| SEQ ID: 33 | VKISKTLSLRIIRPYYTPEVESAIKAEKDKREAQGQTRNLDAKFFNELKKK |
| | HPQIILSGEFYSLLFEMQRQLTSIYNRAMSSLYHKIIVEGEKTSTSKALSD |
| | IGYDECKSVFPSYIALGLRQKIQSNFRRKELKGFRMAVPTAKSDKFPIPIY |
| | KQVDDGKGGFKISENKEGDFIVELPLVEYTAEDVKTAKGKFTKINISKPPK |
| | IKNIPVILSTLRRKQSGQWFSDEGTNAEIRRVISGEYKVSWIEVVRRTRFG |
| | KHDDWFLNIVIKYDKTEDGLDPEVVGGIDVGVSTPLVCAVNNSLDRYFVKS |
| | SDIIAFKKRAMARRRTLLRQNRFKRSGHGSKSKLEPITILTEKNERFKKSI |
| | MQRWAKEVAEFFKGERASVVQMEELSGLKEKDNFFGSYLRMYWNYGQLQQI |
| | IENKLKEYGIKVNYVSPKDTSKKCHSCGYINEFFTFEFRQKNNFPLFKCKK |
| | CGVECNADYNAAKNIAIA |
| SEQ ID: 34 | VKLPILKPLHKQILSEEYSTSFKIKAFENDNEVLKAIDTFWNEHIEKSIHP |
| | VTGNKFNILSKIENLCDQLQKYKDKDLEKLFIERKNLSTVSHQVYGQWNII |
| | RDALRMHLEMNNKNTKEKDTDKYLDNDAFSWKEIKDSTKTYKEHVEDAKEL |
| | NENGIIKYFSAMSINEEDDEKEYSISLIKNINEKYNNVKSILQEDRTGKSD |
| | LHQDKEKVGIIKEFLDSLKQLQWFLRLLYVTVPLDEKDYEFYNELEVYYEA |
| | LLPLNSLYNKVRNYMTRKPYSVEKFKLNFNSPTLLDGWDKNKETANLSIIL |
| | RKNGKYYLGIMNKENNTIFEYYPGTKSNDYYEKMIYKLLPGPNKMLPKVFF |
| | SKKGLEYYNPPKEILNTYEKGEFKKDKSGNFKKESLHTLTDFYKEAIAKNE |
| | DWEVFNFKFKNTKEYEDISQFYRDVEEQGYLITFEKVDANYVDKLVKEGKL |
| | YLFQIYNKDFSENKKSKGNPNLHTIYWKGLYDSENLKNVVYKLNGEAEVFY |
| | RKKSIDYPEEIYNHGHHKEELLGKFNYPIIKDRRYTQDKFLFHVPITMNFI |
| | SKEEKRVNQLACEYLSATKEDVHIIGIDRGERHLLYLSLIDKEGNIKKQLS |
| | LNTIKNENYDKEIDYRVKLDEKEKKRDEARKNWDVIENIKELKEGYMSQVI |
| | HIIAKMMVEEKAILIMEDLNIGFKRGRFKVEKQVYQKFEKMLIDKLNYLVF |
| | KNKNPLEPGGSLNAYQLTSKFDSFKKLGKQSGFIFYVPSAYTSKIDPTTGF |
| | YNFIQVDVPNLEKGKEFFSKFEKIIYNTKEDYFEFHCKYGKFVSEPKNKDN |
| | DRKTKESLTYYNAIKDTVWVVCSTNHERYKIVRNKAGYYESHPVDVTKNLK |
| | DIFSQANINYNEGKDIKPIIIESNNAKLLKSIAEQLKLILAMRYNNGKHGD |
| | DEKDYILSPVKNKQGKFFCTLDGNQTLPINADANGAYNIALKGLLLIEKIK |
| | KQQGKIKDLYISNLEWFMFMMSR |

In some instances, the Type V CRISPR/Cas protein has been modified (also referred to as an engineered protein). For example, a Type V CRISPR/Cas protein disclosed herein or a variant thereof may comprise a nuclear localization signal (NLS). In some cases, the NLS may comprise a sequence of KRPAATKKAGQAKKKKEF. Type V CRISPR/Cas proteins may be codon optimized for expression in a specific cell, for example, a bacterial cell, a plant cell, a eukaryotic cell, an animal cell, a mammalian cell, or a human cell. In some embodiments, the Type V CRISPR/Cas protein is codon optimized for a human cell.

In some instances, the Type V CRISPR/Cas protein has been modified (also referred to as an engineered protein). For example, a Type V CRISPR/Cas protein disclosed herein or a variant thereof may comprise a nuclear localization signal (NLS). In some cases, the NLS may comprise a sequence of KRPAATKKAGQAKKKKEF. Type V CRISPR/Cas proteins may be codon optimized for expression in a specific cell, for example, a bacterial cell, a plant cell, a eukaryotic cell, an animal cell, a mammalian cell, or a human cell. In some embodiments, the Type V CRISPR/Cas protein is codon optimized for a human cell.

As used herein, a programmable nuclease generally refers to any enzyme that can cleave nucleic acid. The programmable nuclease can be any enzyme that can be or has been designed, modified, or engineered by human contribution so that the enzyme targets or cleaves the nucleic acid in a sequence-specific manner. Programmable nucleases can include, for example, zinc-finger nucleases (ZFNs), transcription activator-like effector nucleases (TALENs), and/or RNA-guided nucleases such as the bacterial clustered regularly interspaced short palindromic repeat (CRISPR)-Cas (CRISPR-associated) nucleases or Cpfl. Programmable nucleases can also include, for example, PfAgo and/or NgAgo.

Programmable nucleases described herein are compatible for use in the devices described herein and may be used in conjunction with compositions disclosed herein (e.g., guide nucleic acids, reagents for in vitro transcription, reagents for amplification, reagents for reverse transcription, reporters, or any combination thereof) to carry out highly efficient, rapid, and accurate reactions for detecting whether a target nucleic acid is present in a sample (e.g., DETECTR reactions).

The programmable nuclease system used to detect a modified target nucleic acid can comprise CRISPR RNAs (crRNAs), trans-activating crRNAs (tracrRNAs), Cas proteins, and/or reporters.

Described herein are reagents comprising a programmable nuclease capable of being activated when complexed with the guide nucleic acid and the target nucleic acid segment or portion. A programmable nuclease can be capable of being activated when complexed with a guide nucleic acid and the target sequence. The programmable nuclease can be activated upon binding of the guide nucleic acid to its target nucleic acid and degrades non-specifically nucleic acid in its environment. The programmable nuclease has trans cleavage activity once activated. A programmable nuclease can be a Cas protein (also referred to, interchangeably, as a Cas nuclease). A crRNA and Cas protein can form a CRISPR enzyme.

Several programmable nucleases are consistent with the methods and devices of the present disclosure. For example, CRISPR/Cas enzymes may be programmable nucleases used in the methods and systems disclosed herein. CRISPR/Cas enzymes can include a variety of Classes and Types of CRISPR/Cas enzymes and modified or engineered versions thereof. Programmable nucleases disclosed herein can include Class 1 CRISPR/Cas enzymes, such as the Type I, Type IV, or Type III CRISPR/Cas enzymes. Programmable nucleases disclosed herein also may include the Class 2 CRISPR/Cas enzymes, such as the Type II, Type V, and Type VI CRISPR/Cas enzymes. Preferable programmable nucleases included in the several devices disclosed herein (e.g., a microfluidic device such as a pneumatic valve device or a sliding valve device or a lateral flow assay) and methods of use thereof include one or more Type V or Type VI CRISPR/Cas enzymes.

In some embodiments, the Type V CRISPR/Cas enzyme can be a programmable Type V CRISPR/Cas enzyme (e.g., Cas12 Cas14, CasΦ, CasM08). In some embodiments the programmable nuclease may lack an HNH domain. In some embodiments the programmable nuclease may comprise a RuvC domain (e.g., comprised of three RuvC subdomains). A programmable nuclease of the present disclosure can cleave a nucleic acid via a single catalytic RuvC domain. The RuvC domain can be within a nuclease, or “NUC” lobe of the protein. The programmable nuclease can further comprise a recognition, or “REC” lobe. The REC and NUC lobes can be connected by a bridge helix (e.g., Cas12). The programmable nuclease can include a PAM recognition domain. In some embodiments the nuclease can comprise two domains for PAM recognition, (e.g., termed the PAM interacting (PI) domain and the wedge (WED) domain).

In some embodiments, the Type V CRISPR/Cas programmable nuclease can be a Cas12 protein. Some non-limited examples of programmable nucleases can include a Cas12a protein, a Cpf1 protein, a Cas12b protein, Cas12c protein, Cas12d protein, or a Cas12e protein.

In some embodiments, the programmable nuclease can be Cas13. In some embodiments the programmable nuclease can be a Cas13a, Cas13b, Cas13c, Cas13d, or Cas13e. In some cases, the programmable nuclease can be Mad7 or Mad2. In some cases, the programmable nuclease can be Cas12. In some embodiments, the programmable nuclease can be Cas12a, Cas12b, Cas12c, Cas12d, or Cas12e. In some cases, the Cas12 can be Cas12 SEQ. 1D: 17. In some cases, the programmable nuclease can be Csm1, Cas9, C2c4, C2c8, C2c5, C2c10, C2c9, or CasZ. The programmable nuclease can be smCms1, miCms1, obCms1, or suCms1. The programmable nuclease can also be C2c2. The programmable nuclease can be CasZ. The programmable nuclease can be Cas14a, Cas14b, Cas14c, Cas14d, Cas14e, Cas14f, Cas14g, or Cas14h, or a Cas14u. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. Sometimes the programmable nuclease can be an engineered nuclease that is not from a naturally occurring CRISPR-Cas system. In some cases, the programmable nuclease can be from a bacteria. In some cases, the programmable nuclease can be from a bacteriophage. In some cases, the programmable nuclease can be human engineered. In some embodiments, the programmable nuclease is recombineered. In some embodiments, the programmable nuclease is derived from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), Eubacterium rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target nucleic acid. The target nucleic acid can be RNA or DNA.

In some embodiments, a programmable nuclease as disclosed herein can be an RNA-activated programmable RNA nuclease. In some embodiments, a programmable nuclease as disclosed herein is a DNA-activated programmable RNA nuclease. In some embodiments, a programmable nuclease is capable of being activated by a target RNA to initiate trans cleavage of an RNA reporter and is capable of being activated by a target nucleic acid to initiate trans cleavage of an RNA reporter, such as a Type VI CRISPR/Cas enzyme (e.g., a Cas13 nuclease). For example, Cas13a of the present disclosure can be activated by a target RNA to initiate trans cleavage activity of the Cas13a for the cleavage of an RNA reporter and can be activated by a target nucleic acid to initiate trans cleavage activity of the Cas13a for trans cleavage of an RNA reporter. An RNA reporter can be an RNA-based reporter molecule. In some embodiments, the Cas13a recognizes and detects ssDNA to initiate transcleavage of RNA reporters. Multiple Cas13a isolates can recognize, be activated by, and detect target nucleic acid, including ssDNA, upon hybridization of a guide nucleic acid with the target nucleic acid. For example, Lbu-Cas13a and Lwa-Cas13a can both be activated to transcollaterally cleave RNA reporters by target DNA. Thus, Type VI CRISPR/Cas enzyme (e.g., a Cas13 nuclease, such as Cas13a) can be DNA-activated programmable RNA nucleases, and therefore can be used to detect a target DNA using the methods as described herein. DNA-activated programmable RNA nuclease detection of ssDNA can be robust at multiple pH values. For example, target ssDNA detection by Cas13 can exhibit consistent cleavage across a wide range of pH conditions, such as from a pH of 6.8 to a pH of 8.2. In contrast, target RNA detection by Cas13 can exhibit high cleavage activity of pH values from 7.9 to 8.2. In some embodiments, a DNA-activated programmable RNA nuclease that also is capable of being an RNA-activated programmable RNA nuclease, can have DNA targeting preferences that are distinct from its RNA targeting preferences. For example, the optimal ssDNA targets for Cas13a have different properties than optimal RNA targets for Cas13a. As one example, gRNA performance on ssDNA can not necessarily correlate with the performance of the same gRNAs on RNA. As another example, gRNAs can perform at a high level regardless of target nucleotide identity at a 3′ position on a target RNA sequence. In some embodiments, gRNAs can perform at a high level in the absence of a G at a 3′ position on a target ssDNA sequence. Furthermore, target nucleic acids detected by Cas13 disclosed herein can be directly taken from organisms or can be indirectly generated by nucleic acid amplification methods, such as PCR and LAMP or any amplification method described herein. Key steps for the sensitive detection of a target nucleic acid, such as a target ssDNA, by a DNA-activated programmable RNA nuclease, such as Cas13a, can include: (1) production or isolation of target nucleic acids to concentrations above about 0.1 nM per reaction for in vitro diagnostics, (2) selection of a target sequence with the appropriate sequence features to enable DNA detection as these features are distinct from those required for RNA detection, and (3) buffer composition that enhances DNA detection.

The detection of a target nucleic acid by a DNA-activated programmable RNA nuclease can be connected to a variety of readouts including fluorescence, lateral flow, electrochemistry, or any other readouts described herein. Multiplexing of programmable DNA nuclease, such as a Type V CRISPR-Cas protein, with a DNA-activated programmable RNA nuclease, such as a Type VI protein, with a DNA reporter and an RNA reporter, can enable multiplexed detection of target ssDNAs or a combination of a target dsDNA and a target ssDNA, respectively. Multiplexing of different RNA-activated programmable RNA nucleases that have distinct RNA reporter cleavage preferences can enable additional multiplexing. Methods for the generation of ssDNA for DNA-activated programmable RNA nuclease-based diagnostics can include (1) asymmetric PCR, (2) asymmetric isothermal amplification, such as RPA, LAMP, SDA, etc. (3) NEAR for the production of short ssDNA molecules, and (4) conversion of RNA targets into ssDNA by a reverse transcriptase followed by RNase H digestion. Thus, DNA-activated programmable RNA nuclease detection of target DNA is compatible with the various systems, kits, compositions, reagents, and methods disclosed herein. For example, target ssDNA detection by Cas13a can be employed in a detection device as disclosed herein.

A programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby nucleic acids by the activated programmable nuclease, such as trans cleavage of reporters with a detection moiety. Once the reporter is cleaved by the activated programmable nuclease, the detection moiety can be released from the reporter and can generate a signal. The signal can be visualized to assess whether a target nucleic acid comprises a modification (such as a SNP).

### Guide Nucleic Acids

Guide nucleic acids are compatible for use in the devices described herein (e.g., pneumatic valve devices, sliding valve devices, rotating valve devices, and lateral flow devices) and may be used in conjunction with compositions disclosed herein (e.g., programmable nucleases, reagents for in vitro transcription, reagents for amplification, reagents for reverse transcription, and reporters, or any combination thereof) to carry out highly efficient, rapid, and accurate reactions for detecting whether a target nucleic acid is present in a sample (e.g., DETECTR reactions). The guide nucleic acid binds to the single stranded target nucleic acid comprising a portion of a nucleic acid from a virus or a bacterium or other agents responsible for a disease as described herein. The guide nucleic acid can bind to the single stranded target nucleic acid comprising a portion of a nucleic acid from a bacterium or other agents responsible for a disease as described herein and further comprising a mutation, such as a single nucleotide polymorphism (SNP), which can confer resistance to a treatment, such as antibiotic treatment. The guide nucleic acid binds to the single stranded target nucleic acid comprising a portion of a nucleic acid from a cancer gene or gene associated with a genetic disorder as described herein. The guide nucleic acid is complementary to the target nucleic acid. Often the guide nucleic acid binds specifically to the target nucleic acid. The target nucleic acid may be a RNA, DNA, or synthetic nucleic acids. A guide nucleic acid can comprise a sequence that is reverse complementary to the sequence of a target nucleic acid. A guide nucleic acid can be a crRNA. Sometimes, a guide nucleic acid comprises a crRNA and tracrRNA. The guide nucleic acid can bind specifically to the target nucleic acid. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid may have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the targeting region of a guide nucleic acid has a length of from about 10 nt to about 60 nt, from about 20 nt to about 50 nt, or from about 30 nt to about 40 nt. In some cases, the targeting region of a guide nucleic acid has a length of from 15 nt to 55 nt, from 25 nt to 55 nt, from 35 nt to 55 nt, from 45 nt to 55 nt, from 15 nt to 45 nt, from 25 nt to 45 nt, from 35 nt to 45 nt, from 15 nt to 35 nt, from 25 nt to 35 nt, or from 15 nt to 25 nt. It is understood that the sequence of a polynucleotide need not be 100% complementary to that of its target nucleic acid to be specifically hybridizable or hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

The guide nucleic acid can be selected from a group of guide nucleic acids that have been tiled against the nucleic acid of a strain of an infection or genomic locus of interest. The guide nucleic acid can be selected from a group of guide nucleic acids that have been tiled against the nucleic acid of a strain of SARS-CoV-2, influenza or other respiratory virus. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled guide nucleic acids along the target nucleic acid. In some instances the tiling of the guide nucleic acids is non-sequential. Often, a method for detecting a target nucleic acid comprises contacting a target nucleic acid to a pool of guide nucleic acids and a programmable nuclease, wherein a guide nucleic acid of the pool of guide nucleic acids has a sequence selected from a group of tiled guide nucleic acid that is reverse complementary to a sequence of a target nucleic acid; and assaying for a signal produce by cleavage of at least some reporters of a population of reporters. Pooling of guide nucleic acids can ensure broad spectrum identification, or broad coverage, of a target species within a single reaction. This can be particularly helpful in diseases or indications, like sepsis, that may be caused by multiple organisms.

### DETECTR

The system may perform detection using a DNA Endonuclease Targeted CRISPR TransReporter (DETECTR) assay. A DETECTR assay can utilize the trans-cleavage abilities of some programmable nucleases to achieve fast and high-fidelity detection of a target nucleic acid in a sample. The target nucleic acid can be DNA or RNA. For example, following target nucleic acid extraction from a biological sample, crRNA comprising a portion that is complementary to the target nucleic acid of interest can bind to the target nucleic acid sequence, initiating indiscriminate ssDNase activity by the programmable nuclease. Upon hybridization with the target nucleic acid, the trans-cleavage activity of the programmable nuclease is activated, which can then cleave an ssDNA fluorescence-quenching (FQ) reporter molecule. Cleavage of the reporter molecule can provide a fluorescent readout indicating the presence of the target nucleic acid in the sample. In some embodiments, the programmable nucleases disclosed herein can be combined, or multiplexed, with other programmable nucleases in a DETECTR assay.

A DETECTR reaction to detect the target nucleic acid sequence may comprise a guide nucleic acid comprising a segment that is reverse complementary to a segment of the target nucleic acid and a programmable nuclease. The programmable nuclease when activated may exhibit sequence-independent cleavage of a reporter (e.g., a nucleic acid comprising a detection moiety that becomes detectable upon cleavage of the nucleic acid by the programmable nuclease). The programmable nuclease may be activated upon the guide nucleic acid hybridizing to the target nucleic acid. A combined LAMP DETECTR reaction may comprise a plurality of primers, dNTPs, a DNA polymerase, a guide nucleic acid, a programmable nuclease, and a substrate nucleic acid. A combined RT-LAMP DETECTR reaction may comprise LAMP primers, reverse transcription primers, dNTPs, a reverse transcriptase, a DNA polymerase, a guide nucleic acid, a programmable nuclease, and a substrate nucleic acid. In some case, the LAMP primers may comprise the reverse transcription primers. In this embodiment, RT-LAMP and DETECTR can be carried out in the same sample volume.

The concentrations of the various reagents in the programmable nuclease DETECTR reaction mix can vary depending on the particular scale of the reaction. For example, the final concentration of the programmable nuclease can vary from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM. The final concentration of the sgRNA complementary to the target nucleic acid can be from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM. The concentration of the reporter (e.g., ssDNA-FQ reporter) can be from from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM.

An example of a DETECTR reaction comprises, consists, or consists essentially of a final concentration of 100 nM CasΦ polypeptide or variant thereof, 125 nM sgRNA, and 50 nM ssDNA-FQ reporter in a total reaction volume of 20 μL. Reactions are incubated in a fluorescence plate reader (Tecan Infinite Pro 200 M Plex) for 2 hours at 37° C. with fluorescence measurements taken every 30 seconds (e.g., λex: 485 nm; λem: 535 nm). The fluorescence wavelength detected can vary depending on the reporter molecule.

In some embodiments, the DETECTR reagents and the amplification reagents can be in two separate phases. In some embodiments, the DETECR reagents can be in a first aqueous layer contacting an immiscible phase that serves to separate a second aqueous layer containing amplification reagents. The two aqueous layers can be contacted via mixing at the appropriate time.

In some embodiments, the DETECTR reagents may include an endogenous control probe. For example, at least one well may comprise a programmable nuclease and guide nucleic acid complex configured to bind to a control target (e.g., RNase P) in order to confirm that the assay has proceeded as expected. The control reaction may he monitored as described herein.

In some embodiments, the amplification reagents may include a fluorescent probe for a second target (e.g., RNase P) may be included as an endogenous control in at least one of the amplification wells in order to facilitate detection of the endogenous control in the amplification well rather than (or in addition to) in a DETECTR well. For example, an internal FAM labeled LAMP BIP primer may be included as an RT-LAMP reagent. There may be a self-quenching effect if the T near 3′ end is labeled. Once the primers are incorporated into amplicons, there may be a de-quenching effect. An end point read in the FAM channel on the plate read may detect the fluorescence increase due to dc-quenching. The RT-LAMP for RNase P may he duplexed with the primary target (e.g., SARS-CoV-2 N gene) RT-LAMP. At the plate reader, an end point read in FAM channel may detect RNase P and a kinetic read in the Alexa594 channel may detect the N gene DETECTR reaction.

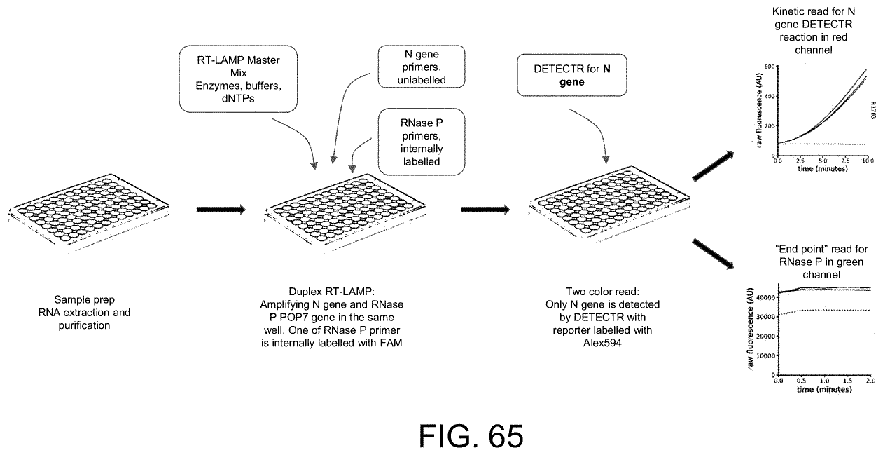

FIG. 65 shows an exemplary workflow including an RNase P endogenous control for RT-LAMP and experimental results showing detection of RNase P and N gene in a single well. RNase P primers were internally labeled with FAM and added to wells with N gene primers for RT-LAMP. A DETECTR N gene reaction was then run with an Alexa594-labeled reporter and the DETECTR reaction was monitored in the red channel over a period of 10 minutes. An end-point ready for RNase P was then taken in the green channel in the same well.

### Signal Detection

The devices, systems, fluidic devices, kits, and methods for detecting the presence of a target nucleic acid in a sample described herein may comprise a generation of a signal in response to the presence or absence of the target nucleic acid in the sample. The generation of a signal in response to the presence or absence of the target nucleic acid in the sample as described herein is compatible with the methods and devices described herein (e.g., pneumatic valve devices, sliding valve devices, rotating valve devices, and lateral flow devices) and may result from the use of compositions disclosed herein (e.g., programmable nucleases, guide nucleic acids, reagents for in vitro transcription, reagents for amplification, reagents for reverse transcription, reporters, or any combination thereof) to carry out highly efficient, rapid, and accurate reactions for detecting whether a target nucleic acid is present in a sample (e.g., DETECTR reactions). As disclosed herein, detecting the presence or absence of a target nucleic acid of interest involves measuring a signal emitted from a detection moiety present in a reporter, after cleavage of the reporter by an activated programmable nuclease. Thus, the detecting steps disclosed herein involve measuring the presence of a target nucleic acid, quantifying how much of the target nucleic acid is present, or, measuring a signal indicating that the target nucleic acid is absent in a sample. In some embodiments, a signal is generated upon cleavage of the reporter by the programmable nuclease. In other embodiments, the signal changes upon cleavage of the reporter by the programmable nuclease. In other embodiments, a signal may be present in the absence of reporter cleavage and disappear upon cleavage of the target nucleic acid by the programmable nuclease. For example, a signal may be produced in a microfluidic device or lateral flow device after contacting a sample with a composition comprising a programmable nuclease.

Often, the signal is a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorimetric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule, where the first detection signal indicates that the sample contained the target nucleic acid. Sometimes the system is capable of detecting more than one type of target nucleic acid, wherein the system comprises more than one type of guide nucleic acid and more than one type of reporter. In some cases, the detectable signal is generated directly by the cleavage event. Alternatively or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

### Particular Implementations

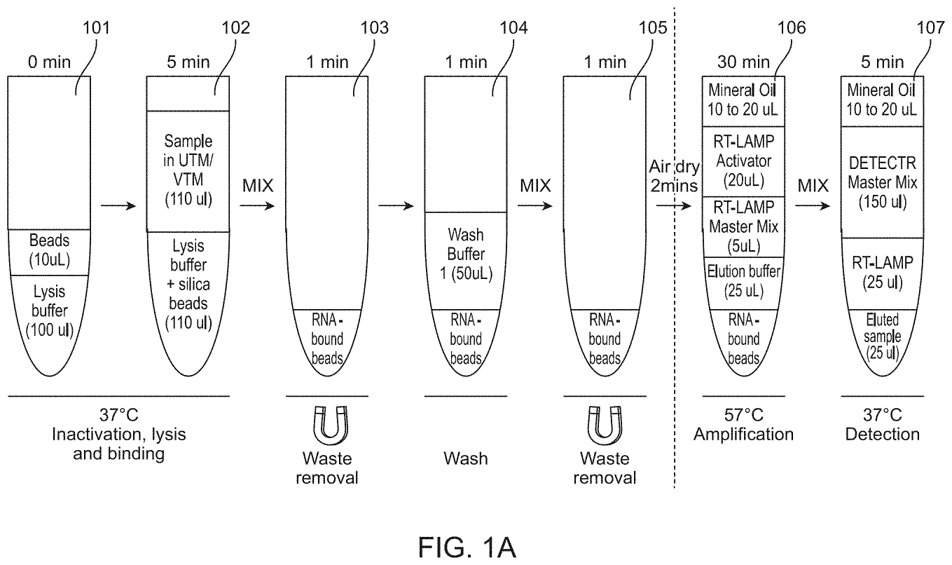

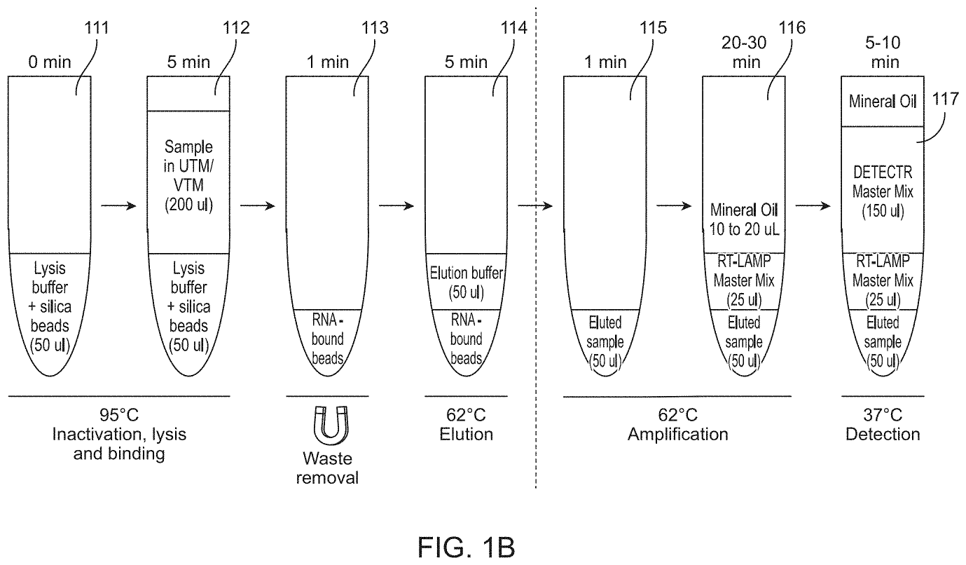

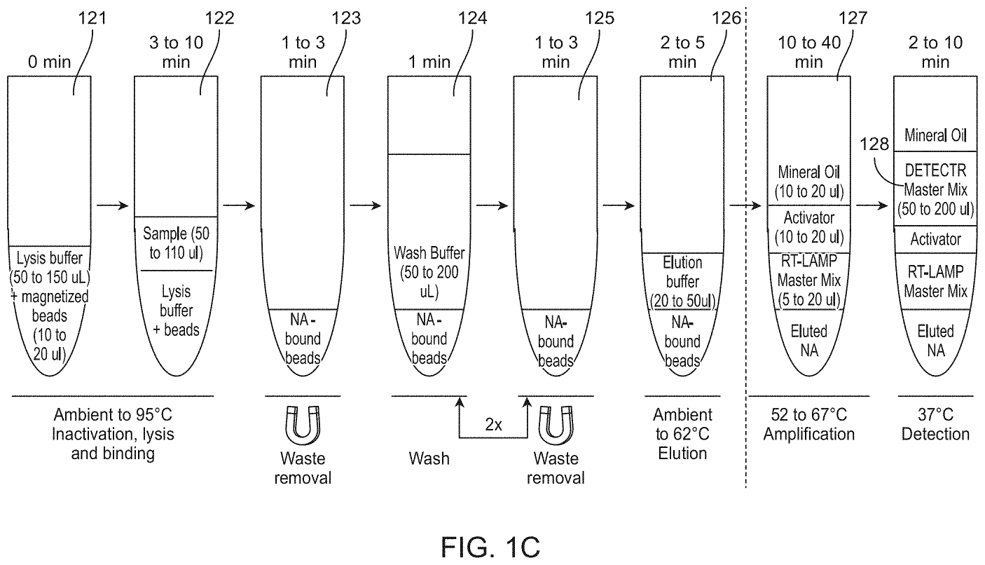

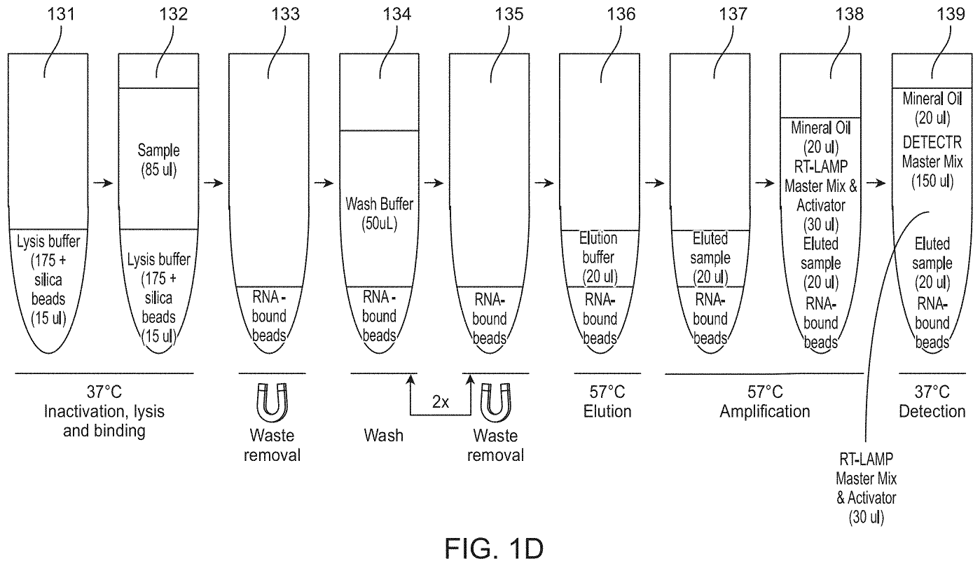

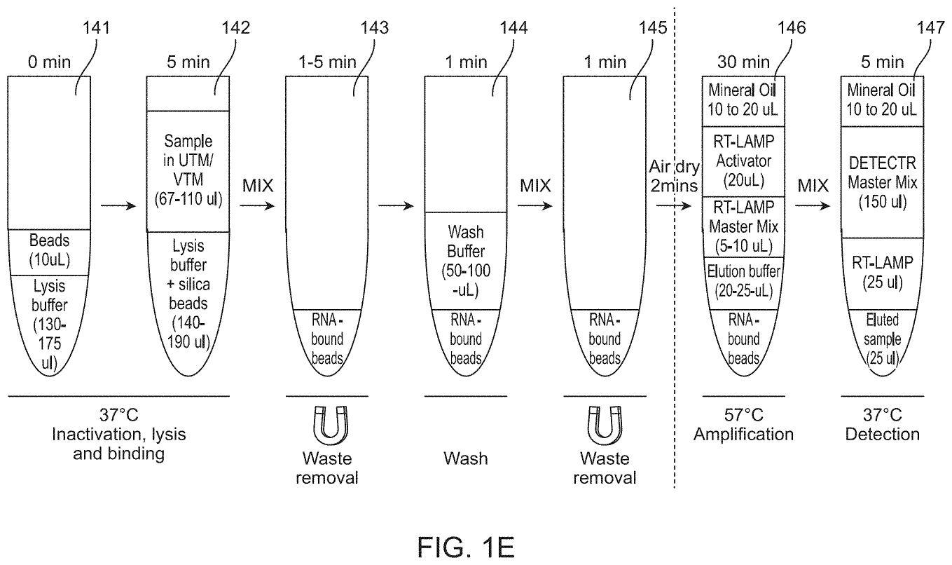

FIGS. 1A, 1B, 1C, 1D and 1E show exemplary methods for programmable nuclease-based detection. The method can comprise collecting a sample. The sample can comprise any type of sample as described herein. The method can comprise preparing the sample. Sample preparation can comprise one or more sample preparation steps. A holder or receptacle carrying the sample may be affixed with an identifier (e.g., barcode, etc.), which may be scanned by an instrument to determine whether the samples are in their correct locations, e.g., placed correctly on a sample deck. In other embodiments, the samples may be scanned manually via a bar code scan method. The sample may be received in a closed tube with a swab. The tube may need to be uncapped and the swab removed prior to commencement of testing. Additionally, positive and negative control samples may be loaded into the system. The turnaround time to complete all operations may be under an hour. The sample may be a lower nasal swab sample or a saliva sample. The volumes represented in the figures are examples of typical volumes. FIG. 1D illustrates an automated assay.

The method can comprise generating one or more droplets, aliquots, or subsamples from the sample. The one or more droplets, aliquots, or subsamples can correspond to a volumetric portion of the sample. The sample can be divided into 1, 2, 3, 4, 5, 6, 7, 8, 9, 10, 20, 30, 40, 50, 60, 70, 80, 90, 100, 200, 300, 400, 500, 600, 700, 800, 900, 1000, or more droplets, aliquots, or subsamples. The sample can be divided into between 1 and 10, between 10 and 20, between 20 and 50, between 50 and 100, between 100 and 200, between 200 and 500, or between 500 and 1000 or more droplets, aliquots, and or subsamples.

In some embodiments, the method may have a limit of detection of less than 2000 copies/mL, less than 1000 copies/mL, less than 500 copies/mL, less than 200 copies/mL, less than 100 mL, or less than 50 mL. In some embodiments, the method may have a limit of detection of more than 2000 copies/mL, more than 1000 copies/mL, more than 500 copies/mL, more than 200 copies/mL, more than 100 mL, or more than 50 mL. The method may have a limit of detection of between 20 and 50 copies/mL, between 50 and 100 copies/mL, between 100 and 200 copies/mL, between 200 and 500 copies/mL, or between 500 and 1000 copies/mL. The method may provide a test with a sensitivity of above 75%, above 80%, above 85%, above 90%, above 95%, above 99%, or above 99.9%. The method may provide a test with a specificity of above 75%, above 80%, above 85%, above 90%, above 95%, above 99%, or above 99.9%. Implementing the test on a workstation may provide a testing capacity of greater than 50 per one hour period, greater than 100 per one hour period, or greater than 200 per one hour period. Implementing the test on a workstation may provide a testing capacity of greater than 1500 tests per 8 hour period or greater than 4500 tests per 24 hour period. The high-throughput testing system may provide about 400 results every 1.75 hours.

In some embodiments, the sample can be provided manually to the chamber of the present disclosure. For example, a swab sample can be dipped into a solution and the sample/solution can be pipetted into the chamber. In other embodiments, the sample can be provided via an automated syringe. The automated syringe can be configured to control a flow rate at which the sample is provided to the chamber. The automated syringe can be configured to control a volume of the sample that is provided to the detection device over a predetermined period.

In some embodiments, the single-chamber reaction may use an RT-LAMP probe or probes in order to detect amplification controls in addition to a reporter used for disease detection. The reporter used for disease detection would be acted on by the programmable nuclease to create a signal during the DETECTR reaction. The RT-LAMP probe or probes would hybridize and create a target-specific signal during the RT-LAMP amplification and would be used for one or more sample and amplification controls (e.g., RnaseP) that require less sensitivity/specificity. Detection of the disease using the probe for disease detection may require higher sensitivity/specificity than can be provided by the RT-LAMP probe. The programmable nuclease may cleave a sample target nucleic acid or a reporter molecule.

### Workflows

FIGS. 1A-1E illustrate different exemplary workflows for providing a single-chamber detection reaction. In the example embodiments, the sample in the chamber is first lysed to release nucleic acids. The nucleic acids in the sample are then isolated using magnetic beads. Following this, amplification occurs. Then detection occurs, where the sample is contacted with a detection reagent. The detection reagent may include the programmable nuclease, the reporter molecule, the guide nucleic acid, or a combination thereof.

In some embodiments, the assays can be performed using a 96-well plate or 384-well microplate. In some embodiments, the assays may be performed using a 6, 12, 24, 48, 96, 384, or 1536-well microplate. The assays may be performed using another type of rectangular microplate with a 2:3 array of wells, or a 4:3 array of wells, or more. The wells in the microplate may have fill volumes of more than 50 μL, more than 100 μL, more than 150 μL, more than 200 μL, more than 250 μL, or more than 300 μL. A plurality of wells may be empty to prevent cross-contamination. For example, more than 10%, more than 20%, more than 30%, more than 40%, more than 50%, more than 60%, more than 70%, more than 80%, or more than 90% of the wells may be empty during a run. Samples may be spaced such that any two adjacent wells do not both contain samples. In some embodiments, no wells may be empty and cross-contamination may be limited by other means (e.g., careful liquid handling, etc.).

Embodiments of the disclosure may use four assay processing stations in order to improve the speed of sample processing. In some embodiments, there may be more than two, more than three, more than four, more than five, more than ten, or more than 15 assay processing stations.

A particular assay may be completed in fewer than 30 minutes, fewer than 40 minutes, fewer than 50 minutes, less than an hour, or less than two hours. Multiple assays may be conducted in parallel. Multiple assays may be staggered in time. Assays may be staggered between 1 and 10 minutes apart, between 10 and 20 minutes apart, between 20 and 30 minutes apart, between 30 and 40 minutes apart, between 40 and 50 minutes apart, between 50 minutes and one hour apart, or between one hour and two hours apart, or any range in between 1 minute and two hours.

In some embodiments, inactivation, lysis, isolation, elution, amplification, and detection are performed at a single station. In some embodiments, the six operations are performed at between one and six stations, where the microplate is transported between stages during the assay process. In some embodiments, there are six stations or more. In some embodiments, some operations are omitted. For example, in some embodiments, elution may be omitted. In some embodiments, depending on the type of sample analyzed, elution may be omitted. In some embodiments, some operations may be performed simultaneously. In some embodiments, one or more of the following combinations of operations may be performed simultaneously. In some embodiments, portions of operations are split. For example, in some embodiments, inactivation, lysis, and a portion of the isolation operation are performed together, while the remaining isolation steps (washing and waste removal) are performed later. In some embodiments, elution and amplification are performed simultaneously or near-simultaneously. In other embodiments, elution and amplification are performed sequentially. In some embodiments waste removal is performed twice and washing is performed after a first waste removal step and before a second. In some embodiments, waste removal is performed three times and washing is performed after the first and again after the second waste removal operation.

The assay may proceed as follows. During inactivation, lysis, and binding, nucleic acid molecules may be released from the raw sample and bound to microparticles to produce a microparticle complex including the nucleic acid molecules and the microparticles. Then, during isolation, the un-complexed portion of the sample may be separated from the microparticle complex. During elution, the nucleic acid molecules may be separated from the microparticle complex. Then, the nucleic acid molecules may be amplified. Finally, in a detection reaction, a programmable nuclease and guide nucleic acid complex may contact the nucleic acid molecules, which may activate transcleavage activity of the programmable nuclease and enable the programmable nuclease to cleave a reporter and produce a fluorescent signal upon release of a detection moiety therefrom.

During the amplification and detection operations, the one or more microparticles may remain in the single chamber.

In some embodiments, nucleic acid molecules may bind to microparticles, producing multiple complexes containing nucleic acid molecules and microparticles. The assay may then isolate the complexes and elute the nucleic acid molecules from the complexes, before contacting them with an amplification agent to amplify the nucleic acid molecules, comprising an amplified product. Then, the assay may contact the amplified product with a complex comprising a guide nucleic acid, a reporter molecule, and a programmable nuclease. If a target nucleic acid is present in the amplified product, the programmable nuclease may cleave the reporter molecule, which may emit a detectable signal indicative of the presence of the target nucleic acid. If a target nucleic acid is not present in the amplified product, the programmable nuclease may cleave the reporter molecule, which may emit a detectable signal indicative of the absence of the target nucleic acid. During the amplification and detection operations, the microparticles from the complexes may remain in the single chamber.

In some embodiments, the method may include multiplexing. During or prior to the amplification step, the nuclease may be contacted with a first probe, which produces a detectable signal during or following amplification, from the contacting. If the amplification is RT-LAMP amplification, the probe may be a dye configured to produce a colorimetric signal when the pH changes during the amplification process. Alternatively, the probe may be a label configured to produce a fluorescent signal at a first wavelength. During the detection step, the programmable nuclease may be complexed with a second probe, which may be a guide nucleic acid. When the second probe binds a segment of the target nucleic acid, the programmable nuclease may cleave a reporter molecule, which may produce a second detectable signal. The second detectable signal may be a fluorescent signal. When the first signal is a fluorescent signal, the second detectable signal may be at a distinct wavelength from the first signal.

In some embodiments, amplification and contacting the nucleic acid molecules with the programmable nuclease complex may occur simultaneously.

In first operations 101, 111, 121, 131, 141 the system provides a lysis agent and microparticles in the single chamber. The single chamber may be a particular tube or particular well of a microplate, which may be 96 or 384 wells. The microparticles may be silica-coated beads or magnetized beads. The first operation 101 may be completed in under one minute. Various volumes of lysis buffer solution and microparticles may be provided. For example, in operation 101, 10 μL of beads may be added to 100 μL of lysis buffer. In operation 111, a 50 μL lysis buffer bead mix may be provided in a chamber. In operation 121, 50-150 μL lysis buffer may be added to 10 to 20 μL beads. In operation 131, 175 μL of lysis buffer solution may be inserted into the chamber, with 15 μL of beads. In operation 141, 130-175 μL lysis buffer may be added to 10 μL beads in the microchamber. One of ordinary skill in the art will appreciate that the volume of beads and lysis buffer may be optimized to achieve a desired total reaction volume, to achieve a desired capture efficiency, to adapt the workflow to different lysis buffers and/or microparticles, etc.

In second operations 102, 112, 122, 132, 142, the sample is added to the single chamber containing lysis buffer and microparticles. The system may increase the temperature of the chamber up to 95° C. or 37° C. in order to initiate lysis. Lysis may be performed at a temperature within a range of ambient temperature to 95° C. In some embodiments, lysis may be performed at room temperature, 37° C., 62° C., or 95° C. The sample may be in a uniform transport medium (UTM) or a viral transport medium (VTM). Various volumes of samples may be provided. For example, in operation 102, 110 μL of sample may be added to the chamber, for a total lysis volume of 200 μL. In operation 112, 200 μL of sample may be added to the chamber, for a total lysis volume of 250 μL. In operation 122, 50-110 μL of sample may be added to the chamber, for a total lysis volume of 110-280 μL. In operation 132, 85 μL of sample may be added to the chamber, for a total lysis volume of 275 μL. In operation 142, 67-110 μL of sample may be added to the chamber, for a total lysis volume of 207-300 μL. One of ordinary skill in the art will appreciate that the volume of sample may be optimized to achieve a desired total reaction volume, to achieve a desired capture efficiency, to adapt the workflow to different sample types or media, etc.

The second operations 102, 112, 122, 132, 142 may also comprise heat inactivation. In some embodiments, a processed/lysed sample can undergo heat inactivation to inactivate, in the lysed sample, the proteins used during lysing (e.g., a PK enzyme or a lysing reagent). Heat inactivation may also inactivate or kill any live viruses or bacteria in the sample. In some cases, a heating element in proximity to the chamber in which the reaction is performed may be used for heat-inactivation. The heating element can be powered by a battery or another source of thermal or electric energy that is integrated with the detection device. Heat inactivation may be performed at a temperature within a range of ambient temperature to 95° C. In some embodiments, heat inactivation may be performed at room temperature, 37° C., 62° C., or 95° C. The operations 102, 112, 122, 132, and 142 may be completed in between three and ten minutes. Lysis, inactivation, and binding may occur in about 3 minutes, about 4 minutes, about 5 minutes, about 6 minutes, about 7 minutes, about 8 minutes, about 9 minutes, or about 10 minutes. For example, operations 102, 112, 132, and 142 may be completed in 5 minutes. Operations 101, 102, 131, 132, 141, and 142 may be performed at 37° C. Operations 111 and 112 may be performed at 95° C. Operations 121 and 122 may be performed within a range from ambient temperature to 95° C. One of ordinary skill in the art will appreciate that the reaction temperatures and times may be optimized to achieve a desired capture efficiency, to adapt the workflow to different sample types or media, etc.

In third operations 103, 104, 105, 113, 123, 124, 125, 133, 134, 135, 143, 144, and 145, the nucleic acid-microparticle complex is isolated from unbound components of the sample. When the microparticles are magnetic, this may be performed by bringing a magnet into contact with the microplate, or by bringing a microplate into contact with the magnet. This pulls the microparticle complex including the nucleic acid molecules and the microparticles down towards the bottom of the microplate well. While the microparticle complex is retained at the bottom of the well by the magnet, the system or an operator may aspirate waste liquid from the top of the well, away from the particles. Waste removal operations 103, 105, 113, 123, 125, 133, 135, 143, and 145 may be completed in between one and five minutes. For example, operation 133 may be completed in one minute. Wash operations 104, 124, 134, and 144 may be completed in between one and five minutes. Waste removal and wash operations may be performed at ambient temperature or without heat applied to the chambers. One of ordinary skill in the art will appreciate that the wash and waste removal temperatures and times, as well as the number of wash steps, may be optimized to achieve a desired capture efficiency, to adapt the workflow to different sample types or media, to remove undesired buffer components which may interfere with downstream operations (e.g., amplification, detection), etc.

In operations 106, 114-115, 126, 136-137, and 146, the system or an operator elutes the nucleic acid molecules from the microparticles. The elution may be performed using an elution buffer. 20-50 μL of elution buffer may be added to the microparticle complex within the chamber. For example, in operation 106, 25 μL of elution buffer may be added. In operation 114, 50 μL of elution buffer may be added. In operation 126, 20-50 μL of elution buffer may be added. In operation 136, 20 μL of elution buffer may be added. In operation 146, 20-25 μL of elution buffer may be added.

Prior to elution, the temperature of the well may be altered, e.g., to 57-62° C., or 45-65° C., for improved elution efficiency. The temperature of the chamber may be held within a range of about ambient temperature to about 67° C. for the elution and amplification stages, and a wash buffer may be added. In operations 105, 125, and 135, the system performs waste removal. The elution process may produce an eluted nucleic acid sample disposed above a layer comprising the microparticles. The elution process may produce eluted nucleic acids intermixed with the microparticles. Operations 104, 114, 124, 134, and 144 and 105, 115, 125-6, 135-136, and 145 may be completed in between four and ten minutes. In the embodiments of FIG. 1C and 1D, washing and waste removal steps 124, 134 and 125-126, 135 are performed twice. In FIGS. 1A and 1E, elution 106, 146 is performed at 57° C. simultaneously with amplification for 30 minutes. In FIG. 1B, elution 114 is performed at 62° C. for 5 minutes prior to amplification. In FIG. 1C, elution 126 is performed within a range from ambient temperature to 67° C. In FIG. 1D, elution 136 is performed at 57° C. for five minutes. Ambient temperature may be a range from 20-25° C. Amplification may occur between 52-67° C. In some embodiments, amplification may comprise polymerase chain reaction (PCR). In these embodiments, amplification may take place between 45° C. and 95° C., during denaturing, annealing, and extension PCR stages. One of ordinary skill in the art will appreciate that the elution temperatures, times, and volumes may be optimized to achieve a desired total reaction volume, to achieve a desired elution efficiency, to adapt the workflow to different sample types or media, etc.

In operations 106, 116, 127, 138, and 146, the nucleic acid molecules are contacted with an amplification agent to amplify them. In an exemplary embodiment, amplification may be accomplished using reverse transcription loop-mediated amplification (RT-LAMP). An RT-LAMP master mix may be added to the nucleic acid molecules in the chamber following isolation and/or elution. In some embodiments, an RT-LAMP activator may also be added to the chamber. For example, in operation 106, 20 μL of RT-LAMP activator and 5 μL of RT-LAMP master mix may be added to the chamber. In operation 116, 25 μL of RT-LAMP master mix may be added. In operation 127, 10-20 μL of RT-LAMP activator and 5-20 μL of RT-LAMP master mix may be added. In operation 138, 30 μL of RT-LAMP master mix and activator may be added. In operation 146, 20 μL of RT-LAMP activator and 5-10 μL of RT-LAMP master mix may be added. The nucleic acid amplification reaction can be performed at a temperature of within a range of 52-67° C. For example, isothermal amplification may occur at 52° C., 57° C., 62° C., or 67° C. One skilled in the art will recognize that the amplification reaction temperature can be optimized based on specific reaction components. For example, in operation 116, amplification is performed at 62° C. for 20-30 minutes. In operation 127, amplification is performed at 52-67° C., and mineral oil is added during amplification for 10-40 minutes. In operation 138, mineral oil is also added, and amplification is performed at 57° C. for 10 to 40 minutes. Operation 146 is performed at 57° C. for 30 minutes. Amplification operations 106, 116, 138, 146, and 127 may be completed in about 10-40 minutes. For example, operation 138 may be completed in 30 minutes. One of ordinary skill in the art will appreciate that the amplification temperatures, times, master mix volumes, and master mix components may be optimized to achieve a desired total reaction volume, to achieve a desired amplification efficiency, to adapt the workflow to different sample types or media, to adapt the workflow to different target nucleic acid types, to adapt the workflow to different amplification methods, etc.

In some embodiments, the RT-LAMP amplification reaction may produce a colorimetric signal. The reaction causes pH levels and Mg₂+ levels to drop. These drops may be measured using indicators such as Phenol red (for pH) and hydroxynaphthol blue (for magnesium). Alternatively, nucleic acid stains such as SYBR Green I or SYTO 9 may be used.

In operations 107, 117, 147, 128, 139, the system or operator contacts the amplified nucleic acid molecules in the chamber with a programmable nuclease and a guide nucleic acid. The guide nucleic acid and programmable nuclease may be supplied as a complex (e.g., a ribonucleoprotein complex) or in situ (without prior complex formation). In an embodiment, the complex is a Cas-gRNA complex. The complex may be in a DETECTR master mix. In some embodiments, the Cas enzyme may cleave a reporter nucleic acid linker, thereby releasing a fluorophore from its quencher molecule if a target region has been amplified as described herein. The rise of fluorescence detection may indicate a positive detection. In operations 107, 117, 147, 128, 139, the guide nucleic acid and programmable nuclease may bind to a complementary nucleic acid target from the amplified sample and may be activated into a non-specific nuclease, which may cleave a nucleic acid-based reporter molecules within the chamber to generate a signal readout. In the absence of a complementary nucleic acid target, the Cas-gRNA complex does not cleave the nucleic acid-based reporter molecule. Detection of the signal can be achieved by multiple methods, which can detect a signal that is calorimetric, potentiometric, amperometric, optical (e.g., fluorescent, colorimetric, etc.), or piezo-electric, as non-limiting examples. Various volumes of DETECTR master mix may be added to the chamber. For example, in operations 107, 117, 139, and 147, 150 μL of DETECTR master mix may be added to the chamber to initiate the detection reaction. In operation 50-200 μL of DETECTR master mix may be added. Operations 107, 117, 147, 139, and 128 may be completed in 2-10 minutes. For example, operation 139 may be completed in 10 minutes. Operations 107 and 147 may be completed in 5 minutes. Operation 117 may be completed in 5-10 minutes. Operation 128 may be completed in 2 to 10 minutes. Operations 107, 117, 128, and 139 may be completed at 37° C. One of ordinary skill in the art will appreciate that the detection temperatures, times, master mix volumes, and master mix components may be optimized to achieve a desired total reaction volume, to achieve a desired detection efficiency, to adapt the workflow to different sample types or media, to adapt the workflow to different target nucleic acid types, to achieve a desired detection speed, etc.

In some embodiments, the amplification of the nucleic acid molecules and the contacting of the molecules with a programmable nuclease may occur simultaneously. Mineral oil may be added during amplification and detection steps of FIGS. 1A, 1B, 1C, 1D, and 1E to prevent or reduce evaporation. The nucleic acid bound microparticles may be air dried for 2-5 minutes after washing in order to allow for evaporation of remaining lysis or wash buffer solution components (e.g., IPA from lysis or wash buffers).

The components of the reaction may have the following volumes. The volume of the microbeads can range from 10 μL to 20 μL. The volume of the lysis reagent can range from 50 μL to 150 μL. The volume of the sample can range from 50 μL to 110 μL. The volume of the wash buffer can range from 50 μL to 200 μL. The volume of the mineral oil may range from 10 μL to 20 μL. The volume of the elution buffer can be 20 μL to 50 μL. The volume of the RT Lamp master mix can be 5 μL to 20 μL. The volume of the activator reagent can be 10 μL to 20 μL. The DETECTR master mix may be from 50 to 200 μL.

The signal produced may be a fluorescent signal, which may be read by a fluorescent plate reader. Signals may be collected after the DETECTR reaction has been completed (e.g., at an endpoint). Signals may be collected on a periodic basis (e.g., every 20 seconds). A computing device may be configured to plot the collected signals and find their slope. The computing device may compare the slope to those determined by performing the same set of operations on a positive control and a negative control. In other embodiments, different signals may be collected. For example, the signals collected may be associated with physical, chemical, or electrochemical changes or reactions. The signals may be optical signals, potentiometric or amperometric signals, piezoelectric signals, may be associated with a change in an index of refraction of a solid or gel volume in which at least one programmable nuclease probe is disposed.

For example, an interaction between the programmable nuclease and a target nucleic acid may induce a probe to produce an oxidation signal, which may be measured by a sensing device such as a potentiostat or biosensor. The sensing device may produce a measurable output voltage in response to the oxidation signal.

In another example, an interaction between the programmable nuclease and a target nucleic acid may produce a change in pH of the sample, producing a colorimetric signal. This change in pH may be measured using a dye, such as Phenol red, or a nucleic acid stain.

The signals may be used to detect pathogenic viruses, pathogenic bacteria, pathogenic worms, pathogenic fungi, or cancer cells. The pathogenic viruses may be respiratory viruses, adenoviruses, parainfluenza viruses, severe acute respiratory syndrome (SARS), coronavirus, SARS-CoV, SARS-CoV-2 and variants thereof, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, and papillomavirus.

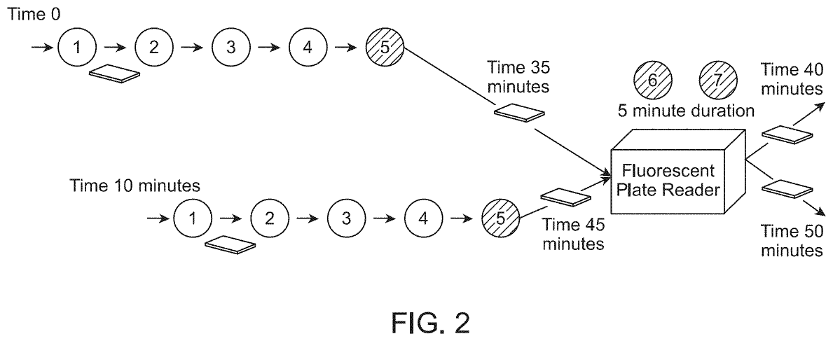

FIG. 2 illustrates the process of FIGS. 1A-1E being implemented on two samples in a staggered fashion. In this embodiment, a first single-chamber process is initiated at Time 0 and a second single-chamber process is initiated at Time 10 minutes. The first and second processes remain staggered by ten minutes throughout their entireties. In other embodiments, the processes may be staggered by half the time it takes for a single process to complete (e.g., 20 minutes). In at least some instances, staggering the processes may facilitate faster overall workflow versus running different processes in sequence.

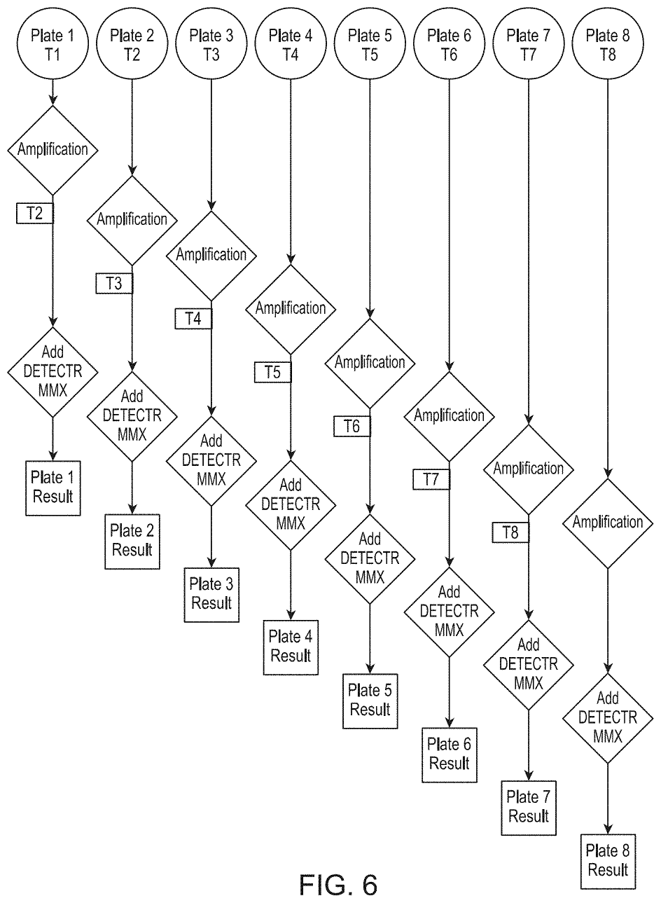

FIG. 6 illustrates an additional embodiment of a high-throughput single-chamber detection assay. In the embodiment of FIG. 6, eight single chambers from eight microplates are processed in parallel. In this embodiment, the plates may be tested in a staggered fashion, with each subsequent plate being tested at a time during the previous plate's amplification phase. For example, plate 2 may begin being tested five minutes into the amplification stage of plate T1. In this fashion, all eight plates may be tested within a four-hour timeframe, from the commencement of testing of plate 1 to the completion of testing of plate 8.

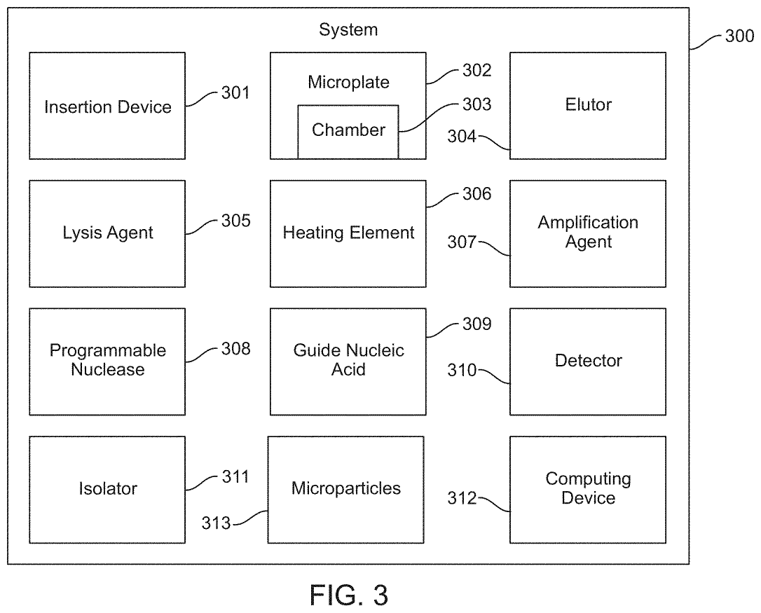

FIG. 3 illustrates a block diagram for a system 300 configured to implement a high-throughput DETECTR assay. The system components include agents which may be reagents, mechanical components, or other tools by which the steps of lysis, heating, amplification, elution, and detection are implemented. The system 300 may be an in vitro diagnostic (IVD) system. The modular components of the system 300 may belong to one or more pieces of laboratory equipment. The laboratory equipment may be automated liquid handling equipment or manual liquid handling equipment. The system 300 may include a computing device on which an operator may control assay operations manually or may program them to be completed automatically (e.g., by executing a script comprising instructions for the computer processor). For example, many aspects of the system 300 may be implemented using automated liquid handling equipment, such as the Agilent BRAVO or Hamilton STAR. The liquid handling equipment may be configured to operate continuously. The liquid handling equipment may be configured to perform some or all of the workflow of FIGS. 1A-1E.

The system 300 may be deployed in many types of sites. The system 300 may be deployed in a large-scale site, such as a manufacturing plant, a large school, or a large event gathering. The system 300 may be deployed within a small-scale site, such as a day care center, elementary school, or a facility housed by a business closed to the public. The system 300 may be deployed in a healthcare setting, such as a retail health facility, among a group of physicians, in a nursing home, in an assisted living facility, or in a hospital. The system 300 may be deployed in a clinical laboratory. The system may be deployed in a home, such as a single-family dwelling, apartment building, or condominium.

The system 300 may be configured to produce a result from a single-chamber process in 40 minutes or less. System components may be manipulated to perform the assay manually or robotically. For example, humans may ensure that reagents and samples are placed and labeled correctly, while robots may operate pipette heads, control heating, and transfer reagents between stations. The system 300 includes an insertion device 301, a microplate 302 including the single chamber 303 for the reaction, an elutor 304, a lysis agent 305, a heating element 306, an amplification agent 307, a programmable nuclease 308, a guide nucleic acid 309, a detector 310, an isolator 311, a computing device 312, and a microparticle 313.

The computing device 312 of the system 300 may automatically direct system components to perform, or prompt a user to manually perform, pre-processing tasks prior to the detection process. For example, the computing device 312 may prompt placement of reagents into specified locations or into specified vessels. The reagents may be color-coded or labeled in order to facilitate their proper placements. Some reagents, such as a lysis buffer, an RT-LAMP master mix, and a DETECTR reagent, may require mixing prior to being used for the detection assay. The system 300 may mathematically track reagent volumes or use fluid pipette liquid sensing to detect when reagents are empty or not properly loaded. When either of these situations occurs, the system may pause and wait for manual intervention. When reagents are properly loaded, the operator may then signal for the system to continue. The system 300 may be optimized for parallel tip aspiration with a multi-tip pipette by including multiple troughs for the reagents used during the assay. The system 300 may scan identifiers of sample tubes (e.g., by using a bar code scanner) to ensure that the samples are loaded properly. The sample may be held in a tube with a round bottom or a conical tube. There may also be a tube for holding positive and negative controls. The system 300 may be configured to operate with a 16×100 mm sample tube or a 12×80 mm sample tube. The system 300 may scan to check that all of the correct samples are in place prior to their usage during the detection assay. Sample loading may be performed in parallel with preparing reagents (e.g., lysis and amplification reagents).

The insertion device 301 inserts the sample and various reagents into the chamber 303 in order to carry out the detection. The insertion device 301 may be a multi-tip pipette head. Use of a multi-tip pipette head may minimize timing for reagent addition and removal as well as prevent cross-contamination.

The microplate 302 comprises the wells that serve as reaction chambers 303 for the single-chamber process. The microplate 302 may be 96 or 384 wells deep. To prevent cross-contamination, alternate microplate wells may be left empty in order to keep pipette fluid from dripping into adjacent microplate channels. If 96-deep well plates are used, more (e.g., four times as many as for 384-deep well plate) stations may need to be performing the assay. If the stations process staggered in time, this may provide advantages. For example, if the amplification stage takes 20-30 minutes, and the detection stage takes ten minutes, running two processes in a staggered fashion may result in less idle time during the detection step. Each chamber of the microplate 302 may have a 250 to 300 μL fill volume. The system 300 may be configured to operate with off-the-shelf microplates, such as the BRAND BR701355 and Nunc 269390.

In alternative embodiments, the system may use alternate sample containment vessels, or arrays of vessels. For example, the sample may be contained within a tube. The sample may be contained in a bottle. The sample may be contained in a vial. The sample container may be made from one or more of plastics, polymers, or metals. A sample container may be made at least in part from aluminum, brass, copper, or another metal or metal alloy. A sample container may be made at least in part from a polymer such as polypropylene. A sample array may comprise one or more types of vials, bottles, tubes, or wells.

The lysis agent 305 breaks up the cells in the sample to release the nucleic acid molecules. The lysis agent 305 may be a solution, such as a lysis buffer. The lysis buffer may comprise one or more lysis reagents in solution. The lysis may also be performed mechanically. The system 300 may have a reservoir to collect lysate waste liquid.

The microparticles 313 may be beads. The beads may be silica-coated magnetic beads. The beads could be made from carbohydrate copolymers, hydroxy functionalized copolymers, or carboxylic acid functionalized copolymers. Because the nucleic acid molecules are charged, the microparticles serve to immobilize the nucleic acid molecules within the chamber 303. The microparticles may be designed to have large surface areas to enable superior binding to the target nucleic acids and enhanced washing efficiency. The microparticles may be magnetic beads from a viral isolation kit, such as MagMAX, ChargeSwitch, or other nucleic acid purification kits (e.g., QuickRNA MagBead, SPRIselect, Dynabeads, or SiMAG-N-DNA magnetic beads).

The isolator 311 isolates the nucleic acid molecules and microparticles 313 in the chamber 303. The isolator 311 comprises a magnet and/or an aspiration device (e.g., a syringe, pipette, etc.). The magnet may be moved into contact with the bottom well of the microplate 302 in which the sample is being held, or the microplate 302 may be contacted with the magnet. In at least some instances, the temperatures of the microplate 302 may be adjusted in order to perform one or more reactions as described herein. In some embodiments, if the magnet is brought into contact with the plate, the temperature of the plate may be set to 57-62° C. In some embodiments, if the plate is brought into contact with the magnet, the isolation may occur at ambient temperature. In some embodiments, either the plate or the magnet or both, or another element within the system, may be heated.

The elutor 304 (herein referred to interchangeably with “elution agent”) separates the nucleic acid molecules from the microparticles 313. The elutor 304 may be an elution buffer solution. Elution may proceed after 30 seconds from the magnet's contact with the chamber 303. The elutor 304 may dispense an elution buffer into the chamber 303. The nucleic acid molecules and magnetic particles may be a clump, which is dispersed by the elution buffer.

The amplification agent 307 may produce many copies of the nucleic acid molecules to make detection of the target nucleic acid easier. The amplification agent 307 may be an RT-lamp solution. A combined RT-LAMP reaction may comprise LAMP primers, reverse transcription primers, dNTPs, a reverse transcriptase, and a DNA polymerase. In some case, the LAMP primers may comprise the reverse transcription primers. In some embodiments, the dNTPs may comprise dTTP, dATP, dGTP, and dCTP. In some embodiments, the dNTPs may comprise dUTP, dATP, dGTP, and dCTP. A combined RT-LAMP DETECTR reaction may comprise LAMP primers, reverse transcription primers, dNTPs, a reverse transcriptase, a DNA polymerase, a guide nucleic acid 309, a programmable nuclease 308, and a target nucleic acid. In some case, the LAMP primers may comprise the reverse transcription primers.

The amplification agent 307 may amplify the nucleic acid molecules using reverse transcription polymerase chain reaction (RT-PCR). RT-PCR may be used to identify SARS-CoV-2 RNA, for example. The RNA may be isolated in the sample. Then, the RNA may be reverse-transcribed to cDNA. RT-PCR may comprise temperature cycling the sample between denaturing (melting), annealing, and extension temperatures to amplify the cDNA. The denaturing temperature may be about 95° C., the annealing temperature may be about 50-60° C., and the extension temperature may be about 68-72° C. The amplification process may produce a detectable signal (e.g., a fluorescent signal).

The programmable nuclease 308 may be an enzyme which may be used to detect a target nucleic acid. The programmable nuclease 308 may be a CRISPR/Cas enzyme. In order to detect the target nucleic acid, the programmable nuclease 308 may be complexed with a guide nucleic acid 309. During a detection reaction, the target nucleic acid may hybridize to the guide nucleic acid 309. This may activate trans-cleavage of a single-stranded DNA (ssDNA), such as an ssDNA reporter.

The guide nucleic acid 309 may include a region comprising a nucleotide sequence complementary to the target nucleic acid and which may bind to the complementary target nucleic acid sequence. The guide nucleic acid 309 may bind to the programmable nuclease, forming a complex. The complementary sequence may then guide the complex via pairing to a specific location on the target nucleic acid, where the programmable nuclease 308 may perform endonuclease activity by cutting the target nucleic acid strand.

In some embodiments, the programmable nuclease 308 and guide nucleic acid 309 are provided in a detector reagent mix composition. The detector reagent mix may further comprise a labeled reporter. The programmable nuclease 308 may be a programmable Cas12 nuclease, a programmable Cas13 nuclease, a programmable Cas14 nuclease, a programmable CasΦ nuclease, a programmable CasX nuclease, a programmable CasY nuclease, a programmable thermostable Cas nuclease, a programmable CasZ nuclease, or the like.

The heating element 306 is configured to heat the contents of the chamber 303 in which the detection assay occurs. The heating element 306 may be under the microplate 302. The heating element 306 may be configured to cycle between 95° C., 52-67° C., and 37° C. temperatures, and/or, for PCR heating during amplification, may cycle between 45° C. and 95° C. In a single-station embodiment, the heating element 306 may change its temperature at different stages of the reaction, with a ramp time of less than two minutes. In a multi-station embodiment, multiple heating elements may be employed to heat the contents of the reaction chamber 303 as it travels between stations.

The detector 310 may collect detection signals from the detection assay. The detector may be a fluorimeter (e.g., a fluorescent plate reader) positioned directly above the detection and incubation chamber 303. The fluorimeter may be a commercially available instrument, the optical sensor of a mobile phone or smart phone, or a custom-made optical array comprising of fluorescence excitation means, e.g., CO2, other, laser and/or light emitting diodes (LEDs), and fluorescence detection means e.g., photodiode array, phototransistor, or others. A device may comprise a chamber comprising transparent or translucent materials that allow light to pass in and out of the chamber.

The computing device 312 may analyze one or more signals from the detector 310 to determine a presence or absence of a target nucleic acid. The computing device 312 may include the detector (e.g., if the detector is the optical sensor of a mobile phone or smartphone) or may be a separate device. The computing device 312 may determine the presence of the target nucleic acid by performing statistical analysis on data from the signal reader. For example, the computing device 312 may calculate a slope from multiple readings from the chamber collected over a time period. Then, the computing device 312 may compare the slope against that of a positive test result and that of a negative test result. In some implementations, when the target nucleic acid is a viral antigen, criteria for the prediction the computing device 312 may make of a presence or absence of the target nucleic acid may be configured to minimize false positive values.

The system 300 may process an assay in a single station. When processing in a single station, the system 300 may include a heating element 306 to heat the sample in the chamber 303 to 95+2/−5° C., 57-62+/−2° C., and 37+/−2° C. The ramp time to switch between temperatures may be less than 2 minutes. During the end of the incubation with the lysis agent 305, a magnet may be brought into contact with the chamber to capture the nucleic acid-bound microparticles. The magnet may retract near the beginning of the 57-62° C. incubation during the amplification operation. The magnet may be brought into contact with the plate after a period of time from the temperature reaching 95° C., e.g., five minutes. The assay processing station may have the ability to monitor the plate's wells every 20 seconds with a fluorescent plate reader during the 37° C. incubation during the detection operation. Plate mixing/agitation may occur during the 37 C incubation period. Plate mixing during the elution operation may disperse the magnetic beads. In this embodiment the system 300 may have two stations that can run in staggered parallel operations to increase throughput.

The system 300 may also process the assay in multiple stations. The stations may include a lysis station, a capture station, an elution station, an amplification station, and a detection station. At the lysis station, the system 300 may dispense the lysis agent 305 and microparticles into the single chamber 303 and heat to 95° C., in order to lyse the sample, release the nucleic acid molecules, and bind them to the microparticles to produce the complex. At the capture station, the isolator 311 may be used to isolate the nucleic acid molecules and microparticles. At the elution station, the system 300 may use plate or pipette mixing to separate the microparticles and nucleic acid molecules. The lysis, capture, and elution stations may be co-located or separate, as they are all performed at the same temperature. At the target amplification station, the amplification agent 307 is added and the temperature is lowered to 57-62° C. At the detection station, the temperature is 37° C. and detection is performed with a fluorescence plate reader and plate mixer. The multi-station embodiment may include a robot to transfer the chamber 303 with sample between stations. Each station may include a heating element 306 to maintain a preferred temperature for the station. Throughput in this embodiment may be increased by beginning processing of an additional batch of samples after a current batch of samples has reached approximately a halfway processing point. In some implementations, this may be when a first reaction is undergoing amplification. In other embodiments, throughput may be increased by beginning processing of an additional batch of samples after 10% of processing has completed, after 20% of processing has completed, after 30% of processing has completed, after 40% of processing has completed, after 60% of processing has completed, after 70% of processing has completed, after 80% of processing has completed, or after 90% of processing has completed for a first batch of samples.

### Example Programmable Nuclease Probe

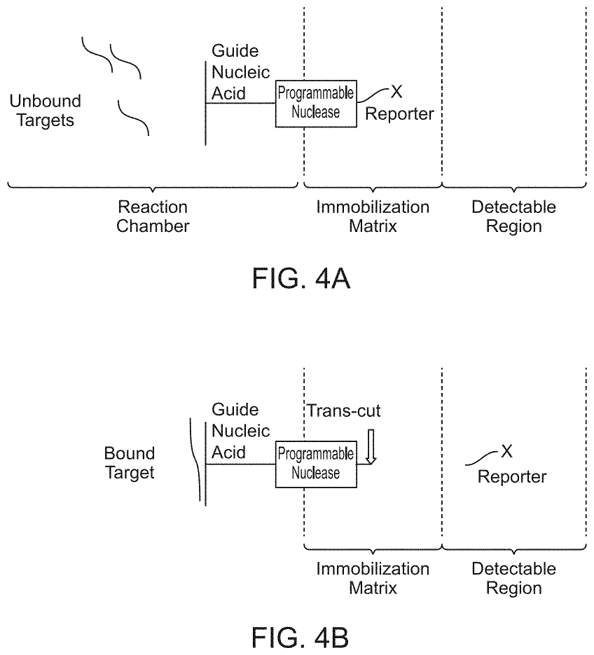

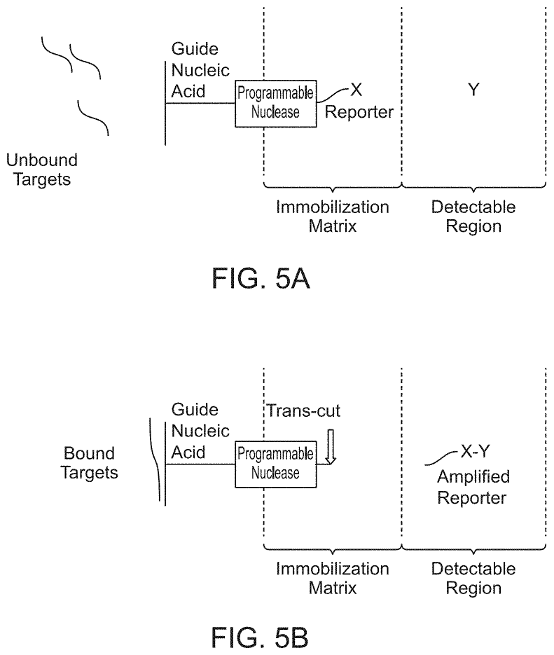

FIGS. 4A, 4B, 5A, and 5B illustrate an exemplary programmable nuclease probe that can be used in a compatible manner with the devices of the present disclosure. The programmable nuclease probe can comprise a programmable nuclease probe that comprises a guide nucleic acid complexed with a programmable nuclease. The programmable nuclease can comprise any type of programmable nuclease as described herein. In some cases, the programmable nuclease probe comprises a guide nucleic acid complexed with a CRISPR enzyme. For example, FIG. 4A shows unbound target amplicons in the chamber prior to binding to a Guide RNA, which in turn is contacted to a programmable nuclease (e.g., a CRISPR enzyme). The Guide RNA-CRISPR enzyme complex also includes a reporter. The guide nucleic acid or Guide RNA is exposed to the target amplicons inside the chamber. In some embodiments, the programmable nuclease probe (e.g., a CRISPR probe) may be immobilized to an immobilization matrix, where the interior side of the immobilization matrix is exposed to the inside wall of the chamber. The reporter may be in proximity to the “exterior” side of the immobilization matrix, wherein the exterior side of the immobilization matrix is in proximity to the detector. In other embodiments, the programmable nuclease probe may not be immobilized within the chamber. FIG. 4B illustrates a programmable nuclease probe (e.g., a CRISPR probe) after binding with a complementary target amplicon. The binding event triggers a trans-cut that releases the reporter or changes the reporter. Detection mechanisms can involve interferometry, surface plasmon resonance, electrochemical detection such as potentiometry, chemiluminescence, or scattering.

In certain instances, as seen in FIGS. 5A and 5B, the reporter of the programmable nuclease probe can initiate a signal amplification reaction with another molecular species after the complementary binding induced trans-cutting. Such species can be a reactive solid or gel matrix, or other reactive entity to generate an amplified signal during detection. The reporter compounds can freely participate in one or more cascading amplification reactions that generate an amplified signal. The signal amplification reaction can be physical or chemical in nature. In certain instances, as seen in FIGS. 5A and 5B, after a complementary binding induced trans-cut, the released reporter, ---X, can initiate an interaction and/or a reaction with another entity, Y, to produce an amplified or modified signal. Such entities can comprise a molecular species, a solid, a gel, or other entities. The signal amplification interaction can be a physical or chemical reaction. In some embodiments, the interaction involves free-radical, anionic, cationic or coordination polymerization reactions. In other embodiments the cut reporter can trigger aggregation, or agglutination, of molecules, cells or nanoparticles. In some instances, the cut reporter can interact with a semiconductor material. In some embodiments the chemical or physical change caused by the interaction is detected by optical detection means such as interferometry, surface plasmon resonance, reflectivity or other. In other embodiments the chemical or physical change caused by the interaction is detected by potentiometric, amperometric, field effect transistor, or other electronic means.

The programmable nuclease probe can comprise a programmable nuclease and/or a guide nucleic acid. The guide nucleic acid can bind to a target nucleic acid, as described herein. In some cases, to minimize off-target binding (which can slow down detection or inhibit accurate detection), the device can be configured to generate an electro-potential gradient or to provide heat energy to one or more regions proximal to the programmable nuclease probe, to enhance targeting. In at least some instances, electro-potential gradient generation or heating can increase diffusion of reactants (probe and target) and thus increase the rate of specific binding between the guide nucleic acid and the target nucleic acid.

In some embodiments, programmable nucleases can be used to carry out highly efficient, rapid, and accurate reactions for detecting whether a target nucleic acid is present in a sample. A programmable nuclease may be activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of reporters with a detection moiety. Once the reporter is cleaved by the activated programmable nuclease, the detection moiety can be released from the reporter and can generate a signal. A signal can be a calorimetric, potentiometric, amperometric, optical (e.g., fluorescent, colorimetric, etc.), or piezo-electric signal. Often, the signal is present prior to reporter cleavage and changes upon reporter cleavage. Sometimes, the signal is absent prior to reporter cleavage and is present upon reporter cleavage. The programmable nuclease can be a CRISPR-Cas (clustered regularly interspaced short palindromic repeats—CRISPR associated) nucleoprotein complex with trans cleavage activity, which can be activated by binding of a guide nucleic acid with a target nucleic acid. The CRISPR-Cas nucleoprotein complex can comprise a Cas protein (also referred to as a Cas nuclease) complexed with a guide nucleic acid, which can also be referred to as CRISPR enzyme. A guide nucleic acid can be a CRISPR RNA (crRNA). Sometimes, a guide nucleic acid comprises a crRNA and a trans-activating crRNA (tracrRNA).

The programmable nuclease system used to detect modified target nucleic acids can comprise CRISPR RNAs (crRNAs), trans-activating crRNAs (tracrRNAs), Cas proteins, and reporters.

In any of the embodiments described herein, the programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid, which can initiate trans cleavage activity. In some cases, the trans cut or trans cleavage can cut and/or release at least a portion of a reporter molecule. In other cases, the trans cut or trans cleavage can produce an analog of a target, which can be directly detected. Trans cleavage activity can be non-specific cleavage of nearby nucleic acids by the activated programmable nuclease, such as trans cleavage of reporters with a detection moiety. Once the reporter is cleaved by the activated programmable nuclease, the detection moiety can be released from the reporter and can generate a signal. The detection moiety can correspond to the element, or moiety, (X) shown in FIGS. 4A, 4B, 5A, and 5B. The signal can be visualized to assess whether a target nucleic acid is present or absent.

Reporters, which can be referred to interchangeably reporter molecules, or reporters, can be used in conjunction with the compositions disclosed herein (e.g., programmable nucleases, guide nucleic acids, etc.) to carry out highly efficient, rapid, and accurate reactions for detecting whether a target nucleic acid is present in a sample. The reporter can be suspended in solution or immobilized on a surface. For example, the reporter can be immobilized on the surface of a chamber in a device as disclosed herein. In some cases, the reporter can be immobilized on beads, such as magnetic beads, in a chamber of a device as disclosed herein where they are held in position by a magnet placed below the chamber. The reporter can capable of being cleaved by the activated programmable nuclease, thereby generating a detectable signal. The detectable signal can correspond to a release of one or more elements (X) as illustrated in FIGS. 5A and 5B. The release of the one or more elements (X) can initiate a reaction with another element (Y) when the element (Y) is in the presence of the element (X). The reaction between the element (Y) and the element (X) can initiate a chemical chain reaction in a solid phase material. Such a chemical chain reaction can produce one or more physical or chemical changes. In some cases, the physical or chemical changes can be optically detected. In some embodiments, one or more cascade amplification reactions can occur to further amplify the signal before sensing or detection. There can be a single point of attachment between the reporter molecule and the element (X). Cutting the single point of attachment can release a macro molecule (X), which can undergo a series of reactions based on the macro molecule (X) itself. In any of the embodiments described herein, the reporter can comprise a single stranded reporter comprising a detection moiety.

### Viral Lysis Buffer Experiments

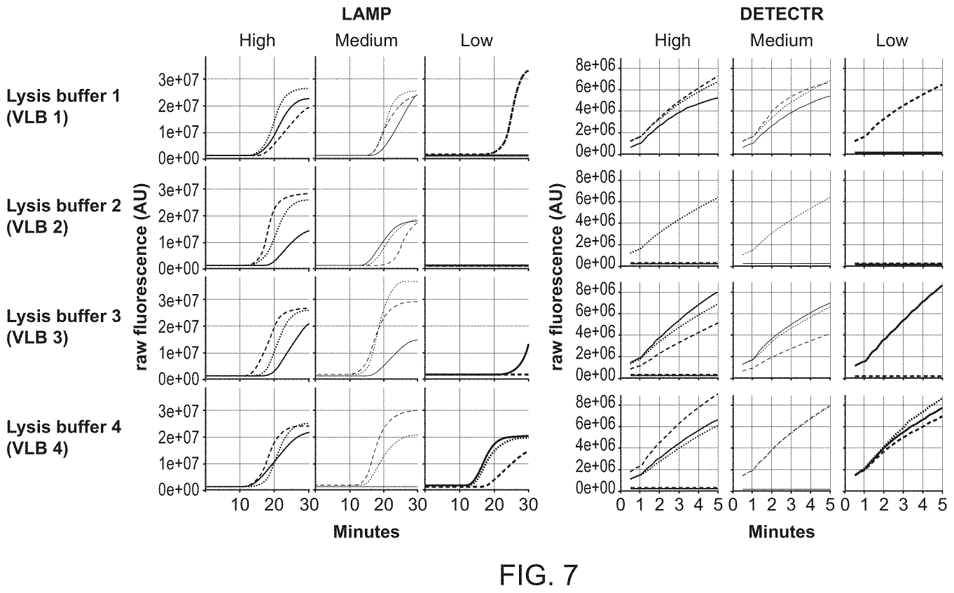

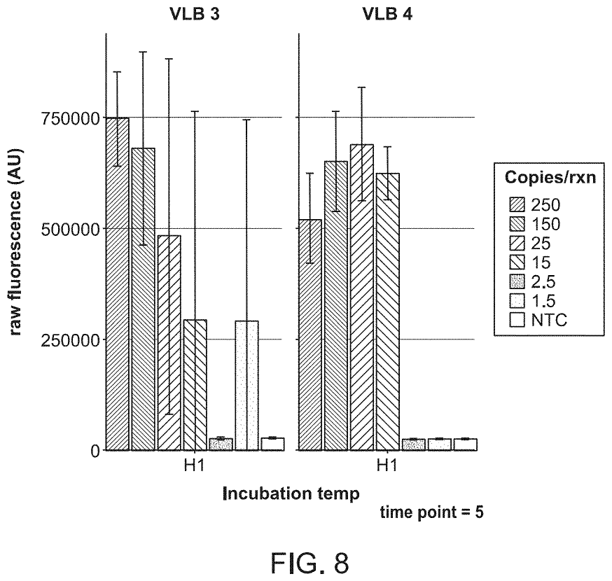

FIGS. 7-9 illustrate experiments performed using different buffer solutions during the lysis stage to determine the solutions' effects on detection reactions using RT-LAMP and DETECTR solutions. The experiments compared raw fluorescence signals (arbitrary units or AUs) from the detection reactions to determine which buffers were best suited for the detection methods disclosed herein. SeraCare encapsulated SARS-CoV-2 positive control samples were lysed for 5 minutes followed by 30 minutes RT-LAMP amplification at 62 C and 15 minutes DETECTR reaction at 37 C.

Lysis buffers disclosed herein may be compatible with detection solutions used in this application. The following lysis buffers disclosed may be effective at promoting detection of viruses in samples of varying concentrations. The buffer solutions disclosed may be compatible with nasal and saliva sample matrices from nasal swab samples and saliva swab samples.

FIG. 7 illustrates a comparison of four different viral lysis buffer solutions (VLB 1, VLB 2, VLB 3, and VLB 4) with respect to their effects on fluorescence signals produced using LAMP and DETECTR, testing three replicates. FIG. 7 illustrates that VLB 4, particularly at low concentrations of virus, promoted both amplification and detection of three different viral titers, for both LAMP and DETECTR reactions. responses for samples prepared using two different lysis buffer solutions VLB 3 and VLB 4 at room temperature for different copy numbers per reaction. The charts demonstrated improvements from using the buffer solutions in detections of varying concentrations of virus titer, even when tests were conducted at lower temperatures than those of an earlier formation of the buffer solution. Shown are raw fluorescence values from detections of seven different concentrations of virus (in copies per reaction), where NTC refers to “no template control”. The graph illustrates the maximum recorded fluorescence at five minutes after initiating the DETECTR reaction. The legend illustrates starting viral copy numbers/reaction. The samples were lysed by incubation at room temperature.

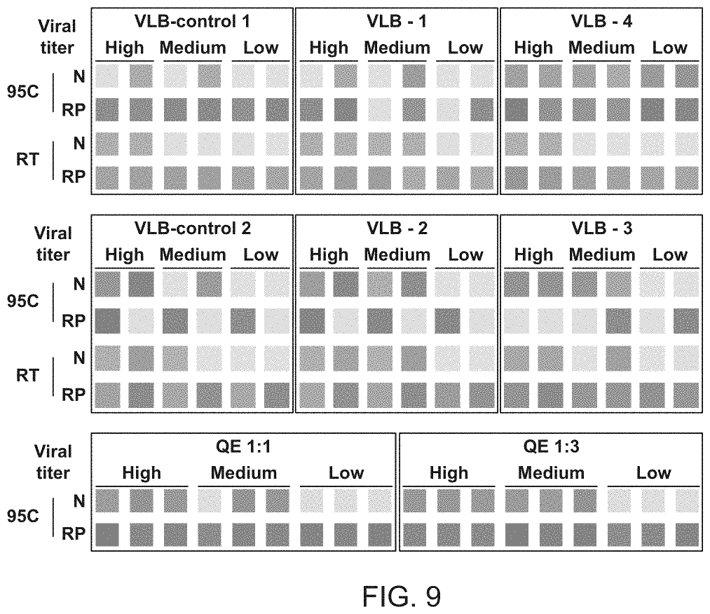

FIG. 9 shows the efficacies of different lysis buffer solutions and lysis temperatures at promoting detection of RNase P (RP) control and N Gene (N) virus titers. VLB-4 promoted detection of N and RP at 95° C. and detection of RP at room temperature for high, medium, and low titers. VLB-1 and VLB-2 promoted detection of N at room temperature at high and medium titers. VLB-4 may be compatible with nasal and saliva sample matrices. VLB-4 outperformed Lucigen QuickExtract (QE) at 95° C. for both RP and N titers. All lysis buffers promoted detection of RP at room temperature. Darker values signify stronger signals (higher fluorescence).

Additional experiments confirmed that all lysis buffers effectively inactivated SARS-CoV-2 in PBS and saliva after 5 minutes at 95° C.

### DETECTR Assay Configurations

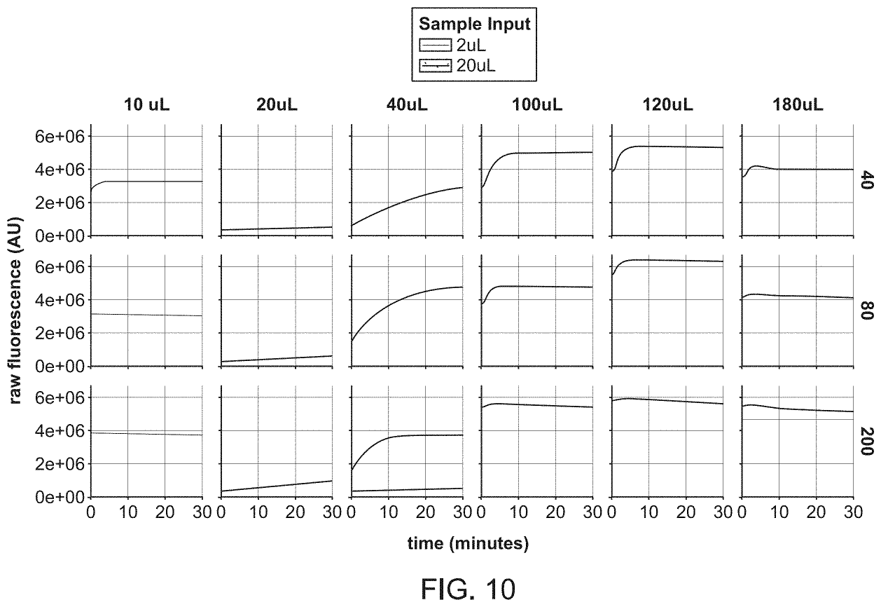

FIGS. 10-17 illustrate experiments investing improved formulations for DETECTR-based SARS-CoV-2 detection reactions. Twist CoV synthetic RNA was amplified using RT-LAMP for 30 min at 62 C and then a volume of the amplified product was added to the DETECTR reagents for the DETECTR reaction for 30 min at 3 C. Twist copy numbers, RT-LAMP:DETECTR volume ratios, programmable nuclease complex concentrations, reaction volumes, and buffer formulations were varied for formulation/condition optimization.

FIG. 10 illustrates fluorescence signals detected from using varying amounts of DETECTR solution on 20 μL of samples that have been amplified using RT-LAMP, enabling testers to determine an optimal ratio between RT-LAMP and DETECTR volume in a larger volume (40-200 μL) DETECTR reaction. 1000 copies of Twist RNA was amplified in a 25 μL RT-LAMP reaction volume. Following amplification, 20 μL of the amplified sample was added to varying volumes of DETECTR reagents. The plots show raw fluorescence values for such samples with 20 μL, 40 μL, 100 μL, 120 μL, and 180 μL of DETECTR reagents added, for three molar concentrations (40, 80, 200 nM) of a Cas12 programmable nuclease complex. Additionally, plots are illustrated for a positive control reaction combining 2 μL RT-LAMP product and 18 μL DETECTR solution (20 μL total volume). The plots show increasing performance for larger amounts of detector solution added, with the 180 μL plot closely resembling the positive control 18 μL results. The plots show that, for all molar concentrations of the programmable nuclease complex, a ratio of 1:6 of RT-LAMP to DETECTR yielded the largest fluorescence value, and may be an optimal ratio of RT-LAMP to DETECTR for use in testing with the tested conditions, reagents, etc. Reactions with a ratio below 1:2 were largely inhibited, likely by an excess of RT-LAMP reagents in the mix.

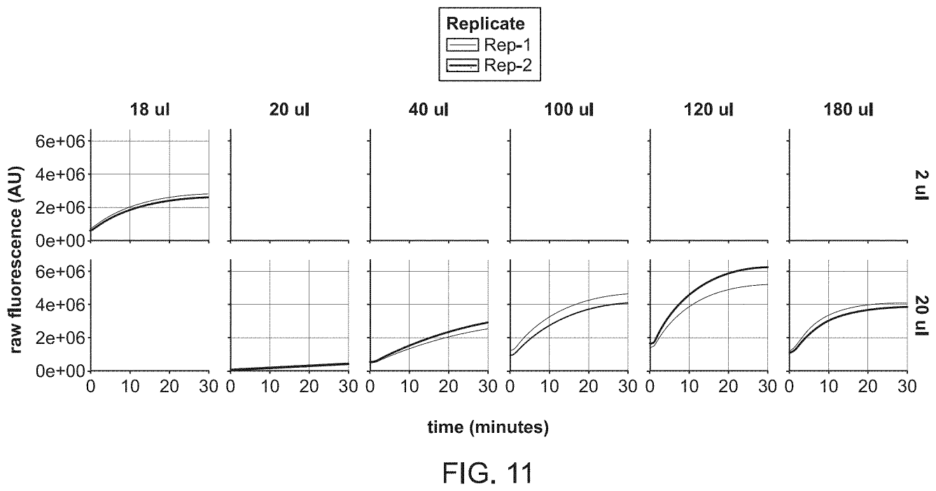

FIG. 11 additionally illustrates detection results for two different replicates at different ratios of RT-LAMP to DETECTR in a larger volume (40-200 μL) DETECTR reaction, mirroring the optimal result from FIG. 10. The charts illustrate that N-gene DETECTR reactions were inhibited at 1:1 (1× RT-LAMP+1× DETECTR master mix) dilution. The results indicate that 1:3 dilution DETECTR saturated more slowly than 1:6 dilution. The results indicated that the N-gene DETECTR worked well with all 1:5, 1:6, and 1:9 dilutions.

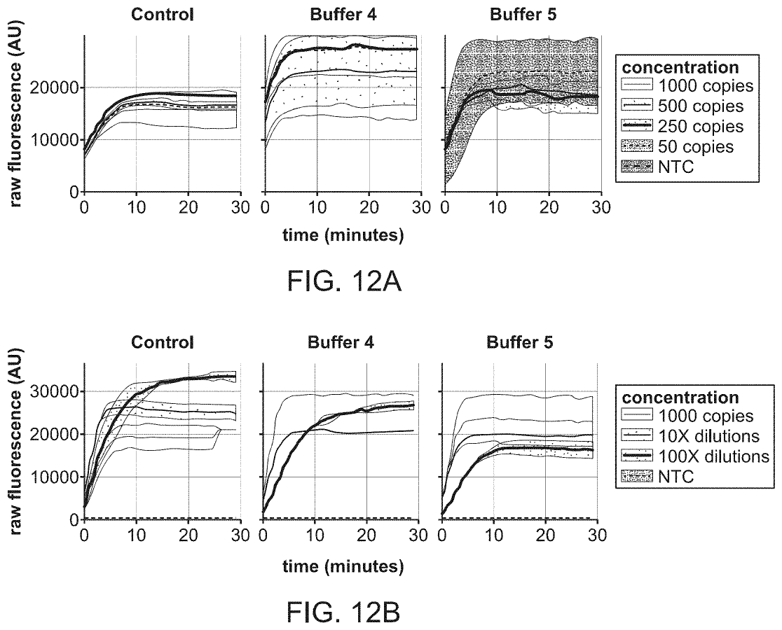

FIGS. 12A and 12B illustrate fluorescence measurements from DETECTR reactions with various buffer solutions. In some embodiments, the system may dilute the sample with a buffer (e.g., one of buffers 4 and 5) prior to application to the detection system. Specifically, FIG. 12A illustrates enhancements of using buffer solutions with RT-LAMP product into N-gene DETECTR and FIG. 12B illustrates enhancements from 10×-100× dilution of RT-LAMP product into N-gene DETECTR. The plots show that buffers 4 and 5 enabled detection to be performed faster than the positive control buffer and produced larger fluorescence signals for sample input concentrations (pre-amplification) of 50-1000 copies per reaction. The enhancements were more pronounced for lower concentrations of virus. Dilution of the sample had little effect on the DETECTR reactions.

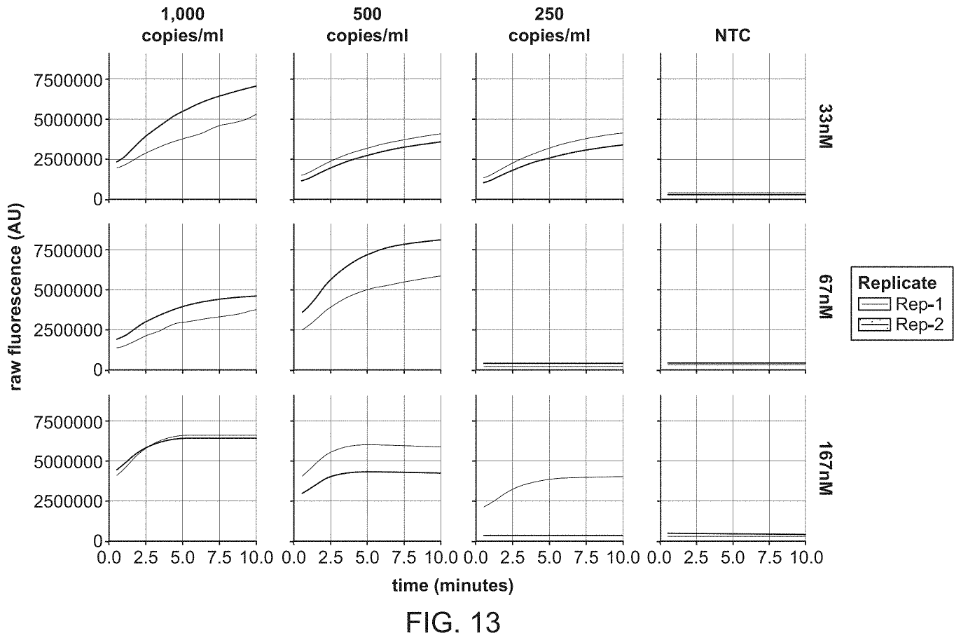

FIG. 13 illustrates fluorescence signal generation for varying concentrations of virus and DETECTR complex in DETECTR reactions. 50 ul of RT-LAMP product (1000 copies/ml, 500 copies/ml, 250 copies/ml, or NTC) generated using NEB isothermal amplification buffer was added to 150 ul of DETECTR complex (33 nM, 67 nM, or 167 nM). FIG. 13 shows strong performance for concentrations of 1,000 copies/mL and 500 copies/mL, indicating that these concentrations may be among optimal concentrations of virus for performing tests. The tests were conducted for 33 nM, 67 nM, and 167 nM amounts of programmable nuclease complex in the samples.

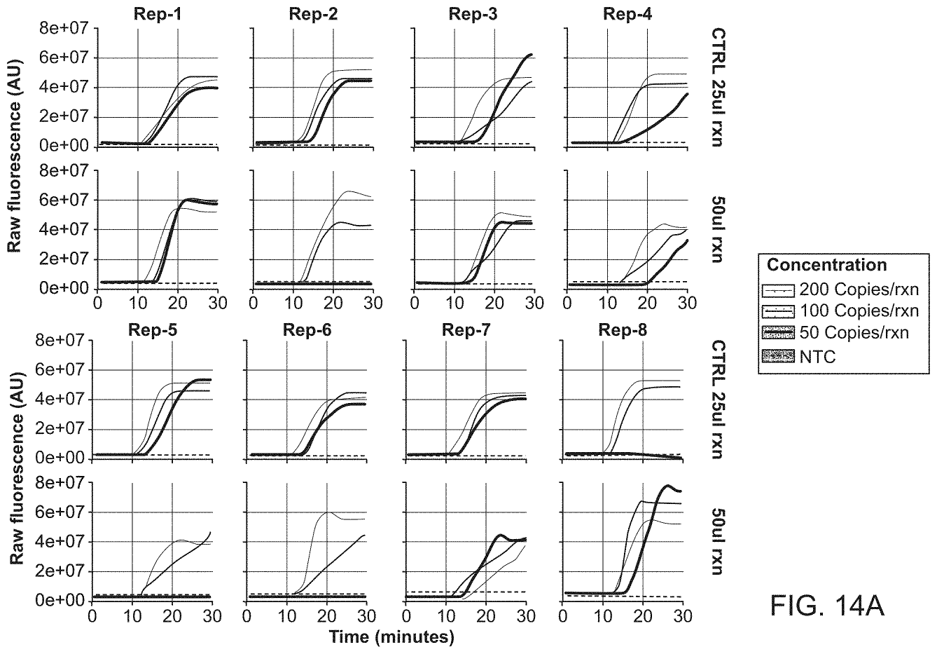

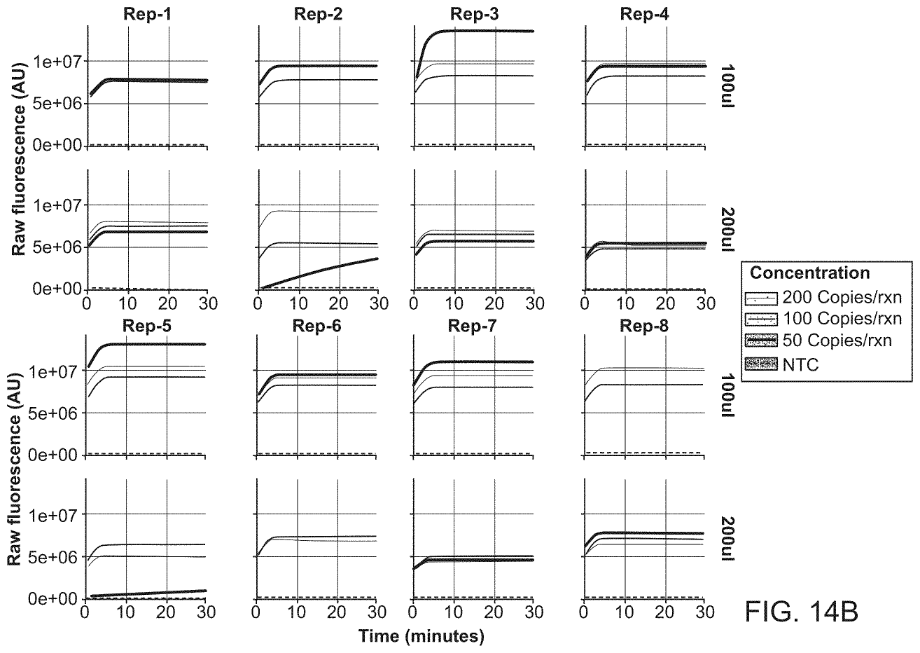

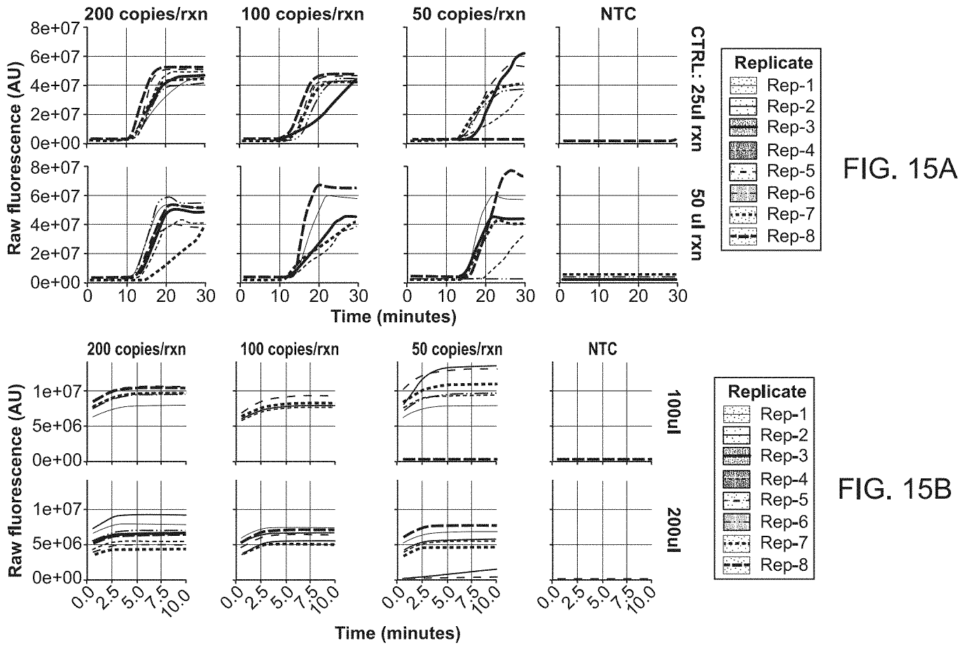

FIGS. 14A-14B and 15A-15B illustrate fluorescence signals of N-genes at small volumes (25 μL) and large volumes (50 μL) of RT-LAMP volume and corresponding 100 μL and 200 μL of DETECTR reaction volume. The 25 μL RT-LAMP reaction and 5 μL sample was a positive control and the test reactions included 5 μL of sample and 5 μL water in a 50 μL RT-LAMP reaction. For 100 μL μL DETECTR reaction volumes, 75 μL DETECTR mastermix was added to the 25 μL small volume RT-LAMP sample. For 200 μL μL DETECTR reaction volumes, 150 μL DETECTR mastermix was added to the 50 μL large volume RT-LAMP sample. With respect to detection, robust results were generated for both the 100 μL and 200 μL volumes for all 8 replicates.

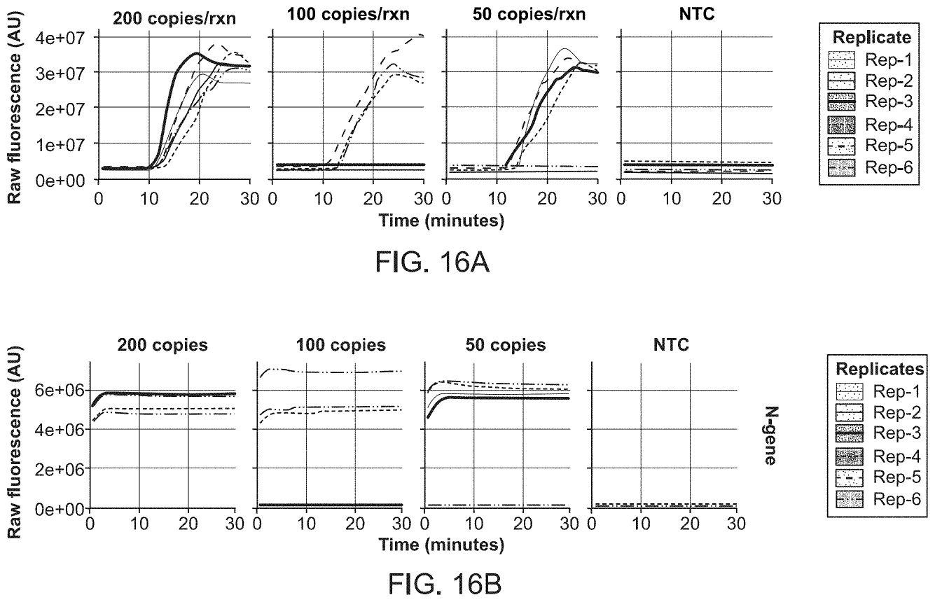

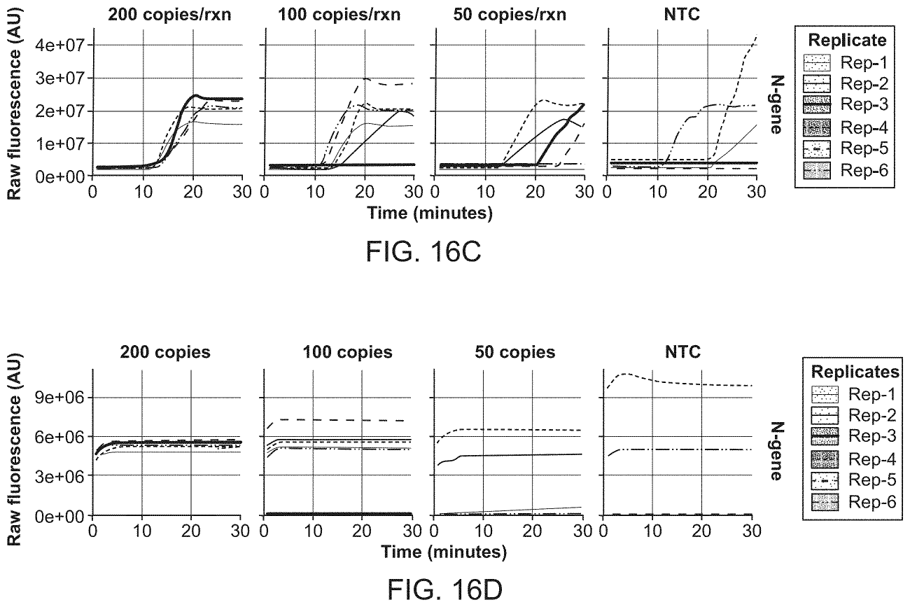

FIGS. 16A-16B illustrate fluorescence results from RT-LAMP reactions (top) and corresponding DETECTR reaction (bottom) using 25 μL of virus sample and 25 μL of a 2× RT-LAMP master mix formulation. The RT-LAMP master mix was formulated as a single master mix containing all amplification reagents. FIG. 16A illustrates that with 200 copies/rxn, six positive values (out of six) were correctly identified, with 100 copies/rxn, three were correctly identified, and with 50 copies, four were correctly identified.

FIGS. 16C-16D illustrate fluorescence results from using a different RT-LAMP master mix comprising two sub-master mixes to separate salts and enzymes, again with the top plot showing results for RT-LAMP and the bottom showing corresponding DETECTR results. In the 25 μL final mix, one sub-master mix included 15 μL of buffer and salts and a second sub-master mix included 10 μL enzymes, primers, and dNTPs. 25 μL of sample was added to 25 82 L of the combined master mix for RT-LAMP amplification. The charts show that, using RT-LAMP (FIG. 16C), six (out of six) positive values were detected with 200 copies/rxn, five out of six were detected for 100 copies/rxn, and four out of six were detected for 50 copies/rxn. Using DETECTR (FIG. 16D) confirmed six out of six positives in 200 copies and five out of six positives in 100 copies, with two positives in 50 copies/rxn (suggesting two of the RT-LAMP positives may have been false positives). The results of the sub-buffer formulation were similar to those of the single master mix formulation shown in FIGS. 16A-16B.

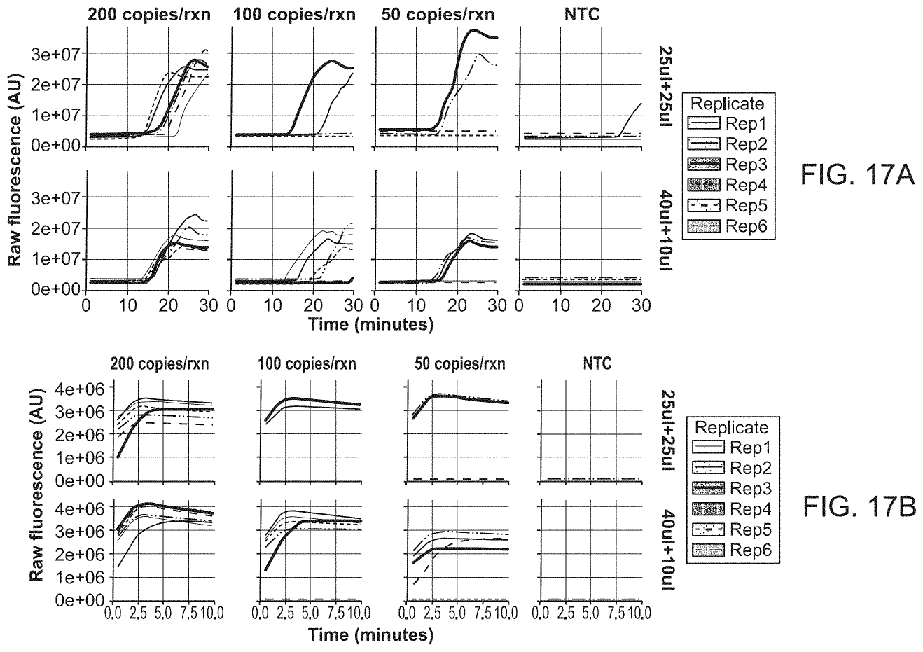

FIGS. 17A and 17B illustrate fluorescence results from using a master mix separating salts and enzymes at 2× or 5× concentration for RT-LAMP (FIG. 17A) and DETECTR (FIG. 17B) reactions. The 2× formulation included 20 μL salts and primers sub-master mix and 5 μL enzyme sub-master mix. The 5× formulation included 5 μL salts and primers sub-master mix and 5 μL enzyme sub-master mix. 25 μL sample was added to 25 μL of the 2× formulation or 40 μL of sample was added to 10 μL of the 5× formulation for a total reaction volume of 50 μL. Both master mix formulations performed well at 200 input copies per reaction. Interestingly, although the RT-LAMP reaction detected only three out of six positives at 50 copies/reaction, the DETECTR reaction picked up four out of six positives at 50 copies/reaction, indicating that the DETECTR system was able to generate a robust signal even when there was not enough RT-LAMP product for a detectable SYTO signal.

Table 2 shows results of SeraCare and Twist viral titration studies for the full workflow using VTM, UTM, nasal matrix, and/or saliva matrix. Samples concentrated using MagMAX beads and eluted into 25 μL. 25 μL of RT-LAMP master mix was added to the elution and RT-LAMP amplification was performed. 150 μL of DETECTR master mix was added to the completed RT-LAMP reaction. Fluorescence values were measured on a QS5 qPCR instrument. The test shows a 100% positive rate with 68 replicates tested across three independent experiments for samples as low as 1 copy/μL input volume.

TABLE 2
Replicate tests of full workflow

| Experiment | Sample | Contrived sample | Concentration | |
|---|---|---|---|---|
| No. | input | preparation | method | Positive |
| 1 | UTM (BD) + | 200 copies of SeraCare | MagMAX ™ | 12/12 |
| | Nasal | SARS-CoV-2 control in 200 μL | | |
| | | of UTM + Nasal | | |
| | UTM (BD) + | 200 copies of SeraCare | MagMAX ™ | 12/12 |
| | Saliva | SARS-CoV-2 control in 200 μL | | |
| | | of UTM + Saliva | | |
| | RNA | 500 copies RNA control | N/A | 2/2 |
| | control | | | |
| | (Twist) | | | |
| 2 | UTM (BD) + | 200 copies of SeraCare | ChargeSwitch ™ | 6/6 |
| | Nasal | SARS-CoV-2 control in 200 μL | | |
| | | of UTM + Nasal | | |
| | UTM (BD) + | 500 copies of SeraCare | ChargeSwitch ™ | 6/6 |
| | Nasal | SARS-CoV-2 control in 200 μL | | |
| | | of UTM + Nasal | | |
| | UTM (BD) + | 200 copies of SeraCare | ChargeSwitch ™ | 6/6 |
| | Saliva | SARS-CoV-2 control in 200 μL | | |
| | | of UTM + Saliva | | |
| | UTM (BD) + | 500 copies of SeraCare | ChargeSwitch ™ | 6/6 |
| | Saliva | SARS-CoV-2 control in 200 μL | | |
| | | of UTM + Saliva | | |
| | RNA | 200 copies RNA control | N/A | 2/2 |
| | control | | | |
| | (Twist) | | | |
| | RNA | 500 copies RNA control | N/A | 2/2 |
| | control | | | |
| | (Twist) | | | |
| 3 | VTM | 200 copies of SeraCare | MagMAX ™ | 6/6 |
| | (Corning) + | SARS-CoV-2 control in 200 μL | | |
| | Nasal | of VTM + Nasal | | |
| | VTM | 200 copies of SeraCare | MagMAX ™ | 6/6 |
| | (Corning) + | SARS-CoV-2 control in 200 μL | | |
| | Saliva | of VTM + Saliva | | |
| | RNA | 500 copies RNA control | N/A | 2/2 |
| | control | | | |
| | (Twist) | | | |

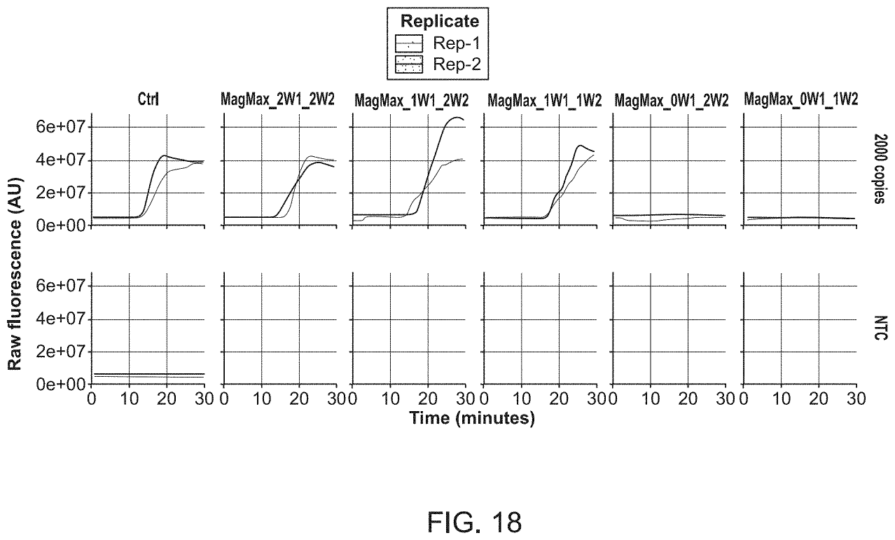

FIGS. 18-59 show data from experiments relating to the continued development of an accurate, fast, and easy-to-use COVID-19 testing system. The experiments from FIGS. 18-59 led to adoption of an improved assay that could be completed more quickly with lower volumes of reagents. The development of the assay is summarized in Table 3.

TABLE 3
Assay optimization and improvement summary

| | | | Improvement and |
|---|---|---|---|
| Step | Previous Assay | Updated Assay | modification |
| Sample/LOD | 200 μL in UTM/ | 110 μL in UTM/75 copies or | Reduced volume. |
| | 200 copies of SeraCare | 55 copies of SeraCare | Reduced the LOD <1000 |
| | | | copies/μL |
| Lysis | Lysis buffer: 200 μL | Lysis buffer: 100 μL | Reduced volume. |
| | (50% isopropyl | (50% IPA) | Increased proK and temp |
| | alcohol (IPA)) | Beads: 10 μL | for more efficient lysis |
| | Beads: 20 μL | (1:3, beads:proK, | Reduced beads to |
| | (1:1, beads:proK) | eg, 2.5 ul beads) | minimize potential |
| | 5 min RT | 5 min at 37 C. | inhibition and quenching |
| | | | to downstream. |
| Wash | 2X 200/μL W1, 2X | 2X 50 μL W1 | Removed W2. Reduced |
| | 200 μL W2 | | W1 volume to reduce |
| | | | pipetting time on deck |
| Elution | 25 μL Elution | 25 μL Elution buffer, add | No difference between |
| | buffer, 2 min | RT-LAMP reagents directly | elution and direct |
| | at 62 C. | to the beads. No separate | approach. Temp changed |
| | | elution step | to 57 C. |
| RT-LAMP | No beads. 40 min at | With beads. 75% Bst, 50% | Reduced the enzymes. |
| | 62 C. | RTx, 40 min at 57 C. | |
| DETECTR | No beads, 10 min at | With beads. 10 min at 37 C. | Time can be reduced |
| | 37 C. | | to 2-5 min |

FIGS. 18-59 illustrate results from modifying a test of the sample by adding various reagents, adding beads, or modifying environments in which various processes of the disclosed method take place. Results are herein summarized. For example, magnetic bead or viral isolation kits were optimized. For example, under particular conditions, additions of magnetic bead kits (viral isolation kits) and nucleic acid purification kits showed improved fluorescence readings. Reactions were successfully performed with different magnetic bead kits, different lysis buffers, different RT-LAMP buffers, and/or different DETECTR buffers. The number of wash steps was optimized to improve signal and reduce operation complexity and durations. A DETECTR reaction performed in a 384-deep well plate format showed good performance even in adjacent wells with little or no crosstalk between wells. Experiments performed with nasal and saliva matrices also showed improved performance over controls under the conditions tested. High copy numbers of N-genes did not inhibit the DETECTR reaction. RT-LAMP and DETECTR reactions were stable after reagents in the reactions had undergone multiple freeze-thaw cycles. Reagents showed stability when incubated in dry ice and held at room temperature for extended periods of time. Evaporation did not affect assay sensitivity.

FIG. 18 illustrates a illustrates results for determining a minimal wash condition using workflow with the MagMAX virus isolation kit for sample preparation. The viral isolation kit may include one or more wash solutions (e.g., W1 and W2), magnetic beads, and a lysis buffer. As part of the standard MagMAX workflow, the sample may be washed with two different solutions W1 and W2 up to four times (two times each, denoted as 2×W1+2×W2)). The purpose of the experiment was to determine the best performance achievable with minimal washing of the sample for the conditions tested. The RT-LAMP experiment was performed with 2000 copies per reaction of Twist synthetic SARS-CoV-2 RNA control 2 (Twist Biosciences) in UTM or a NTC control. For the 2000 copies samples, fluorescence signal was detected with as few as one wash with W1 and one wash with W2 (1×W1+1×W2). The positive control RT-LAMP reactions and negative control reaction (NTC) did not include MagMAX beads for sample preparation.

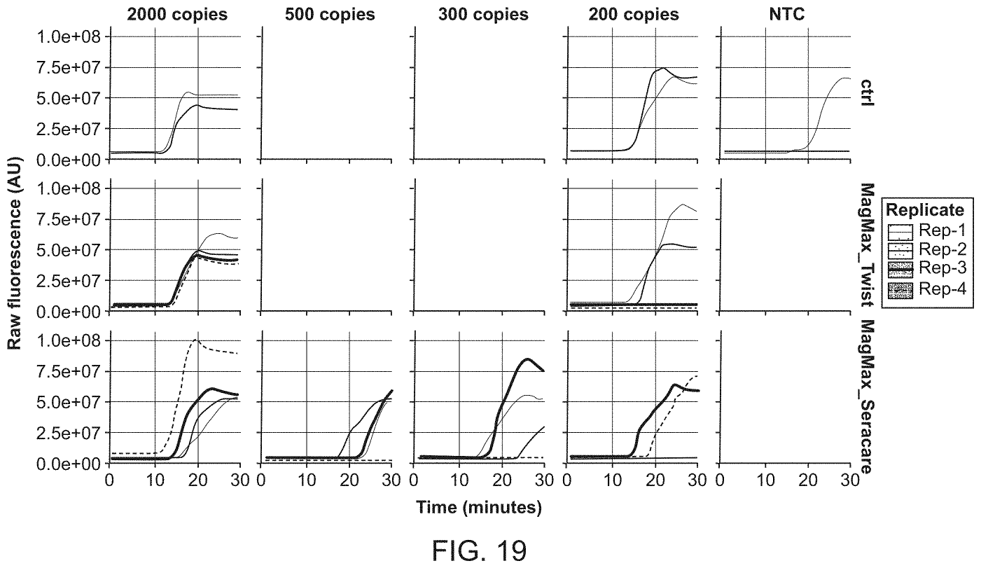

FIG. 19 illustrates fluorescence RT-LAMP plots of various samples that have been treated with the MagMAX viral isolation kit. A positive control sample included Twist synthetic RNA used with an RT-LAMP reaction, at 2000 and 200 copies per reaction, but without bead-based isolation. The MagMAX sample included the synthetic RNA processed with the viral isolation kit, also at 2000 and 200 copies per reaction. The SeraCare sample included SeraCare encapsulated SARS-CoV-2 RNA processed with the viral isolation kit, at 2000, 500, 300, and 200 copies per reaction. The samples were lysed for five minutes, bound with beads for three minutes, and washed twice each with W1 and W2 (2×W1+2×W2). The samples were eluted at 62 C for two minutes. Eluted samples were then added to RT-LAMP master mix and RT-LAMP was run for 30 minutes at 62 C. The samples treated with the viral isolation kit exhibited as good or better performance than the control. For the control, for 2000 and 200 copies, 2/2 replicates were positive. Both the SeraCare encapsulated RNA and Twist synthetic RNA samples in UTM tested positive for all four replicates at 2000 copies per reaction, The SeraCare treated samples showed three out of four results positive at 500 copies and 300 copies as well. Additional experimental testing with different lysis volumes (200, 300, 400 μL) showed improved consistency of capturing as low as 200 copies per reaction of SeraCare samples with higher wash volumes, suggesting that increased wash volumes may help reduce or eliminate carryover inhibitors of RT-LAMP. These results show that MagMAX beads can be used for sample prep with at least 2000 copies per reaction under the conditions tested, and that optimization may further improve results at lower copy numbers.

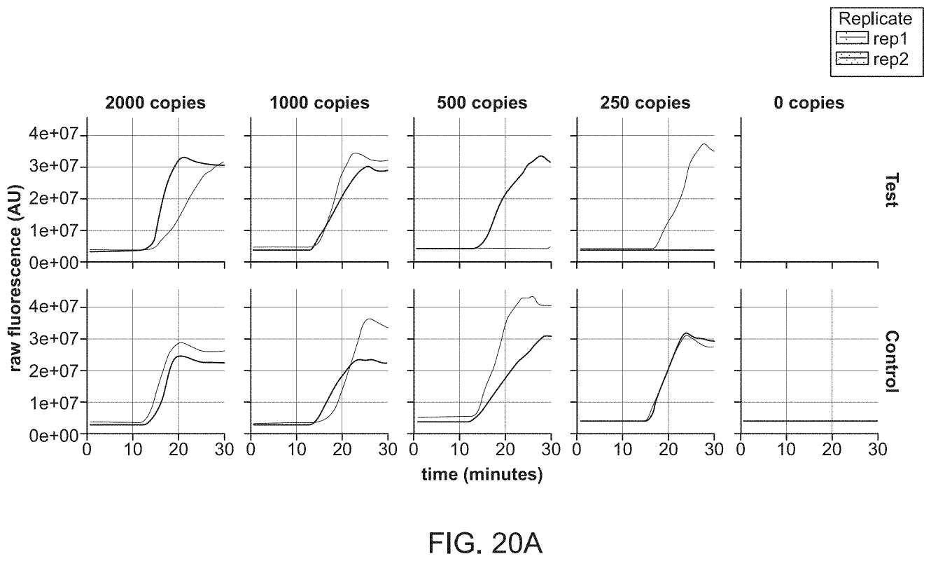

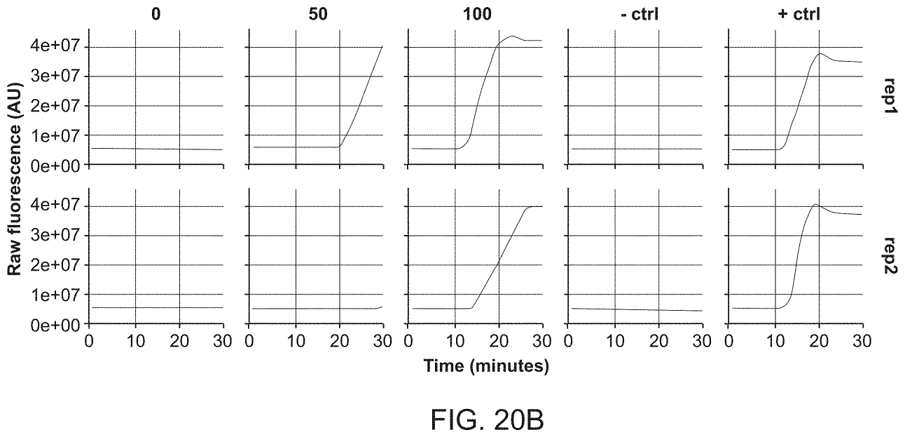

FIG. 20A illustrates a set of RT-LAMP plots showing samples treated with ChargeSwitch RNA purification kit. Using a lysis buffer comprising urea and a detergent, the RNA purification kit effectively captured RNA for RT-LAMP amplification at concentrations of at least 1000 copies per reaction. This is evidenced in the fluorescence plots for 2000 copies and 1000 copies, where both replicates provided strong fluorescence signals.

FIG. 20B illustrates a set of RT-LAMP plots showing samples treated with ChargeSwitch RNA purification kit. A different procedure was implemented. In a previous procedure (e.g., the procedure of FIG. 20A), a lysis buffer was added first to the sample, followed by RNA, followed by microparticles, followed by the binding buffer. In this procedure, the lysis buffer was added to the universal transport medium (UTM), followed by the binding buffer, sample RNA, and beads. Changing the order of addition of the reagents enabled capture and amplification of the sample at lower copy numbers. Additionally, the RNA purification kit was more effective with UTM at lower copy numbers than with lysis buffer alone.

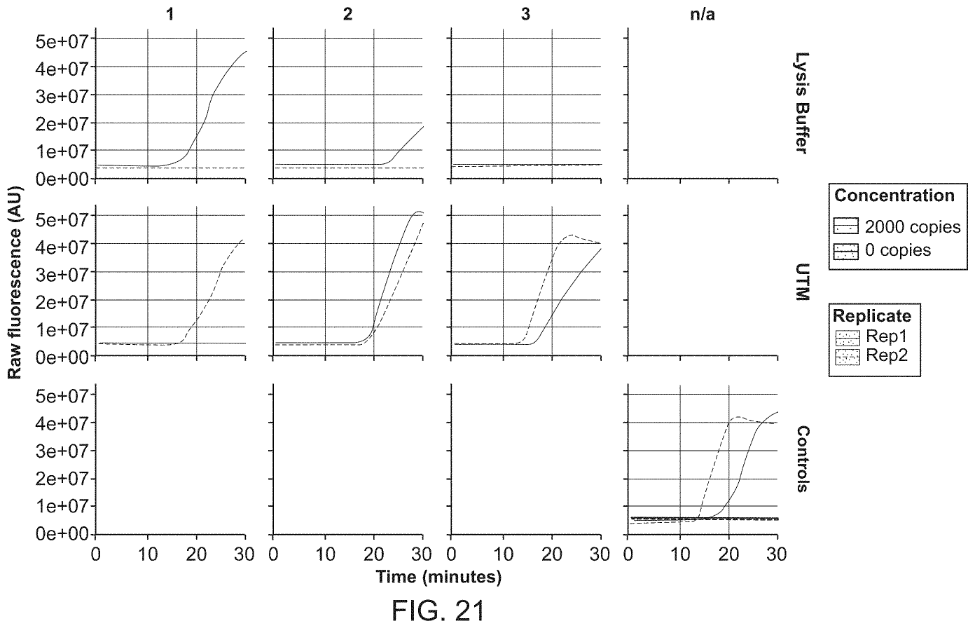

FIG. 21 illustrates similar results, to FIGS. 20A-20B, for two replicates at 200 copies per reaction. FIG. 21 shows that when UTM is added first along with the lysis buffer and binding buffer was added before the beads, better performance was obtained. Plots in columns 1, 2, and 3 show results for three different orders of addition. Column 1 shows results for addition of lysis buffer alone (top row) or UTM/lysis buffer (middle row) followed by sample RNA, then beads, then binding buffer (per ChargeSwitch standard protocol). Column 2 shows results for addition of lysis buffer alone (top row) or UTM/lysis buffer (middle row) followed by sample RNA, then binding buffer, then beads. Column 3 shows results for addition of lysis buffer alone (top row) or UTM/lysis buffer (middle row) followed by binding buffer, then sample RNA, then beads. Strong performance was found with UTM-containing samples and adding the binding buffer to the sample before adding the beads (Conditions 2 and 3) compared to the standard protocol (Condition 1).

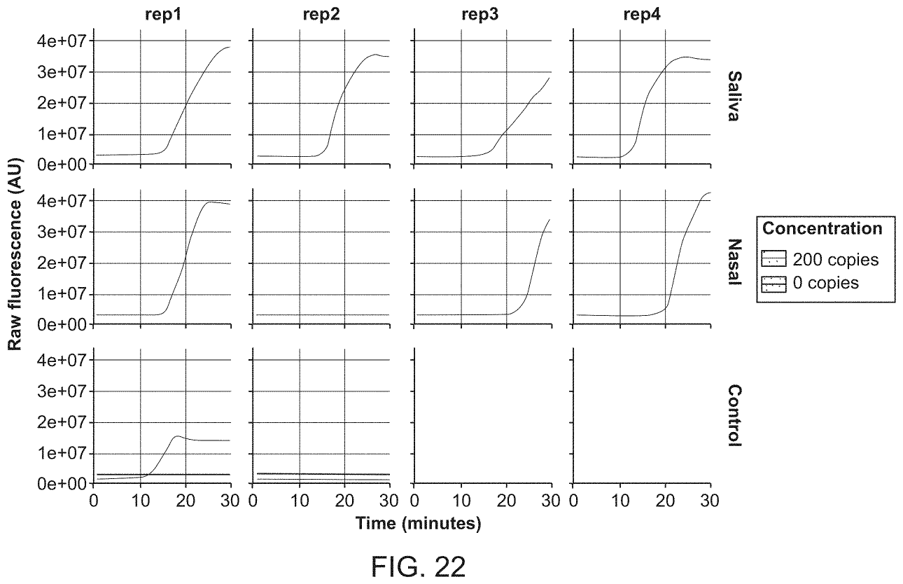

FIG. 22 illustrates plots of RT-LAMP results from samples treated with the ChargeSwitch RNA purification kit. The samples include four replicates of Twist RNA at concentrations of 200 copies (and a control of 0 copies). The top row of graphs shows results for saliva matrices and the middle row of graphs shows results from nasal matrices. ChargeSwitch purification successfully captured RNA for RT-LAMP from both contrived saliva and nasal matrices. Additional experiments were run with non-target E. coli spiked samples and 200 copies or 0 copies of target Twist RNA and both RT-LAMP and DETECTR were able to distinguish between the two in both nasal and saliva matrices.

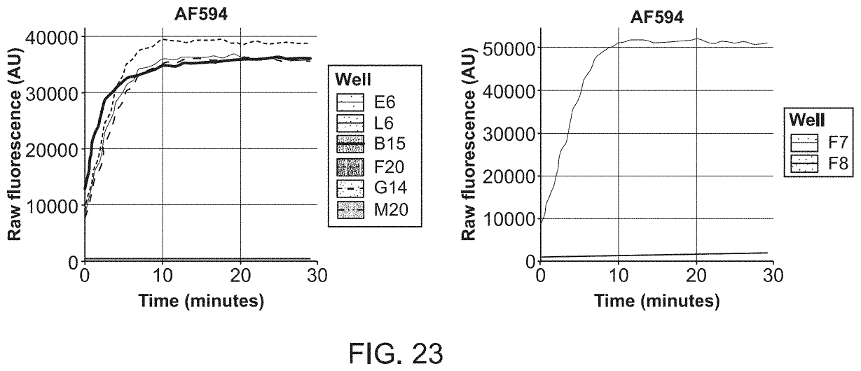

FIG. 23 shows plots of DETECTR reactions in a 384 deep-well microplate. In this experiment, the workflow used for detection included a 50 μL RT-LAMP reaction product and 150 μL DETECTR master mix added to the same sample chamber. Sample preparation was performed in a separate chamber prior to amplification and detection. The left-hand plot shows strong DETECTR fluorescence signals from samples placed in randomly-spaced wells of the microplate (wells E6, B15, F20, and G14) with little/no signal from wells without sample (wells L6 and M20). The right plot shows a plot of fluorescence signals from adjacent wells F7 and F8. As can be seen, there was minimal bleed-through (i.e., fluorescent signal from an adjacent well where a reaction is not taking place) from well F7, where a reaction took place, into well F8, where a reaction did not take place.

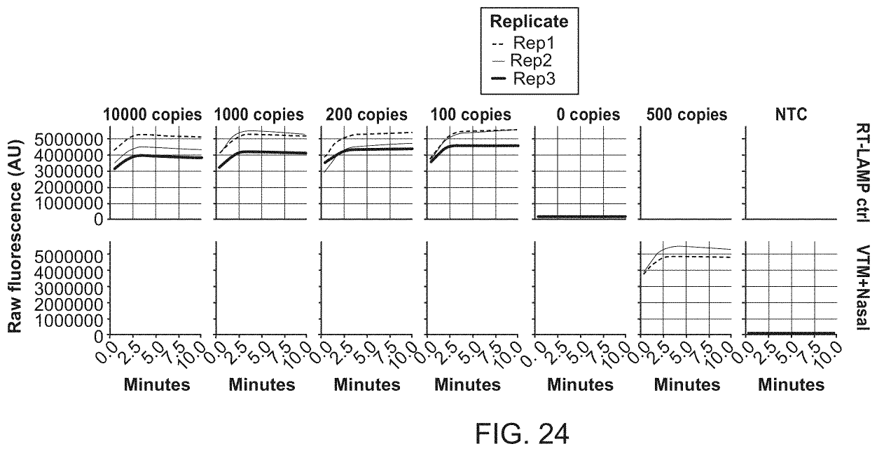

FIG. 24 shows plots comparing detection reactions with samples prepared from SeraCare SARS-CoV-2 positive reference RNA material in a Viral Transport Medium (VTM) and for an RT-LAMP positive control at 500 copies Twist RNA per reaction. The SeraCare in VTM showed strong fluorescence signal for a broad dynamic range of concentrations including 10000 copies, 1000 copies, 200 copies, and 100 copies for three replicates.

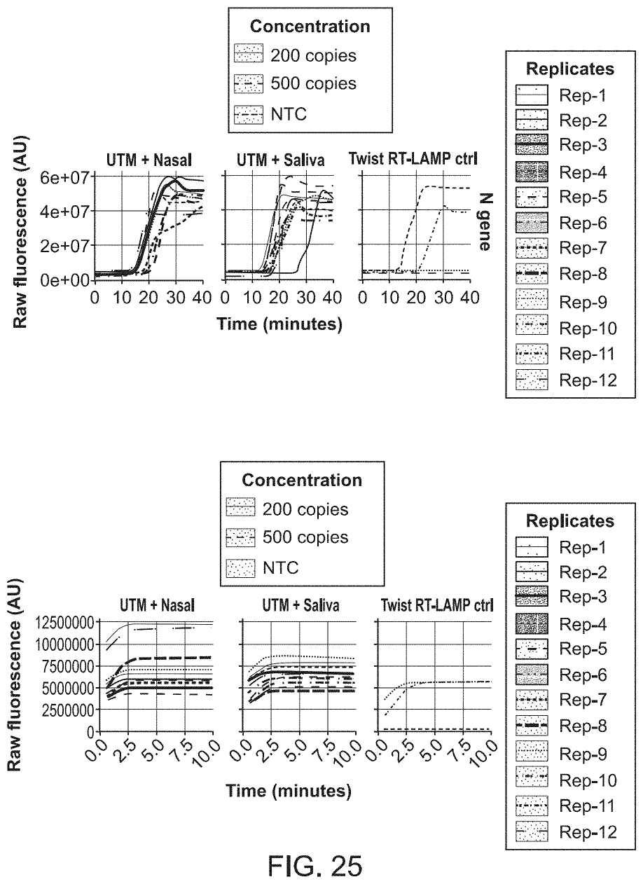

FIG. 25 illustrates a test of a complete workflow from sample to detection using MagMAX beads and SeraCare sample RNA in UTM with nasal or saliva matrix. The test shows RT-LAMP (left) and DETECTR (right) fluorescence signals collected from detection reactions for 200 copies/reaction SeraCare samples in UTM+Nasal matrix (12 replicates), 200 copies/reaction SeraCare samples in UTM+Saliva (twelve replicates), 500 copies/reaction Twist RT-LAMP (as a positive control, two replicates), or NTC (two replicates). The tests showed strong fluorescence signals for SeraCare replicates at 200 copies per reaction in UTM with saliva or nasal matrix.

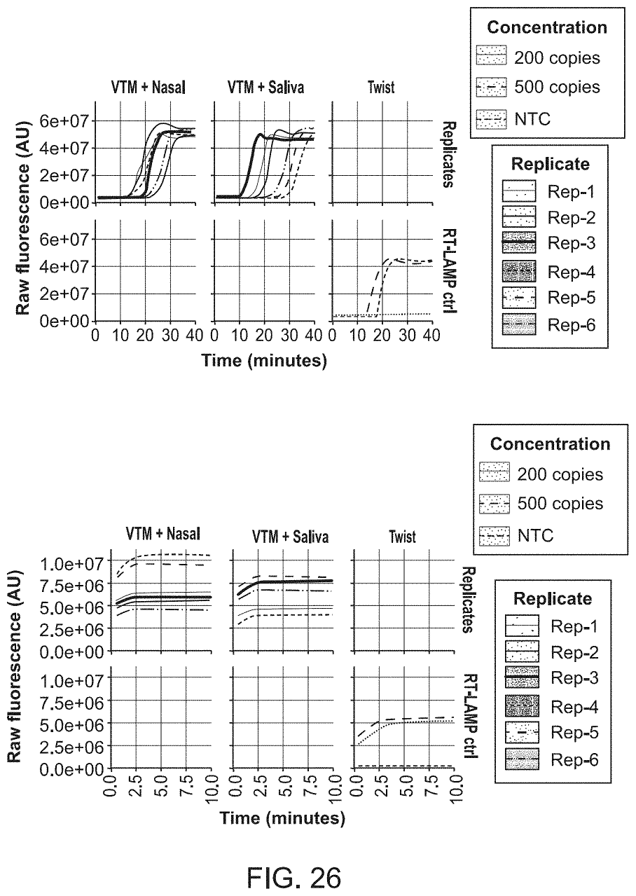

FIG. 26 illustrates a test of a complete workflow from sample to detection using MagMAX beads and SeraCare sample RNA in VTM with nasal or saliva matrix. The test shows RT-LAMP (left) and DETECTR (right) fluorescence signals collected from detection reactions for 200 copies/reaction SeraCare samples in VTM+Nasal (6 replicates), 200 copies/reaction SeraCare samples in VTM+Saliva (6 replicates), Twist RT-LAMP (as a positive control, 2 replicates), or NTC (two replicates The tests showed strong fluorescence signals for SeraCare replicates at 200 copies per reaction in VTM with saliva or nasal matrix.

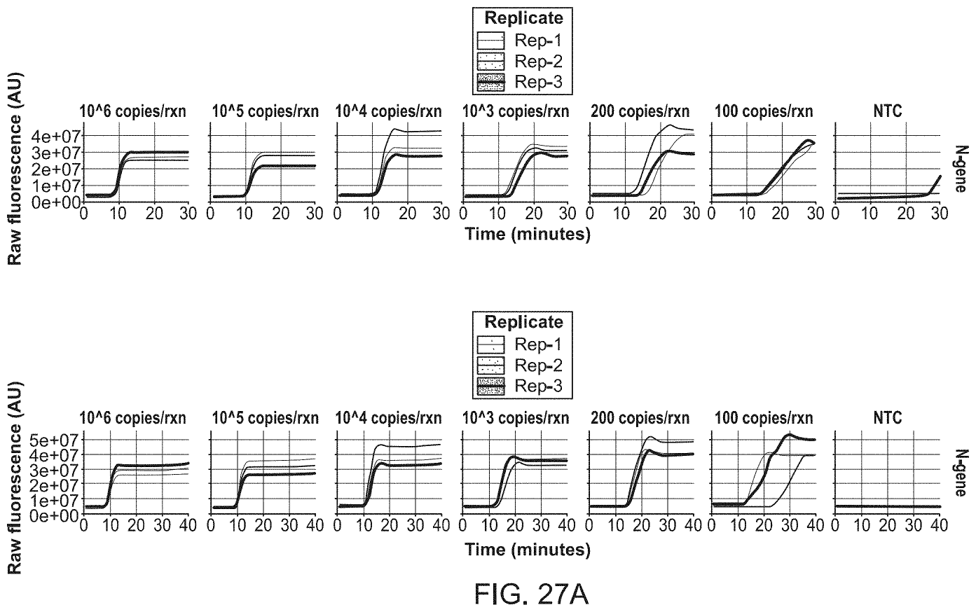

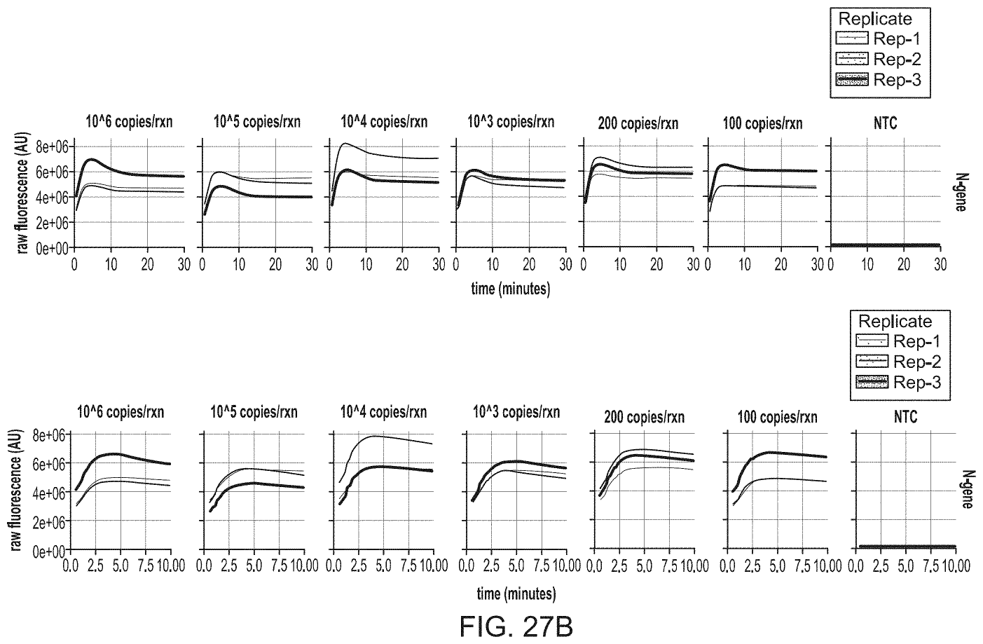

FIG. 27A illustrates plots showing the effects on detection signals when large copy numbers of N-gene are used in RT-LAMP reactions. In these reactions, the wells included the 25 μL Twist sample and 25 μL master mix comprising two sub-master mixes. 25 μL Twist sample having between 10⁶ and 100 copies per reaction, 20 μL salts and primers sub-master mix, and 5 μL enzymes master mix were added together and incubated at 62 C for 30 minutes (top) or 40 minutes (bottom). In all copy numbers, all positive RT-LAMP reactions were successfully detected.

FIG. 27B illustrates plots showing the effects on detection signals when large copy numbers of N-gene are used in DETECTR reactions. In these reactions, the wells included 150 μL of DETECTR master mix added to 50 μL RT-LAMP reaction from FIG. 27A. DETECTR reactions were run for 30 minutes at 37 C (top) or 10 minutes at 37 C (bottom). Corresponding to the results from FIG. 27A, in reactions for all concentrations of the target, all positive reactions were successfully detected using DETECTR, as evidenced by the strong fluorescence signals for the replicates.

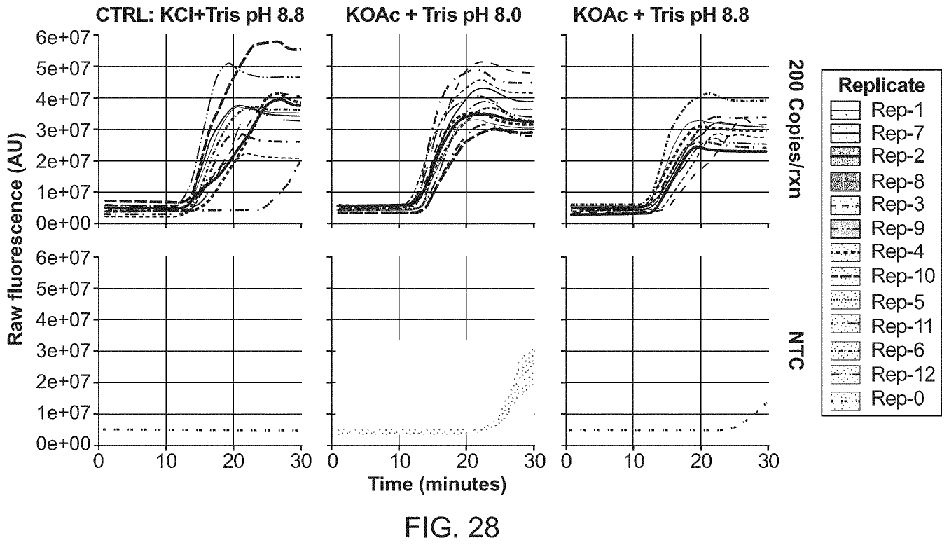

FIG. 28 illustrates detection results from a new reagent buffer formulation for RT-LAMP, where KCl was replaced with KOAc and Tris pH 8.8 was compared to Tris pH 8.0. The detection mix used was the 25 μL Twist sample at 200 copies/reaction+25 μL RT-LAMP master mix, which comprised 25 μL sample, 20 μL salts and primers, and 5 μL enzymes. With 200 copies, 100% of the positive samples were successfully detected with all buffer conditions after 30 minutes RT-LAMP at 62 C.

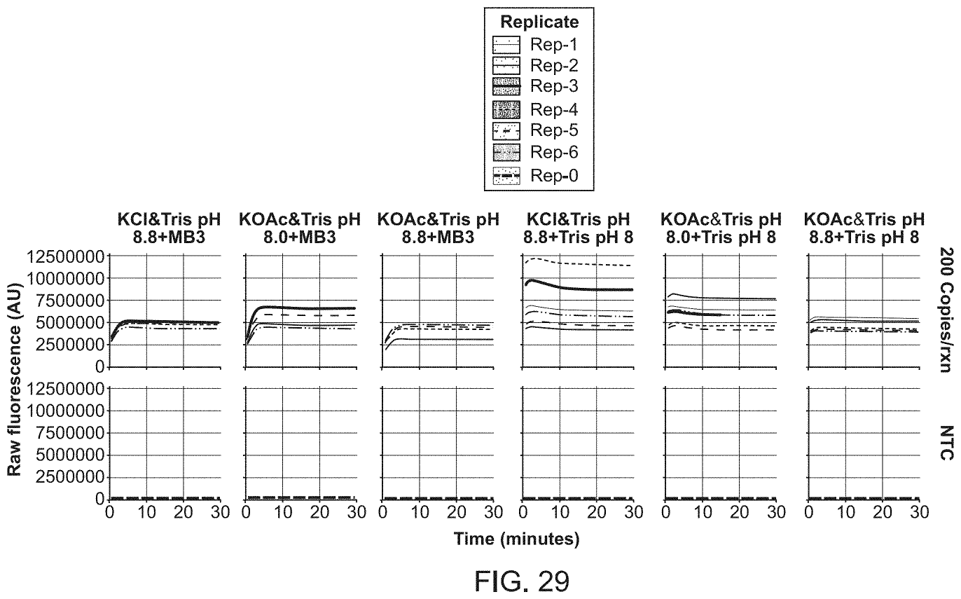

FIG. 29 illustrates detection results from a new reagent buffer formulation for DETECTR, where HEPES pH 7.5 (in MB3) was replaced with Tris pH 8. In these reactions, 150 μL DETECTR Master mix was added to 50 ul RT-LAMP product from FIG. 28 and provided strong detection results for 200 copies of all replicates and buffer conditions tested. The DETECTR data correlated with the amplification data.

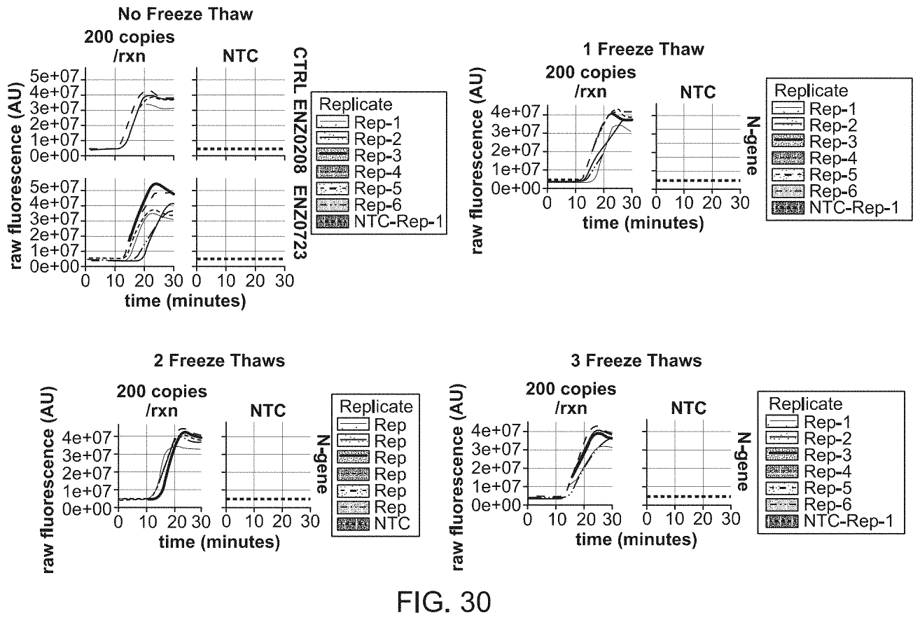

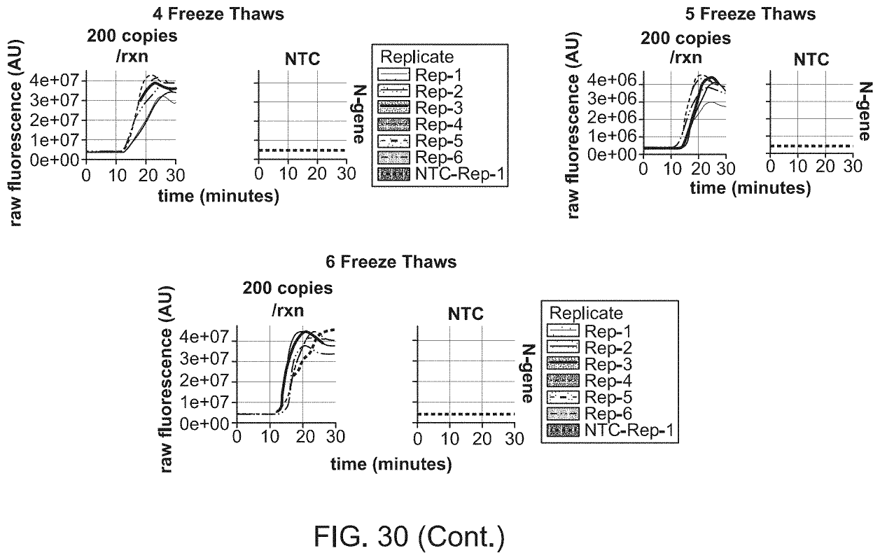

FIG. 30 illustrates results from a reagent stability study. RT-LAMP ENZ (Enzymes+dNTPs) master mix was frozen and thawed multiple times (0-6 freeze thaws) prior running RT-LAMP reactions. The plots show that, even after six freeze-thaw cycles, the ENZ reagent remained stable. This is demonstrated by the fact that fluorescence RT-LAMP signals are detected for replicates at 200 copies after each freeze thaw cycle.

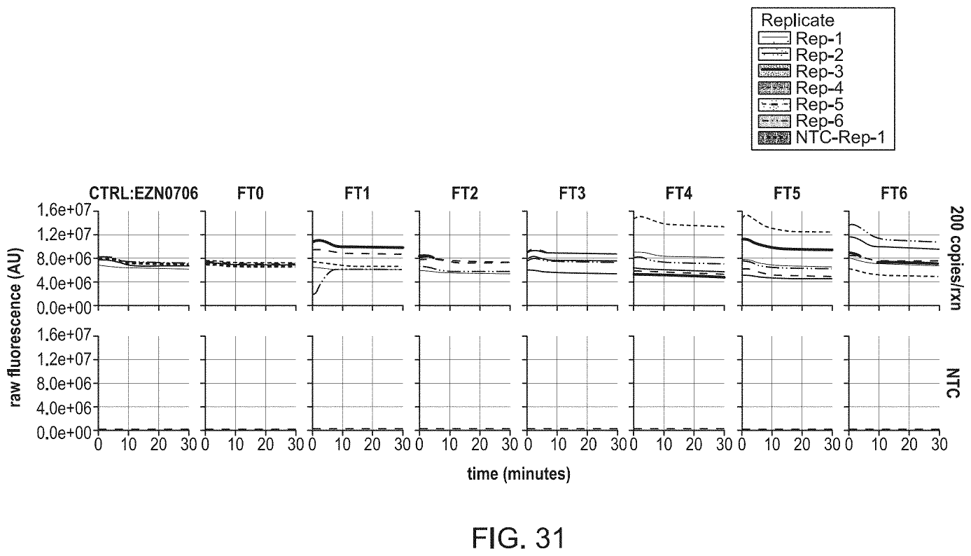

FIG. 31 illustrates DETECTR results from the stability study of FIG. 30. In these experiments, all RT-LAMP products were transferred into a new plate. 150 μLμL DETECTR Master mix was added to 50 μLμL RT-LAMP reaction from FIG. 30. Based on the DETECTR data, all replicates were successfully detected, signifying that the ENZ is stable up to at least six times of freeze-thaw cycle.

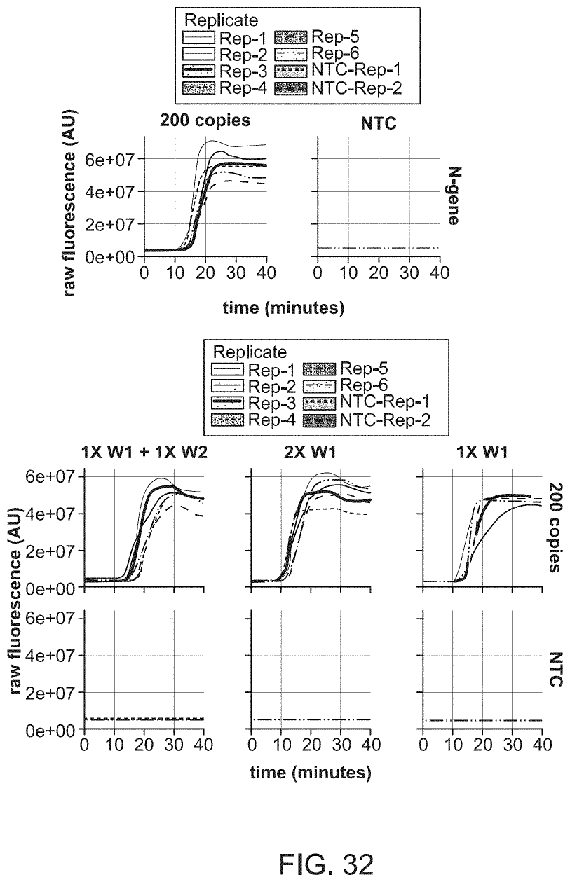

FIG. 32 illustrates a wash protocol optimization for a reaction using MagMAX viral isolation kit. This experiment shows that washing twice with solution W1 (2×W2) yielded faster detection than washing using the standard protocol of twice each with W1 and W2 (left), once each with W1 and W2 (1×W1+1×W2) or only once with W1 (1×W1). This could be due to Guanidine hydrochloride (GuHCl) carryover from the wash buffer W1 into the RT-LAMP reaction.

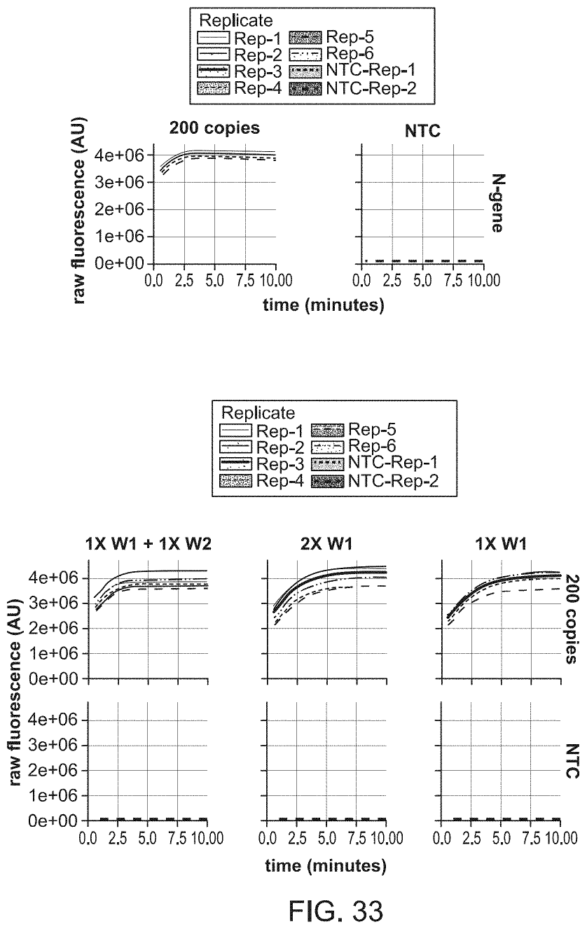

FIG. 33 illustrates DETECTR reactions following the RT-LAMP reactions shown in FIG. 32 with sample preparation using the MagMAX viral isolation kit. As can be seen from the fluorescence plots, all wash protocols produced strong DETECTR signal. Washing once each with W1 and W2 yielded comparable results to using the standard protocol.

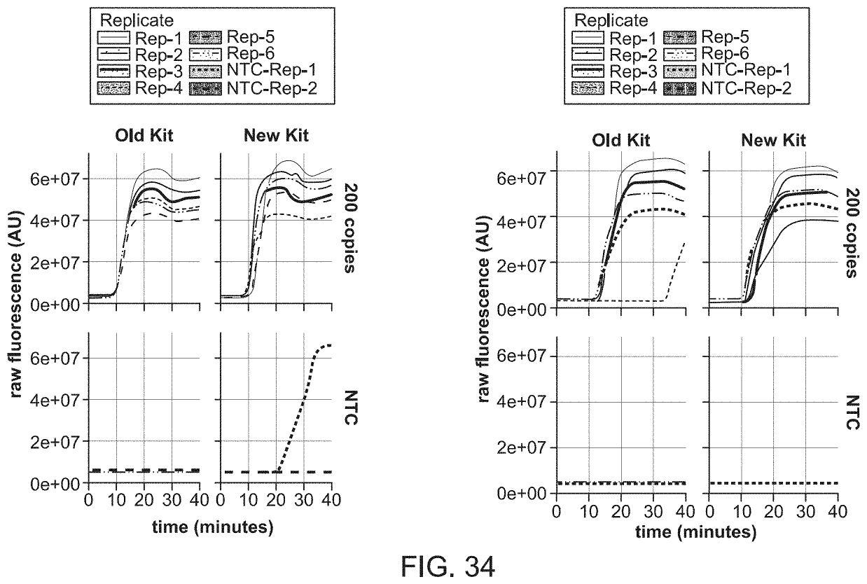

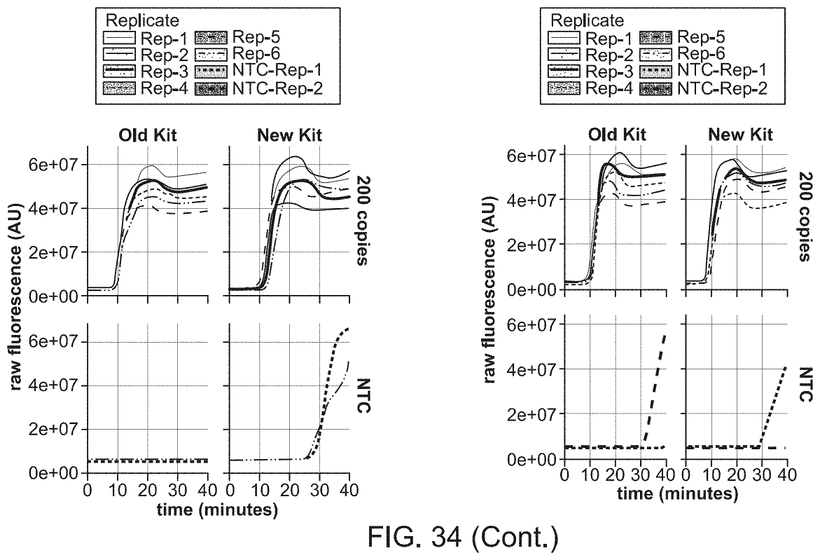

FIG. 34 illustrates plots showing results of a MagMAX kit stability study used for sample preparation before an RT-LAMP reaction. Two MagMAX kits were prepared at different times to determine the effect of using older reagents (“old kit”) versus freshly prepared reagents (“new kit”). Both kits were stable and enable strong amplification via RT-LAMP over a period of 1 to 6 days after MagMAX kit reagent preparation. Reagents were stored at room temperature during the study.

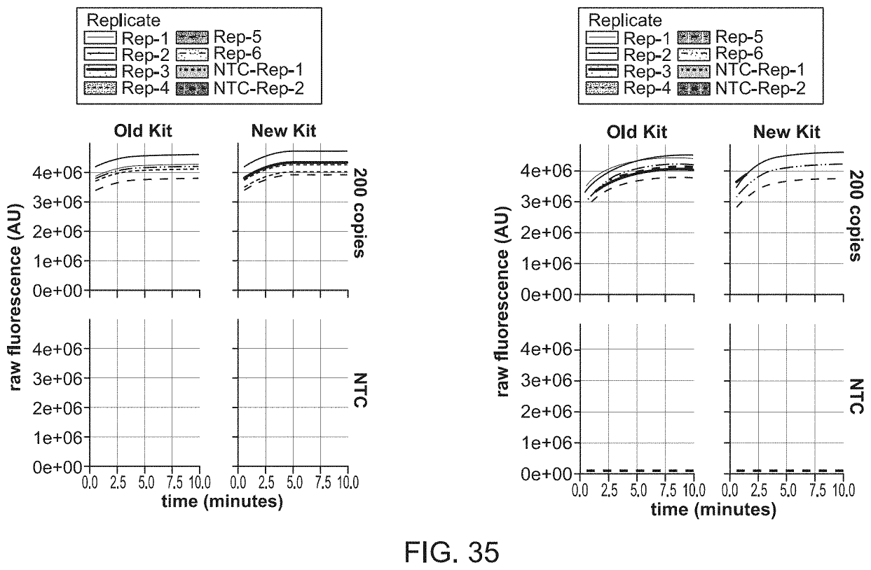

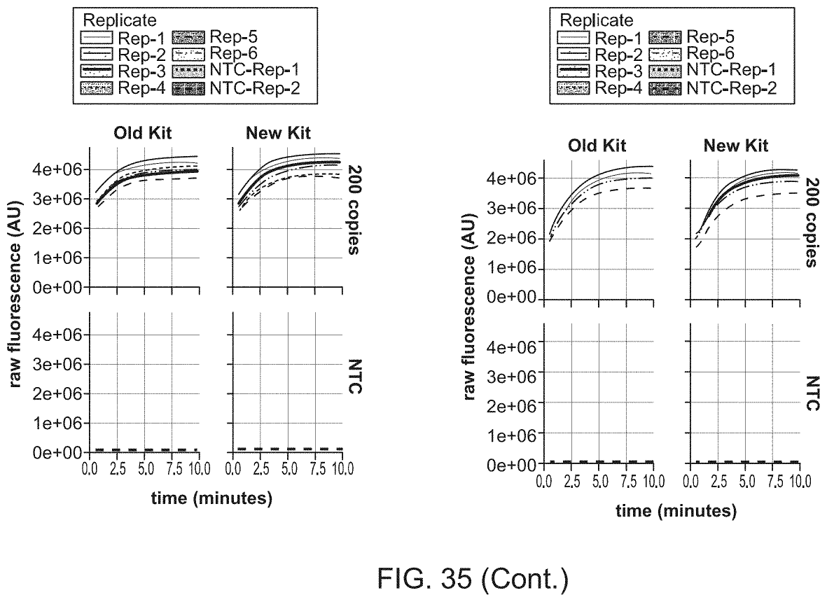

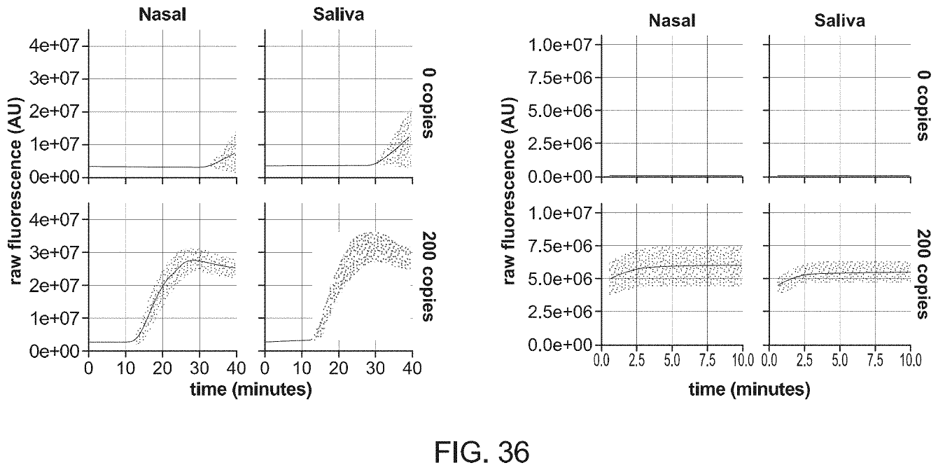

FIG. 35 illustrates plots showing DETECTR results of the stability study of FIG. 34. The MagMAX kit remained stable at room temperature for up to at least six days.

FIG. 36 illustrates fluorescence results from using a 5× acetate lysis/binding buffer with nasal and saliva samples in UTM prepared using ChargeSwitch beads. Samples were lysed at 62 C for 5 minutes then incubated with the ChargeSwitch beads at room temperature for 3 minutes. The beads were then washed twice and the nucleic acids were eluted before being transferred to a fresh chamber for RT-LAMP amplification and DETECTR detection. The results show that, for 200 copy samples per reaction, the acetate-containing lysis buffer was compatible with both RT-LAMP and DETECTR reactions, as strong fluorescence signals were achieved. The results show that the 6 true negatives/matrix and 12 positives/matrix were detected.

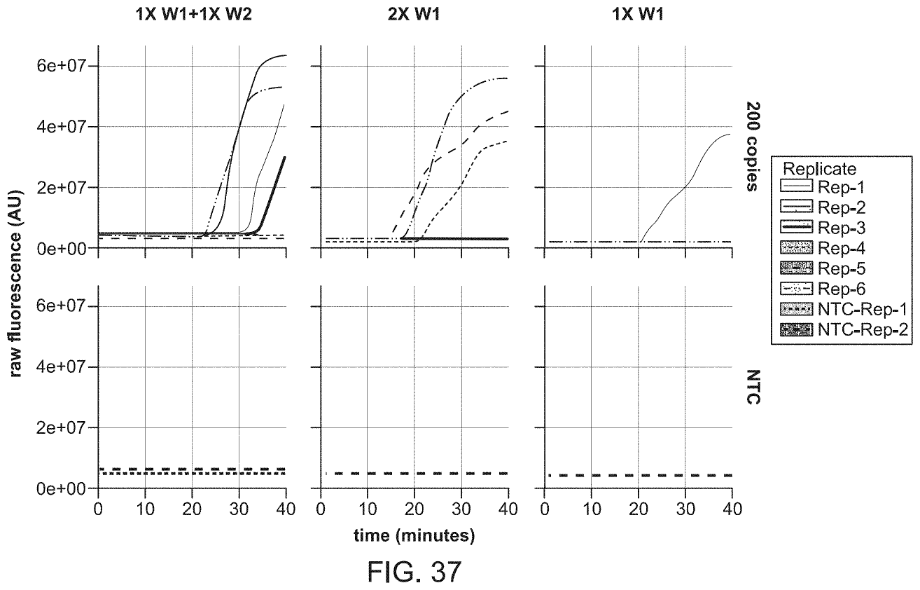

FIG. 37 illustrates results from RT-LAMP reactions following sample prep with reduced washing (down from standard twice each with W1 and W2) protocols with the MagMAX viral isolation kit, using a sample containing UTM+200 copies SeraCare sample per reaction. Based on the RT-LAMP data, 1× W1+1× W2 yielded better results than the other washing conditions tested (2×W1 or 1×W1) for the workflow tested. 2×W1 performed well but some cell clumps were observed (possibly from inefficient lysis) which may have artificially reduced the performance of the sample preparation and RT-LAMP reactions.

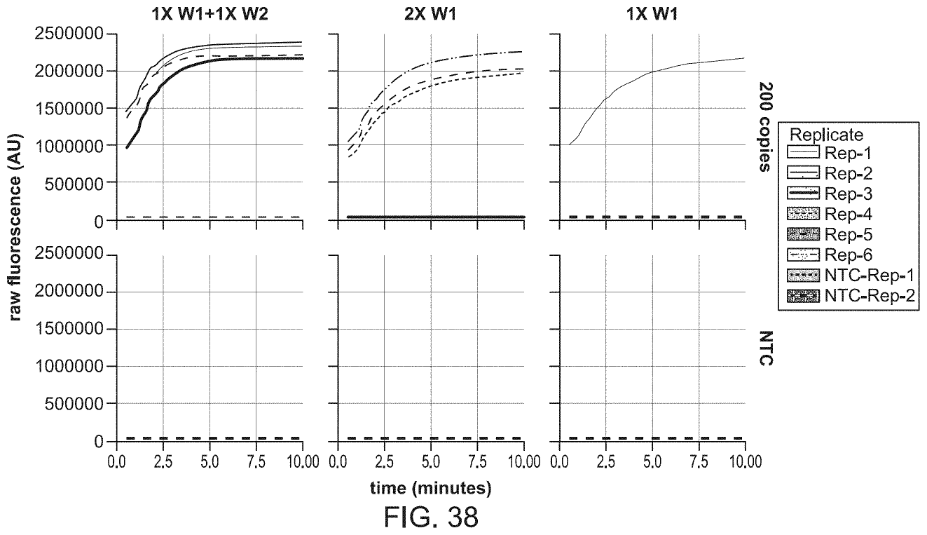

FIG. 38 illustrates results from DETECTR reactions following the RT-LAMP reaction of FIG. 37. Based on the DETECTR data, 1× W1+1× W2 yielded better results than the other washing conditions. 2×W1 performed well but some cell clumps were observed (possibly from inefficient lysis) which may have artificially reduced the performance of the sample preparation and RT-LAMP reactions preceding the DETECTR reaction.

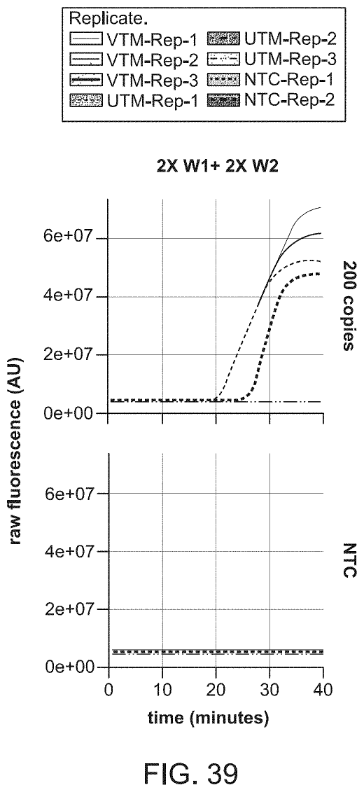

FIG. 39 illustrates RT-LAMP results of samples containing 200-copies SeraCare sample per reaction and nasal matrices in UTM compared with VTM prepared with MagMAX beads and the standard (2×W1+2×W2) wash regime. RT-LAMP reactions were successfully run in 2/3 replicates for both UTM and VTM containing samples.

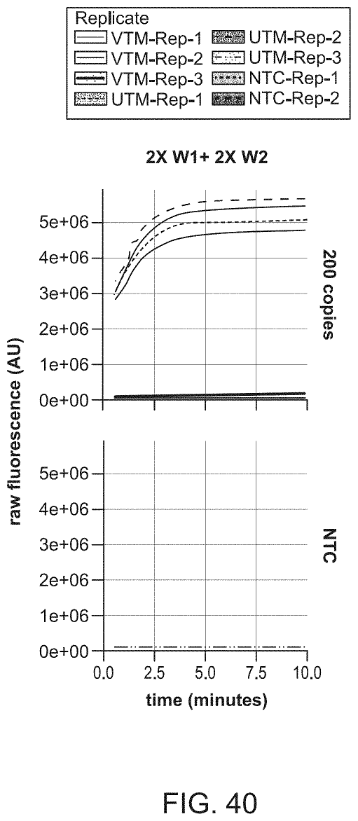

FIG. 40 illustrates DETECTR results of the samples of FIG. 39. DETECTR reactions were successfully run in 2/3 replicates for both UTM and VTM containing samples, mirroring the RT-LAMP reaction results.

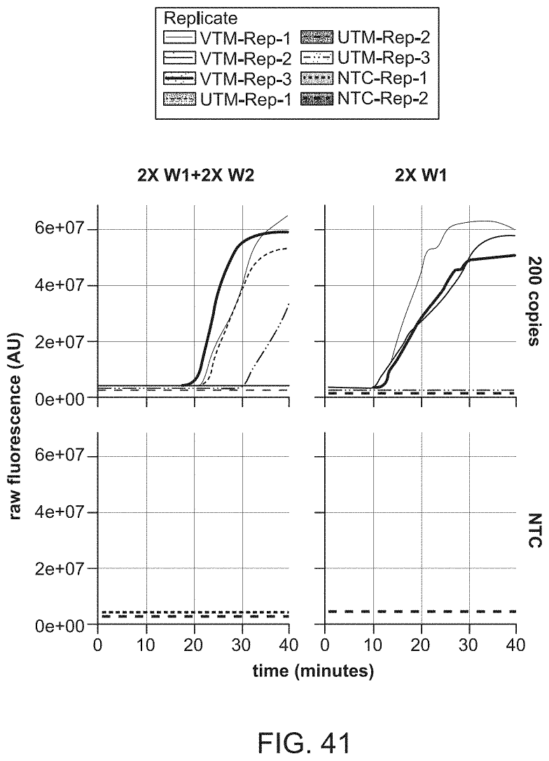

FIG. 41 illustrates fluorescence results for RT-LAMP reactions using samples prepared with different washing conditions in either VTM and UTM with the MagMAX viral isolation kit. The plots show that 2× W1 was the best washing condition with VTM. For 200-copy reactions with VTM, the number of positive test samples correctly detected follows: 2/3 with 2× W1+2× W2, 3/3 with 2× W 1. For UTM-containing samples, the number correctly detected were 2/3 with 2× W1+2× W2.

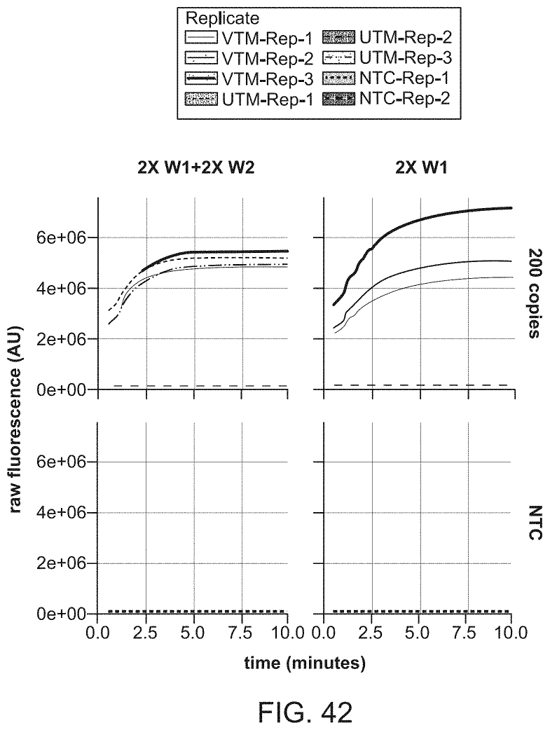

FIG. 42 illustrates a comparison of fluorescence results for DETECTR reactions for RT-LAMP samples from FIG. 41 prepared from samples containing VTM and UTM without nasal/saliva matrices. In sample with a nasal matrix, synthetic mucus is added to better simulate diagnostic conditions. In a sample with a saliva matrix, synthetic saliva is added to the samples to better simulate diagnostic conditions. These results correspond to the RT-LAMP results, with 2× W1 favored for VTM and 2× of each wash favored for UTM under the conditions tested.

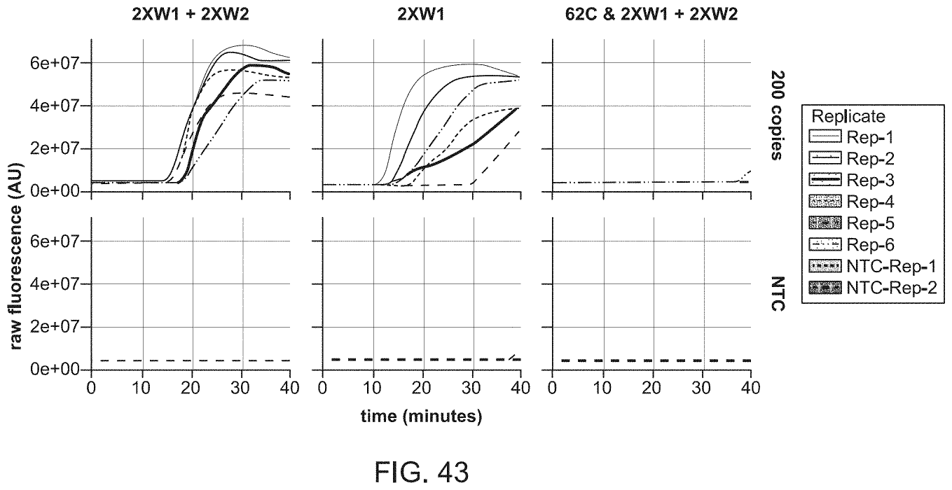

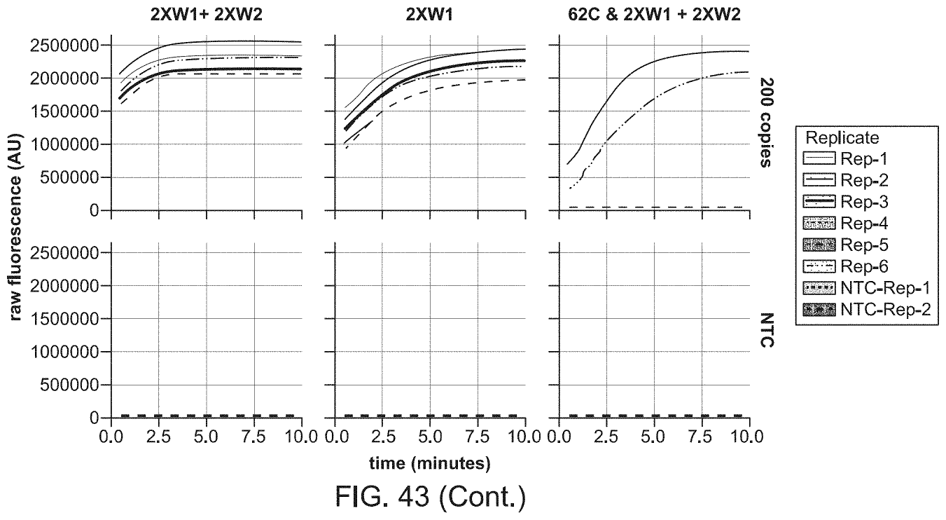

FIG. 43 illustrates comparisons of RT-LAMP (top) and DETECTR (bottom) results following sample preparation with different wash protocols for MagMAX viral isolation kit for samples not containing nasal and saliva matrices. Using 200-copy SeraCare UTM samples per reaction as the input, the standard wash protocol (2×W1+2×W2) detected 6/6 positives and a reduced washing protocol (2×W1) detected 6/6 positives. Interestingly, 2×W1+2×W2 with 62° C. lysis incubation reduced extraction efficiency, as RT-LAMP was unable to generate a detectable signal but the DETECTR reaction was still able to identify 2/6 positives (indicating robust DETECTR activity even in the presence of low copy number following amplification). Further experimentation with lysis temperatures at 37 C, 45 C, and 55 C for 5 minutes prior to isolation using MagMAX beads showed strong RT-LAMP and DETECTR signals for 2/3 replicates tested. These results indicate that the lysis incubation temperature and wash conditions may be optimized in order to improve the RNA extraction and/or the amplification reaction.

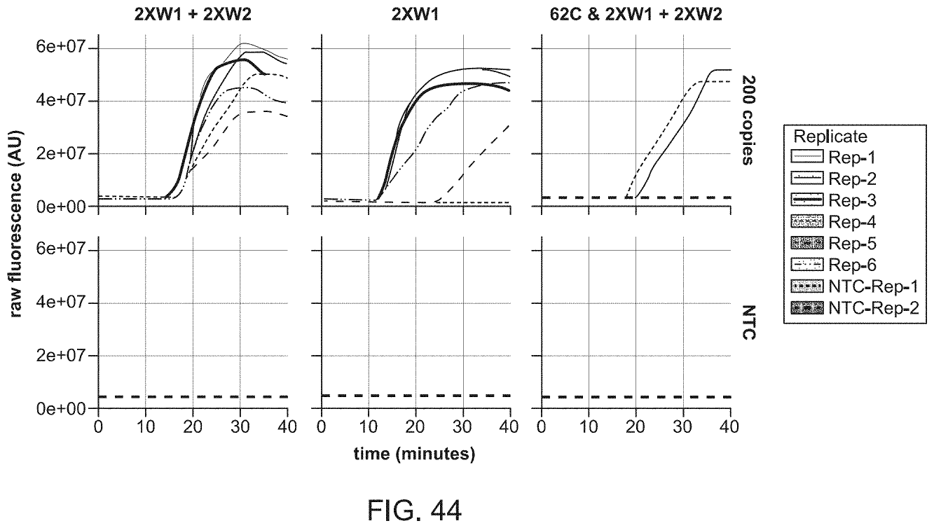

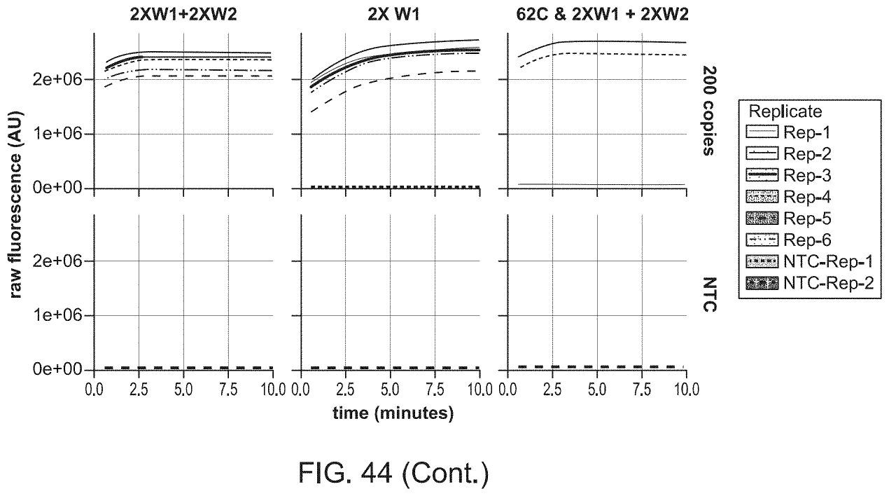

FIG. 44 illustrates comparisons of RT-LAMP (top) and DETECTR (bottom) results following sample preparation with different wash protocols for MagMAX viral isolation kit for samples containing a nasal matrix. Using 200-copy SeraCare UTM samples per reaction as the input, the standard wash protocol (2×W1+2×W2) detected 6/6 positives and a reduced washing protocol (2×W1) detected 5/6 positives. 2×W1+2×W2 with 62° C. lysis incubation detected 2/6 positives. Further experimentation with lysis temperatures at room temperature and 37 C with Proteinase K showed improved detection of 6/6 copies. These results indicate that the lysis incubation temperature and wash conditions may be optimized in order to improve the RNA extraction and/or the amplification reaction.

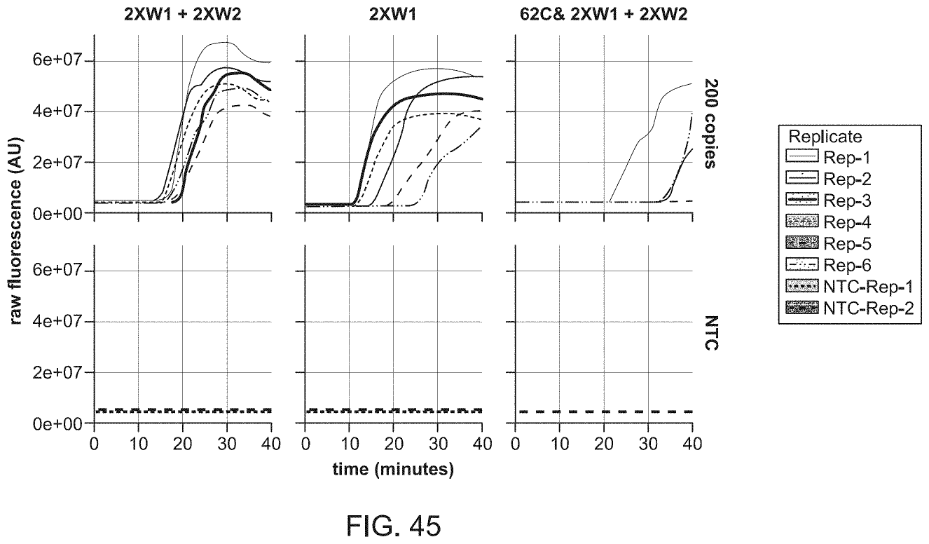

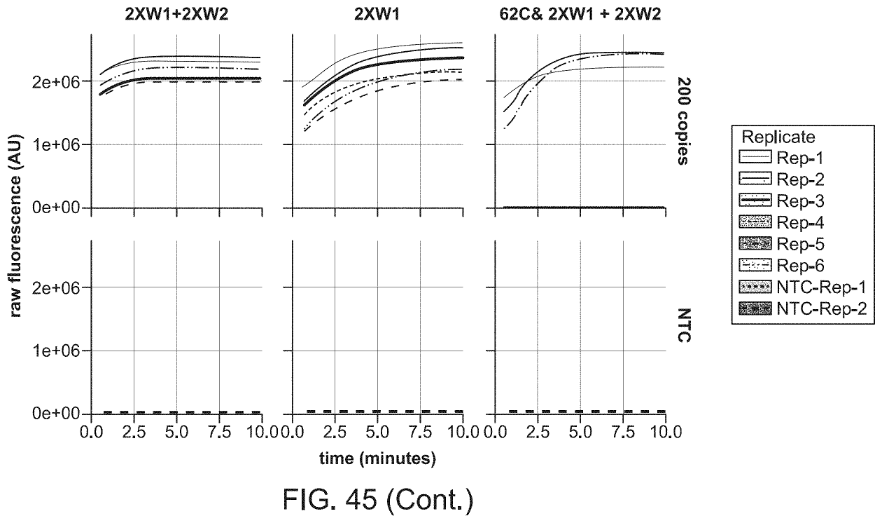

FIG. 45 illustrates comparisons of RT-LAMP (top) and DETECTR (bottom) results following sample preparation with different wash protocols for MagMAX viral isolation kit with for samples containing a saliva matrix. Using 200-copy SeraCare UTM samples per reaction as the input, the standard wash protocol (2×W1+2×W2) detected 6/6 positives and a reduced washing protocol (2×W1) detected 6/6 positives., 2×W1+2×W2 with 62° C. lysis incubation detected 3/6 positives. These results indicate that the lysis incubation temperature and wash conditions may be optimized in order to improve the RNA extraction and/or the amplification reaction.

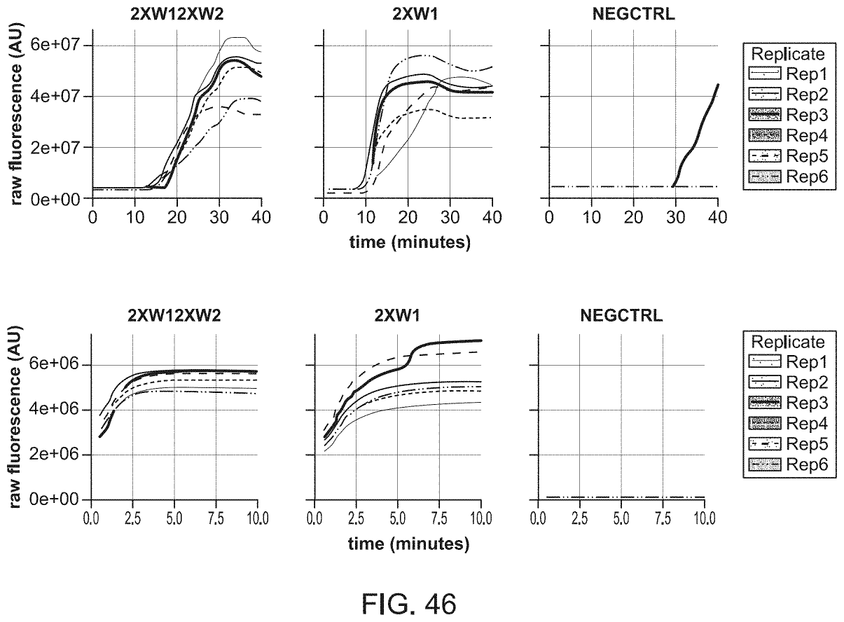

FIG. 46 illustrates comparisons of RT-LAMP (top) and DETECTR (bottom) results following sample preparation with different wash protocols for MagMAX testing with mock clinical samples in VTM with a nasal matrix lysed at room temperature. Results indicated that 6/6 replicates were correctly indicated to be positive for both 2×W1+2×W2 and 2×W1 wash protocols.

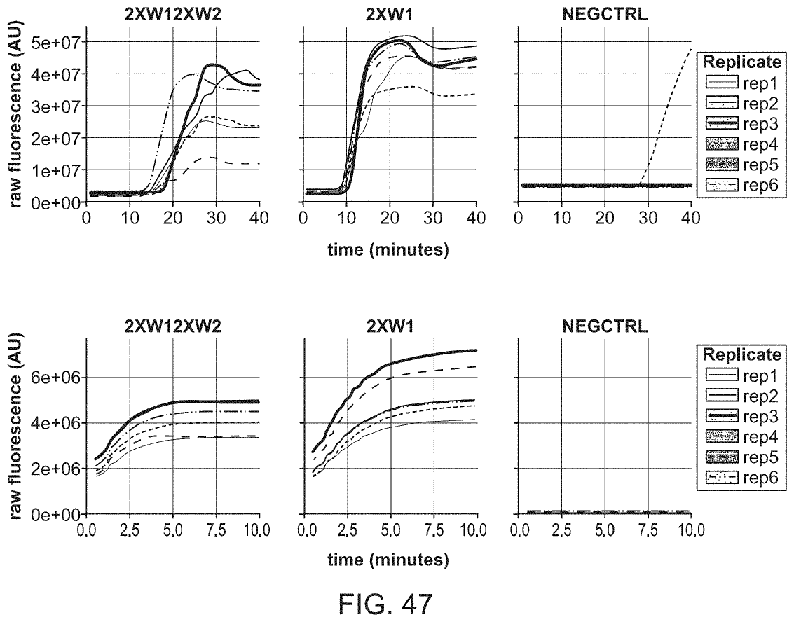

FIG. 47 illustrates comparisons of RT-LAMP (top) and DETECTR (bottom) results following sample preparation with different wash protocols for MagMAX testing with mock clinical samples in VTM with a saliva matrix lysed at room temperature. Results indicated that 6/6 replicates were correctly indicated to be positive for 2×W1+2×W2 and 2×W1 wash protocols.

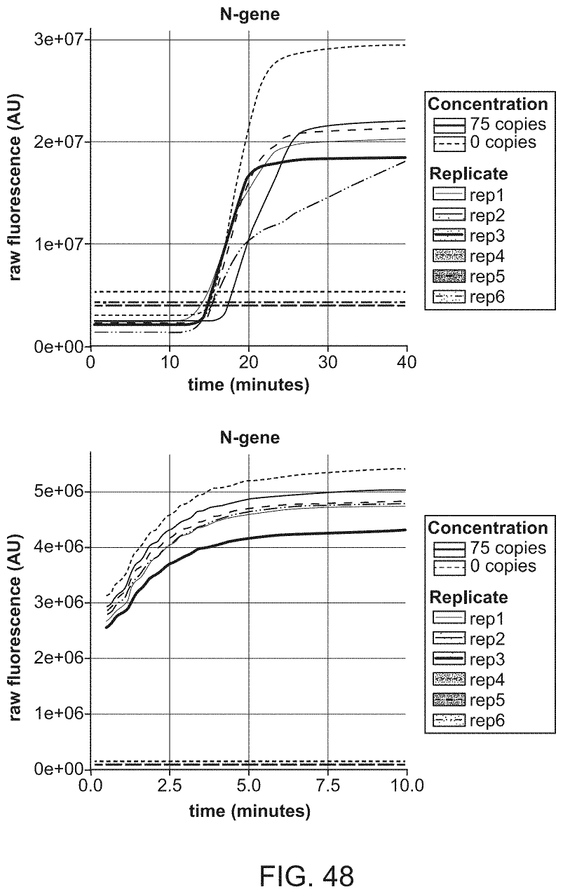

Limit of detection (LOD) experiments were run for 200, 150, 100, 75, and 50 copies of SeraCare sample processed in UTM with nasal matrix and 2× proteinase K with MagMAX beads for sample preparation and 2×W1 wash prior to elution. Lysis was performed at 37 C for 5 minutes. RT-LAMP and DETECTR reactions were performed on eluted samples in a separate chamber. Strong results were detected for 75 copies and above for the assay conditions tested. FIG. 48 illustrates fluorescence results from a high-performing MagMAX workflow for RT-LAMP and DETECTR reactions. The workflow included adding a 110 μL UTM SeraCare sample (containing 75 copies/reaction), 100 μL Lysis/Binding Buffer (w/50% v/v IPA), and 10 μL beads into a reaction chamber. Lysis was run for 5 minutes at 37 C before the beads were captured with the magnet for 3 minutes. The beads were then washed twice with W1 and air dried for two minutes before elution. 25 μL elution buffer was added to the beads and elution was performed at 62 C for two minutes. Eluted nucleic acids were then transferred to a second reaction chamber for RT-LAMP at 57 C for 40 minutes followed by DETECTR at 37 C for 10 minutes. The workflow provided strong positive signals for all six replicates.

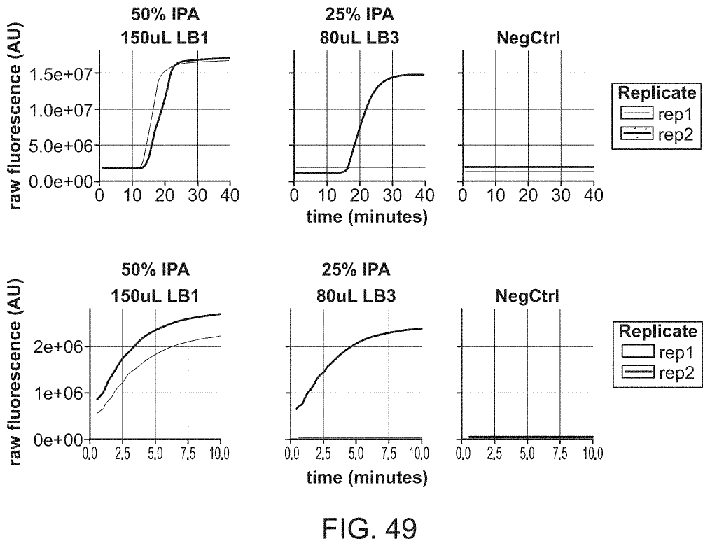

FIG. 49 illustrates RT-LAMP (top) and DETECTR (bottom) test results after reducing lysis buffer volume to sample volume and/or the amount of IPA in the lysis buffer. LB1 included 400 μL lysis buffer concentrate and 400 μL IPA and 6.4 μL carrier RNA and served as a positive control. LB3 included 300 μL lysis buffer concentration and 150 μL IPA and 3.2 μL carrier RNA. The best results were obtained at 50% IPA.

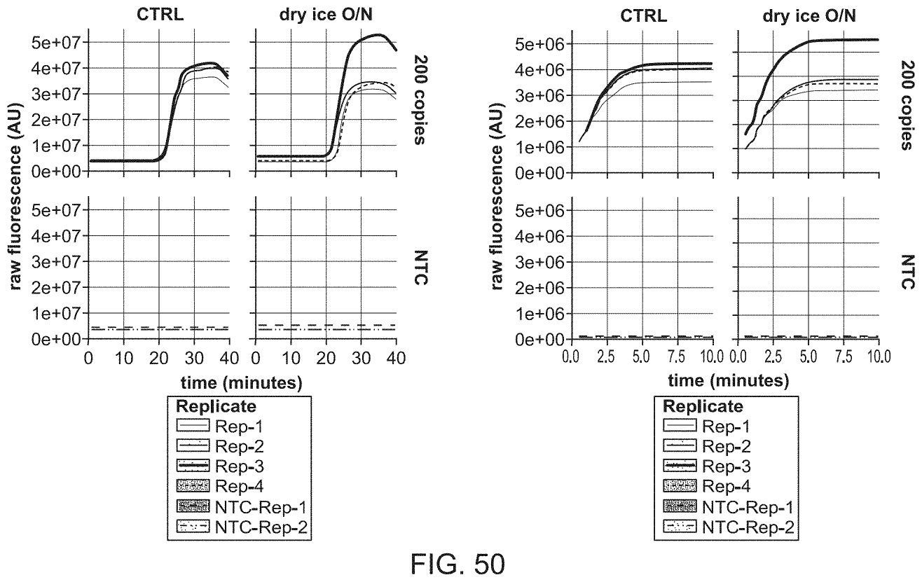

FIG. 50 illustrates results from a shipping stability study for a set of N gene reagents including RT-LAMP activator, RT-LAMP master mix, and DETECTR master mix. The RT-LAMP and DETECTR master mixes were incubated on dry ice overnight. The results show that the reagents were stable for at least 24 hours when shipped with dry ice.

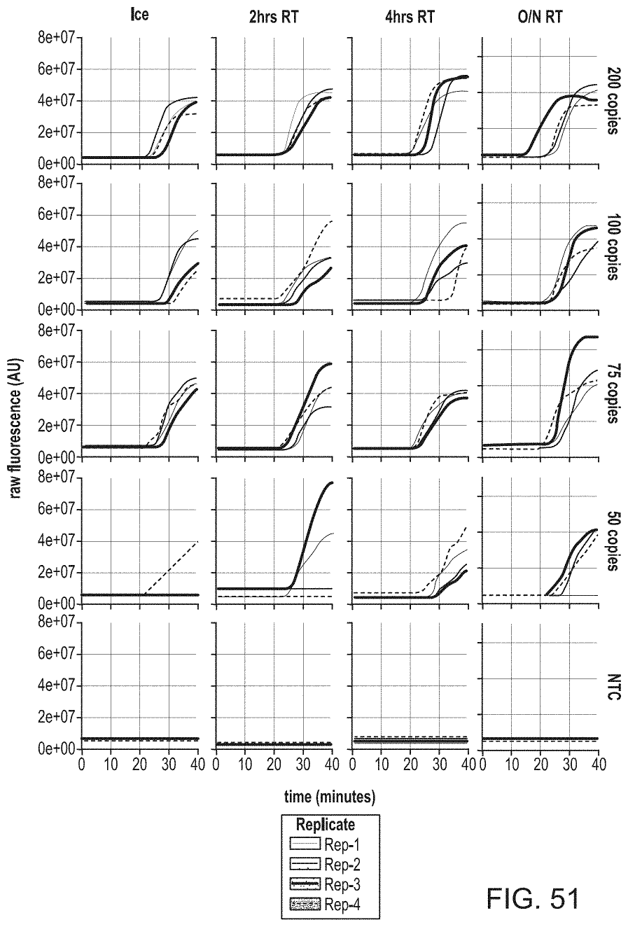

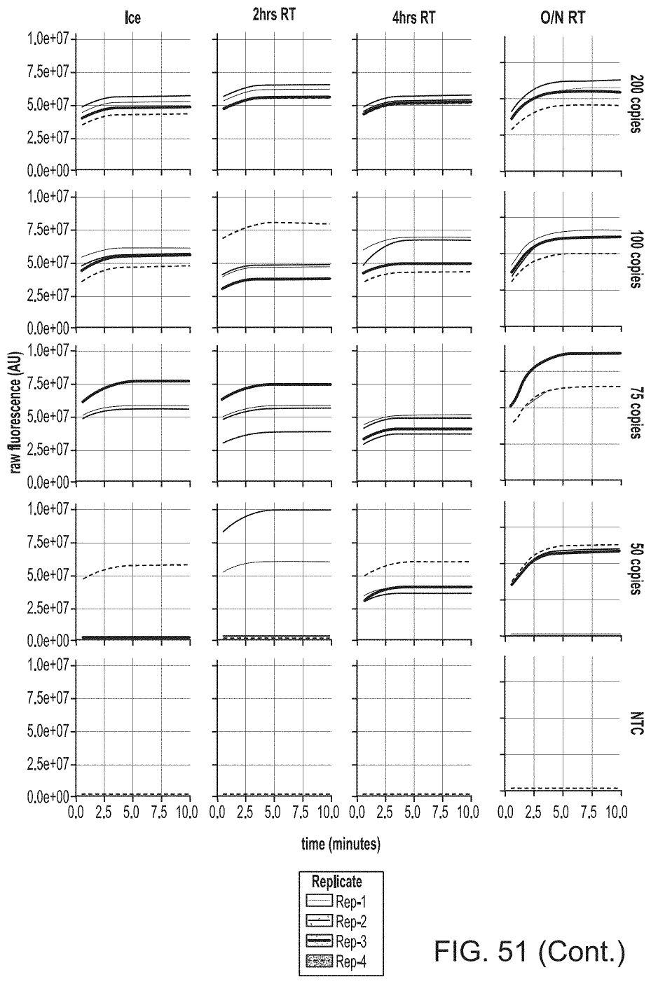

FIG. 51 illustrates results from an on deck stability study, with buffer salt primer (BSP) and enzyme (ENZ) mixes incubated separately. The RT-LAMP ENZ mixes containing 0.75× GF Bst DNA polymerase & 0.5× GF reverse transcriptase (RTx) were incubated on ice for 4 hours and at RT for two hours, four hours, and overnight. The data shows that RT-LAMP reagents were stable on ice for 4 hours or at room temperature up to 24 hrs. Additional experiments done with the BSP and ENZ mixes incubated as a single master mix showed similar results.

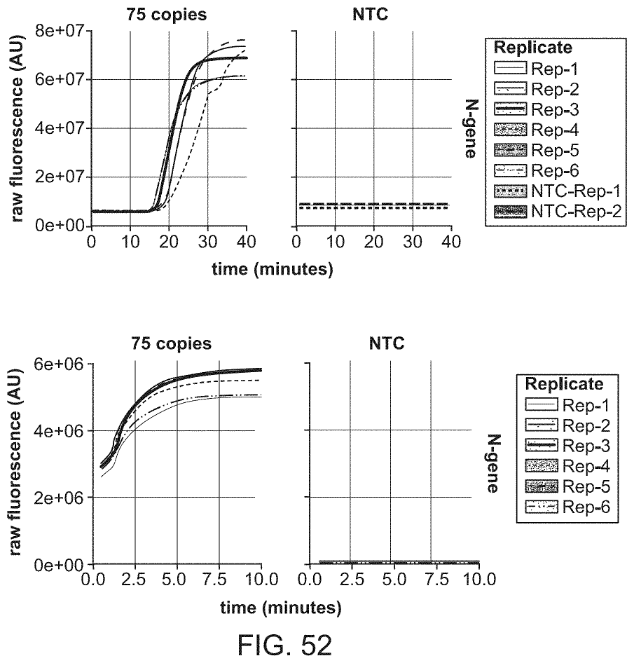

FIG. 52 illustrates RT-LAMP (left) and DETECTR (right) results from a high-performing MagMAX workflow (similar to the workflow of FIG. 48 but with reduced RT-LAMP reaction reagents). The workflow included a 110 μL UTM sample (with a concentration of 75 copies per reaction), 100 μL lysis/binding Buffer, and 10 μL beads (with 2× ProK) incubated at 37° C. for 5 min. Two washes of W1 wash buffer were used before elution of the nucleic acids from the beads. The workflow used a reduced ENZ buffer of 75% glycerol free (GF) of the previous concentration of Bst & 50% of the previous concentration of GF RTx and RT-LAMP reactions were incubated at 57 C for 40 minutes. DETECTR reactions were performed for 10 minutes at 37 C. The data showed strong performance for all six replicates at 75 copies per reaction. Additional experiments done with samples containing nasal or saliva matrices showed similar results.

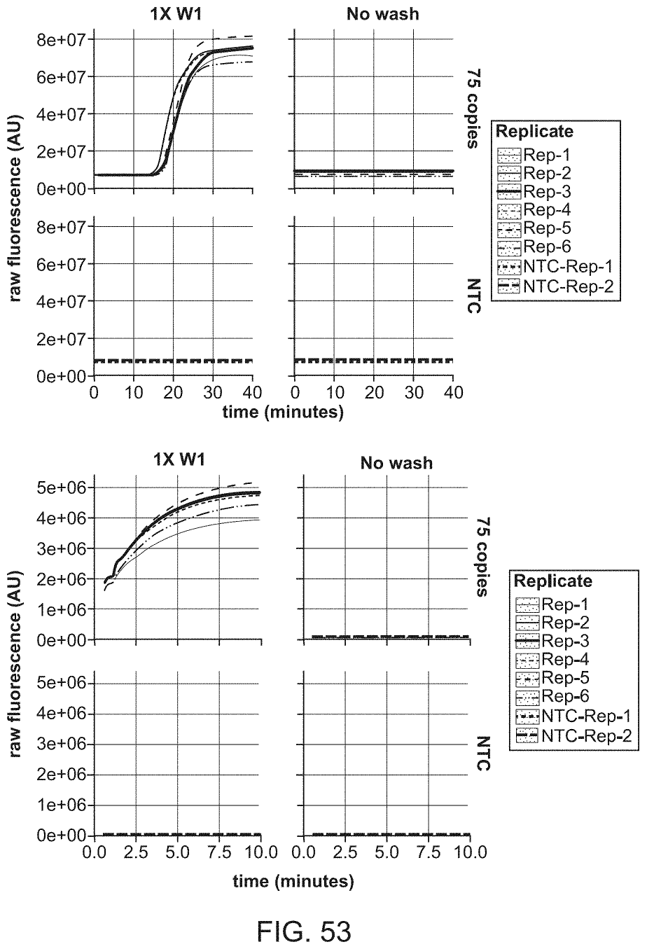

FIG. 53 illustrates results for a MagMAX workflow similar to that of FIG. 52 with one wash with 50 μL W1. For this workflow, the results show that 6/6 replicates had correct positive detection.

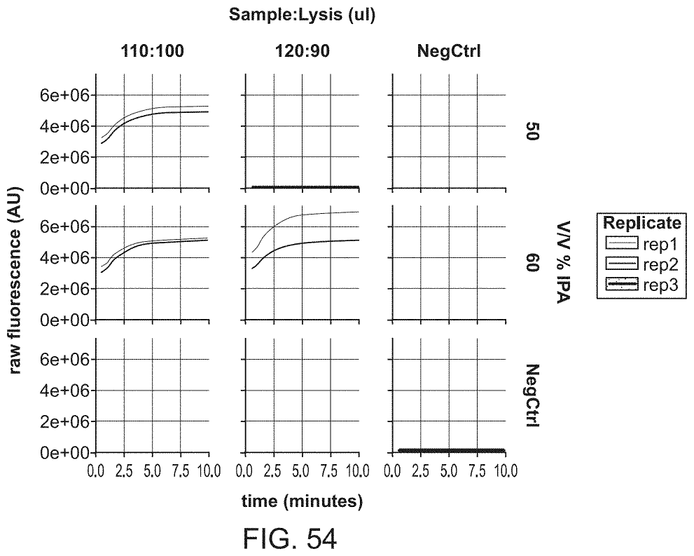

FIG. 54 illustrates example experiments where ratios of sample volume to lysis buffer volume were modified, in addition to % IPA titrations. Similar to the results shown in FIG. 49, results were strongest with 110 μL sample and 100 μL lysis buffer w/50% IPA. 120:90 with 60% IPA appeared to show strong performance as well.

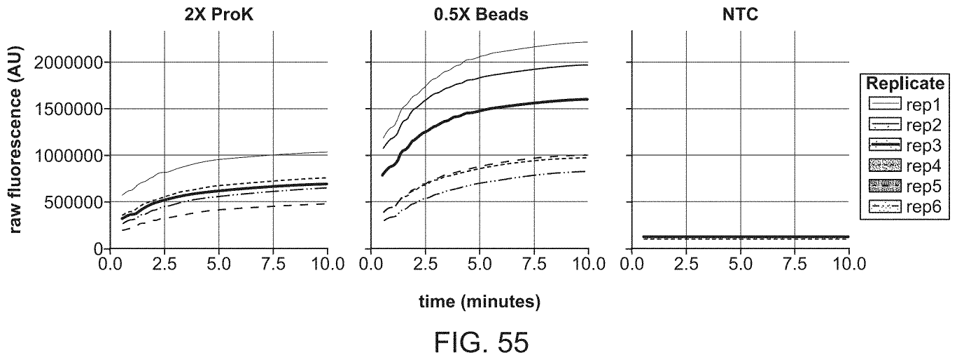

FIG. 55 illustrates results of an experiment where bead concentration in MagMAX viral isolator was decreased to mitigate fluorescence quenching during RT-LAMP and DETECTR reactions. As shown from the plots, reducing beads by half provided better signals overall, but had a larger spread. The 0.5× bead solution comprised 1 part beads to 3 parts lysis/binding enhancer (ProK), as opposed to 2 parts beads to 3 parts ProK in the previous protocol. 2× proteinase K was also tested with the original bead concentration and was found to enhance signal to noise as well. Additional experiments were performed with varying bead concentrations, from 1:4 beads to ProK to 1:1.5 beads to ProK, and showed that signal to noise improved as the concentrations of beads was reduced to as low as 1:4. Further experiments were performed with varying sample concentrations and were found to provide significant detection with copies as low as 55 copies per reaction. Gel-based analysis confirmed that bead-based quenching was responsible for reduced signal in 1× reactions rather than inhibition of the RT-LAMP or DETECTR reactions.

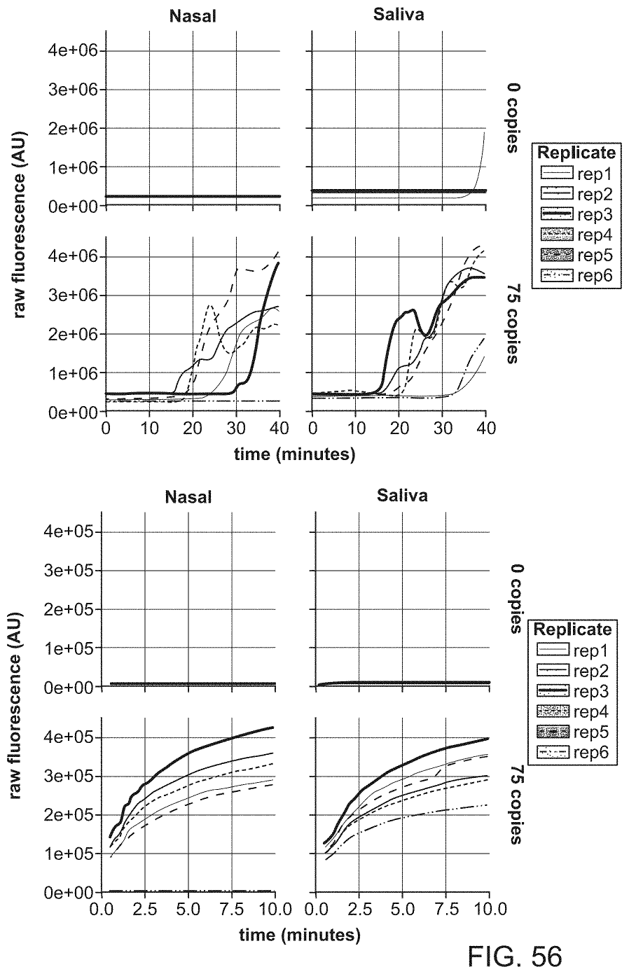

FIG. 56 illustrates RT-LAMP (left) and DETECTR (right) results of an experiment taken to verify performing a detection reaction with a reduced number of beads. DETECTR reactions were performed at 37 C for 10 minutes. In the experiment, 5/6 replicates in nasal matrix (repeat) and 6/6 in saliva correctly tested positive with the beads retained in the chamber during the RT-LAMP and DETECTR reactions.

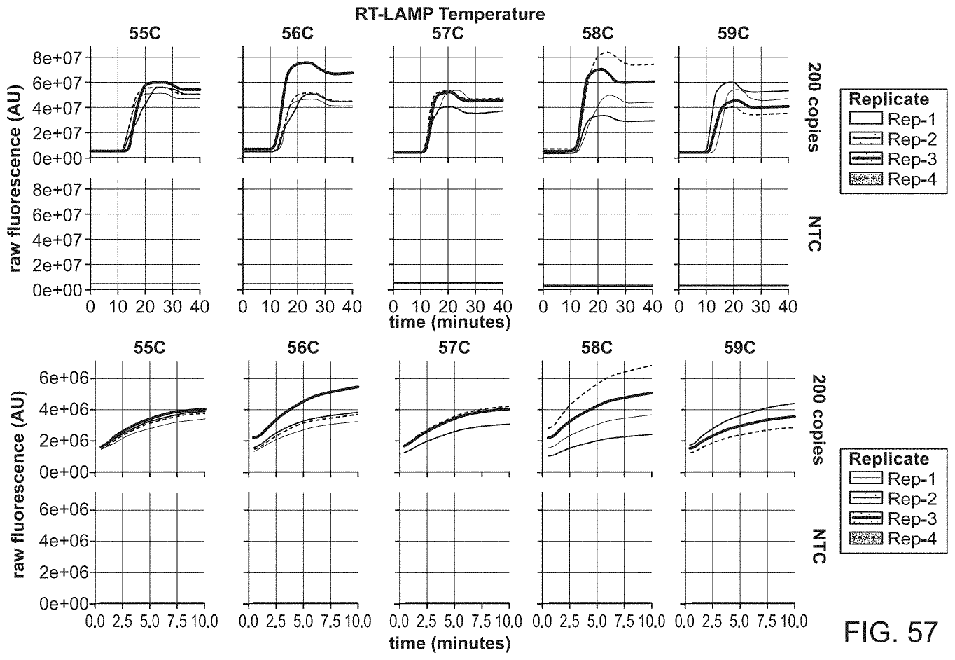

FIG. 57 illustrates RT-LAMP (left) and DETECTR (right) results of a RT-LAMP temperature guardbanding study (55 C-59 C). RT-LAMP reactions were performed at 57 C for 40 minutes. All replicates were amplified for 200 copies of Twist at each RT-LAMP temperature and were detected by DETECTR reactions.

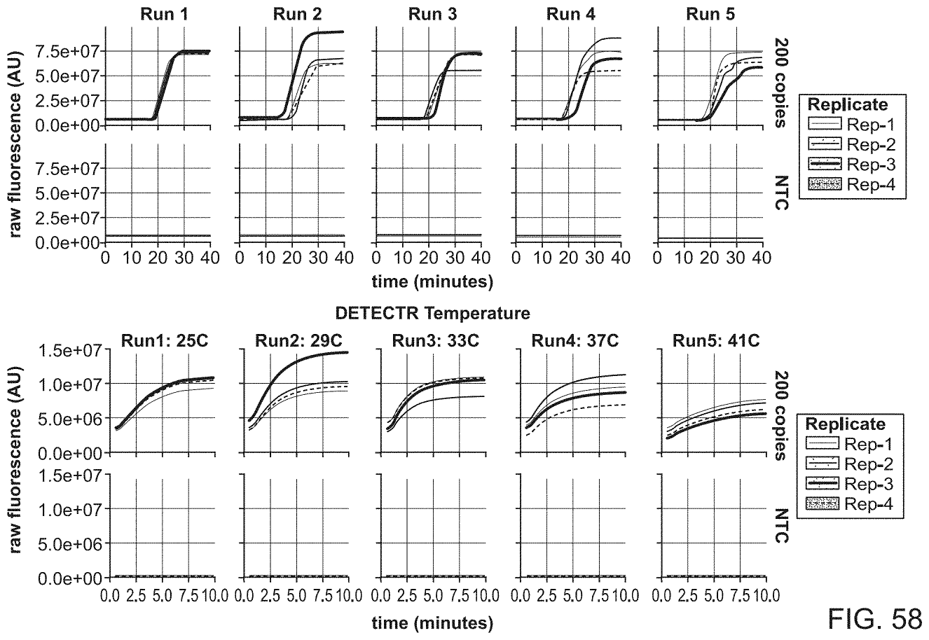

FIG. 58 illustrates RT-LAMP (left) and DETECTR (right) results of a DETECTR temperature guardbanding study (25 C-41 C). All replicates of 200 copies/reaction produced strong signals for all DETECTR temperatures tested.

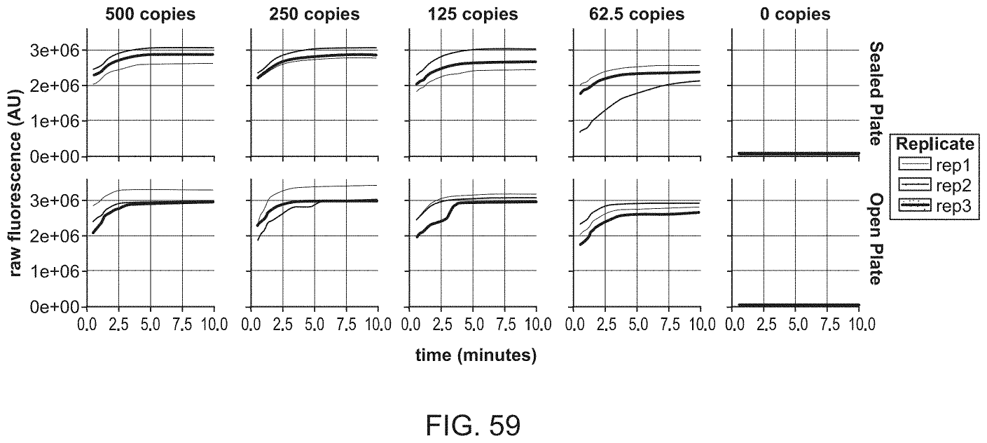

FIG. 59 illustrates an LOD test in an open-plate format. The test compared a template titration in standard QS5 format against a reaction in an open plate to determine if evaporation negatively affected assay sensitivity. The RT-LAMP QS5 controls were tested at 57° C., while the Agilent plate samples were tested at 62° C. Then, DETECTR was performed at 37 C. No beads were added to the sample. The samples comprised Twist RNA controls. All other wells filled w/50 μL water. The experiments showed positive results for all replicates without cross-contamination or evaporation related effects.

### Automated Workflow

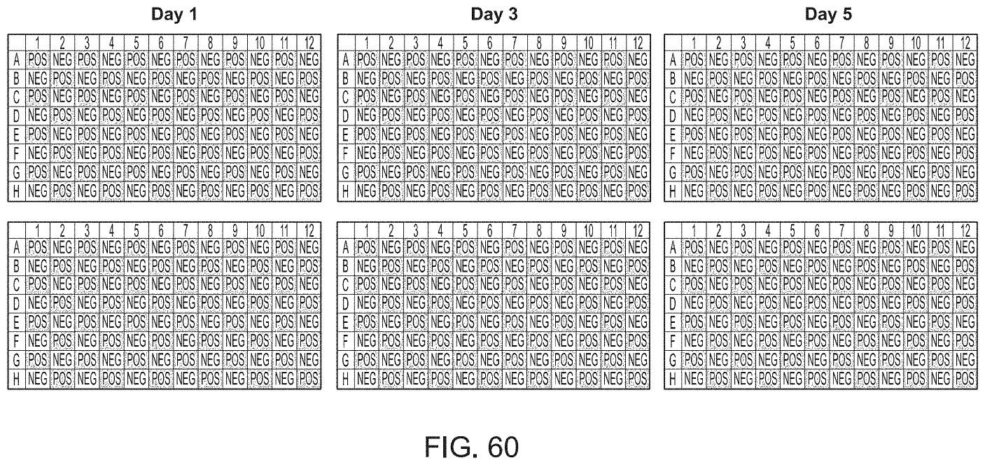

FIGS. 60-63 illustrate reproducibility performance of the automated workflow for a high-throughput single chamber assay similar to that disclosed in FIG. 1D. Using this workflow, up to 1,500 samples may be tested in an eight-hour shift. In the reproducibility study, multiple microplates were processed in a staggered fashion. For a particular plate, an extraction set of operations (inactivation, lysis, binding with microparticles, washing, and eluting) lasted about 25 minutes, a pre-amplification set of operations (adding pre-amplification master mix) lasted about 30 minutes, and a DETECTR step (adding DETECTR master mix) lasted about 10 minutes. Staggering the assays in time yielded a set of 192 results in under two hours. The assay may be executed in a manual fashion or an automated fashion. The assay may be executed on equipment such as the Agilent BRAVO system. the Hamilton STAR system, or other automated liquid handling equipment. The reagent kit may include a lysis/binding solution, a wash solution, an elution buffer, RNA binding beads, carrier RNA, a lysis/binding enhancer, RT-LAMP master mix, RT-LAMP activator, DETECTR master mix, a positive control, and a negative control.

The automated workflow may leverage CRISPR technology. A Cas protein, complexed with a guide RNA (gRNA), when interacting with a target nucleic acid, may cleave a reporter (e.g., fluorescently-labeled probe with a quencher), to release a signal into the sample, indicating the target is present within the sample. Examples of Cas nucleases include Cas12, Cas13, and Cas14. Cas12 can recognize a double-stranded DNA (dsDNA) targets, and can cleave an ssDNA reporter through transcollateral cleavage. A person of skill in the art will recognize that different Cas nucleases can work differently. For example, some Cas13 nucleases can target RNA and can cleaves a single stranded nucleic acid reporter. Some Cas14 nucleases can target dsDNA and cleave an ssDNA reporter. Guide RNA molecules may be generated to enable the system to detect a presence of many types of targets.

FIG. 60 illustrates a five-day reproducibility study for the automated workflow. In this example, the study used a multi-chamber microplate with each well having a positive or negative sample. No positive samples were next to other positive samples, and no negative samples were next to other negative samples, which produced a checkerboard pattern of positive and negative sample wells. During each day of the study, four plates were run, using multiple operators. The reproducibility study resulted in an accuracy rate of 99.7%, detecting 960 positives and 160 negatives.

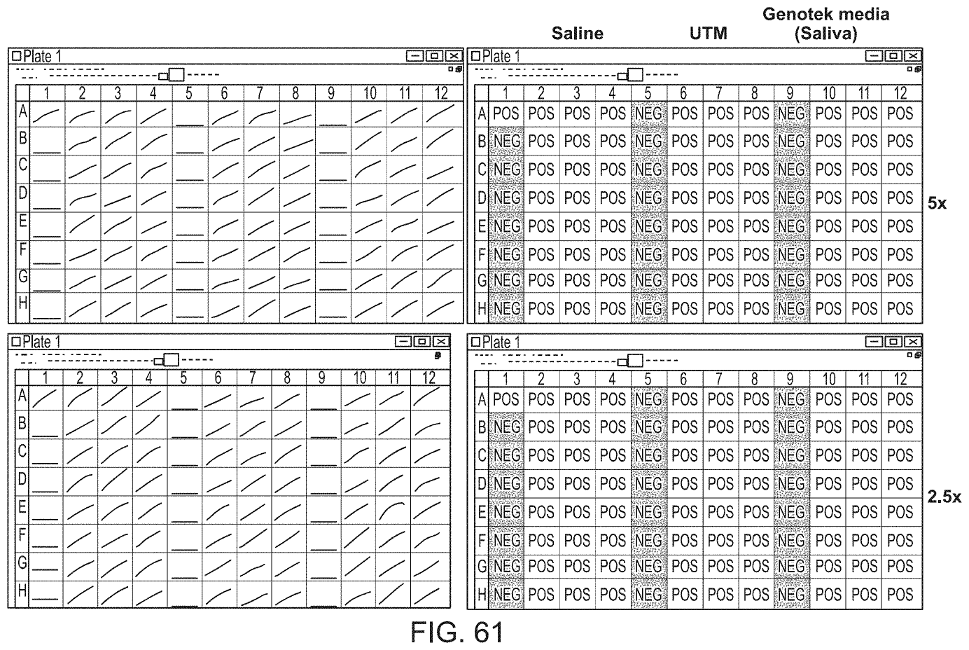

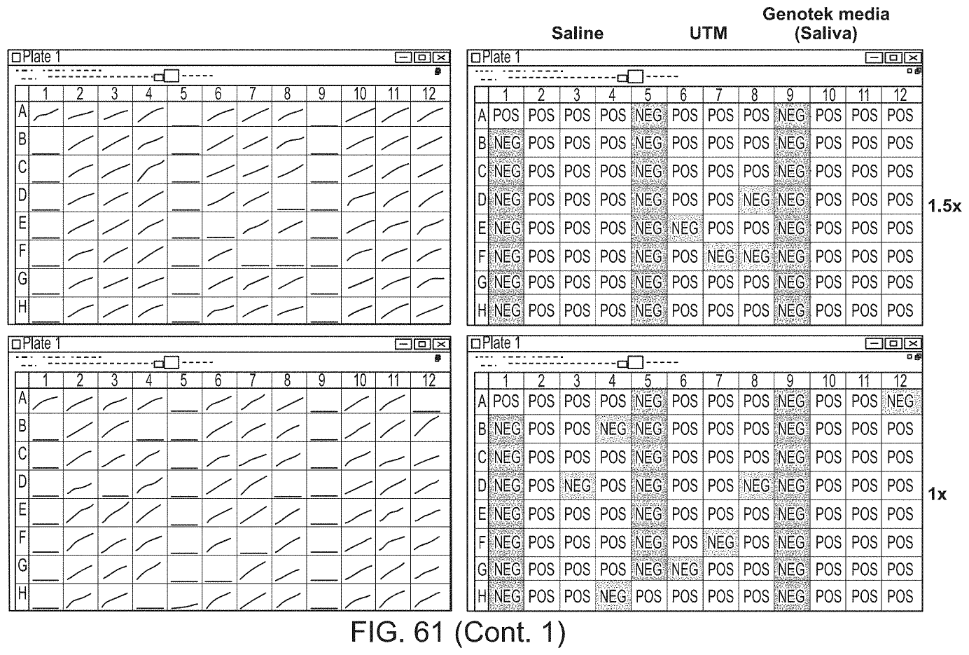

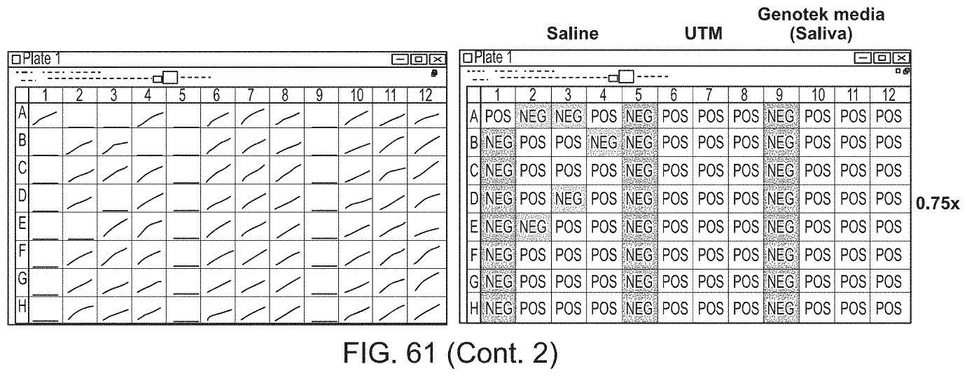

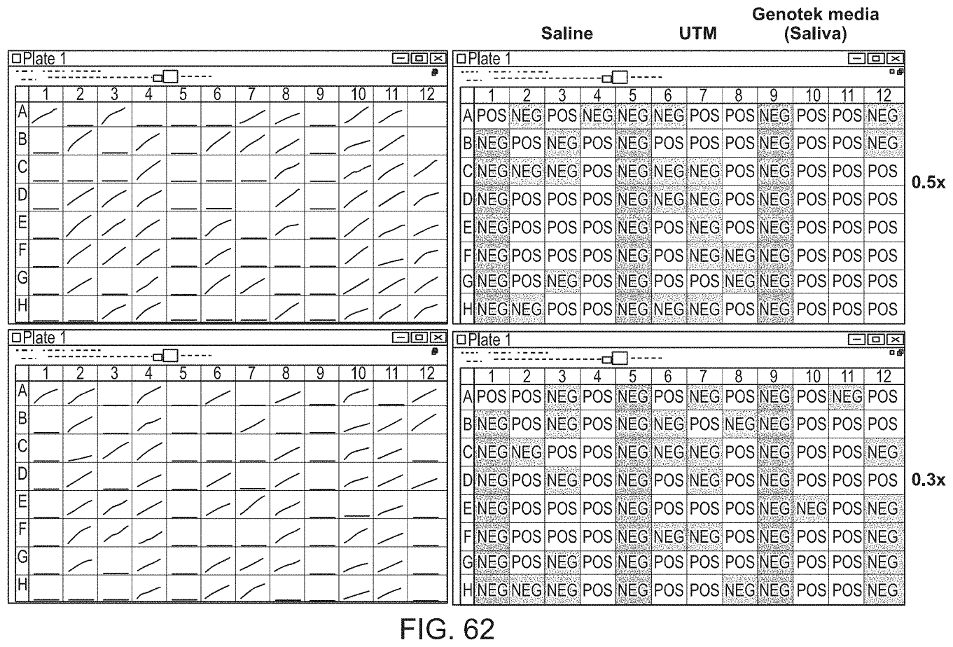

FIGS. 61 and 62 illustrate results of a development study for the automated workflow. The workflow was implemented on three types of samples: a saline sample, a UTM sample, and a saliva sample. For large concentrations of the SeraCare target (5×), assays with all sample types performed well. But for lower concentrations of the target, tests of saliva and saline samples continued to perform well while UTM sample results were mixed. The illustrated plots on the left show kinetic curves plotting signal intensity (e.g., fluorescence) over time corresponding to the single-chamber tests to the right. Positive tests showed increasing signal intensity, while negative tests showed flat lines (e.g., no signal increase during the reactions).s

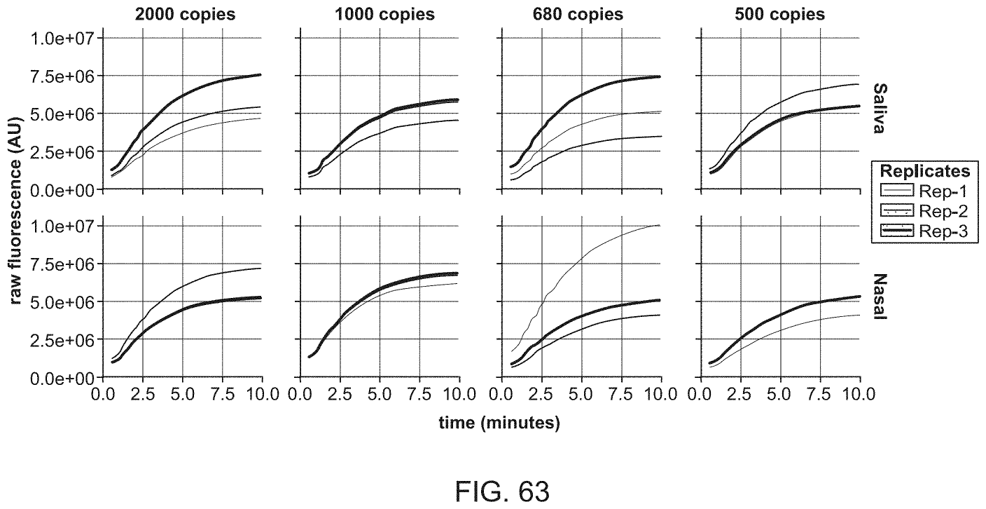

FIG. 63 illustrate additional results from the automated workflow. FIG. 63 illustrates fluorescence data collected from automated assays with saliva and nasal samples. The results showed strong performance for nasal and saliva samples at concentrations of at least 500 copies of SeraCare sample/reaction.

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### EXPERIMENTAL EXAMPLES

### Example 1

### COVID Variant Example

The following example discusses components used in detection assays for coronavirus variants, including the B.1.1.7 lineage and the B.1.351 lineage. The assays are conducted using RT-PCR DETECTR reactions for amplification and detection.

Rapid RT-PCR DETECTR reactions are used for the detection of a coronavirus variant, particularly the variant known as 20B/501Y.V1, VOC 202012/01, or B.1.1.7 lineage, or the variant known as: 20C/501Y.V2 or B.1.351 lineage. The genetic characteristics of these variants are discussed in Leung et. al, Early transmissibility assessment of the N501Y mutant strains of SARS-CoV-2 in the United Kingdom, October to November 2020, Euro Surveill. 2021; 26(1) and in Tegally et. al., Emergence and rapid spread of a new severe acute respiratory syndrome-related coronavirus 2 (SARS-CoV-2) lineage with multiple spike mutations in South Africa, MedRxiv 2020.12.21. A sample containing a target nucleic acid corresponding to these variants is amplified using one or more of the primers listed in Table 5, and a mutation in the nucleic acid (corresponding to the B.1.1.7 and/or B.1.351 variant) is detected using Cas12 and one of the gRNAs described in Table 7. RT-LAMP may alternatively be used for the amplification method.

### Mutations

Table 4 lists certain mutations characterizing the B.1.1.7 and/or B.1.351 variants, one or more of which are selected as targets for the RT-PCR DETECTR reaction. Regarding Table 4, mutations are described in the form of: [wild type amino acid][amino acid number][mutant amino acid]. The lowercase nucleotides in parenthesis correspond to the wild-type and the uppercase nucleotides in parenthesis correspond to the mutant. Additionally, “xxx” refers to an unknown single-nucleotide polymorphism (SNP). SARS-CoV-2 target sequences have been obtained using all available genomes available from GISAID.

TABLE 4
Genetic changes characterizing theB.1.1.7 and/or B.1.351 variants.

| “UK” Variant Mutations | “South African” Variant Mutations |
|---|---|
| del69/del70 (68-70 ata cat gtc) | L18F (ctt/xxx) |
| del144 (143-144 gtt tat) | D80A (gat/GCT) |
| N501Y (aat/TAT) | D215G (gat/GGT) |
| A570D (gct/GMT) | del242-244 (240-243 actttacttgct) |
| P681H (Nigerian)(cct/CAT) | R246I (aga/ATA) |
| T716H (aca/ATA) | K417N (aag/AAT) |
| D1118H (gac/CAC) | E484K (gaa/AAA) |
| | N501Y (aat/TAT) |
| | D614G (gat/GGT) |
| | A701V (gca/GTA) (edited) |

Selected as an amplicon may be any of the regions of the Spike gene comprising the groups of mutations detailed in Table 5. Table 5 lists mutations present in the Spike gene (reference name MN908947.3). The spike region is the most variable and is a major region for vaccine design. The start and stop nucleotide (nt) positions on the Spike gene are given in the 2nd and 3rd columns of Table 5, respectively. The three columns to the right show the mutations found in the B.1.1.7, B.1.351 and both variants.

TABLE 5
Exemplary mutations for combined strains of
the Spike gene (reference name MN908947.3).

| | | | | Found | |
|---|---|---|---|---|---|
| | | | Found | in the | |
| | | | in the | B.1.351 | |
| Mutation | Start | Stop | B.1.1.7 | Variant | Found |
| Name | position | position | Variant | (501Y.V2) | in Both |
| E484K, | 23012 | 23270 | A570D | E484 | N501Y |
| N501Y, | | | | | |
| A570D | | | | | |
| P681H, | 23603 | 23708 | P681H, | A701V | |
| A701V, | | | T716H | | |
| T716H | | | | | |
| P681H, | 23603 | 23663 | P681H | A701V | |
| A701V | | | | | |
| A701V, | 23663 | 23708 | T716H | A701V | |
| T716H | | | | | |
| 69-70del, | 21764 | 21802 | 69-70del | D80A | |
| D80A | | | | | |

### Primers

DETECTR assays are performed using RT-PCR for pre-amplification. Particularly, the assays use an extreme PCR technique in which the speed of the PCR reaction is decreased to less than 5 minutes by near-instantaneous changes in the reaction temperature. This rapid temperature change may be accomplished by moving the reaction between heat-zones (water baths, heat blocks, etc.) of various temperatures in a thin-walled vessel, instead of cooling or heating the entire instrument for each cycle. Additional speed increases of the PCR reaction can be achieved by increasing the primer, polymerase, and Mg2+ concentrations of the reaction. One or more of the primers described in Table 6 are used. The primers have been designed using PrimerExplorer v5 (https://primerexplorer.jp/e/). Table 6 provides the sequence of each primer, along with the mutations comprised in the amplicon with which they are compatible.

TABLE 6
Primers designed for the RT-PCR-DETECTR assay

| | | Description/ |
|---|---|---|
| Name | Sequence | Note |
| M6112 triple501- | CTGAAATCTATCAGGCCGGTAGCA | E484K, N501Y, |
| v1-F | | A570D |
| M6113 triple501- | GTCAAGAATCTCAAGTGTCTGTGGAT | E484K, N501Y, |
| v1-R | | A570D |
| M6114 triple501- | TGAAATCTATCAGGCCGGTAGCAC | E484K, N501Y, |
| v2-F | | A570D |
| M6115 triple501- | TGAAATCTATCAGGCCGGTAGCAC | E484K, N501Y, |
| v2-R | | A570D |
| M6116 triple501- | TCAACTGAAATCTATCAGGCCGGTA | E484K, N501Y, |
| v3-F | | A570D |
| M6117 triple501- | ATCTCAAGTGTCTGTGGATCAC | E484K, N501Y, |
| v3-R | | A570D |
| M6118 triple501- | TATCAGGCCGGTAGCACACCTT | E484K, N501Y, |
| v4-F | | A570D |
| M6119 triple501- | GTGTAATGTCAAGAATCTCAAGTGTCT | E484K, N501Y, |
| v4-R | | A570D |
| M6120 triple701- | TGCAGGTATATGCGCTAGTTATCAGA | P681H, A701V, |
| v1-F | | T716H |
| M6121 triple701- | GCAACAAAAGATTGCTGCATTCAGTTG | P681H, A701V, |
| v1-R | A | T716H |
| M6122 triple701- | CAGGTATATGCGCTAGTTATCAGACTC | P681H, A701V, |
| v2-F | A | T716H |
| M6123 triple701- | GCAACAAAAGATTGCTGCATTCAGTTG | P681H, A701V, |
| v2-R | A | T716H |
| M6124 triple701- | GGTGCAGGTATATGCGCTAGTTATCA | P681H, A701V, |
| v3-F | | T716H |
| M6125 triple701- | GCAACAAAAGATTGCTGCATTCAGTTG | P681H, A701V, |
| v3-R | A | T716H |
| M6126 triple701- | ATTGGTGCAGGTATATGCGCTAGTTA | P681H, A701V, |
| v4-F | | T716H |
| M6127 triple701- | GCAACAAAAGATTGCTGCATTCAGTTG | P681H, A701V, |
| v4-R | A | T716H |
| M6128 N501Y-V1-F | AGGTTTTAATTGTTACTTTCCTTTACA | N501Y |
| M6129 N501Y-V1-R | GCTGGTGCATGTAGAAGTTCAAAAGAA | N501Y |
| M6130 K417N-V1-F | TTGTAATTAGAGGTGATGAAGTCAGA | K417N |
| M6131 K417N-V1-R | ATTCCAAGCTATAACGCAGCCTGTAAA | K417N |
| M6132 K417N-v2-F | TGTAATTAGAGGTGATGAAGTCAGACA | K417N |
| M6133 K417N-v2-R | GAATTCCAAGCTATAACGCAGCCTGTA | K417N |
| M6134 Y144del- | ATTGTTAATAACGCTACTAATGTTGTT | Y144del |
| v1-F | | |
| M6135 Y144del- | CACTTTCCATCCAACTTTTGTTGTT | Y144del |
| v1-R | | |
| M6136 Y144del- | AACGCTACTAATGTTGTTATTAAAGT | Y144del |
| v2-F | | |
| M6137 Y144del- | ACTCTGAACTCACTTTCCATCCAACTT | Y144del |
| v2-R | | |
| M6138 P681H-v1-F | GGTGCAGGTATATGCGCTAGTTATCA | P681H |
| M6139 P681H-v1-R | AATGATGGATTGACTAGCTACACTA | P681H |
| M6140 P681H-v2-F | TGCAGGTATATGCGCTAGTTATCAGA | P681H |
| M6141 P681H-v2-R | AATGATGGATTGACTAGCTACACTA | P681H |
| M6142 A570D-v1-F | AGGTGTTCTTACTGAGTCTAACAAAAA | A570D |
| M6143 A570D-v1-R | GTCAAGAATCTCAAGTGTCTGTGGA | A570D |
| M6144 A570D-v2-F | AGGCACAGGTGTTCTTACTGAGTCTA | A570D |
| M6145 A570D-v2-R | CTCAAGTGTCTGTGGATCACGGA | A570D |
| M6146 double80- | CAGTTTTACATTCAACTCAGGACTTGT | 69-70del, D80A |
| v1-F | | |
| M6147 double80- | TATGTTAGACTTCTCAGTGGAAGCAAA | 69-70del, D80A |
| v1-R | | |
| M6148 double80- | AGTTTTACATTCAACTCAGGACTTGTT | 69-70del, D80A |
| v2-F | | |
| M6149 double80- | TGTTAGACTTCTCAGTGGAAGCAA | 69-70del, D80A |
| v2-R | | |
| M6150 double80- | GTTTTACATTCAACTCAGGACTTGTTC | 69-70del, D80A |
| v3-F | | |
| M6151 double80- | GTTAGACTTCTCAGTGGAAGCA | 69-70del, D80A |
| v3-R | | |
| M6152 double681- | GACATACCCATTGGTGCAGGTATAT | P681H, A701V |
| v1-F | | |
| M6153 double681- | GTGGGTATGGCAATAGAGTTATTAGA | P681H, A701V |
| v1-R | | |
| M6154 double681- | GACATACCCATTGGTGCAGGTATA | P681H, A701V |
| v2-F | | |
| M6155 double681- | GTGGGTATGGCAATAGAGTTATTAGAG | P681H, A701V |
| v2-R | | |
| M6156 double681- | TGACATACCCATTGGTGCAGGTA | P681H, A701V |
| v3-F | | |
| M6157 double681- | GGGTATGGCAATAGAGTTATTAGAGTA | P681H, A701V |
| v3-R | | |

### Guide Nucleic Acids

After completion of the amplification step, the amplicon is combined with a Cas12-gRNA complex, and a fluorescence-based trans-cleavage assay, as described in prior examples herein, for example, is allowed to proceed. Sequences are detected using any of the gRNA sequences disclosed in Table 7. Table 7 provides exemplary guides for the B.1.1.7 and/or B.1.351 variants of the crRNA type and compatible with a Cas12 protein. The Cas12 protein may recognize any of the following protospacer adjacent motifs (PAM): ttcc, tcca, tttg, tta, cttg, cctt, tta, tttc, ttcc, tcca, ttg, tttg, ttg, tca, ctca, ttct, cttg, tttc, tcta, ctct and ttg. Regarding Table 7, in the names of the guides, “d6-7” refers to deletion 60 to 70; “wt” refers to the original, wild-type virus; “m” refers to a guide for a mutant variant, and “mp” refers to mutant poison. The mutant poison guides are designed to further destabilize the guides from recognizing the wild type sequence, as some guides designed to recognize the mutant may also recognize the wild type, but at a lower rate. In other words, the mutant poison guides promote stronger recognition of the mutant over the wildtype. The numbering in the “Name” column provides the amino acid position of the mutation.

TABLE 7
Exemplary guide RNA's for the B.1.1.7 and/or
B.1.351 variants of the crRNA type and compatible
with the Cas12 protein.

| Name | RNA Sequence | Spacer Sequence | Notes |
|---|---|---|---|
| d6-7-1m | UAAUUUCUACUAAGUGUAGAU | atgctgtctctgggaccaat | SARS-CoV-2 |
| | augcugucucugggaccaau | | B.1.1.7 Variant |
| d6-7-2m | UAAUUUCUACUAAGUGUAGAU | tgctgtctctgggaccaatg | SARS-CoV-2 |
| | ugcugucucugggaccaaug | | B.1.1.7 Variant |
| 80-1w | UAAUUUCUACUAAGUGUAGAU | ataaccctgtcctaccattt | SARS-CoV-2 |
| | auaacccuguccuaccauuu | | wild-type |
| 80-1m | UAAUUUCUACUAAGUGUAGAU | Ctaaccctgtcctaccattt | SARS-CoV-2 |
| | Cuaacccuguccuaccauuu | | B.1.351 Variant |
| 80-1mp | UAAUUUCUACUAAGUGUAGAU | CtaaccctAtcctaccattt | SARS-CoV-2 |
| | CuaacccuAuccuaccauuu | | B.1.351 Variant |
| 80-2w | UAAUUUCUACUAAGUGUAGAU | tcaaacctcttagtaccatt | SARS-CoV-2 |
| | ucaaaccucuuaguaccauu | | wild-type |
| 80-2m | UAAUUUCUACUAAGUGUAGAU | Gcaaacctcttagtaccatt | SARS-CoV-2 |
| | Gcaaaccucuuaguaccauu | | B.1.351 Variant |
| 80-2mp | UAAUUUCUACUAAGUGUAGAU | GcaaacTtcttagtaccatt | SARS-CoV-2 |
| | GcaaacUucuuaguaccauu | | B.1.351 Variant |
| 484-1w | UAAUUUCUACUAAGUGUAGAU | taatggtgttgaaggtttta | SARS-CoV-2 |
| | uaaugguguugaagguuuua | | wild-type |
| 484-1m | UAAUUUCUACUAAGUGUAGAU | taatggtgttAaaggtttta | SARS-CoV-2 |
| | uaaugguguuAaagguuuua | | B.1.351 Variant |
| 484-1mp | UAAUUUCUACUAAGUGUAGAU | taatgAtgttAaaggtttta | SARS-CoV-2 |
| | uaaugAuguuAaagguuuua | | B.1.351 Variant |
| 484-2w | UAAUUUCUACUAAGUGUAGAU | gtaatggtgttgaaggtttt | SARS-CoV-2 |
| | guaaugguguugaagguuuu | | wild-type |
| 484-2m | UAAUUUCUACUAAGUGUAGAU | gtaatggtgttAaaggtttt | SARS-CoV-2 |
| | guaaugguguuAaagguuuu | | B.1.351 Variant |
| 484-2mp | UAAUUUCUACUAAGUGUAGAU | gtaatgAtgttAaaggtttt | SARS-CoV-2 |
| | guaaugAuguuAaagguuuu | | B.1.351 Variant |
| 484-3w | UAAUUUCUACUAAGUGUAGAU | aaaccttcaacaccattaca | SARS-CoV-2 |
| | aaaccuucaacaccauuaca | | wild-type |
| 484-3m | UAAUUUCUACUAAGUGUAGAU | aaaccttTaacaccattaca | SARS-CoV-2 |
| | aaaccuuUaacaccauuaca | | B.1.351 Variant |
| 484-3mp | UAAUUUCUACUAAGUGUAGAU | aaaccttTaacaTcattaca | SARS-CoV-2 |
| | aaaccuuUaacaUcauuaca | | B.1.351 Variant |
| 501-1w | UAAUUUCUACUAAGUGUAGAU | caacccactaatggtgttgg | SARS-CoV-2 |
| | caacccacuaaugguguugg | | wild-type |
| 501-1m | UAAUUUCUACUAAGUGUAGAU | caacccactTatggtgttgg | SARS-CoV-2 |
| | caacccacuUaugguguugg | | UK/B1.351 |
| | | | Variant |
| 501-1mp | UAAUUUCUACUAAGUGUAGAU | caacccTetTatggtgttgg | SARS-CoV-2 |
| | caacccUcuUaugguguugg | | UK/B1.351 |
| | | | Variant |
| 501-2 | UAAUUUCUACUAAGUGUAGAU | aacccactaatggtgttggt | SARS-CoV-2 |
| | aacccacuaaugguguuggu | | wild-type |
| 501-2m | UAAUUUCUACUAAGUGUAGAU | aacccactTatggtgttggt | SARS-CoV-2 |
| | aacccacuUaugguguuggu | | UK/B1.351 |
| | | | Variant |
| 501-2mp | UAAUUUCUACUAAGUGUAGAU | aacccTetTatggtgttggt | SARS-CoV-2 |
| | aacccUcuUaugguguuggu | | UK/B1.351 |
| | | | Variant |
| 501-3w | UAAUUUCUACUAAGUGUAGAU | acccactaatggtgttggtt | SARS-CoV-2 |
| | acccacuaaugguguugguu | | wild-type |
| 501-3m | UAAUUUCUACUAAGUGUAGAU | acccactTatggtgttggtt | SARS-CoV-2 |
| | acccacuUaugguguugguu | | UK/B1.351 |
| | | | Variant |
| 501-3mp | UAAUUUCUACUAAGUGUAGAU | acccTetTatggtgttggtt | SARS-CoV-2 |
| | acccUcuUaugguguugguu | | UK/B1.351 |
| | | | Variant |
| 501-4w | UAAUUUCUACUAAGUGUAGAU | gtaaccaacaccattagtgg | SARS-CoV-2 |
| | guaaccaacaccauuagugg | | wild-type |
| 501-4m | UAAUUUCUACUAAGUGUAGAU | gtaaccaacaccatAagtgg | SARS-CoV-2 |
| | guaaccaacaccauAagugg | | UK/B1.351 |
| | | | Variant |
| 501-4mp | UAAUUUCUACUAAGUGUAGAU | gtaaccGacaccatAagtgg | SARS-CoV-2 |
| | guaaccGacaccauAagugg | | UK/B1.351 |
| | | | Variant |
| 570-1w | UAAUUUCUACUAAGUGUAGAU | gcagagacattgctgacact | SARS-CoV-2 |
| | gcagagacauugcugacacu | | wild-type |
| 570-1m | UAAUUUCUACUAAGUGUAGAU | gcagagacattgAtgacact | SARS-CoV-2 |
| | gcagagacauugAugacacu | | B.1.1.7 Variant |
| 570-1mp | UAAUUUCUACUAAGUGUAGAU | gcagagGcattgAtgacact | SARS-CoV-2 |
| | gcagagGcauugAugacacu | | B.1.1.7 Variant |
| 570-2w | UAAUUUCUACUAAGUGUAGAU | ctgacactactgatgctgtc | SARS-CoV-2 |
| | cugacacuacugaugcuguc | | wild-type |
| 570-2m | UAAUUUCUACUAAGUGUAGAU | Atgacactactgatgctgtc | SARS-CoV-2 |
| | Augacacuacugaugcuguc | | B.1.1.7 Variant |
| 570-2mp | UAAUUUCUACUAAGUGUAGAU | AtgacaTtactgatgctgtc | SARS-CoV-2 |
| | AugacaUuacugaugcuguc | | B.1.1.7 Variant |
| 570-3w | UAAUUUCUACUAAGUGUAGAU | ccatacccacaaattttact | SARS-CoV-2 |
| | guagugucagcaaugucucu | | wild-type |
| 570-3m | UAAUUUCUACUAAGUGUAGAU | ccatacccaTaaattttact | SARS-CoV-2 |
| | guagugucaUcaaugucucu | | B.1.1.7 Variant |
| 570-3mp | UAAUUUCUACUAAGUGUAGAU | ccatacTcaTaaattttact | SARS-CoV-2 |
| | guagugCcaUcaaugucucu | | B.1.1.7 Variant |
| 681-1w | UAAUUUCUACUAAGUGUAGAU | ccatacccacaaattttact | SARS-CoV-2 |
| | ccucggcgggcacguagugu | | wild-type |
| 681-1m | UAAUUUCUACUAAGUGUAGAU | ccatacccaTaaattttact | SARS-CoV-2 |
| | cAucggcgggcacguagugu | | B.1.1.7 Variant |
| 681-1mp | UAAUUUCUACUAAGUGUAGAU | ccatacTcaTaaattttact | SARS-CoV-2 |
| | cAucggUgggcacguagugu | | B.1.1.7 Variant |
| 681-2w | UAAUUUCUACUAAGUGUAGAU | ccatacccacaaattttact | SARS-CoV-2 |
| | gugcagaaaauucaguugcu | | wild-type |
| 681-2m | UAAUUUCUACUAAGUGUAGAU | ccatacccaTaaattttact | SARS-CoV-2 |
| | gugUagaaaauucaguugcu | | B.1.1.7 Variant |
| 681-2mp | UAAUUUCUACUAAGUGUAGAU | ccatacTcaTaaattttact | SARS-CoV-2 |
| | gugUagGaaauucaguugcu | | B.1.1.7 Variant |
| 701-1w | UAAUUUCUACUAAGUGUAGAU | ccatacccacaaattttact | SARS-CoV-2 |
| | ugcaccaagugacauagugu | | wild-type |
| 701-1m | UAAUUUCUACUAAGUGUAGAU | ccatacccaTaaattttact | SARS-CoV-2 |
| | uAcaccaagugacauagugu | | B 1.351 Variant |
| 701-1mp | UAAUUUCUACUAAGUGUAGAU | ccatacTcaTaaattttact | SARS-CoV-2 |
| | uAcaccaaAugacauagugu | | B.1.351 Variant |
| 701-2w | UAAUUUCUACUAAGUGUAGAU | ccatacccacaaattttact | SARS-CoV-2 |
| | uugccauacccacaaauuuu | | wild-type |
| 701-2m | UAAUUUCUACUAAGUGUAGAU | ccatacccaTaaattttact | SARS-CoV-2 |
| | uugccauacccaUaaauuuu | | B.1.351 Variant |
| 701-2mp | UAAUUUCUACUAAGUGUAGAU | ccatacTcaTaaattttact | SARS-CoV-2 |
| | uugccaCacccaUaaauuuu | | B.1.351 Variant |
| 716-1w | UAAUUUCUACUAAGUGUAGAU | ccatacccacaaattttact | SARS-CoV-2 |
| | auugccauacccacaaauuu | | wild-type |
| 716-1m | UAAUUUCUACUAAGUGUAGAU | ccatacccaTaaattttact | SARS-CoV-2 |
| | auugccauacccaUaaauuu | | B.1.1.7 Variant |
| 716-1mp | UAAUUUCUACUAAGUGUAGAU | ccatacTcaTaaattttact | SARS-CoV-2 |
| | auugccGuacccaUaaauuu | | B.1.1.7 Variant |
| 716-2w | UAAUUUCUACUAAGUGUAGAU | ccatacccacaaattttact | SARS-CoV-2 |
| | ccauacccacaaauuuuacu | | wild-type |
| 716-2m | UAAUUUCUACUAAGUGUAGAU | ccatacccaTaaattttact | SARS-CoV-2 |
| | ccauacccaUaaauuuuacu | | B.1.1.7 Variant |
| 716-2mp | UAAUUUCUACUAAGUGUAGAU | ccatacTcaTaaattttact | SARS-CoV-2 |
| | ccauacUcaUaaauuuuact | | B.1.1.7 Variant |

In some cases, the detection devices described herein can be configured to implement process control procedures to ensure that the sample preparation, target amplification, and target detection processes are performed accurately and as intended.

### Example 2

### Analytical Specificity Validation—Cross-Reactivity

Disclosed are experimental setups for testing cross-reactivity and potential interferents for nasal samples. These tests were conducted to determine whether the presence of common pathogen organisms would interfere with the performance of the DETECTR assay. The tests were conducted in silico (simulated using a computer) as well as tested in a wet lab with samples including concentrations of the tested organisms. The results indicated that there was not significant confounding in the assay results due to cross-reactivity.

The assays disclosed herein demonstrated no cross reactivity with organisms listed in the FDA Emergency Use Authorization Molecular Diagnostic Template for Commercial Manufacturers and minimal or no interference from endogenous substances such as blood or medications such as decongestants or antihistamines.

### DETECTR Assay

An RT-LAMP based DETECTR assay was configured as a CRISPR-Cas enzymatic detection for SARS-CoV-2 with a reverse transcription loop-mediated isothermal amplification (RT-LAMP) from patients suspected of COVID-19 by their healthcare provider. The RT-LAMP primers were designed to target a sequence in the SARS-CoV-2 N-gene. The CRISPR enzyme was configured to target the SARS-CoV-2 N-gene to detect the isothermal amplification amplicons.

SARS-CoV-2 nucleic acid was first extracted, isolated, and purified from the specimens. The purified nucleic acid was simultaneously reverse transcribed into cDNA then amplified using loop-mediated amplification (RT-LAMP). The CRISPR-Cas12-based detection cleaved the DNA linker releasing a fluorophore from its quencher molecule if an amplicon of the SARS-CoV-2 N-gene target region was created. The rise of fluorescence signal indicated a positive detection of SARS-CoV-2.

The DETECTR assay SARS-CoV-2 Reagent Kit, included the following materials: Lysis/Binding Solution, Wash Solution 1, Elution Buffer, RNA Binding Beads, Carrier RNA, Lysis/Binding Enhancer, RT-LAMP Activator, RT-LAMP Master Mix, DETECTR™ Master Mix, SARS-CoV-2 Positive Control, and SARS-CoV-2 Negative Control.

The DETECTR assay SARS-CoV-2 Reagent Kit tested was a CRISPR-based, reverse transcription and loop-mediated amplification (RT-LAMP) test. The SARS-CoV-2 primer and probe set(s) was designed to detect RNA from the SARS-CoV-2 in from patients suspected of COVID-19 by their healthcare provider.

The assay was run manually or on a laboratory automated liquid handling system.

### The primers and reporter probe sequences tested were:

| | |
|---|---|
| Primer F3 | AACACAAGCTTTCGGCAG |
| Primer B3 | GAAATTTGGATCTTTGTCATCC |
| Primer FIP | TGCGGCCAATGTTTGTAATCAG |
| | CCAAGGAAATTTTGGGGAC |
| Primer BIP | CGCATTGGCATGGAAGTCACTT |
| | TGATGGCACCTGTGTAG |
| Primer LF | TTCCTTGTCTGATTAGTTC |
| Primer LB | ACCTTCGGGAACGTGGTT |
| Reporter | (CR610)-TTATTATT- |
| | (BHQ2) |

### The guide RNA sequence is (SARS-CoV-2 target site underlined):

| | |
|---|---|
| gRNA | UAAUUUCUACUAAGUGUAGAUCCCCCAGCGCUUCAGCG |
| | UUC |

The assay was run as shown in FIG. 1D.

The assay was run manually or on a laboratory liquid handling platform (e.g., Agilent BRAVO or Hamilton STAR) that can: add and remove liquids to a 96 well or 384 well microplate; control the temperature of the microplate wells at 37° C. for the lysis and binding (step 1), at 57° C. for the RT-LAMP (step 6), and at 37° C. for the CRISPR detection (step 7); and detect signal from a fluorescent reporter probe.

### Reagent Kit Preparation

The lysis buffer was created by combining kit components carrier RNA and lysis/binding Solution lab supplied isopropanol.

The bead solution was created by combining kit components RNA binding beads and lysis/binding enhancer.

The wash solution was created by combining kit component wash solution 1 with laboratory supplied isopropanol.
Step 1: The user or the laboratory's liquid handling platform transferred 85 μL of the specimen sample in phosphate buffered saline (PBS) solution into the tube or microplate. A molecular diagnostic compatible sample tube, 96 well microtiter u-shaped plate, or a 384 well u-shaped microplate having a working capacity of a minimum of 275 μL was used.
Step 2: The Lysis Buffer and the Bead Solution were added to the microplate's well or tube. The tube or plate was placed on a heater that warmed the well's contents to 37° C. The tube or plate was then moved to a magnetic block that attracted the beads bound with nucleic acids to the walls of the tube/well where the lysate waste was removed by a pipette.
Steps 3 and 4: The tube or plate was removed from the magnetic block. The Wash Solution was added to the well and the beads were resuspended in the wash. The plate was returned to the magnetic block and the beads were collected on the wall of the microplate's well or tube. The wash waste was removed by a pipette from the well or tube.
Step 5: The tube or plate was removed from the magnetic block and placed on a heater that warms the contents to 57° C. The elution buffer was added to the well and the beads were resuspended with the pipette. The elution buffer eluted nucleic acid from the beads in preparation of the amplification and detection.
Step 6: The RT-LAMP master mix and activator were added to the tube or well followed by the addition of mineral oil, to prevent evaporation during the amplification and detection steps. The 30-minute simultaneous reverse transcription and isothermal amplification using loop-mediated amplification (RT-LAMP) of a target region on the SARS-CoV-2 N-gene was activated.
Step 7: After the RT-LAMP incubation was completed, the plate was removed from the 57° C. heater. The DETECTR master mix was added to the tube or well and was inserted into a fluorescence plate reader set at 37° C. The CRISPR-Cas12-based detection cleaved the DNA linker releasing a fluorophore from its quencher molecule if an amplicon of the SARS-CoV-2 N-gene target region was created in step 6. If the gRNA hybridized to the target sequence, then the CRISPR enzyme would cut the DNA tether of the reporter separating the quencher molecule from the fluorescence probe dye. Fluorescence data was collected every 30 seconds for 10 minutes of incubation at 37° C. using a fluorescence plate reader that can excite at 584 nm and read at 616 nm. The rise of fluorescence signal indicated a positive detection of SARS-CoV-2.

### Definitions

DETECTR: DNA endonuclease-targeted CRISPR trans reporter

RT-LAMP: Reverse Transcription Loop-mediated Isothermal Amplification

gRNA: Guide RNA

LoD: Limit of Detection

### Methods of Validating Cross-Reactivity

Cross reactivity was validated by two methods. In a first method, in silico (Basic Local Alignment Search Tool-nucleotide) BLASTn analysis queries of the DETECTR SARS-CoV-2 Reagent Kit N gene primers and gRNAs were performed against public domain nucleotide sequences in NCBI (National Center for Biotechnology Information) nucleotide collection (nt) using default parameters for the viruses and bacteria listed in Table 10 below. In a second method, the same lists of organisms analyzed were tested with the DETECTR kit at concentrations of 10⁶ colony-forming units (CFU)/mL or higher for bacteria and 10⁵ plaque-forming units (pfu)/mL or at highest titer possible (depending on titer of commercially available source) for virus.

The list of organisms analyzed and tested with nasal samples is provided in Table 8: Cross-Reactivity Organisms for Nasal Samples.

Cross-reactivity was also tested in a wet lab. For the wet lab testing, a manual protocol was used to extract and analyze RNA samples from the specimens using the DETECTR SARS-CoV-2 Reagent Kit in BSL 2 hoods. Each organism was tested in triplicates at concentrations of 10⁶ CFU/ml or higher for bacteria and 10⁵ pfu/ml or higher for viruses if this concentration could be obtained. If the available concentration was lower, the maximum concentration available was tested.

The substances tested for interference were spiked at a specified concentration into nasal matrix and then the heat inactivated SARS-CoV-2 was added at 3× LoD.

The list of endogenous interferents tested with nasal samples is provided in Table 9:

Sample size of the studies follows the recommendations in the FDA EUA molecular diagnostics template for manufacturers.

TABLE 8
Cross-Reactivity Organisms for Nasal Samples

| Other high priority pathogens | High priority organisms likely present in |
|---|---|
| from the same genetic family | a respiratory specimen |
| Human coronavirus 229E | Adenovirus (eg., C1 Ad. 71) |
| Human coronavirus OC43 | Human Metapneumovirus (hMPV) |
| Human coronavirus HKU1 | Parainfluenza virus 1-4 |
| Human coronavirus NL63 | Influenza A & B |
| SARS-coronavirus | Enterovirus (e.g., EV68) |
| MERS-coronavirus | Respiratory syncytial virus |
| | Rhinovirus |
| | Chlamydia pneumoniae |
| | Haemophilus influenzae |
| | Legionella pneumophila |
| | Mycobacterium tuberculosis |
| | Streptococcus pneumoniae |
| | Streptococcus pyogenes |
| | Bordetella pertussis |
| | Mycoplasma pneumoniae |
| | Pneumocystis jirovecii (PJP) |
| | Pooled human nasal wash - to represent |
| | diverse microbial flora in the human |
| | respiratory tract |
| | Candida albicans |
| | Pseudomonas aeruginosa |
| | Staphylococcus epidermis |
| | Streptococcus salivarius |

TABLE 9
Potential Interferents for Nasal Samples
Interfering substances for Anterior Nares samples

Mucin
Whole Blood
NeoSynephrine Cold and Sinus Extra Strength Spray
Afrin
Zicam Allergy Relief
Flonase (Fluticasone)
Dexamethasone
Mupirocin
Zanamivir (Relenza)
Tamiflu (Oseltamivir phosphate)
Tobramcyin

### Reaction Components:

The assays and reagents disclosed herein can be used as a companion diagnostic with any of the diseases disclosed herein, or can be used in reagent kits, point-of-care diagnostics, or over-the-counter diagnostics. The disclosed reaction vessels may be used as a point of care diagnostic or as a lab test for detection of a target nucleic acid and, thereby, detection of a condition in a subject from which the sample was taken. The reaction vessels may be used in various sites or locations, such as in laboratories, in hospitals, in physician offices/laboratories (POLs), in clinics, at remotes sites, or at home. Sometimes, the present disclosure provides various reaction vessels for consumer genetic use or for over the counter use.

### Contents of DETECTR SARS-CoV-2 Kits:

LYSIS/BINDING SOLUTION:
Lysis/binding solution concentrate
WASH SOLUTION:
Wash Solution 1 Concentrate
ELUTION SOLUTION:
10 mM Tris HCl, pH 8.0 from Thermo
RNA BINDING BEADS:
MagMAX RNA binding beads
RT-LAMP MASTER MIX:
Bst 2.0 DNA polymerase
WarmStart RTx
RNase inhibitor, Murine
Elution Buffer
100 mM dATP
100 mM dCTP
100 mM dGTP
100 mM dTTP
ACTIVATOR:
KOAc (P1190)
MgOAc(M5661)
NH4OAc (A1542)
Tris HCl, pH 9.0 (T2819)
Tween 20 (P9416)
Primer F3
Primer B3
Primer FIP
Primer BIP
Primer LF
Primer LB
DETECTR:
Guide RNA
Cas12
Reporter
Tris HCl, pH 8.0 (T2694)
KOAc (P1190)
1M MgOAc (63052)
Glycerol (G5516)
Tween
PLATE POSITIVE:
Plate Positive Control
PLATE NEGATIVE:
Plate Positive Control
CARRIER RNA:
Carrier RNA
LYSIS/BINDING ENHANCER:
Lysis/Binding Enhancer

### Basic Contents of DETECTR SARS-CoV-2 Kit

DETECTR SARS-CoV-2 Kit
kit = 768 tests

Room Temp Kit
Lysis/Binding Solution
Wash solution
Elution Buffer
Cooler Kit
RNA Binding Beads
Freezer Kit
RT-LAMP master mix
RT-LAMP activator
DETECTR master mix
Plate positive control
Plate negative control
Carrier RNA
Lysis/Binding Enhancer

### Results

The assays tested for cross-reactivity with the list of organisms in Tables 10 and 11 below. As is shown in Tables 10 and 11, all of the samples eventually tested negative for the organisms listed.

Additionally, the assays tested for interference from endogenous substances. As is shown in Table 12, the endogenous substances did not inhibit successful detection of SARS-CoV-2. Results are further summarized below.

### Summary of Analysis

| Description | Pass/Fail Criteria | Results |
|---|---|---|
| The assay shall | No cross reactivity | Pass. |
| demonstrate no | detected with the | See Table 10 and 8 |
| cross reactivity with | listed organisms from | for wet testing |
| the organisms | the in silico analysis. | results summary. |
| listed in the FDA | No cross reactivity | See In silico analysis |
| Emergency Use | detected with the | summary below. |
| Authorization | listed organisms in | |
| Molecular | the wet lab testing. | |
| Diagnostic Template | | |
| for Commercial | | |
| Manufacturers. | | |
| The assay shall | No detected | Pass. See Table 12 |
| demonstrate | interference from | for wet testing |
| minimal or no | endogenous | resultssummary. |
| interference from | substances. | |
| endogenous substances | | |
| such as blood or | | |
| medications such as | | |
| decongestants or | | |
| antihistamines. | | |

### Nasal Matrix Results

TABLE 10
Viral Culture in Nasal Matrix

| | | | Detected/ |
|---|---|---|---|
| | | Final | Tested |
| | Sample | concentration | Negative |
| Organism | Type | (pfu/mL) | Nasal Matrix |
| Coronavirus (Strain: NL63) | Nasal | 4.94 × 10⁴ | 0/3 |
| Coronavirus (Strain: 229E) | Nasal | 4.94 × 10⁴ | 0/3 |
| Coronavirus (Strain: OC43) | Nasal | 1.46 × 10⁵ | 0/3 |
| Coronavirus (Strain: HKU-1) | Nasal | Unknown^{c} | 0/3 |
| MERS-CoV | Nasal | 1.24 × 10⁵ | 0/3 |
| SARS-CoV-1 | Nasal | Unknown^{d} | 0/3 |
| Influenza A H1N1 | Nasal | 4.94 × 10⁴ | 0/3 |
| Influenza B | Nasal | 3.86 × 10⁴ | 0/3 |
| Adenovirus Type 4 | Nasal | 1.75 × 10⁵ | 0/3 |
| Adenovirus Type 3 | Nasal | 2.53 × 10⁵ | 0/3 |
| Adenovirus Type 7 | Nasal | 4.41 × 10⁵ | 0/3 |
| hMPV 16 Type A1 | Nasal | 4.41 × 10⁵ | 0/3 |
| hMPV 27 Type A2 | Nasal | 1.75 × 10⁵ | 0/3 |
| hMPV 3 Type B1 | Nasal | 1.36 × 10⁴ | 0/3 |
| hMPV 4 Type B2 | Nasal | 3.68 × 10⁵ | 0/3 |
| Parainfluenza Virus Type 1 | Nasal | 1.75 × 10⁵ | 1/6^{a} |
| Parainfluenza Virus Type 2 | Nasal | 1.75 × 10⁵ | 0/3 |
| Parainfluenza Virus Type 3 | Nasal | 5.95 × 10⁴ | 0/3 |
| Parainfluenza Virus Type 4A | Nasal | 1.46 × 10⁵ | 0/3 |
| Parainfluenza Virus Type 4B | Nasal | 5.95 × 10⁴ | 0/3 |
| Enterovirus Type 68 | Nasal | 7.67 × 10⁵ | 1/6^{b} |
| RSV-A | Nasal | 1.75 × 10⁵ | 0/3 |
| RSV-B | Nasal | 5.95 × 10⁴ | 0/3 |
| Rhinovirus Type 1A | Nasal | 1.46 × 10⁵ | 0/3 |

^{a}The testing was repeated using a fresh set of Parainfluenza Virus Type 1 samples and all 3 repeats were negative.
^{b}The testing was repeated using a fresh set of Enterovirus Type 68 samples and all 3 repeats were negative.
c. Coronavirus HKU-1 was tested using a commercially available respiratory pathogen qualitative panel containing CoV HKU-1. The viral titer of the CoV HKU-1 is not available. The manufacturer provided a Ct range of 25-28 as the target concentration range for CoV HKU-1 from the manufacturer's real-time PCR assay for CoV HKU-1.
d. SARS-CoV-1 was tested using Coronavirus SARS Stock (Qualitative). The viral titer of the SARS-CoV-1 is not available. The manufacturer provided a Ct range of 25-28 from the manufacturer's real time PCR assay for SARS-CoV-1.

TABLE 11
Bacterial Culture in Nasal Matrix

| | | | Detected/ |
|---|---|---|---|
| | | Final | Tested |
| | Sample | concentration | Negative |
| Organism | Type | (CFU/mL) | Nasal Matrix |
| Chlamydophila pneumoniae | Nasal | 4.12 × 10^{6 a} | 0/3 |
| Haemophilus influenzae type b | Nasal | 1.28 × 10⁷ | 0/3 |
| Haemophilus parainfluenzae | Nasal | 3.29 × 10⁶ | 0/3 |
| Z492 | | | |
| Legionella pneumophila | Nasal | 3.34 × 10⁸ | 0/3 |
| Mycobacterium tuberculosis | Nasal | 1.47 × 10⁶ | 0/3 |
| Streptococcus pneumoniae | Nasal | 9.79 × 10⁶ | 0/3 |
| Streptococcus pyogenes | Nasal | 6.26 × 10⁷ | 0/3 |
| Bordetella pertussis | Nasal | 1.51 × 10⁸ | 0/3 |
| Mycoplasma pneumoniae | Nasal | 7.44 × 10⁶ | 0/3 |
| Candida albicans | Nasal | 1.06 × 10⁷ | 1/6^{b} |
| Pseudomonas aeruginosa | Nasal | 2.02 × 10⁸ | 0/3 |
| Streptococcus salivarius | Nasal | 9.86 × 10⁶ | 0/3 |
| Staphylococcus epidermidis | Nasal | 2.18 × 10⁸ | 0/3 |
| Pneumocystis jirovecii | Nasal | Unknown^{c} | 0/3 |

^{a} Reported as IFU/mL on the certificate of analysis from the vendor.
^{b}The testing was repeated using a fresh set of Candida albicans samples and all 3 repeats were negative.
^{c}Pneumocystis jirovecii was tested using a recombinant control material. The bacterial titer of the test sample is not available. The manufacturer provided a Ct range of 23-25 from the manufacturer's real time PCR assay targeting the P. jirovecii mitochondrial gene for large subunit ribosomal RNA.

A panel of potential endogenous interferents were tested by spiking at the specified concentration in the table below into nasal matrix and then the heat inactivated SARS-CoV-2 was added at 2400 copies/mL (˜3× LOD).

TABLE 12
Potential Interfering Substances in Nasal Matrix

| | | | Detected/Tested |
|---|---|---|---|
| Interfering substances | | | Positive Nasal Matrix |
| for Anterior Nares | Test | | (SARS-CoV-2 RNA |
| Swab samples | Concentration | | present at 3x LoD) |
| Mucin | 0.5% | (w/v) | 3/3 |
| Whole Blood | 1% | (v/v) | 3/3 |
| NeoSynephrine Cold | 20% | (v/v) | 3/3 |
| and Sinus Extra Strength | | | |
| Spray | | | |
| Afrin | 20% | (v/v) | 3/3 |
| Zicam Allergy Relief | 20% | (v/v) | 3/3 |
| Flonase (Fluticasone) | 0.04 | mg/mL | 3/3 |
| Dexamethasone | 0.5 | mg/mL | 3/3 |
| Mupirocin | 10 | mg/mL | 3/3 |
| Zanamivir (Relenza) | 0.3 | mg/mL | 3/3 |
| Tamiflu (Oseltamivir | 0.01 | mg/mL | 3/3 |
| phosphate) | | | |
| Tobramcyin | 2.5 | mg/mL | 3/3 |

### Cross-Reactivity In Silico Analysis Results Summary

N Gene Primers and gRNA:

F3: 83.3% homology to a sequence in the Haemophilus influenzae genome and the Homo sapiens genome. No significant homology to other organisms of interest.

B3: 81.8% identity to a sequence in the Homo sapiens genome.

FIP (F2-F1c): 100% homology to SARS-CoV. No significant homology to other organisms of interest.

BIP (B2-B1c): 100% homology for the B1c portion of the BIP primer to SARS-CoV. No significant homology to other organisms of interest.

LF: homology to SARS-CoV (94%), Chlamydia pneumoniae (84%), Streptococcus pyogenes (84%), and Homo sapiens (89% genomes).

LB: No significant homology to other organisms of interest.

N-gene gRNA: 80% homology to a sequence in the Homo sapiens genome however the required proto-spacer adjacent motif (PAM) to the target sequence is not present. The Cas12 enzyme will not activate due to the lack of PAM adjacent to the target sequence.

Although some primers have partial homology to the organisms of interest, it is unlikely for cross-reactivity to occur with these organisms as RT-LAMP requires complementarity to at least 4 of the 6 primers. In addition, the specificity of the RT-LAMP amplicon is benefited by the sequence specificity of the Cas enzyme with its gRNA. With respect to SARS-CoV, 3 N gene primers (BIP, FIP, LF) have high homology (94%-100%) to SARS coronavirus, cross-reactivity would not be expected given the lack of sequence homology in the other 3 primers and the N-gene gRNA.

### Example 3

### Analytical Sensitivity Experiments—Limit of Detection (LoD)

This test demonstrates the analytical sensitivity of the DETECTR SARS-CoV-2 Reagent Kit. The results of testing the limit of detection (LoD) for nasal samples is reported.

LoD studies were conducted with contrived samples using the manual assay (WI005), or using laboratory equipment that automates assay execution such as the Agilent BRAVO Assay Workstation. Heat inactivated SARS-CoV-2 virus was spiked into phosphate buffered saline (PBS) with nasal matrix and the DNA Genotek OM-505 media with saliva matrix.

LoD studies used DETECTR SARS-CoV-2 Reagent kits.

The LoD studies were performed in two parts: A study determining the preliminary LoD and a confirmation study defining the LoD of the assay.

### Results

To measure the assay's LoD, a heat inactivated virus of SARS-CoV-2 from strain 2019-nCoV/USA-WA1/2020 procured from ATCC, catalogue number, VR-1986HK, was obtained and quantified using digital droplet PCT. Dilutions of the heat inactivated SARS-CoV-2 virus were made in phosphate buffered saline (PBS) and a human nasal matrix. Different assay execution methods were used, a manual execution method and a programmed method using an automated liquid handler (Agilent BRAVO or Hamilton STAR). Other automated liquid handlers can be used to execute the assay.

The preliminary LoD was assessed with 3 replicates. The preliminary LoD was defined as the lowest concentration that achieves 100% positivity. assessed using the manual assay and the Agilent BRAVO based liquid handling platform and concluded to be 500 virus copies/mL under the specific testing conditions. The results of each concentration tested are summarized in Table 16 below:

TABLE 13
Preliminary LoD

| | | Positive Results |
|---|---|---|
| | Positive Results | (BRAVO, |
| Virus Copies/mL | (Manual) | Automated/Robotic) |
| 4000 | 3/3 | 3/3 |
| 2000 | 3/3 | 3/3 |
| 1000 | 3/3 | 3/3 |
| 680 | 3/3 | 3/3 |
| 500 | 3/3 | 3/3 |
| 300 | 1/3 | 2/3 |
| 250 | 2/3 | 2/3 |

The LoD was confirmed with additional tests of three concentrations, including the preliminary LoD, one level above and one level lower. Each sample was run in 20 replicates. The LoD is defined as the lowest concentration that achieves 19/20 positive results. The results of each concentration tested to confirm LoD are summarized in Table 14 below:

TABLE 14
Confirmed LoD, Manual Execution

| | Positive | Positive Results | Positive Results |
|---|---|---|---|
| Virus | Results | (BRAVO, | (STAR, |
| Copies/mL | (Manual) | Automated/Robotic) | Automated/Robotic) |
| 680 | 20/20 | 20/20 | 20/20 |
| 500 | 18/20 | 16/20 | 20/20 |
| 300 | 10/20 | 10/20 | 20/20 |

Under the specific conditions tested, the LoD was 680 copies/mL with the manual assay execution and with the automated Agilent BRAVO liquid handling platform. The LoD was 300 copies/mL with the automated Hamilton STAR liquid handling platform.

### Example 4

### Analytical Sensitivity Experiments—Inclusivity

In silico analysis was conducted to confirm that SARS-CoV-2 sequences published in the GISAID database are detectable by DETECTR SARS-CoV-2 Kit RT-LAMP primers and DETECTR gRNA.

The results of the analysis are summarized below:

| Description | Pass/Fail Criteria | Results |
|---|---|---|
| The kit shall test for | 100% homology of | Pass: 28,639 strains |
| the presence of the | primers and gRNA to | with 100% |
| SARS- CoV-2 in a | the SARS-CoV-2 | homology |
| biological sample. The | sequences | Pass: |
| assay shall detect all | Or | See Risk assessment |
| the SARS-CoV-2 strains | Risk assessment of | below. |
| that are represented as | any mismatch that | |
| SARS-CoV-2 genome | may impact assay | |
| sequences in the | performance | |
| GISAID database | | |

### Risk Assessment:

To demonstrate the predicted inclusivity, in silico analysis of the primer and gRNA sequences was performed with SARS-CoV-2 genomes. These sequences represent all complete (defined as >29 kbp of sequence), high-coverage sequences collected from humans in the GISAID database.

A total of 490,303 high-quality and full-length sequences were available from GISAID at the time of analysis. Of the analyzed genomes, 3.70% (18,149 of 490,303) were found containing single nucleotide variants (SNVs) in the primer and gRNA regions in the N gene target amplicon used by the DETECTR SARS-CoV-2 Reagent Kit. Among the variants, 16,443 had a single SNV within one of the 6 primer regions (F3, B3, LF, LB, FIP, or BIP), 259 had two or more SNVs within one of the 6 primer regions, and 1,447 had at least one single SNV within the gRNA region. A summary of the results of this analysis for each assay component is presented in Table 18.

TABLE 15
Summary of mutations GISAID sequences that overlap assay components. Results are broken
down by number of mismatches (mm) between strain sequence and primer sequence.

| | | | | | | | 3′ |
|---|---|---|---|---|---|---|---|
| | | 100% | 1 mm | 2 | 3 | >3 | end |
| | Component | match | (SNV) | mm | mm | mm | mm |
| LAMP | F3 | 98.89% | 1.073% | 0.005% | 0% | 0.001% | 0.008% |
| primers | B3 | 99.64% | 0.353% | 0.001% | 0% | 0% | 0.047% |
| | FIP | 98.61% | 1.345% | 0.002% | 0% | 0% | 0.023% |
| | BIP | 99.19% | 0.772% | 0.003% | 0% | 0.029% | 0.0035% |
| | LF | 99.27% | 0.723% | 0.001% | 0% | 0% | 0.001% |
| | LB | 99.22% | 0.645% | 0.006% | 0% | 0% | 0.047% |
| CRISPR | gRNA | 99.56% | 0.404% | 0% | 0.003% | 0.003% | N/A |

A single SNV in a primer region was unlikely to affect the sensitivity of the assay unless it was at the 3′ end of the primer. Among the variants with a single SNV within one of the 6 primer regions, only 0.001%-0.047% had an SNV at the 3′ end of the primer. Not every primer was able to be aligned in all 490,303 sequences due to sequencing errors thus the percentage is different for each of the 6 primers.

A sequence containing a SNV in the gRNA region may also affect sensitivity given the single nucleotide specificity of the gRNA for a CRISPR-Cas12 reaction. Thus, the sensitivity of detection of the DETECTR SARS-CoV-2 Reagent Kit would possibly be affected in only 0.4% of the 490,303 analyzed genomes. Note that the SNV in the gRNA region may also affect sensitivity of the detection of the CNC N2 assay as well, as it also overlaps with the N2 probe region.

### Risk Assessment of Variants of Interest (B.1.1.7, B.1.351, P.1):

To understand the impact of the new SARS-CoV-2 variants that have arisen around the world, we performed an alignment of the primer and gRNA sequences against full-length, high-quality SARS-CoV-2 genomes for B.1.1.7 (UK variant, n=56,329), B.1.351 (South Africa variant, n=910), and P.1 (Brazil variant, n=113) from GISAID. Our analysis of the SARS-CoV-2 variants shows that observed SNVs occurred at frequencies similar to those seen for our assay primers on the SARS-CoV-2 population as a whole and were unlikely to impact the performance of the DETECTR SARS-CoV-2 Reagent Kit. Results are summarized in Tables 16, 17, and 18.

TABLE 16
Summary of mutations in B.1.1.7 strains (n =
56,329) from GISAID that overlap assay components.

| | | | | | | | 3′ |
|---|---|---|---|---|---|---|---|
| | | 100% | 1 mm | 2 | 3 | >3 | end |
| | Component | match | (SNV) | mm | mm | mm | mm |
| LAMP | F3 | 99.68% | 0.32% | 0% | 0 | 0% | 0.007% |
| primers | B3 | 99.78% | 0.22% | 0% | 0% | 0% | 0.04% |
| | FIP | 99.46% | 0.54% | 0% | 0% | 0% | 0.007% |
| | BIP | 99.19% | 0.81% | 0.003% | 0% | 0% | 0% |
| | LF | 99.82% | 0.18% | 0% | 0% | 0% | 0% |
| | LB | 99.64% | 0.36% | 0% | 0% | 0% | 0% |
| CRISPR | gRNA | 99.79% | 0.21% | 0% | 0% | 0% | N/A |

TABLE 17
Summary of mutations in B.1.351 strains (n =
910) from GISAID that overlap assay components.

| | | | | | | | 3′ |
|---|---|---|---|---|---|---|---|
| | | 100% | 1 mm | 2 | 3 | >3 | end |
| | Component | match | (SNV) | mm | mm | mm | mm |
| LAMP | F3 | 99.89% | 0.11% | 0% | 0% | 0% | 0% |
| primers | B3 | 99.67% | 0.33% | 0% | 0% | 0% | 0% |
| | FTP | 99.45% | 0.55% | 0% | 0% | 0% | 0% |
| | BIP | 99.56% | 0.44% | 0% | 0% | 0% | 0% |
| | LF | 100% | 0% | 0% | 0% | 0% | 0% |
| | LB | 99.56% | 0.44% | 0% | 0% | 0% | 0% |
| CRISPR | gRNA | 99.89% | 0.11% | 0% | 0% | 0% | N/A |

TABLE 18
Summary of mutations in P.1 strains (n =
113) from GISAID that overlap assay components.

| | | | | | | | 3′ |
|---|---|---|---|---|---|---|---|
| | | 100% | 1 mm | 2 | 3 | >3 | end |
| | Component | match | (SNV) | mm | mm | mm | mm |
| LAMP | F3 | 100% | 0% | 0% | 0% | 0% | 0% |
| primers | B3 | 100% | 0% | 0% | 0% | 0% | 0% |
| | FIP | 100% | 0% | 0% | 0% | 0% | 0% |
| | BIP | 100% | 0% | 0% | 0% | 0% | 0% |
| | LF | 100% | 0% | 0% | 0% | 0% | 0% |
| | LB | 100% | 0% | 0% | 0% | 0% | 0% |
| CRISPR | gRNA | 100% | 0% | 0% | 0% | 0% | N/A |

To further assess the risk of B.1.1.7 non-detection, two available synthetic RNA controls from Twist Biosciences generated to emulate two strains of B.1.1.7 were obtained and tested with the DETECTR SARS-CoV-2 Reagent Kit. Table 19 provides the results of this testing. The two B.1.1.7 test samples were detected 100% of the time even near assay's expected LoD.

TABLE 19
Wet testing of B.1.1.7 synthetic samples

| | Detected/ | Detected/ | Detected/ |
|---|---|---|---|
| Catalogue Number RNA | Tested | Tested | Tested |
| Controls Tested | at 500 copies | at 250 copies | at 100 copies |
| 102024: Control 2 | 3/3 | 3/3 | 2/3 |
| (MN908947.3) | | | |
| 103907: Control 14 | 3/3 | 3/3 | 3/3 |
| (B.1.1.7_710528) | | | |
| 103909: Control 15 | 3/3 | 3/3 | 3/3 |
| (B.1.1.7_601443) | | | |

### Example 5

### Clinical Evaluation

The clinical evaluation compared the results from the DETECTR assay to an FDA-accepted comparator assay. The evaluation tested for positive percent agreement and negative percent agreement (with respect to positive and negative test results) between the DETECTR assay and the comparator assay.

### Experimental Setup:

total of two hundred and thirty (230) nasopharyngeal (NP) swab specimens in Phosphate Buffered Saline (PBS) were collected and tested retrospectively with the DETECTR SARS-CoV-2 Reagent Kit and a comparator EUA RT-PCR assay, Panther Fusion® SARS-CoV-2 Assay. One hundred and ten (110) were from frozen dry NP swab specimens stored at −80° C., removed from −80° C. storage and eluted in PBS and one hundred and twenty (120) NP swab specimens were collected directly into PBS.

### Results

Results from 80 individuals who tested positive by the comparator assay and 150 individuals who tested negative by the comparator assay are provided in Table 20. The positive percent agreement (PPA) between the two assays was 94.7% (72/76) and the negative percent agreement (NPA) was 94.8% (146/154). The binomial proportion confidence interval provided in Table 20 was calculated using the Clopper-Pearson (exact) method. There was 100% concordance between the DETECTR SARS-CoV-2 Reagent Kit and the Panther Fusion® SARS-CoV-2 Assay on specimens with a SARS-CoV-2 Assay Ct of less than 35.7 (n=67). Eight (8) discordant results occurred between the SARS-CoV-2 Assay Ct of 35.7 to 38.8. The DETECTR SARS-CoV-2 Reagent Kit called four (4) specimens positive that were negative with the SARS-CoV-2 Assay. Discordant samples were repeat tested on both platforms twice. See Table 21 for repeat testing results with both methods of the twelve specimens that had discordant results. The inconsistent results indicate that these specimens may be below the limit of detection for both the candidate and comparator method.

TABLE 20
Evaluation with nasopharyngeal Specimens

| | | Comparator EUA RT-PCR Assay | | |
|---|---|---|---|---|
| | | Positive | Negative | Total |
| DETECTR ™ | Positive | 72 | 4^{b} | 76 |
| SARS-CoV-2 | Negative | 8^{a} | 146 | 154 |
| Reagent | | | | |
| Kit | Total | 80 | 150 | 230 |

| | |
|---|---|
| Positive Percent | 94.7% (95% CI: 87.1%-98.6%) |
| Agreement (PPA) | |
| Negative Percent | 94.8% (95% CI: 90.0%-97.7%) |
| Agreement (NPA) | |

^{a}The eight discordant results occurred with a comparator assay Ct from 35.7 to 38.8. See Table 20 for repeat testing.
^{b}The specimens with four discordant results were repeat tested. See Table 21 for repeat testing.

TABLE 21
Repeat tests of specimens with discordant results

| | | | | | First repeat test | | | Second repeat test | | |
|---|---|---|---|---|---|---|---|---|---|---|
| | | Original test | | | | Panther Fusion | | | Panther Fusion | |
| Table 22 | Study | DETECTR | Panther Fusion | | DETECTR | result and Ct | | DETECTR | result and Ct | |
| footnote | Sample ID | result | result with Ct | | result | if positive | | result | if positive | |
| a | MBS0015 | Negative | Positive | 38.1 | Negative | Negative | N/A | Negative | Negative | N/A |
| a | MBS0020 | Negative | Positive | 37.1 | Positive | Positive | 39.0 | Positive | Negative | N/A |
| a | MBS0034 | Negative | Positive | 38.0 | Negative | Negative | N/A | Negative | Negative | N/A |
| a | MBS0042 | Negative | Positive | 38.1 | Positive | Positive | 38.1 | Negative | Positive | 36.1 |
| a | MBS0057 | Negative | Positive | 35.7 | Negative | Negative | N/A | Negative | Positive | 36.8 |
| a | MBS0079 | Negative | Positive | 38.4 | Negative | Negative | N/A | Negative | Positive | 38.3 |
| a | MBS0089 | Negative | Positive | 38.8 | Negative | Negative | N/A | Positive | Negative | N/A |
| a | MBS0106 | Negative | Positive | 36.3 | Positive | Positive | 31.0 | Positive | Positive | 32.8 |
| b | MBS0065 | Positive | Negative | N/A | Negative | Negative | N/A | Negative | Negative | N/A |
| b | MBS0070 | Positive | Negative | N/A | Negative | Negative | N/A | Negative | Positive | 37.3 |
| b | MBS0082 | Positive | Negative | N/A | Negative | Negative | N/A | Negative | Positive | 38.2 |
| b | MBS0114 | Positive | Negative | N/A | Positive | Positive | 37.8 | Positive | Positive | 38.2 |

### Example 6

### Multiplexing

An RT-LAMP based DETECTR assay is configured as a CRISPR-Cas enzymatic multiplexed detection for a panel of respiratory agents including SARS-CoV-2, SARS-CoV-2 variants, Influenza A, Influenza B, and RSV from patients presenting with respiratory symptoms by their healthcare provider. The multiplexed assay is performed manually or using an automated liquid handling platform such as the Agilent Bravo or Hamilton STAR. In addition to Influenza A, Influenza B and RSV, the respiratory panel detects three of the most important SARS-CoV-2 mutations (L452R, E484K, N501Y) that pick up all the variants of concern (Alpha (B.1.1.7), Beta (B.1.351), Delta (B.1.617.2), Epsilon (B.1.427/B.1.429), and Gamma (P.1)). The panel utilizes the speed, sensitivity, and specificity of CRISPR chemistry to assist in the differential diagnosis of patients presenting with respiratory symptoms and inform public health authorities of the prevalence of variants in a population.

Nucleic acid sample is collected from nasopharyngeal swabs, nasal, mid-turbinate, or oropharyngeal from individuals suffering from respiratory distress. After nucleic acid extraction from swab elution, the isothermal amplification primers target gene sequences of interest of the pathogenic virus or bacteria. The CRISPR-Cas enzyme uses guide RNAs that target the isothermal amplification amplicons for sensitive and specific detection.

In at least some instances, isothermal amplification is prone to non-specific amplification while the CRISPR-Cas enzyme is unaffected by the non-specific amplification and detects only its target of interest. Isothermal amplification alone is not typically capable of a high level of analytical sensitivity, but when paired with CRISPR-Cas enzymatic detection, can achieve high levels of analytical and clinical performance. In at least some instances, this panel is well suited for disease surveillance to identify individuals who should quarantine to mitigate the spread of respiratory diseases during outbreaks.

The multiplexing assay kit includes the reagents necessary for nucleic acid extraction, isothermal amplification and CRISPR-based detection using a workflow similar to that shown in FIG. 1D in order to multiplex the detection of the common pathogens that cause similar respiratory disease symptoms in patients experiencing respiratory distress. Accurately identifying which pathogen is causing respiratory distress in a patient is important in controlling disease outbreaks, e.g., as we move into the next phase of the COVID-19 pandemic. Due to the infectiousness and disease severity of SARS-CoV-2, quickly and accurately stratifying patients experiencing respiratory distress, especially as circulating variants like the Delta variant begin to enter communities in the US, is increasingly important.

The CRISPR-Cas enzyme's programmable guide RNA allows for specific and sensitive multiplexing. The CRISPR-Cas enzymes are coupled to different guide RNA sequences that are complementary to each of the different nucleic acid target sequences to be detected in the respiratory disease panel. A different guide RNA is designed for each target sequence of interest. A 384-well microplate is pre-filled with CRISPR-Cas enzyme reagents, one target for each well. The microplate is divided in columns by sample specimens and in rows for target sequences to be detected.

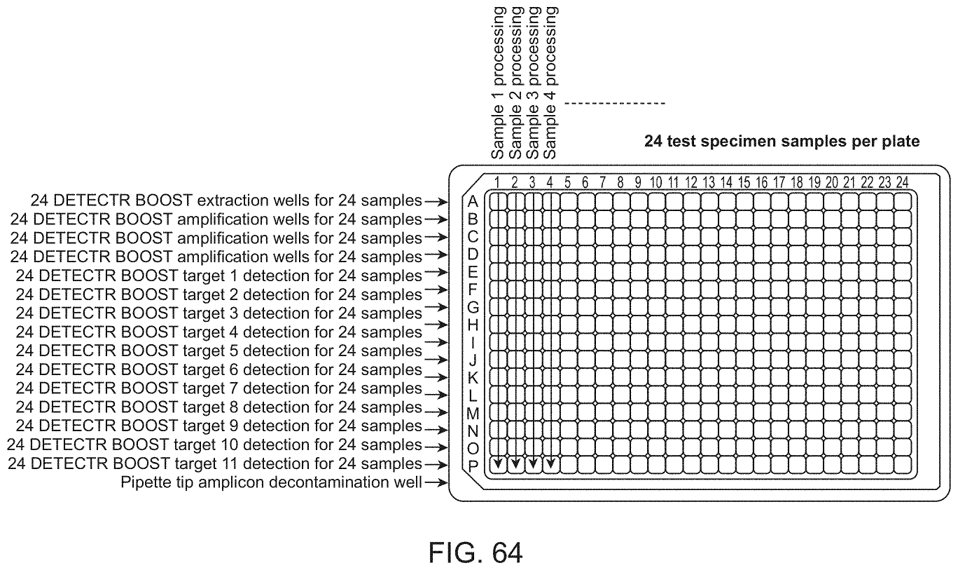

The multiplex respiratory disease panel uses the same reagent kit and integration designs as the previously-described DETECTR SARS-CoV-2 Reagent Kit, except that different DETECTR master mixes are located in separate microplate wells for the different targets. Isothermal amplification occurs in multiple wells. FIG. 64 shows a microplate format for the DETECTR Respiratory Disease Panel. The isothermal amplification occurs in one or more wells depending on amplification primer complexity. One amplification well may be linked to one or more DETECTR wells. Each DETECTR well provides one specific target result. For SARS-CoV-2, a “wild type” well detects the SARS-CoV-2 virus that is most commonly in circulation. More than one guide RNA may be used in the SARS-CoV-2 wild type well for comprehensive viral coverage. Each SARS-CoV-2 variant has a DETECTR well. As shown in FIG. 64, the liquid handling platforms pipette process each specimen through the assay workflow down each microplate well column. The extraction occurs in a single well as described herein. The elution is then moved to the amplification well(s). The number of amplification wells needed may be dependent on the number of targets for the panel and the primer designs. The amplified product is then moved to the corresponding DETECTR wells for the same target nucleic acid. The format of moving only within a column may reduce or eliminate amplicon cross-contamination issues. To further minimize any amplicon cross-contamination, an amplicon decontamination reagent is included in row P at the bottom of the plate so that each tip can be cleaned and decontaminated before leaving its sample-specific column.

The multiplex respiratory disease panel may include any of the targets described herein. In an example, the respiratory disease panel includes Influenza A, Influenza B, SARS-CoV-2, RSV-A, and RSV-B. Table 22 shows exemplary Influenza A, Influenza B, RSV-A, RSV-B, and RNaseP (endogenous control) LAMP primers and guide RNAs which can be included in the respiratory disease panel test.

TABLE 22
Influenza A, Influenza B, RSV-A, RSV-B, and
LRNaseP AMP primers and guide RNA

| | |
|---|---|
| Influenza A | CTCAAAGCCGAGATCGC |
| Primer F3 | |
| Influenza A | CTGCTCTGTCCATGTTRTT |
| Primer B3 | |
| Influenza A | TCAGAGGTGACAGGATTGGTCTGAAGATGTCTTT |
| Primer FIP | GCAGGGAA |
| Influenza A | TCACCGTGCCCAGTGAGCCATTCCCATTGAGGGC |
| Primer BIP | ATT |
| Influenza A | ATTCCATGAGAGCCTCAAGATC |
| Primer LF | |
| Influenza A | GAGGACTGCAGCGTAGAC |
| Primer LB | |
| Influenza A | UAAUUUCUACUAAGUGUAGAUGACAAAGCGUCUA |
| gRNA | CGCUGCA |
| Influenza B | TGCTAAACTTGTTGCTACTG |
| Primer F3 | |
| Influenza B | TTCTTCCGTGACCAGTCT |
| Primer B3 | |
| Influenza B | CGCTCGAAGAGTGAATTGAGGATGATCTTACAGT |
| Primer FIP | GGAGGATG |
| Influenza B | CTGCGGTGGGAGTCTTATCCGTCTCCCTCTTCTG |
| Primer BIP | GTGAT |
| Influenza B | ATCCGATGGCCATCTTCTT |
| Primer LF | |
| Influenza B | CAATTTGGTCAAGAGCACCG |
| Primer LB | |
| Influenza B | UAAUUUCUACUAAGUGUAGAUAGCUGCUCGAAUU |
| gRNA | GGCUUUG |
| RSV-A Primer | AACATACGTGAACAAACTTCA |
| F3 | |
| RSV-A Primer | GCACATATGGTAAATTTGCTGG |
| B3 | |
| RSV-A Primer | ACCCATATTGTAAGTGATGCAGGATAGGGCTCCA |
| FIP | CATACACAG |
| RSV-A Primer | CTAGTGAAACAAATATCCACACCCAAGCACTGCA |
| BIP | CTTCTTGAG |
| RSV-A Primer | TTTCTAGGACATTGTATTGAACAGC |
| LF | |
| RSV-A Primer | GGGACCCTCATTAAGAGTCATG |
| LB | |
| RSV-A gRNA | UAAUUUCUACUAAGUGUAGAUCUUAUAAAAGAAC |
| | UAGCCAA |
| RSV-B Primer | TTGCAATGATCATAGTTTACCT |
| F3 | |
| RSV-B Primer | GCATCTATTTACAGAAGAACAGTA |
| B3 | |
| RSV-B Primer | GTTGCATCTGTAGCAGGAATGGTTAATTGAATTT |
| FIP | CTAAGGTTATACAACG |
| RSV-B Primer | AGTATCTTTGTCTGCGATGCTGTGCACTTTCTTA |
| BIP | CATGCTTAC |
| RSV-B Primer | TCTCACCATAATCTATGTTTATATGCC |
| LF | |
| RSV-B Primer | AATTACCTGTCACAGCCAATTGGAG |
| LB | |
| RSV-B gRNA | UAAUUUCUACUAAGUGUAGAUCUUAUAAAAGAAC |
| | UAGCCAA |
| RNase P | CACATCCGAGTCTTCAGG |
| Primer F3 | |
| RNase P | GGCAATAGTTACAGACCGC |
| Primer B3 | |
| RNase P | TCCAGTACTCAGCATGCGAAGCACCCAAGTAATT |
| Primer FTP | GAAAAGACAC |
| RNase P | CTGGAAGCCCAAAGGACTCTATACACACACTCAG |
| Primer BIP | GAAGG |
| RNase P | CGGAGGGGATAAGTGGAGGA |
| Primer LF | |
| RNase P | GCATTGAGGGTGGGGGT |
| Primer LB | |
| g RNase P | UAAUUUCUACUAAGUGUAGAUUUACAUGGCUCUG |
| RNA | GUCCGAG |

A fluorescent probe for RNase P is included as an endogenous control in at least one of the amplification wells in order to facilitate detection of the endogenous control in the amplification well rather than (or in addition to) in a DETECTR well, as shown in FIG. 65. An internal FAM labeled LAMP RIP (M620) primer is included as an RT-LAMP reagent. There is a self-quenching effect when the T near 3′ end is labeled. Once the primers are incorporated into amplicons, there is a de-quenching effect. An end point read in the FAM channel on the plate reader detects the fluorescence increase due to de-quenching. The RT-LAMP for RNase P is duplexed with the N gene RT-LAMP. At the plate reader, an end point read in FAM channel detects RNase P and a kinetic read in the Alexa594 channel detects the N gene DETECTR reaction if the N gene is present in the well.

The panel is run on a laboratory automated liquid handling platform that has been designed to run laboratory tests and report results to the lab's information management system, such as the Agilent Bravo and the Hamilton STAR laboratory automated liquid handling systems. Many different laboratory automated liquid handling systems are capable of performing this assay.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein can be employed. It is intended that the following claims define the scope of the invention and that methods and structures within the scope of these claims and their equivalents be covered thereby.

What is claimed is:

1. A high-throughput single-chamber process for detecting a presence of a target nucleic acid, comprising:
(a) providing a single chamber;
(b) binding a plurality of nucleic acids with a microparticle within the single chamber to form a microparticle complex;
(c) isolating the microparticle complex within the single chamber;
(d) amplifying the plurality of nucleic acids within the single chamber to form an amplified product;
(e) contacting the amplified product with a guide nucleic acid complexed to a programmable nuclease within the single chamber such that, when the amplified product comprises a target nucleic acid, the guide nucleic acid contacts the target nucleic acid to form an activated programmable nuclease, thereby cleaving a reporter molecule by the activated programmable nuclease to produce a cleaved reporter molecule; and
(f) assaying for a detectable signal emitted within the single chamber by the cleaved reporter molecule, thereby detecting a presence or absence of the target nucleic acid.

2. The process of claim 1, further comprising lysing a sample to release the plurality of nucleic acids within the single-chamber, thereby enabling the plurality of nucleic acids to bind with the microparticle.

3. The process of claim 1, further comprising eluting the plurality of nucleic acids from the microparticle complex.

4. The process of claim 3, wherein the eluting is performed using an elution buffer.

5. The process of claim 3, further comprising removing waste liquid from the single chamber prior to eluting the nucleic acid molecules from the microparticle complex.

6. The process of claim 3, wherein eluting the nucleic acid molecules is performed using pipette mixing or using a plate mixer.

7. The process of claim 1, wherein the guide nucleic acid binds with a segment of the target nucleic acid.

8. The process of claim 1, wherein the microparticle remains in the single chamber during steps (d)-(f).

9. The process of claim 1, wherein (a) is performed at 37+/−2° C., (d) is performed at 57+/−2° C. or 62+/−2° C., and (e) is performed at 37+/−2° C.

10. The process of claim 1, wherein the microparticle comprises a silica-coated magnetic bead, carbohydrate copolymer, hydroxy functionalized copolymer, carboxylic acid functionalized copolymer, or a combination thereof.

11. The process of claim 1, wherein the target nucleic acid is an antigen or fragment thereof.

12. The process of claim 11, wherein the antigen is a viral antigen, a bacterial antigen, or a cancer antigen.

13. The process of claim 1, wherein the process is performed in the single chamber as it is transported to between one and six stations.

14. The process of claim 13, wherein (a)-(b) are performed at a first station, (c) is performed at a second station, eluting the plurality of nucleic acids from the microparticle complex is performed at a third station, (d) is performed at a fourth station, and (e)-(f) are performed at a fifth station.

15. The process of claim 13, wherein a robot moves the single chamber between stations.

16. The process of claim 1, wherein (a)-(f) are performed at one station.

17. The process of claim 1, wherein (a) is performed between an ambient temperature and 95+/−5° C., (d) is performed at a temperature of 57+/−2° C. or 62+/−2° C., and (e)-(f) is performed at a temperature from 37+/−2° C.

18. The process of claim 1, wherein isolating the microparticle complex comprises capturing the microparticle with a magnet.

19. The process of claim 18, wherein capturing comprises bringing the magnet in magnetic contact with the chamber and changing a temperature of the chamber to about 57° C. or about 62° C. prior to eluting the nucleic acid molecules from the microparticle.

20. The process of claim 18, wherein capturing comprises bringing the chamber in magnetic contact with the magnet and changing the temperature to an ambient temperature.

21. The process of claim 1, wherein the reporter molecule comprises a detection moiety for generating the signal.

22. The process of claim 21, wherein the detection moiety comprises a fluorophore.

23. The process of claim 1, wherein the reporter molecule comprises a protein for generating the signal.

24. The process of claim 1, wherein amplifying the nucleic acid molecules comprises performing RT-LAMP.

25. The process of claim 1, wherein the detectable signal comprises a fluorescence signal.

26. The process of claim 25, wherein assaying for the detectable signal comprises detecting the fluorescence signal and obtaining a fluorescence value periodically via a detector.

27. The process of claim 26, wherein obtaining the fluorescence value periodically comprises obtaining a fluorescence value every 20 seconds to produce a plurality of obtained fluorescence values.

28. The process of claim 27, wherein detecting the presence of the target nucleic acid comprises plotting slope values from the plurality of obtained fluorescence values.

29. The process of claim 28, further comprises comparing the slope values to slope values of a positive control and to slope values of a negative control.

30. The process of claim 25, wherein assaying for the detectable signal comprises detecting the fluorescence signal and obtaining a fluorescence value after a predetermined period of time via a detector.

31. The process of claim 1, wherein (a)-(f) are completed in under about 40 minutes.

32. The process of claim 1, wherein (a) is completed in under about one minute, wherein (b) is completed between about four and about ten minutes, wherein (c) is completed in under about one minute, wherein eluting the plurality of nucleic acids from the microparticle complex is completed in between about four and about ten minutes, wherein (d) is completed in about 20-30 minutes, and wherein (e)-(f) is completed in about 5-10 minutes.

33. The process of claim 1, wherein the cleaved reporter molecule is RNA or DNA.

34. The process of claim 1, wherein the single chamber is a first well in a microplate.

35. The process of claim 34, further comprising, in a second well of the microplate, performing steps (a)-(f) on an additional sample.

36. The process of claim 35, wherein performing steps (a)-(f) on the additional sample in the second well occurs after a period of time from initiating (a) in the first well.

37. The process of claim 36, wherein the period is less than or equal to half of a length of time for completion of steps (a)-(f) in the first well.

38. The process of claim 37, wherein the period is about ten minutes.

39. The process of claim 1, wherein the programmable nuclease comprises a CRISPR/Cas enzyme.

40. The process of claim 1, wherein the guide nucleic acid is supplied as a complex with the programmable nuclease.

41. The process of claim 40, wherein the complex of the guide nucleic acid and the programmable nuclease is a ribonucleoprotein complex.

42. The process of claim 1, wherein the guide nucleic acid is supplied in situ with the programmable nuclease.

43. The process of claim 1, wherein the guide nucleic acid comprises a guide RNA.

44. The process of claim 1, wherein the signal is associated with a physical, chemical, electrochemical change or reaction, or combinations thereof.

45. The process of claim 1, wherein the signal comprises an optical signal.

46. The process of claim 1, wherein the signal comprises a potentiometric or amperometric signal.

47. The process of claim 1, wherein the signal comprises a piezoelectric signal.

48. The process of claim 1, wherein the signal is associated with a change in an index of refraction of a solid or gel volume in which the programmable nuclease probe is disposed.

49. The process of claim 4, further comprising providing the programmable nuclease, the reporter molecule, the guide nucleic acid, or a combination thereof, through a detection reagent.

50. The process of claim 1, further comprising using the signal to detect pathogenic viruses, pathogenic bacteria, pathogenic worms, pathogenic fungi, or cancer biomarkers.

51. The process of claim 50, wherein the pathogenic viruses are respiratory viruses, adenoviruses, parainfluenza viruses, severe acute respiratory syndrome (SARS), coronavirus, SARS-CoV, SARS-CoV-2, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, papillomavirus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, or a combination thereof.

52. The process of claim 50, wherein the pathogenic bacteria are selected from the group consisting of Mycobacterium tuberculosis, Klebsiella pneumoniae, Acinetobacter baumannii, Burkholderia cepacia, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Bordetella parapertussis, Bordetella pertussis, Chlamydia pneumoniae, Mycoplasma pneumoniae, Mycobacterium leprae, and Brucella abortus.

53. The process of claim 50, wherein the pathogenic worms are selected from the group consisting of roundworms, heartworms, phytophagous nematodes, flukes, Acanthocephala, and tapeworms.

54. The process of claim 50, wherein the pathogenic fungi are selected from the group consisting of Cryptococcus neoformans, Histoplasma capsulatum, Coccidioides immitis, Blastomyces dermatitidis, Chlamydia trachomatis, and Candida albicans.

55. The process of claim 50, wherein the cancer biomarkers are selected from the group consisting of lung cancer biomarkers and prostate cancer biomarkers.

56. The process of any one of claims 1-55, wherein the programmable nuclease is a programmable Cas12 nuclease, a programmable Cas13 nuclease, a programmable Cas14 nuclease, a programmable thermostable Cas nuclease, or a CasΦ nuclease.

57. The process of any one of claims 1-56, wherein the target nucleic acid is DNA.

58. The process of any one of claims 1-56, wherein the target nucleic acid is RNA.

59. The process of any one of claims 1-58, wherein steps (a)-(g) are performed in a high-throughput manner.

60. The process of claim 59, wherein the high-throughput manner comprises detecting about 400 target nucleic acids in 1.75 hrs or detecting about 192 target nucleic acids in 110 minutes.

61. A high-throughput single-chamber system for detecting a presence of a target nucleic acid, the system comprising:
(a) a lysis agent for lysing a sample, thereby releasing nucleic acid molecules;
(b) one or more microparticles for binding with the nucleic acid molecules to form one or more microparticle complexes therewith;
(c) an isolator to isolate the one or more microparticle complexes in the single chamber;
(d) an elutor to elute the nucleic acid molecules from the one or more microparticle complexes;
(e) an amplification agent for amplifying the nucleic acid molecules via contact thereto, resulting in amplified nucleic acid molecules;
(f) a programmable nuclease;
(g) a reporter molecule,
(h) a guide nucleic acid that is capable of binding at least a segment of a target nucleic acid when present in the amplified nucleic acid molecules, wherein the guide nucleic acid is coupled to the programmable nuclease and wherein binding of the guide nucleic acid to the target nucleic acid activates the programmable nuclease, thereby cleaving the reporter molecule via the programmable nuclease to produce a cleaved reporter molecule, wherein a signal is configured to be emitted using the cleaved reporter molecule, wherein the signal corresponds to a presence of the target nucleic acid; and
(i) a single chamber configured to i) lyse the sample via the lysis agent; ii) form the one or more microparticle complexes; iii) isolate the one or more microparticle complexes; iv) elute the nucleic acid molecules from the one or more microparticle complexes; v) amplify the nucleic acid molecules while the one or microparticles remain in the single chamber; and vi) detect the signal while the one or more microparticles remain in the single chamber.

62. The system of claim 61, wherein the single chamber is a well of a microplate.

63. The system of claim 62, wherein the microplate has at least 384 wells.

64. The system of claim 62, wherein the microplate has at least 96 wells.

65. The system of claim 61, wherein the single chamber has from about a 250 to about a 300 μL fill volume.

66. The system of claim 61, further comprising a multi-tip pipette head that delivers the elutor or the amplification agent to the single chamber.

67. The system of claim 61, further comprising a heating element.

68. The system of claim 67, wherein the heating element is capable of shifting between a first temperature and a second temperature in under two minutes.

69. The system of claim 61, wherein the reporter molecule comprises a detection moiety configured to generate the signal.

70. The system of claim 69, wherein the detection moiety comprises a fluorophore.

71. The system of claim 61, further comprising a tube for holding a positive control and a tube for holding a negative control.

72. The system of claim 61, further comprising a detector for detecting the emitted signal.

73. The system of claim 72, wherein the detector comprises a fluorimeter.

74. The system of claim 61, further comprising a computing device to identify the presence or an absence of the target nucleic acid via the signal.

75. The system of claim 74, wherein the computing device identifies a presence or absence of the target nucleic acid by comparing a signal slope against a signal slope from a positive control and a signal slope from a negative control.

76. The system of claim 74, wherein the computing device is in operative communication with a detector for detecting the emitted signal.

77. The system of claim 61, wherein the lysis agent comprises a physical, mechanical, thermal, enzymatic agent, or a combination thereof.

78. The system of claim 61, wherein the lysis agent comprises a lysis buffer solution.

79. The system of claim 78, wherein the lysis buffer solution comprises a chaotropic agent, detergent, salt, or a combination thereof.

80. The system of claim 79, wherein the lysis buffer solution comprises 4 M guanidinium isothiocyanate, 25 mM sodium citrate.2H20, 0.5% (w/v) sodium lauryl sarcosinate, and 0.1 M β-mercaptoethanol.

81. The system of claim 61, wherein the microparticles comprise silica-coated beads or magnetized beads.

82. The system of claim 61, wherein the elutor comprises a buffer solution.

83. The system of claim 61, wherein the elutor comprises a chaotropic salt or a detergent.

84. The system of claim 83, wherein the elutor comprises a detergent, wherein the detergent comprises Tween 20, Triton X-100, Deoxycholate, Sodium laurel sulfate, 3-[(3-cholamidopropyl)dimethylammonio]-1-propanesulfonate (CHAPS), or combinations thereof.

85. The system of claim 61, wherein the amplification agent comprises a DNA sequence, dNTPs, a forward primer, a reverse primer, a polymerase, or combinations thereof.

86. The system of claim 61, wherein the amplification agent comprises a reagent for RT-LAMP amplification.

87. The system of claim 86, wherein the amplification agent includes an RNA, a plurality of primers (e.g., four, five, or six primers), a primer having a T7 promoter, dNTPs, NTPs, a polymerase enzyme, a reverse transcriptase enzyme, a RNA polymerase, or combinations thereof.

88. The system of claim 87, wherein the RNA polymerase is T7 RNA polymerase.

89. The system of claim 61, wherein the programmable nuclease comprises a CRISPR/Cas enzyme.

90. The system of claim 89, wherein the CRISPR/Cas enzyme is a Cas12, a Cas13, a Cas14, a programmable thermostable Cas nuclease, or a CasΦ effector protein.

91. The system of claim 61, wherein the guide nucleic acid is sgRNA.

92. The system of claim 61, wherein the reporter molecule is ssDNA-FQ reporter and the detection moiety is a fluorophore or a quencher.

93. The system of claim 61, wherein the signal comprises a calorimetric, potentiometric, amperometric, fluorescent, or colorimetric signal.

94. The system of claim 61, wherein the signal comprises a fluorometric signal generated using a fluorophore.

95. The system of claim 61, wherein the signal is generated using a nucleic acid conjugated to an affinity molecule and the affinity molecule conjugated to a fluorophore.

96. The system of claim 61, wherein the system comprises a concentration of 100nM CasΦ polypeptide or variant thereof, 125 nM sgRNA, and 50 nM ssDNA-FQ reporter in a total reaction volume of 20 μL.

97. The system of claim 61, wherein the reporter molecule comprises a protein configured to generate the signal.

98. A high-throughput single-chamber process for detecting a presence of a target nucleic acid, comprising:
(a) providing a single chamber;
(b) binding the plurality of nucleic acids with a microparticle within the single chamber to form a microparticle complex;
(c) isolating the microparticle complex within the single chamber;
(d) amplifying the plurality of nucleic acids within the single chamber to form an amplified product while the microparticle remains within the single chamber;
(e) assaying the amplified product for a detectable signal emitted within the single chamber, thereby detecting a presence or absence of the target nucleic acid, while the microparticle remains within the single chamber.

99. The process of claim 98, further comprising, prior to (b), lysing a sample to release the plurality of nucleic acids within the single chamber, thereby enabling the plurality of nucleic acids to bind with the microparticle.

100. The process of claim 98, further comprising, prior to (d), eluting the plurality of nucleic acids from the microparticle complex.

101. The process of claim 98, further comprising, prior to (e), contacting the amplified product with a guide nucleic acid complexed to a programmable nuclease within the single chamber such that, when the amplified product comprises a target nucleic acid, the guide nucleic acid contacts the target nucleic acid to form an activated programmable nuclease, thereby cleaving a reporter molecule by the activated programmable nuclease to produce a cleaved reporter molecule.

102. The process of claim 101, wherein the reporter molecule comprises a detection moiety for generating the signal.

103. The process of claim 102, wherein the detection moiety comprises a fluorophore.

104. The process of claim 98, wherein (b) is performed at 37+/−2° C., (d) is performed at 57+/−2° C., and (e) is performed at 37+/−2° C.

105. The process of claim 98, wherein (b) is performed at 95+/−2° C., (d) is performed at 62+/−2° C., and (e) is performed at 37+/−2° C.

106. The process of claim 98, wherein (b) is performed at between 20° C. and 95° C., (d) is performed at between 52° C. and 67° C., and (e) is performed at 37+/−2° C.

107. The process of claim 98, wherein the programmable nuclease is a programmable Cas12 nuclease, a programmable Cas13 nuclease, a programmable Cas14 nuclease, a programmable thermostable Cas nuclease, or a CasΦ nuclease.

108. The process of claim 98, wherein isolating the microparticle complex comprises capturing the microparticle with a magnet.

109. The process of claim 98, wherein amplifying the nucleic acid molecules comprises performing RT-LAMP.

110. The process of claim 98, wherein the signal comprises a fluorescence signal.

111. The process of claim 98, wherein (a)-(f) are completed in under about 40 minutes.

112. The process of claim 98, wherein the single chamber is a first well in a microplate.

113. The process of claim 104, further comprising, in a second well of the microplate, performing steps (a)-(f) on an additional sample.

114. The process of any one of claims 98-113, wherein steps (a)-(g) are performed in a high-throughput manner.

115. The process of claim 114, wherein the high-throughput manner comprises detecting about 400 target nucleic acids in 1.75 hrs or detecting about 192 target nucleic acids in 110 minutes.

116. A high-throughput single-chamber process for detecting a presence of a target nucleic acid, comprising:
(a) providing a lysis agent and microparticles in a single chamber;
(b) providing a sample in the single chamber and lysing the sample by contacting the lysis agent with the sample, thereby releasing nucleic acid molecules;
(c) allowing the nucleic acid molecules to bind to the microparticles to produce complexes comprising the nucleic acid molecules and the microparticles;
(d) isolating the complexes comprising the nucleic acid molecules and the microparticles in the single chamber;
(e) eluting the nucleic acid molecules from the complexes;
(f) amplifying the nucleic acid molecules to form an amplified product, wherein the amplifying is by contacting the nucleic acid molecules with an amplification agent;
(g) contacting, in the single chamber, the amplified product with:
(i) a programmable nuclease,
(ii) a reporter molecule, and
(iii) a guide nucleic acid that is capable of binding with a target nucleic acid,
wherein, in the presence of the target nucleic acid in the amplified product, the guide nucleic acid binds with the target nucleic acid, such that the programmable nuclease cleaves the reporter molecule to produce a cleaved reporter molecule, and
wherein a detectable signal is emitted by the cleaved reporter molecule, wherein the detectable signal is indicative of the presence or absence of the target nucleic acid.

117. The process of claim 116, wherein (b) is performed at 37+/−2° C., (d) is performed at 57+/−2° C., and (e) is performed at 37+/−2° C.

118. The process of claim 116, wherein (b) is performed at 95+/−2° C., (d) is performed at 62+/−2° C., and (e) is performed at 37+/−2° C.

119. The process of claim 116, wherein (b) is performed at between 20° C. and 95° C., (d) is performed at between 52° C. and 67° C., and (e) is performed at 37+/−2° C.

120. The process of claim 116, wherein the microparticle remains in the single chamber during steps (f)-(g).

121. The process of claim 116, wherein the programmable nuclease is a programmable Cas12 nuclease, a programmable Cas13 nuclease, a programmable Cas14 nuclease, a programmable thermostable Cas nuclease, or a CasΦ nuclease.

122. The process of claim 116, wherein isolating the microparticle complex comprises capturing the microparticle with a magnet.

123. The process of claim 116, wherein amplifying the nucleic acid molecules comprises performing RT-LAMP.

124. The process of claim 116 wherein the signal comprises a fluorescence signal.

125. The process of claim 116, wherein (a)-(g) are completed in under about 40 minutes.

126. The process of claim 116, wherein the single chamber is a first well in a microplate.

127. The process of claim 126, further comprising, in a second well of the microplate, performing steps (a)-(f) on an additional sample.

128. The process of claim 116, wherein (f) and (g) occur simultaneously.

129. The process of any one of claims 116-128, wherein steps (a)-(g) are performed in a high-throughput manner.

130. The process of claim 129, wherein the high-throughput manner comprises detecting about 400 target nucleic acids in 1.75 hrs or detecting about 192 target nucleic acids in 110 minutes.

131. A high-throughput single-chamber process for detecting the presence of a first target nucleic acid and a second target nucleic acid in a sample, comprising:
(a) providing a single chamber;
(b) binding a plurality of nucleic acids with a microparticle within the single chamber to form a microparticle complex;
(c) isolating the microparticle complex within the single chamber;
(d) contacting, in the single chamber, the plurality of nucleic acid molecules with a first probe, wherein the first probe is configured for binding with the first target nucleic acid;
(e) amplifying the plurality of nucleic acids within the single chamber to form an amplified product, wherein a first detectable signal is emitted i) prior to amplifying the plurality of nucleic acids, ii) while amplifying the plurality of nucleic acids, iii) after forming the amplified product, or iv) a combination thereof, thereby detecting the presence of the first target nucleic acid;
(f) contacting the amplified product with a second probe complexed to a programmable nuclease within the single chamber such that, when the amplified product comprises the second target nucleic acid, the second probe contacts the target nucleic acid to form an activated programmable nuclease, thereby cleaving a reporter molecule by the activated programmable nuclease to produce a cleaved reporter molecule; and
(g) assaying for a second detectable signal emitted within the single chamber by the cleaved reporter molecule, thereby detecting the presence of the second target nucleic acid.

132. The process of claim 131, wherein i) the first target nucleic acid comprises RNAse P, ii) the second target nucleic acid comprises SARS-CoV-2 N gene, or iii) a combination thereof.

133. The process of claim 131, wherein the first probe comprises a dye configured to produce a colorimetric signal when the pH changes during amplification of the plurality of nucleic acids.

134. The process of claim 131, wherein the first probe comprises a label configured to produce a fluorescent signal at a first wavelength.

135. The process of claim 131, wherein the second probe comprises a guide nucleic acid.

136. The process of claim 131, wherein the one or both of the first signal and the second signal comprises a fluorescent signal.

137. The process of claim 131, wherein both the first signal and the second signal comprise a fluorescent signal and wherein the second signal comprises a wavelength different from a wavelength of the first signal.

138. The process of claim 131, wherein the microparticle remains in the single chamber during steps (d)-(g).

139. The process of claim 131, further comprising, prior to (b), lysing a sample to release the plurality of nucleic acids within the single chamber, thereby enabling the plurality of nucleic acids to bind with the microparticle.

140. The process of claim 131 further comprising, prior to (d), eluting the plurality of nucleic acids from the microparticle complex.

141. The process of claim 131, wherein the programmable nuclease is a programmable Cas12 nuclease, a programmable Cas13 nuclease, a programmable Cas14 nuclease, a programmable thermostable Cas nuclease, or a CasΦ nuclease.

142. The process of claim 131, wherein isolating the microparticle complex comprises capturing the microparticle with a magnet.

143. The process of claim 116, wherein amplifying the nucleic acid molecules comprises performing RT-LAMP.

144. The process of claim 116 wherein the signal comprises a fluorescence signal.

145. The process of claim 116, wherein (a)-(g) are completed in under about 40 minutes.

146. The process of claim 116, wherein the single chamber is a first well in a microplate.

147. The process of claim 126, further comprising, in a second well of the microplate, performing steps (a)-(f) on an additional sample.

148. The process of claim 116, wherein (f) and (g) occur simultaneously.

149. The process of any one of claims 116-128, wherein steps (a)-(g) are performed in a high-throughput manner.

150. The process of claim 129, wherein the high-throughput manner comprises detecting about 400 target nucleic acids in 1.75 hrs or detecting about 192 target nucleic acids in 110 minutes.

151. The process of claim 51, wherein the pathogenic viruses comprise SARS-CoV-2 variants.

152. The process of claim 151, wherein the variants are B.1.1.7, B.1.351, B.1.617,.2, B.1.427, B.1.429, P.1., or SARS-CoV-2 wild-type.

153. The process of claim 1, wherein the single chamber is a well of a microplate or a tube.

154. The process of claim 1, wherein the target nucleic acid comprises a gene.

155. The process of claim 154, wherein the gene is a SARS-CoV-2 N-gene.

156. The process of claim 1, wherein the plurality of nucleic acids is collected from nasopharyngeal swabs or from nasal, mid-turbinate, or oropharyngeal sources.

157. The process of claim 51, wherein the pathogenic viruses comprise SARS-CoV-2 mutations.

158. The process of claim 157, wherein the mutations are L452R, E484K, or N501Y.

159. The process of claim 1, wherein (c) comprises adding a wash solution to the single chamber.

160. The process of claim 1, wherein (d) further comprises adding mineral oil to prevent evaporation.

161. The process of claim 1, wherein (a) is performed in a laboratory, hospital, physician office, clinic, a remote site, or in a home.
